(19) United States
(12) Patent Application Publication
(10) Pub. No.: US 2022/0120736 A1
(43) Pub. Date: Apr. 21, 2022

(54) METHODS AND ASSAYS FOR MODULATING GENE TRANSCRIPTION BY MODULATING CONDENSATES

(71) Applicants: Whitehead Institute for Biomedical Research, Cambridge, MA (US); Massachusetts Institute of Technology, Cambridge, MA (US)

(72) Inventors: Richard A. Young, Boston, MA (US); Phillip A. Sharp, Newton, MA (US); Arup K. Chakraborty, Lexington, MA (US); Alessandra Dall'Agnese, Cambridge, MA (US); Krishna Shrinivas, Cambridge, MA (US); Brian J. Abraham, Cambridge, MA (US); Ann Boija, Somerville, MA (US); Eliot Coffey, Cambridge, MA (US); Daniel S. Day, Redwood City, CA (US); Yang E. Guo, Somerville, MA (US); Nancy M. Hannett, Medford, MA (US); Tong Ihn Lee, Cambridge, MA (US); Charles H. Li, Waban, MA (US); Isaac Klein, Milton, MA (US); John C. Manteiga, Boston, MA (US); Benjamin R. Sabari, Watertown, MA (US); Jurian Schuijers, Somerville, MA (US); Abraham S. Weintraub, Cambridge, MA (US); Alicia V. Zamudio, Cambridge, MA (US); Lena K. Afeyan, Boston, MA (US); Ozgur Oksuz, Worcester, MA (US); Jonathan E. Henninger, Somerville, MA (US)

(21) Appl. No.: 17/040,967

(22) PCT Filed: Mar. 22, 2019
(86) PCT No.: PCT/US2019/023694

§ 371 Date: Sep. 23, 2020

Related U.S. Application Data
(60) Provisional application No. 62/820,237, filed on Mar. 18, 2019.
(60) Provisional application No. 62/819,662, filed on Mar. 17, 2019.
(60) Provisional application No. 62/752,332, filed on Oct. 29, 2018.
(60) Provisional application No. 62/722,825, filed on Aug. 24, 2018.
(60) Provisional application No. 62/648,377, filed on Mar. 26, 2018.
(60) Provisional application No. 62/647,613, filed on Mar. 23, 2018.

(51) Int. Cl. G01N 33/535; C12N 15/63; G01N 33/50

(52) U.S. Cl. CPC G01N 33/535; G01N 33/5005; C12N 15/63

(57) ABSTRACT

Described herein are compositions and methods for modulating gene regulation by modulating condensate formation, composition, maintenance, dissolution and regulation.

### RELATED APPLICATIONS

This application claims the benefit of U.S. Provisional Application Ser. No. 62/647,613, filed Mar. 23, 2018, U.S. Provisional Application Ser. No. 62/648,377, filed Mar. 26, 2018, U.S. Provisional Application Ser. No. 62/722,825, filed Aug. 24, 2018, U.S. Provisional Application Ser. No. 62/752,332, filed Oct. 29, 2018; U.S. Provisional Application Ser. No. 62/819,662, filed Mar. 17, 2019, and U.S. Provisional Application Ser. No. 62/820,237, filed Mar. 18, 2019, the contents of all of which are hereby incorporated by reference in their entirety.

### GOVERNMENT SUPPORT

This invention was made with government support under Grant Nos. HG002668, CA042063, T32CA009172, GM117370, GM008759, and GM123511 awarded by the National Institutes of Health, and Grant No. 1743900 awarded by the National Science Foundation. The government has certain rights in the invention.

### BACKGROUND OF THE INVENTION

Regulation of gene expression requires that the transcription apparatus be efficiently recruited to specific genomic sites. DNA-binding transcription factors (TFs) ensure this specificity by occupying specific DNA sequences at enhancer and promoter-proximal elements and recruiting the transcriptional machinery to these sites. TFs typically consist of one or more DNA-binding domains (DBD) and one or more separate activation domains (AD). While the structure and function of TF DBDs are well-documented, comparatively little is understood about the structure of ADs and how these interact with coactivators to drive gene expression.

The structure of TF DBDs and their interaction with cognate DNA sequences has been described at atomic resolution for many TFs, and TFs are generally classified according to the structural features of their DBDs. For example, DBDs can be composed of zinc-coordinating, basic helix-loop-helix, basic-leucine zipper, or helix-turn-helix DNA-binding structures. These DBDs selectively bind specific DNA sequences that range from approximately 4-12 bp, and the DNA binding sequences favored by hundreds of TFs have been described. Multiple different TF molecules typically bind together at any one enhancer or promoter-proximal element. For example, at least eight different TF molecules bind a 50 bp core component of the IFN-β enhancer (Panne et al., 2007).

Anchored in place by the DBD, the AD interacts with coactivators, which integrate signals from multiple TFs to regulate transcriptional output. In contrast to the structured DBD, the ADs of most TFs are low-complexity amino acid sequences not amenable to crystallography. These intrinsically disordered regions or domains (IDRs) have therefore been classified by their amino acid profile as acidic, proline-, serine/threonine-, or glutamine-rich; or by their hypothetical shape as acid blobs, negative noodles, or peptide lassos (Hahn and Young, 2011; Mitchell and Tjian, 1989; Roberts, 2000; Sigler, 1988; Staby et al., 2017; Triezenberg, 1995). Remarkably, hundreds of TFs are thought to interact with the same small set of coactivator complexes, which include Mediator and p300, among others. ADs that share little sequence homology are functionally interchangeable among TFs; this interchangeability is not readily explained by traditional lock-and-key models of protein-protein interaction. Thus, how the diverse activation domains of hundreds of different TFs interact with a similar small set of coactivators remains a conundrum.

Enhancers are gene regulatory elements bound by transcription factors and other components of the transcription apparatus that function to regulate expression of cell type-specific genes. Super-enhancers (SEs), clusters of enhancers that are occupied by exceptionally high densities of transcription apparatus, regulate genes with especially important roles in cell identity.

Pioneering genetic studies in Drosophila showed that transcription factors and signaling factors play fundamentally important roles in the control of development. Many subsequent studies have led to the understanding that the gene expression programs defining each cell's identity are controlled by lineage- and cell-type-specific master TFs, which establish cell-type specific enhancers, and signaling factors, which carry extracellular information to these enhancers.

The results of transdifferentiation and reprogramming experiments argue that a small number of master TFs dominate the control of cell-type specific gene expression. Although many hundreds of TFs are expressed in each cell type, only a handful are necessary to cause cells to acquire a new identity, as demonstrated by the ability of the TF MyoD to transdifferentiate cells into muscle-like cells (Weintraub, et al (1989) Proc. Natl. Acad. Sci. 86, 5434-5438), and the ability of the TFs Oct4, Nanog, Klf4 and Myc to reprogram fibroblasts into induced pluripotent stem cells (Takahashi, et al. (2006) Cell 126, 663-676). These master TFs dominate the control of gene expression programs by establishing enhancers, and often clusters of enhancers called super-enhancers, at genes with prominent roles in cell identity.

Cells depend on signaling pathways to maintain their identity and to respond to the extracellular environment. The signaling pathways that play prominent roles in control of mammalian developmental processes include the WNT, TGF-β and JAK/STAT pathways. In each of these pathways, an extracellular ligand is recognized by a specific receptor, which transduces the signal through other proteins to a set of signaling factors that enter the nucleus and bind to signal response elements in the genome. In a given cell type, these signaling factors bind to a small subset of a large number of putative signal response elements, preferring to bind those that occur in the active enhancers of that cell type, thus allowing for cell type-specific responses to signaling factors that are expressed in a broad spectrum of cell types.

The synthesis of pre-mRNA by RNA polymerase II (Pol II) involves the formation of a transcription initiation complex and a transition to an elongation complex. The large subunit of Pol II contains an intrinsically disordered C-terminal domain (CTD), which is phosphorylated by cyclin-dependent kinases (CDKs) during the initiation-to-elongation transition, thus influencing the CTD's interaction with different components of the initiation or the RNA splicing apparatus. Recent observations suggest that this model provides only a partial picture of the effects of CTD phosphorylation.

Chromatin is generally classified into categories: euchromatin, which is less compacted and gene-rich, and heterochromatin, which is highly compacted and gene poor1. Constitutive heterochromatin assembles at repetitive elements such as satellite DNA and transposons. Heterochromatin plays important roles in repressing recombination between repeat elements, limiting the transcription of active transposons, structuring centromeric DNA, and repressing gene expression across developmental lineages.

Further study is needed to elucidate the mechanisms of gene expression control as related to the diversity of TFs and signaling factors, as well as for heterochromatin and during mRNA initiation and elongation.

### SUMMARY OF THE INVENTION

Work described herein has identified the existence and utility of condensates having a variety of components and including both naturally-occurring condensates and synthetic or artificial condensates. Described herein are condensates and their components, methods of identifying agents that modulate condensate structure and function, and methods of modulating condensate function/activity for therapeutic effect, as well as other related compositions and methods.

In general, the present disclosure is related to the modulation, formation and use of transcriptional condensates, heterochromatin condensates, and condensates physically associated with mRNA initiation or elongation complexes. The present disclosure is also related to the finding that nuclear receptors, signaling factors, and methyl-DNA binding factors interact and modify condensates. As will be apparent from the below description, condensates can be modulated by, e.g., modifying the type, amount, or attributes of the components of the condensates, or with agents. Using condensates for screening methods provides a useful tool, that may more accurately reflect intracellular gene expression control, for discovering therapeutics.

Transcriptional condensates are phase-separated multi-molecular assemblies that occur at the sites of transcription and are high density cooperative assemblies of multiple components that can include transcription factors, co-factors, chromatin regulators, DNA, non-coding RNA, nascent RNA, and RNA polymerase II (FIG. 1). In some instances, transcriptional condensates are formed by super-enhancer assemblies. Many diseases are caused by, or associated with, alteration in these nucleic acid and protein components, and therapeutic intervention may be afforded by altering transcriptional output of condensates. As used herein, “heterochromatin condensates” are phase-separated multi-molecular assemblies that are physically associated with (e.g., occur on) heterochromatin. In some aspects of the disclosure, condensates physically associated with an mRNA initiation or elongation complex are described. As used herein, these condensates (i.e., condensates physically associated with an mRNA initiation or elongation complex) are phase-separated multi-molecular assemblies occurring at the relevant complex. In some embodiments, a condensate physically associated with an elongation complex comprises splicing factors. As used herein, a synthetic transcriptional condensate refers to a non-naturally occurring condensate comprising transcriptional condensate components.

The results described herein, in part, support a model in which transcription factors interact with Mediator and activate genes by the capacity of their activation domains to form phase-separated condensates with this coactivator. This process of forming phase-separated condensates with coactivators is perturbed in many diseases including autoimmunity, cancer, and neurodegeneration. For example, malignant transformation may occur by, among other processes: the generation of fusion oncogenic transcription factors that inappropriately activate cell survival or proliferation pathways, inappropriate production of transcription factors that are not expressed in the normal tissue, or mutation of an enhancer region that recruits a transcription factors to a previously silent oncogene. Perturbing the function of these activation domains or other components of the condensates provides a mechanism to interrupt the activity of transcription factors.

Described herein are, among other things, diseases that may involve condensates, assays, and methods for modulating transcription by enhancing or decreasing transcriptional condensate formation, composition, maintenance, dissolution and regulation. In some aspects, the transcriptional condensates comprise nuclear receptors, e.g., nuclear hormone receptors or mutant nuclear hormone receptors that activate transcription in the absence of a cognate ligand. In some aspects, the condensates (e.g. transcriptional, heterochromatin, and/or condensates physically associated with mRNA initiation or elongation complexes) comprise signaling factors, methyl-DNA binding proteins (e.g., methyl CpG binding proteins), gene silencing factors (e.g., repressors, repressive heterochromatin factors), RNA polymerase (e.g., Pol II, phosphorylated Pol II, de-phosphorylated Pol II), or splicing factors. Some aspects of the disclosure are related to treating diseases and conditions by administering an agent that modulates condensate formation, composition, maintenance, dissolution, activity, or regulation. In some embodiments of the methods described herein, the administered agent is not known to be useful for treating the targeted disease.

Some aspects of the disclosure are directed to a method of modulating transcription of one or more genes (e.g., one or more genes in a cell), comprising modulating formation, composition, maintenance, dissolution, activity and/or regulation of a condensate (e.g., transcriptional condensate) associated with the one or more genes. In some embodiments, the condensate (e.g., transcriptional condensate) is modulated by increasing or decreasing a valency of a component associated with the condensate.

As used herein, the phrases “a component associated with a condensate” or the like and the phrase “a condensate component” or the like refer to a peptide, protein, nucleic acid, signaling molecule, lipid, or the like that is part of a condensate or has the capability of being part of a condensate (e.g., transcriptional condensate). In some embodiments, the component is within the condensate. In some embodiments, the component is on the surface of the condensate. In some embodiments, the component is necessary for condensate formation or stability. In some embodiments, the component is not necessary for condensate formation or stability. In some embodiments, the component is a protein or peptide and comprises one or more intrinsically ordered domains (e.g., an IDR of an activation domain of a transcription factor, an IDR that interacts with an IDR of an activation domain of a transcription factor, an IDR of a signaling factor, an IDR of a methyl-DNA binding protein, an IDR of a gene silencing factor, an IDR of a polymerase, an IDR of a splicing factor). In some embodiments, the component is a non-structural member of a condensate (e.g., not necessary for condensate integrity) and is sometimes referred to as a client component. In some embodiments, a condensate comprises, consists of, or consists essentially of 1, 2, 3, 4, 5, 6, 7, 8, 9, 10 or more components. In some embodiments, a condensate (e.g., a synthetic transcriptional condensate (a synthetic transcriptional condensate is sometimes referred to herein as an “artificial condensate”) does not comprise a nucleic acid. In some embodiments, a condensate (e.g., a synthetic transcriptional condensate) does not comprise RNA. In some embodiments, the component is a fragment of a protein or nucleic acid.

In some embodiments, the component is selected from the group consisting of a DNA sequence (e.g., an enhancer DNA sequence, a methylated DNA sequence, a super-enhancer DNA sequence, 3′ end of a transcribed gene, a signal response element, a hormone response element), a transcription factor, a gene silencing factor, a splicing factor, an elongation factor, an initiation factor, a histone (e.g., a modified histone), a co-factor, an RNA (e.g., ncRNA), mediator, and RNA polymerase (e.g., RNA polymerase II). In some embodiments, the co-factor comprises an LXXLL motif. In some embodiments, the co-factor comprises an LXXLL motif and has increased valency for a TF (e.g., a nuclear receptor, a master transcription factor) when bound to a ligand (e.g., a cognate ligand, a naturally occurring ligand, a synthetic ligand). Co-factors having LXXLL motifs are known in the art. In some embodiments, the component is a fragment of a co-factor comprising an IDR and LXXLL motif. In some embodiments, the component is not a nuclear receptor ligand. In some embodiments, the component is not a lipid. In some embodiments, the component is a protein or nucleic acid.

In some embodiments, the condensate is modulated by contacting the condensate with an agent that interacts with one or more intrinsic disorder domains of a component of the condensate. In some embodiments, the component of the condensate contacted with the agent is a signaling factor, methyl-DNA binding protein, gene silencing factor, RNA polymerase, splicing factor, BRD4, Mediator, a mediator component, MED1, MED15, a transcription factor, an RNA polymerase, or a nuclear receptor ligand (e.g., a hormone). In some embodiments, the component is a protein listed in Table S1.

In some embodiments, the component of the condensate contacted with the agent is a signaling factor selected from the group consisting of TCF7L2, TCF7, TCF7L1, LEF1, Beta-Catenin, SMAD2, SMAD3, SMAD4, STAT1, STAT2, STAT3, STAT4, STAT5A, STAT5B, STAT6, and NF-κB. In some embodiments, the signaling factor comprises one or more intrinsic disorder domains. In some embodiments, the signaling factor preferentially binds to one or more signal response elements or mediator associated with the condensate. In some embodiments, the condensate comprises a master transcription factor.

In some embodiments, the component of the condensate contacted with the agent is a methyl-DNA binding protein that preferentially binds to methylated DNA. In some embodiments, the methyl-DNA binding protein is MECP2, MBD1, MBD2, MBD3, or MBD4. In some embodiments, the methyl-DNA binding protein is associated with gene silencing. In some embodiments, the component is a suppressor associated with heterochromatin. In some embodiments, the methyl-DNA binding protein is HP1α, TBL1R (transducin beta-like protein), HDAC3 (histone deacetylase 3) or SMRT (silencing mediator of retinoic and thyroid receptor).

In some embodiments, the component of the condensate contacted with the agent is an RNA polymerase associated with mRNA initiation and elongation. In some embodiments, the RNA polymerase is RNA polymerase II or an RNA polymerase II C-terminal region. In some embodiments, the RNA polymerase II C-terminal region comprises an intrinsically disordered region (IDR). In some embodiments, the IDR comprises a phosphorylation site. In some embodiments, the component is a splicing factor selected from SRSF2, SRRM1, or SRSF1.

In some embodiments, the component of the condensate contacted with the agent is a transcription factor. In some embodiments, the transcription factor is OCT4, p53, MYC or GCN4, NANOG, MyoD, KLF4, a SOX family transcription factor, a GATA family transcription factor, or a nuclear receptor (e.g., a nuclear hormone receptor, Estrogen Receptor, Retinoic Acid Receptor-Alpha). In some embodiments of the methods disclosed herein, the transcription factor is a human transcription factor identified in Lambert, et al., Cell. 2018 Feb. 8; 172(4):650-665. In some embodiments, the nuclear receptor activates transcription when bound to a cognate ligand. In some embodiments, the nuclear receptor is a mutant nuclear receptor that activates transcription in the absence of a cognate ligand, or has a higher level of transcription activity (e.g., at least 1.5-fold, at least 2-fold, at least 3-fold, or more) in the absence of a cognate ligand than the wild-type nuclear receptor in the presence of the natural ligand (e.g., cognate ligand). In some embodiments, the nuclear receptor is a mutant nuclear transcription factor that modulates transcription in the presence of a cognate ligand to a different degree than the wild-type nuclear receptor. In some embodiments, the transcription factor is a fusion oncogenic transcription factor or a transcription factor disclosed in Table S3. In some embodiments, the fusion oncogenic transcription factor is selected from MLL-rearrangements, EWS-FLI, ETS fusions, BRD4-NUT, and NUP98 fusions. The oncogenic transcription factor may be any oncogenic transcription factor identified in the art.

In some embodiments, the agent that interacts with one or more intrinsic disorder domains of a component of the condensate is, or comprises, a peptide, nucleic acid, or small molecule. In some embodiments, the agent comprises a peptide enriched for acidic amino acids (e.g., a peptide having a net negative charge, a peptide enriched for glutamic acid and/or aspartic acid). In some embodiments, the agent is a signaling factor mimetic. In some embodiments, the agent is a signaling factor antagonist. In some embodiments, the agent comprises a hypophosphorylated RNA polymerase II C-terminal domain (Pol II CTD) or a functional fragment thereof. In some embodiments, the agent preferentially binds hypophosphorylated Pol II CTD. In some embodiments, the agent binds methylated DNA. In some embodiments, the agent binds a methyl-DNA binding protein.

In some embodiments, contact with the agent stabilizes or dissolves the condensate, thereby modulating transcription of the one or more genes. In some embodiments, the condensate is modulated by modulating the binding of a transcription factor associated with the condensate to a component (e.g., a component associated with the condensate that is not a transcription factor) of the condensate. In some embodiments, the component of the condensate is a coactivator, signaling factor, methyl-DNA binding protein, splicing factor, gene silencing factor, RNA polymerase, or cofactor. In some embodiments, the component of the condensate is a nuclear receptor ligand or signaling factor. In some embodiments, the coactivator, signaling factor, methyl-DNA binding protein, splicing factor, gene silencing factor, RNA polymerase, or cofactor is Mediator, a mediator component, MED1, MED15, p300, BRD4, β-catenin, STAT3, SMAD3, NF-kB, MECP2, MBD1, MBD2, MBD3, MBD4, HP1α, TBL1R, HDAC3, SMRT, RNA polymerase II, SRSF2, SRRM1, SRSF1, or TFIID. In some embodiments, the nuclear receptor ligand is a hormone. In some embodiments, the transcription factor is OCT4, p53, MYC, GCN4, NANOG, MyoD, KLF4, a SOX family transcription factor, a GATA family transcription factor, a nuclear receptor, or a fusion oncogenic transcription factor. In some embodiments, the binding of the transcription factor to a component of the condensate is modulated by contacting the transcription factor or condensate with an agent (e.g., a peptide, nucleic acid, or small molecule). In some embodiments, the binding of the transcription factor to a component of the condensate is modulated by contacting the activation domain (e.g., an IDR of the activation domain) of the transcription factor with an agent (e.g., a peptide, nucleic acid, or small molecule).

In some embodiments, the transcriptional condensate is modulated by modulating the binding of a ligand to a nuclear receptor that is part of, or capable of being part of, a transcriptional condensate. In some embodiments, the ligand is a hormone (e.g., estrogen). In some embodiments, the binding of the ligand is modulated with an agent (e.g., a peptide, nucleic acid, or small molecule). In some embodiments, the transcriptional condensate is modulated by modulating the binding of a nuclear receptor with a component of the transcriptional condensate. In some embodiments, the component of the transcriptional condensate is a coactivator, cofactor, or nuclear receptor ligand (e.g., hormone). In some embodiments, the coactivator, cofactor, or nuclear receptor ligand is a mediator component or a hormone. In some embodiments, the nuclear receptor (e.g., a mutant nuclear receptor) activates transcription without binding to a cognate ligand. In some embodiments, the association of the nuclear receptor with the component is modulated with an agent. In some embodiments, transcriptional activity of a condensate is modulated by modulating the binding of a nuclear receptor with another condensate component (e.g., a mediator component).

In some embodiments, the condensate (e.g., transcriptional condensate) is modulated by modulating the binding of a signaling factor with a component of the transcriptional condensate. In some embodiments, the component is mediator, a mediator component, or a transcription factor. In some embodiments, the condensate is associated with a super-enhancer. In some embodiments, modulating the condensate modulates expression of one or more oncogenes. In some embodiments, the signaling factor is associated with an oncogenic signaling pathway. In some embodiments, the condensate comprises an aberrant level of a signaling factor (i.e., an increased or decreased level of signaling factor as compared to a healthy or non-resistant cell).

In some embodiments, the condensate is modulated by modulating the binding of a methyl-DNA binding protein to a component of the condensate or to methylated DNA. In some embodiments, the condensate is modulated by modulating the binding of a gene silencing factor to a component of the condensate. In some embodiments, the condensate is modulated by modulating the binding of an RNA polymerase to a component of the transcription factor. In some embodiments, the condensate is modulated by modulating the binding of splicing factor to a component of the transcription factor.

In some embodiments, the condensate is modulated by modulating the amount of a component (e.g., a client component, a non-structural component) associated with the condensate. In some embodiments, the component (e.g., transcriptional component) is one or more transcriptional co-factors and/or transcriptions factors (e.g., signaling factors) and/or nuclear receptor ligands (e.g., hormones). In some embodiments, the component is Mediator, a mediator component, MED1, MED15, p300, BRD4, TFIID, β-catenin, STAT3, SMAD3, NF-KB, MECP2, MBD1, MBD2, MBD3, MBD4, HP1α, TBL1R, HDAC3, SMRT, RNA polymerase II, SRSF2, SRRM1, SRSF1, or a hormone. In some embodiments, the component may be Mediator, a mediator component, MED1, MED15, p300, BRD4, TFIID, or a nuclear receptor ligand. In some embodiments, the component is a transcription factor (e.g., OCT4, p53, MYC, GCN4, NANOG, MyoD, KLF4, a SOX family transcription factor, a GATA family transcription factor, a nuclear receptor, or a fusion oncogenic transcription factor).

In some embodiments, the amount of the component associated with the condensate is modulated by contact with an agent that reduces or eliminates interactions between the component and other components associated with the condensate. In some embodiments, the agent targets an interacting domain of a component associated with the condensate. In some embodiments, the interacting domain is an intrinsically disordered domain or region (IDR). In some embodiments, the IDR is in the activation domain of a transcription factor.

In some embodiments, modulating the condensate (e.g., transcriptional condensate) modulates one or more signaling pathways. In some embodiments, the signaling pathway contributes to disease pathogenesis (e.g., cancer pathogenesis). In some embodiments, the signaling pathway involves hormone signaling. In some embodiments, the signaling pathway comprises a signaling factor as a component of the condensate. In some embodiments, the signaling factor is selected from the group consisting of TCF7L2, TCF7, TCF7L1, LEF1, Beta-Catenin, SMAD2, SMAD3, SMAD4, STAT1, STAT2, STAT3, STAT4, STAT5A, STAT5B, STAT6, and NF-κB. In some embodiments, the signaling pathway involves a nuclear receptor (e.g., a nuclear hormone receptor). In some embodiments, modulating the condensate modulates interactions between the condensate and one or more nuclear pore proteins. In some embodiments, modulation of the interactions between the condensate and the one or more nuclear pore proteins can modulate nuclear signaling, mRNA export, and/or mRNA translation. In some embodiments, modulating the condensate modulates interactions between the condensate and methyl-DNA binding proteins. In some embodiments, modulating the condensate modulates interactions between the condensate and gene silencing factors. In some embodiments, modulating the condensate modulates repression or activation of one or more genes located in heterochromatin. In some embodiments, modulating the condensate modulates interactions between the condensate and splicing factors, initiation factors or elongation factor. In some embodiments, modulating the condensate modulates interactions between the condensate and RNA polymerase. In some embodiments, modulating the condensate modulates mRNA initiation or elongation. In some embodiments, modulating the condensate modulates mRNA splicing. In some embodiments, modulating the condensate modulates an inflammatory response (e.g., an inflammatory response to a virus or bacteria). In some embodiments, modulating the condensate modulates (e.g., reduces or eliminates) the viability or growth of cancer. In some embodiments, modulating condensates treats or prevents Rett syndrome or MeCP2 overexpression syndrome. In some embodiments, modulating condensates treats or prevents a condition associated with aberrant mRNA initiation, elongation, or splicing.

In some embodiments, the condensate is modulated by altering a nucleotide sequence associated with the condensate. Alteration can include adding or deleting nucleotides, or epigenetic modification (e.g., increasing or decreasing or modifying DNA methylation). In some embodiments, the alteration of the nucleotide sequence comprises the tethering of a DNA, RNA, or protein to the nucleotide sequence. In some embodiments, a catalytically inactive site specific endonuclease (e.g., dCas) is used to tether the DNA, RNA, or protein to the nucleotide sequence. In some embodiments, the condensate is modulated by tethering a DNA, RNA, or protein to the condensate. In some embodiments, a hormone responsive element or signaling responsive element is modified. In some embodiments, the condensate is modulated by methylating or demethylating DNA associated with the condensate. In some embodiments, the condensate is modulated by phosphorylating or de-phosphorylating a component. In some embodiments, the component is an RNA polymerase.

In some embodiments, the condensate is modulated by contacting the condensate with exogenous RNA. In some embodiments, the condensate is modulated by stabilizing one or more RNAs associated with the condensate (e.g., a condensate component). In some embodiments, the condensate is modulated by modulating the level of an RNA associated with the condensate.

In some aspects, RNA processing in the cell is altered by altering a condensate. In some embodiments, RNA processing is altered by suppressing or enhancing fusion of the transcriptional condensate to one or more RNA processing apparatus condensates. In some embodiments RNA processing comprises splicing, addition of a 5′ cap, 3′ and/or polyadenylation. In some embodiments, the affinity of an RNA polymerase II (Pol II) for a condensate associated with an initiation complex or an elongation complex is modulated. In some embodiments, the affinity is modulated by phosphorylating or dephosphorylating the Pol II (e.g., phosphorylating or dephosphorylating the intrinsically disordered C-terminal domain of Pol II).

In some embodiments, condensates are modulated by modulating the modifier/demodifier ratio of a super-enhancer associated with a condensate (e.g., a super-enhancer within a condensate, a super-enhancer with condensate dependent transcriptional activity). In some embodiments, condensates are modulated by modulating the modification/demodification of a component (e.g., modulating phosphorylation or acetylation of a protein, peptide, DNA, or RNA component). In some embodiments, condensates are modulated by inhibiting or enhancing expression or activity a modifier/demodifier (e.g., thereby modulating the stability, localization and/or binding activity of a condensate component). For example, phosphorylating or dephosphorylating certain proteins can affect their ability to interact with other molecular entities (e.g., condensate components). In some embodiments, such modification/demodification may cause a condensate component to dissociate from proteins that otherwise retain them in the cytoplasm and cause them to translocate to the nucleus where they can participate in a condensate. Thus, in some embodiments, modifying condensate formation, stability, composition, maintenance, dissolution, or activity comprises inhibiting or activating a modifier/demodifier of a condensate component. In some embodiments the modifier is a kinase and the agent that inhibits the modifier is a kinase inhibitor.

In some embodiments, condensates are modulated by contacting the condensate with an agent that binds to an intrinsically disordered domain of a component associated with the condensate. In some embodiments, the component is Mediator, a mediator component, MED1, MED15, p300, BRD4, TFIID, β-catenin, STAT3, SMAD3, NF-KB, MECP2, MBD1, MBD2, MBD3, MBD4, HP1α, TBL1R, HDAC3, SMRT RNA polymerase II, SRSF2, SRRM1, or SRSF1. In some embodiments, the component is a nuclear receptor ligand or fragment thereof (e.g., a hormone). In some embodiments, the component is a signaling factor or fragment thereof. In some embodiments, the component is a methyl-binding protein or suppressor, or fragment thereof. In some embodiments, the component is an RNA polymerase, splicing factor, initiation factor, elongation factor, or fragment thereof. In some embodiments, the component is listed in Table S1. In some embodiments, the component is a transcription factor (e.g., OCT4, p53, MYC, GCN4, NANOG, MyoD, KLF4, a SOX family transcription factor, a GATA family transcription factor, a nuclear receptor, or a fusion oncogenic transcription factor). In some embodiments, the IDR is located in the activation domain of a transcription factor. In some embodiments of the methods and compositions disclosed herein, the component is a nuclear receptor or a fragment of a nuclear receptor comprising an activation domain, or an activation domain IDR. In some embodiments, the agent is multivalent. In some embodiments, the agent is bivalent. In some embodiments, the agent further binds to a non-intrinsically disordered domain of the component or binds to a second component associated with the condensate. In some embodiments, the agent can alter or disrupt interactions between components of the condensates. In some embodiments, the agent can stabilize or enhance interactions between components of the condensates. In some embodiments, the agent binds to non-disordered regions of two or more components (e.g., enhancing IDR interactions of the components).

In some embodiments, formation of the condensate can be caused, enhanced, or stabilized by tethering one or more condensate components to genomic DNA. In some embodiments, these components comprise DNA, RNA, and/or protein. In some embodiments, the components comprise Mediator, a mediator component, MED1, MED15, p300, BRD4, a nuclear receptor ligand, signaling factor, β-catenin, STAT3, SMAD3, NF-KB, MECP2, MBD1, MBD2, MBD3, MBD4, HP1α, TBL1R, HDAC3, SMRT RNA polymerase II, SRSF2, SRRM1, SRSF1, or TFIID. In some embodiments, the component is a transcription factor (e.g., OCT4, p53, MYC, GCN4, NANOG, MyoD, KLF4, a SOX family transcription factor, a GATA family transcription factor, a nuclear receptor, or a fusion oncogenic transcription factor). In some embodiments, the components are tethered using a catalytically inactive site specific endonuclease (e.g., dCas).

In some embodiments, the condensate is modulated by sequestration of one or more components of the condensate in a second condensate. In some embodiments, formation of the second condensate is induced by contacting the cell with an exogenous peptide, nucleic acid and/or protein. In some embodiments, the sequestered component is a transcription factor (e.g., OCT4, p53, MYC, GCN4, NANOG, MyoD, KLF4, a SOX family transcription factor, a GATA family transcription factor, a nuclear receptor, or a fusion oncogenic transcription factor). In some embodiments, the sequestered component is Myc. In some embodiments, the sequestered component is a mutant version of a wild-type protein. In some embodiments, the sequestered component is a component over-expressed in a disease state (e.g., cancer). In some embodiments, the sequestered component is a nuclear receptor (e.g. a mutant version of the nuclear receptor, a mutant version of a nuclear receptor associated with a disease state). In some embodiments, the sequestered component is a nuclear receptor ligand, signaling factor, methyl-DNA binding protein, splicing factor, initiation factor, elongation factor, gene silencing factor, or RNA polymerase.

In some embodiments, the condensate is modulated by modulating a level or activity of ncRNA associated with the condensate (e.g., a component of the condensate). In some embodiments, the level or activity of the ncRNA is modulated by contacting the ncRNA with an anti-sense oligonucleotide, an RNase, or a chemical compound that binds the ncRNA. In some embodiments the ncRNA is an enhancer RNA (eRNA). In some embodiments, the ncRNA is a transfer RNA (tRNA), ribosomal RNA (rRNA), microRNA, siRNA, piRNA, snoRNA, snRNA, exRNA, scaRNA, Xist or HOTAIR.

In some embodiments, the methods described herein treat or reduce the likelihood of a disease caused by, or dependent on, condensate formation, composition, maintenance, dissolution or regulation. In some embodiments, the methods described herein treat or reduce the likelihood of a cancer. In some embodiments, the cancer is associated with a mutation in a condensate component (e.g., a nuclear receptor). In some embodiments, the methods described herein treat or reduce the likelihood of a disease associated with a nuclear receptor (e.g., a mutant nuclear receptor). In some embodiments, the methods described herein treat or reduce the likelihood of a disease associated with aberrant protein expression (e.g., a disease that causes a pathological level of a protein). In some embodiments, the methods described herein treat or reduce the likelihood of a disease associated with aberrant signaling. In some embodiments, the methods described herein reduce inflammation. In some embodiments, methods describe herein modify a cell state. In some embodiments, the methods described herein treat or reduce the likelihood of a disease associated with the generation of fusion oncogenic transcription factors that inappropriately activate cell survival or proliferation pathways, inappropriate production of transcription factors that are not expressed in the normal tissue, or mutation of an enhancer region that recruits a transcription factors to a previously silent oncogene. In some embodiments, methods described herein modify cell identity. In some embodiments, methods described herein treat a disease associated with aberrant expression or activity (e.g., an increased or decreased level as compared to a reference or control level) of a methyl-DNA binding protein. In some embodiments, methods described herein treat a disease associated with aberrant mRNA initiation or elongation (e.g., an increased or decreased mRNA initiation or elongation as compared to a reference or control level). In some embodiments, methods described herein treat a disease associated with aberrant mRNA splicing (e.g., increased or decreased mRNA splicing activity as compared to a reference or control level).

Some aspects of the disclosure are directed to a method of identifying an agent that modulates condensate formation, stability, activity (e.g., mRNA initiation or elongation activity, gene silencing activity) or morphology of a condensate (e.g., transcriptional condensate), comprising providing a cell having a condensate, contacting the cell with a test agent, determining if contact with the test agent modulates formation, stability, activity, or morphology of the condensate. In some embodiments, the condensate has a detectable tag (i.e., detectable label) and the detectable tag is used to determine if contact with the test agent modulates formation, stability, activity, or morphology of the condensate. In some embodiments, the detectable tag is a fluorescent tag. In some embodiments, the detectable tag is an enzymatic tag, e.g., a luciferase. In some embodiments, the detectable tag is an epitope tag. In some embodiments, an antibody selectively binding to the condensate is used to determine if contact with the test agent modulates formation, stability, activity, or morphology of the condensate. In some embodiments, the step of determining if contact with the test agent modulates formation, stability, activity, or morphology of the condensate is performed using microscopy. In some embodiments, the condensate comprises a mutant component (e.g., a mutant version of a nuclear receptor or fragment thereof, a mutant version of a nuclear receptor having a different activity or level of activity when bound to a cognate ligand than the wild-type receptor or a fragment thereof, a mutant signaling factor or fragment thereof, a mutant methyl-DNA binding protein or fragment thereof). In some embodiments of the above, the cell does not have a condensate the method comprises identifying an agent that causes condensate formation in the cell. In some embodiments, a condensate is not detectable in the cell and the method comprises identifying an agent that makes the condensate detectable (e.g., the condensate becomes sufficiently large to be detected). In some embodiments, the cell has a condensate and the method comprises identifying an agent that causes the formation of another condensate.

In some embodiments, the component of the condensate (e.g., transcriptional condensate) is a signaling factor or a fragment thereof comprising an IDR. In some embodiments, the condensate is associated with one or more signal response elements. In some embodiments, the signaling factor is associated with a signaling pathway associated with a disease. In some embodiments, the disease is cancer. In some embodiments, the condensate modulates transcription of an oncogene. In some embodiments, the condensate is associated with a super-enhancer. In some embodiments, the component of the condensate is a methyl-DNA binding protein or a fragment thereof comprising a C-terminal IDR, or a suppressor or fragment thereof comprising an IDR. In some embodiments, the condensate is associated with methylated DNA or heterochromatin. In some embodiments, the condensate comprises an aberrant level or activity of methyl-DNA binding protein. In some embodiments, the cell is any type of cell mentioned herein. In some embodiments, the cell is a nerve cell. In some embodiments, the cell is derived from (e.g, via an induced pluripotent stem cell derived from a subject cell) a subject having Rett syndrome or MeCP2 overexpression syndrome.

In some embodiments, suppression of expression of genes associated with the condensate by the agent are assessed. In some embodiments, the component of the condensate is a splicing factor or a fragment thereof comprising an IDR, or an RNA polymerase or fragment thereof comprising an IDR. In some embodiments, the condensate is associated with a transcription initiation complex or elongation complex. In some embodiments, the cell further comprises a cyclin dependent kinase. In some embodiments, the RNA polymerase is RNA polymerase II (Pol II). In some embodiments, changes in RNA transcription initiation activity associated with the condensate caused by contact with the agent are assessed. In some embodiments, changes in RNA elongation or splicing activity physically associated with the condensate caused by contact with the agent are assessed.

Some aspects of the disclosure are directed to a method of identifying an agent that modulates condensate formation, stability, or morphology, comprising providing an in vitro condensate and assessing one or more physical properties of the in vitro condensate, contacting the in vitro condensate with a test agent, and assessing whether the test agent causes a change in the one or more physical properties of the in vitro condensate. In some embodiments, the one or more physical properties correlate with the in vitro condensate's ability to cause, or increase, or decrease, expression of a gene in a cell. In some embodiments, the one or more physical properties correlate with the in vitro condensate's ability to cause, or increase, or decrease, RNA splicing. In some embodiments, the one or more physical properties comprise size, concentration, permeability, morphology, or viscosity. In some embodiments, the test agent is, or comprises, a small molecule, a peptide, a RNA or a DNA. In some embodiments, the in vitro condensate comprises DNA, RNA and protein. In some embodiments, the in vitro condensate comprises, consists of, or essentially consists of DNA and protein. In some embodiments, the in vitro condensate comprises, consists of, or essentially consists of RNA and protein. In some embodiments, the in vitro condensate comprises, consists of, or essentially consists of protein. In some embodiments, the in vitro condensate comprises intrinsically disordered regions or domains (e.g. proteins, peptides, or a fragment or derivative thereof comprising one or more intrinsically disordered regions or domains). In some embodiments, the in vitro condensate is formed by weak protein-protein interactions (e.g., easily perturbed interactions, easily perturbed and transient interactions, interactions having a K_{d} in a micromolar range, interactions having a K_{d} in a micromolar range and transient). In some embodiments, the in vitro condensate comprises (intrinsically disordered domain)-(inducible oligomerization domain) fusion proteins. In some embodiments, the in vitro condensate simulates a transcriptional condensate found in a cell. In some embodiments, the in vitro condensate simulates a heterochromatin condensate (e.g., a heterochromatin condensate silencing gene expression). In some embodiments, the in vitro condensate comprises methylated DNA. In some embodiments, the in vitro condensate simulates an mRNA initiation or elongation complex. In some embodiments, the in vitro condensate comprises a signal response element. In some embodiments the condensate is in a liquid droplet (e.g., in vitro, a synthetic transcriptional condensate).

In some embodiments, the component of the condensate is a signaling factor or a fragment thereof comprising an IDR. In some embodiments, the condensate is associated with one or more signal response elements. In some embodiments, the signaling factor is associated with a signaling pathway associated with a disease. In some embodiments, the disease is cancer. In some embodiments, the condensate modulates transcription of an oncogene. In some embodiments, the condensate is associated with a super-enhancer. In some embodiments, the component of the condensate is a methyl-DNA binding protein or a fragment thereof comprising a C-terminal IDR, or a suppressor or fragment thereof comprising an IDR. In some embodiments, the condensate is associated with methylated DNA or heterochromatin. In some embodiments, the condensate comprises an aberrant level or activity of methyl-DNA binding protein. In some embodiments the cell is of any cell type mentioned herein or known in the art. In some embodiments, the cell is a nerve cell. In some embodiments, the cell is derived from (e.g, via an induced pluripotent stem cell derived from a subject cell) a subject having Rett syndrome or MeCP2 overexpression syndrome.

In some embodiments, suppression of expression of genes associated with the condensate by the agent is assessed. In some embodiments, the component of the condensate is a splicing factor or a fragment thereof comprising an IDR, or an RNA polymerase or fragment thereof comprising an IDR. In some embodiments, the condensate is associated with a transcription initiation complex or elongation complex. In some embodiments, the cell further comprises a cyclin dependent kinase. In some embodiments, the RNA polymerase is RNA polymerase II (Pol II). In some embodiments, changes in RNA transcription initiation activity associated with the condensate caused by contact with the agent are assessed. In some embodiments, changes in RNA elongation or splicing activity associated with the condensate caused by contact with the agent are assessed.

Some aspects of the disclosure are directed to a method of identifying an agent that modulates condensate formation, stability, function, or morphology, comprising, providing a cell with condensate dependent expression of a reporter gene, contacting the cell with a test agent, and assessing expression of the reporter gene.

In some embodiments of the methods of identifying an agent disclosed herein, the condensate comprises a nuclear receptor (e.g., nuclear hormone receptor) or fragment thereof comprising an activation domain IDR. In some embodiments, the nuclear receptor activates transcription when bound to a cognate ligand. In some embodiments, the nuclear receptor activates transcription without binding to a cognate ligand. In some embodiments, the level of transcription activated by the nuclear receptor (e.g., mutant nuclear receptor) is different (e.g., 1.5-fold, 2-fold, 3-fold, 4-fold, 5-fold different) than a wild-type nuclear receptor or a version of the nuclear receptor not associated with a disease or condition. In some embodiments, the nuclear receptor is a nuclear hormone receptor. In some embodiments, the nuclear receptor has a mutation. In some embodiments, the mutation is associated with a disease or condition. In some embodiments, the disease or condition is cancer (e.g., breast cancer or leukemia).

In some embodiments, the methods disclosed herein comprising a condensate with a nuclear receptor further comprise the presence of a ligand (e.g., a ligand in the condensate, a ligand in the assay mixture). In some embodiments, an assay comprising a ligand is used to identify an agent that inhibits condensate formation that would be promoted by the ligand or act additively or synergistically with the ligand to promote condensate formation/stability, function, or morphology. Ligand may be a naturally occurring endogenous ligand (e.g., cognate ligand) or a ligand (e.g., a synthetic ligand) that is distinct in structure from a naturally occurring endogenous ligand.

In some embodiments of the methods of identifying an agent disclosed herein, the condensate comprises a mutant condensate component (e.g, a mutant TF, mutant NR) that exhibits one or more aberrant properties, e.g., aberrant condensate formation, stability, function, or morphology, and the assay comprises identifying an agent that at least partly normalizes the property. In some embodiments of the methods of identifying an agent disclosed herein, the condensate comprises a mutant NR that exhibits one or more aberrant properties and the assay is performed in the presence of a ligand that, when contacted with the NR causes the aberrant properties to be exhibited. The assay may be used to identify an agent that normalizes the aberrant properties.

Some aspects of the disclosure are directed to an isolated synthetic transcriptional condensate comprising DNA, RNA and protein. Some aspects of the disclosure are directed to an isolated synthetic transcriptional condensate comprising DNA and protein. In some embodiments, a liquid droplet comprises the isolated synthetic transcriptional condensate. Some aspects of the disclosure are directed to an isolated synthetic condensate comprising protein characteristic of a heterochromatin condensate or condensate physically associated with a mRNA initiation or elongation complex. Some aspects of the disclosure are directed to an isolated synthetic condensate comprising DNA and protein characteristic of a heterochromatin condensate or condensate physically associated with an mRNA initiation or elongation complex. In some embodiments, a liquid droplet comprises the isolated synthetic condensate.

Some aspects of the disclosure are directed to a fusion protein comprising a transcriptional condensate component (e.g., a transcription factor or fragment thereof, a fragment of a transcription factor comprising an activation domain or activation domain IDR) and a domain that confers inducible oligomerization. Some aspects of the disclosure are directed to a fusion protein comprising a component of a heterochromatin condensate or a condensate physically associated with a mRNA initiation or elongation complex. The fusion protein can further comprise a detectable tag (e.g., a fluorescent tag). In some embodiments, the domain that confers inducible oligomerization is inducible with a small molecule, protein, or nucleic acid. In some embodiments condensate formation is inducible with a small molecule, protein, nucleic acid, or light.

Some aspects of the disclosure are directed to methods of detecting, e.g., visualizing, condensates, e.g., transcriptional condensates, heterochromatin condensates, condensates associates with mRNA initiation or elongation complex. In some aspects, the formation, morphology or dissolution of a transcriptional condensate may be visualized. In some embodiments visualizing a transcriptional condensate may be useful in screening for agents that modulate said condensate. In some aspects, the formation, morphology or dissolution of a condensate (e.g., heterochromatin condensate or a condensate physically associated with a mRNA initiation or elongation complex) may be visualized. In some embodiments visualizing a condensate (e.g., heterochromatin condensate or a condensate physically associated with a mRNA initiation or elongation complex) may be useful in screening for agents that modulate said condensate. In some embodiments, methods comprise monitoring the rate of condensate formation or dissolution. In some embodiments methods comprise identifying agent that increases or decreases the rate of condensate formation or dissolution.

Some aspects of the disclosure are directed to a method of modulating mRNA initiation, comprising modulating formation, composition, maintenance, dissolution and/or regulation of a condensate physically associated with mRNA initiation. In some embodiments, modulating mRNA initiation also modulates mRNA elongation, splicing or capping. In some embodiments, modulating formation, composition, maintenance, dissolution and/or regulation of the condensate physically associated with mRNA initiation modulates an mRNA transcription rate. In some embodiments, modulating formation, composition, maintenance, dissolution and/or regulation of the condensate physically associated with mRNA initiation modulates a level of a gene product.

In some embodiments, formation, composition, maintenance, dissolution and/or regulation of the condensate physically associated with mRNA initiation is modulated with an agent. The agent is not limited and may be any agent described herein. In some embodiments, the agent comprises a hypophosphorylated RNA polymerase II C-terminal domain (Pol II CTD) or a functional fragment thereof. In some embodiments, the agent preferentially binds hypophosphorylated Pol II CTD.

Some aspects of the disclosure are directed to a method of modulating mRNA elongation, comprising modulating formation, composition, maintenance, dissolution and/or regulation of a condensate physically associated with an mRNA elongation complex. In some embodiments, modulating mRNA elongation also modulates mRNA initiation. In some embodiments, modulating formation, composition, maintenance, dissolution and/or regulation of the condensate physically associated with mRNA elongation modulates co-transcriptional processing of an mRNA. In some embodiments, modulating formation, composition, maintenance, dissolution and/or regulation of the condensate physically associated with mRNA elongation modulates the number or relative proportion of mRNA splice variants. In some embodiments, formation, composition, maintenance, dissolution and/or regulation of the condensate physically associated with mRNA elongation is modulated with an agent. The agent is not limited and may be any agent disclosed herein. In some embodiments, the agent comprises a phosphorylated or hypophosphorylated RNA polymerase II C-terminal domain (Pol II CTD) or a functional fragment thereof. In some embodiments, the agent preferentially binds a phosphorylated or hypophosphorylated Pol II CTD.

Some aspects of the disclosure are related to a method of modulating formation, composition, maintenance, dissolution and/or regulation of a condensate comprising modulating the phosphorylation or dephosphorylation of a condensate component. In some embodiments, the component is RNA polymerase II or an RNA polymerase II C-terminal region.

Some aspects of the disclosure are related to a method of treating or reducing the likelihood of a disease or condition associated with aberrant mRNA processing comprising modulating formation, composition, maintenance, dissolution and/or regulation of a condensate physically associated with mRNA elongation.

Some aspects of the disclosure are related to a method of identifying an agent that modulates formation, stability, or morphology of a condensate, comprising providing a cell having a condensate, contacting the cell with a test agent, and determining if contact with the test agent modulates formation, stability, or morphology of the condensate, wherein the condensate comprises a hypophosphorylated RNA polymerase II C-terminal domain (Pol II CTD), a phosphorylated RNA polymerase II C-terminal domain (Pol II CTD), a splicing factor, or a functional fragment thereof. In some embodiments of the methods disclosed herein of identifying an agent or screening for an agent that formation, composition, maintenance, dissolution, activity, and/or regulation of a condensate associated with (e.g., having an aberrant level, property, or activity) a disease or condition, the agent is not known to be useful for treating the disease or condition.

Some aspects of the disclosure are related to a method of identifying an agent that modulates formation, stability, or morphology of a condensate, comprising providing an in vitro condensate and assessing one or more physical properties of the in vitro condensate, contacting the in vitro condensate with a test agent, and assessing whether the test agent causes a change in the one or more physical properties of the in vitro condensate, wherein the condensate comprises a hypophosphorylated RNA polymerase II C-terminal domain (Pol II CTD), a phosphorylated RNA polymerase II C-terminal domain (Pol II CTD), a splicing factor, or a functional fragment thereof.

Some aspects of the disclosure are related to an isolated synthetic condensate comprising hypophosphorylated RNA polymerase II C-terminal domain (Pol II CTD) or a functional fragment thereof. Some aspects of the disclosure are related to an isolated synthetic condensate comprising phosphorylated RNA polymerase II C-terminal domain (Pol II CTD) or a functional fragment thereof. Some aspects of the disclosure are related to an isolated synthetic condensate comprising a splicing factor or a functional fragment thereof.

Some aspects of the disclosure are related to a method of modulating transcription of one or more genes, comprising modulating formation, composition, maintenance, dissolution and/or regulation of a heterochromatin condensate. In some embodiments, modulating the heterochromatin condensate increases or stabilizes repression of transcription of the one or more genes. In some embodiments, modulating the heterochromatin condensate decreases repression of transcription of the one or more genes. In some embodiments, the transcription of a plurality of genes associated with heterochromatin are modulated. In some embodiments, formation, composition, maintenance, dissolution and/or regulation of the heterochromatin condensate is modulated with an agent. In some embodiments, the agent comprises, or consists of, a peptide, nucleic acid, or small molecule. In some embodiments, the agent binds methylated DNA, a methyl-DNA binding protein, or a gene silencing factor.

Some aspects of the disclosure are related to a method of modulating gene silencing, comprising modulating formation, composition, maintenance, dissolution and/or regulation of a heterochromatin condensate. In some embodiments, gene silencing is stabilized or increased. In some embodiments, gene silencing is decreased. In some embodiments, gene silencing is modulated with an agent.

Some aspects of the disclosure are related to a method of treating or reducing the likelihood of a disease or condition associated with aberrant gene silencing (e.g., increased or decreased gene silencing as compared to a control or reference level) comprising modulating formation, composition, maintenance, dissolution and/or regulation of a heterochromatin condensate. In some embodiments, the disease or condition associated with aberrant gene silencing is associated with aberrant expression or activity of a methyl-DNA binding protein. In some embodiments, the disease or condition associated with aberrant gene silencing is Rett syndrome or MeCP2 overexpression syndrome.

Some aspects of the disclosure are related to a method of identifying an agent that modulates formation, stability, or morphology of a condensate, comprising providing a cell having a condensate, contacting the cell with a test agent, and determining if contact with the test agent modulates formation, stability, or morphology of the condensate, wherein the condensate comprises MeCP2 or a fragment thereof comprising a C-terminal intrinsically disordered region of MeCP2, or a suppressor. In some embodiments, the condensate is associated with heterochromatin. In some embodiments, the condensate is associated with methylated DNA.

Some aspects of the disclosure are related to a method of identifying an agent that modulates formation, stability, or morphology of a condensate, comprising providing an in vitro condensate and assessing one or more physical properties of the in vitro condensate, contacting the in vitro condensate with a test agent, and assessing whether the test agent causes a change in the one or more physical properties of the in vitro condensate, wherein the condensate comprises MeCP2 or a fragment thereof comprising a C-terminal intrinsically disordered region of MeCP2, or a suppressor or functional fragment thereof.

Some aspects of the disclosure are related to an isolated synthetic condensate comprising MeCP2 or a fragment thereof comprising a C-terminal intrinsically disordered region of MeCP2.

Some aspects of the disclosure are related to an isolated synthetic condensate comprising a suppressor (sometimes referred to herein as a gene-silencing factor) or a functional fragment thereof.

Some aspects of the disclosure are related to a method of modulating transcription of one or more genes in a cell, comprising modulating composition, maintenance, dissolution and/or regulation of a condensate associated with the one or more genes, wherein the condensate comprises an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding. In some embodiments, the estrogen receptor fragment comprises a ligand binding domain or a functional fragment thereof. In some embodiments, the MED1 fragment comprises an IDR, an LXXLL motif, or both. In some embodiments, the condensate is contacted with estrogen or a functional fragment thereof. In some embodiments, the condensate is contacted with a selective estrogen selective modulator (SERM). In some embodiments, the SERM is tamoxifen. In some embodiments, modulation of the condensate reduces or eliminates transcription of MYC oncogene. In some embodiments, the cell is a breast cancer cell. In some embodiments, the cell over-expresses MED1. In some embodiments, the transcriptional condensate is modulated by contacting the transcriptional condensate with an agent. In some embodiments, the agent reduces or eliminates interactions between the ER and MED1. In some embodiments, the agent reduces or eliminates interactions between ER and estrogen. In some embodiments, the condensate comprises a mutant ER or fragment thereof and the agent reduces transcription of the one or more genes.

Some aspects of the disclosure are related to a method of identifying an agent that modulates formation, stability, or morphology of a condensate, comprising providing a cell, contacting the cell with a test agent, and determining if contact with the test agent modulates formation, stability, or morphology of a condensate, wherein the condensate comprises an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding. In some embodiments, the estrogen receptor fragment comprises a ligand binding domain or a functional fragment thereof. In some embodiments, the MED1 fragment comprises an IDR, an LXXLL motif, or both. In some embodiments, the condensate is contacted with estrogen or a functional fragment thereof. In some embodiments, the condensate is contacted with a selective estrogen selective modulator (SERM). In some embodiments, the SERM is tamoxifen or an active metabolite thereof. In some embodiments, modulation of the condensate reduces or eliminates transcription of MYC oncogene. In some embodiments, the cell is a breast cancer cell. In some embodiments, the cell over-expresses MED1. In some embodiments, the cell is an ER+ breast cancer cell. In some embodiments, the ER+ breast cancer cell is resistant to tamoxifen treatment. In some embodiments, the condensate comprises a detectable label. In some embodiments, a component of the condensate comprises the detectable label. In some embodiments, the ER or a fragment thereof, and/or the MED1 or a fragment thereof comprises the detectable label. In some embodiments, the one or more genes comprise a reporter gene.

Some aspects of the invention are related to a method of identifying an agent that modulates formation, stability, or morphology of a condensate, comprising providing an in vitro condensate, contacting the condensate with a test agent, and determining if contact with the test agent modulates formation, stability, or morphology of the condensate, wherein the condensate comprises an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding. In some embodiments, the estrogen receptor fragment comprises a ligand binding domain or a functional fragment thereof. In some embodiments, the MED1 fragment comprises an IDR, an LXXLL motif, or both. In some embodiments, the condensate is contacted with estrogen or a functional fragment thereof. In some embodiments, the condensate is contacted with a selective estrogen selective modulator (SERM). In some embodiments, the SERM is tamoxifen. In some embodiments, the condensate is isolated from a cell. In some embodiments, the cell is a breast cancer cell. In some embodiments, the cell over-expresses MED1. In some embodiments, the cell is an ER+ breast cancer cell. In some embodiments, the ER+ breast cancer cell is resistant to tamoxifen treatment. In some embodiments, the condensate comprises a detectable label. In some embodiments, a component of the condensate comprises the detectable label. In some embodiments, the ER or a fragment thereof, and/or the MED1 or a fragment thereof comprises the detectable label.

Some aspects of the disclosure are related to an isolated synthetic transcriptional condensate comprising an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding. In some embodiments, the estrogen receptor fragment comprises a ligand binding domain or a functional fragment thereof. In some embodiments, the MED1 fragment comprises an IDR, an LXXLL motif, or both. In some embodiments, the condensate comprises estrogen or a functional fragment thereof. In some embodiments, the condensate comprises a selective estrogen selective modulator (SERM).

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIG. 1—illustrates a transcriptional condensate as a high density cooperative assembly of multiple components including transcription factors, co-factors, chromatin regulators, DNA, non-coding RNA, nascent RNA, and RNA polymerase II.

FIG. 2A-2B—show the influence of an intrinsically disordered domain or region (IDR) (SEQ ID NO: 13) on transcriptional condensate formation, maintenance, dissolution or regulation. In FIG. 2A, the IDR stabilizes the transcriptional condensate. In FIG. 2B, the introduction of a small molecule that binds or interacts with the IDR destabilizes the transcriptional condensate. The motif YSPTSPS shown in FIGS. 2A-2B is SEQ ID NO: 13.

FIGS. 3A-3C—shows model and features of super-enhancers and typical enhancers. FIG. 3A is a schematic depiction of the classic model of cooperativity for typical enhancers and super-enhancers. The higher density of transcriptional regulators (referred to as “activators”) through cooperative binding to DNA binding sites is thought to contribute to both higher transcriptional output and increased sensitivity to activator concentration at super-enhancers. Image adapted from Lovén et al. (2013). FIG. 3B shows chromatin immunoprecipitation sequencing (ChIP-seq) binding profiles for RNA polymerase II (RNA Pol II) and the indicated transcriptional cofactors and chromatin regulators at the POLE4 and miR-290-295 loci in murine embryonic stem cells. The transcription factor binding profile is a merged ChIP-seq binding profile of the TFs Oct4, Sox2, and Nanog. rpm/bp, reads per million per base pair. Image adapted from Hnisz et al. (2013). FIG. 3C shows ChIA-PET interactions at the RUNX1 locus displayed above the ChIP-seq profiles of H3K27Ac in human T cells. The ChIA-PET interactions indicate frequent physical contact between the H3K27Ac occupied regions within the super-enhancer and the promoter of RUNX1.

FIGS. 4A-4C—shows a Simple Phase Separation Model of Transcriptional Control. FIG. 4A is a schematic representation of the biological system that can form the phase-separated multi-molecular complex of transcriptional regulators at a super-enhancer-gene locus. FIG. 4B is a simplified representation of the biological system, and parameters of the model that could lead to phase separation. “M” denotes modification of residues that are able to form cross-links when modified. FIG. 4C shows the dependence of transcriptional activity (TA) on the valency parameter for super-enhancers (consisting of N=50 chains), and typical enhancers (consisting of N=10 chains). The proxy for transcriptional activity (TA) is defined as the size of the largest cluster of cross-linked chains, scaled by the total number of chains. The valency is scaled such that the actual valency is divided by a reference number of three. The solid lines indicate the mean, and the dashed lines indicate twice the standard deviation in 50 simulations. The value of K_{eq} and modifier/demodifier ratio was kept constant. HC, Hill coefficient, which is a classic metric to describe cooperative behavior. The inset shows the dependency of the Hill coefficient on the number of chains, or components, in the system.

FIGS. 5A-5B—shows Super-Enhancer Vulnerability. FIG. 5A shows enhancer activities of the fragments of the IGLL5 super-enhancer (red) and the PDHX typical enhancer (gray) after treatment with the BRD4 inhibitor JQ1 at the indicated concentrations. Enhancer activity was measured in luciferase reporter assays in human multiple myeloma cells. Note that JQ1 inhibits ˜50% of luciferase expression driven by the super-enhancer at a 10-fold lower concentration than luciferase expression driven by the typical enhancer (25 nM versus 250 nM). Data and image adapted from Lovén et al. (2013). FIG. 5B shows dependence of transcriptional activity (TA) on the demodifier/modifier ratio for super-enhancers (consisting of N=50 chains), and typical enhancers (consisting of N=10 chains). The proxy for transcriptional activity (TA) is defined as the size of the largest cluster of cross-linked chains, scaled by the total number of chains. The solid lines indicate the mean and the dashed lines indicate twice the standard deviation of 50 simulations. K_{eq} and f were kept constant. Note that increasing the demodifier levels is equivalent to inhibiting cross-linking (i.e., reducing valency). TA is normalized to the value at log (demodifier/modifier)=−1.5, and the ordinate shows the normalized TA on a log scale.

FIGS. 6A-6C—shows Transcriptional Bursting. FIG. 6A is representative traces of transcriptional activity in individual nuclei of Drosophila embryos. Transcriptional activity was measured by visualizing nascent RNAs using fluorescent probes. Top panel shows a representative trace produced by a weak enhancer, and the bottom panel shows a representative trace produced by a strong enhancer. Data and image adapted from Fukaya et al. (2016). FIG. 6B is a simulation of transcriptional activity (TA) of super-enhancers (N=50 chains), and typical enhancers (N=10 chains) that over time recapitulates bursting behavior of weak and strong enhancers. FIG. 6C is a model of synchronous activation of two gene promoters by a shared enhancer.

FIG. 7—shows Transcriptional Control Phase Separation In Vivo: A model of a phase-separated complex at gene regulatory elements. Some of the candidate transcriptional regulators forming the complex are highlighted. P-CTD denotes the phosphorylated C-terminal domain of RNA Pol II. Chemical modifications of nucleosomes (acetylation, Ac; methylation, Me) are also highlighted. Divergent transcription at enhancers and promoters produces nascent RNAs that can be bound by RNA splicing factors. Potential interactions between the components are displayed as dashed lines.

FIG. 8—shows dependence of transcriptional activity (TA) on number of chains (N). The proxy for transcriptional activity (TA) is defined as the size of the largest cluster of cross-linked chains, scaled by the total number of chains. The solid lines indicate the mean and the dashed lines indicate twice the standard deviation in 50 simulations. All simulations are done at Modifier/Demodifier=0.1, K_{eq}=1 and f=5. TA levels are very different as long as the values of N (or concentration of components) for a SE and a typical enhancer are sufficiently different.

FIG. 9—shows simulations carried out to study disassembly of the gel after a sharp change in the Modifier/Demodifier balance (mimics change in signals). The proxy for transcriptional activity (TA) is defined as the size of the largest cluster of cross-linked chains, scaled by the total number of chains. As depicted in the inset, the ratio of Modifier/Demodifier levels are flipped (at t=25) from 0.1 to 0.016 and TA is calculated τ=50 time units post change in the Modifier/Demodifier balance. All simulations are done for N=50 (model for SE) and K_{eq}=1. The solid line represents the variation in the maximum value of the calculated TA in 250 replicate simulations as valency (f) is changed. Threshold valencies fₘᵢₙ, for ensuring cluster formation (see FIG. 4C), and fₘₐₓ, to ensure robust disassembly (defined as TA<0.5, dotted line) within τ=50 time units post change in Modifier/Demodifier levels are identified. The specific value of τ=50 time units post change in Modifier/Demodifier values is chosen for illustrative purposes, and determines the value of fₘₐₓ. The qualitative result that there exists a maximal valency above which the gel does not disassemble in a realistic time scale is robust to changes in the chosen value of this time scale.

FIGS. 10A-10B—shows Noise characteristics of super-enhancers and typical enhancers. FIG. 10A shows dependence of fluctuations (or transcriptional noise), measured as variance in Transcriptional activity (TA), on valency for SEs (N=50) and typical enhancers (N=10). The proxy for transcriptional activity (TA) is defined as the size of the largest cluster of cross-linked chains, scaled by the total number of chains. The angular brackets in the definition of the ordinate represent averages over 50 replicate simulations. All simulations are done at Modifier/Demodifier=0.1, K_{eq}=1. The normalized magnitude of the noise, and importantly the range of valencies over which the noise is manifested, are smaller for SEs compared to a typical enhancer. Note, however, that the absolute magnitude of the noise in the vicinity of the phase separation point is larger for bigger values of N. FIG. 10B shows the dependence of fluctuations (or transcriptional noise), measured as variance in Transcriptional activity (TA), on N for f=5 (the minimal valency required for cluster formation for N=50). All simulations are done at Modifier/Demodifier=0.1 and K_{eq}=1. The proxy for transcriptional activity (TA) is defined as the size of the largest cluster of cross-linked chains, scaled by the total number of chains. The angular brackets in the definition of the ordinate represent averages over 50 replicate simulations.

FIGS. 11A-11E—show visualizations of BRD4 and MED1 nuclear condensates. (FIG. 11A) Representative images of BRD4 and MED1 in mouse embryonic stem cells (mESC) by immunofluorescence (IF) using structured illumination microscopy (SIM). Images represent a z-projection of 8 slices (125 nm, each). Scale bar, 5 μm. IgG control in Fig. S1C. (FIG. 11B) Representative images of co-localization between ectopically expressed BRD4-GFP (left panel, green) and IF for MED1 (middle panel, magenta) in fixed mESC imaged by SIM. Merge of two channels is presented in the right panel with overlap displayed as white. Nuclear outline is shown as blue line determined by DAPI staining (not shown). Images represent a single z-slice (125 nm). Scale bar, 5 μm. (FIG. 11C) Representative images co-IF for BRD4 (top left panel, green), HP1a (top middle panel, magenta), and the merge of the two channels (top left panel, overlap in white) imaged by SIM in fixed mESC. Representative images of co-localization between ectopically expressed HP1a-GFP (bottom right panel, green), IF for MED1 (bottom middle panel, magenta), and the merge of the two channels (bottom left panel, overlap in white) imaged by SIM in fixed mESC. Nuclear outline is shown as blue line determined by DAPI staining (not shown). Images represent a single z-slice (125 nm). Scale bar, 5 μm. (FIG. 11D) Representative images of IF for markers of known nuclear condensates, FIB1 (nucleolus), NPAT (histone locus bodies), and HP1a (constitutive heterochromatin), imaged by deconvolution microscopy. Images represent a z-projection of 8 slices (125 nm, each). Scale bar, 5 μm. (FIG. 11E) Typical number and sizes (diameter) of nuclear condensates. Values generated here are in black font; values collected from the literature are in blue (48). Values for size and number were generated using the 3D object counter plugin in FIJI. Scale bar, 5 μm.

FIGS. 12A-12B—show BRD4 and MED1 condensates occur at sites of super-enhancer-associated transcription. (FIG. 12A) ChIP-seq binding profiles for BRD4, MED1, and RNA polymerase II (RNAPII), as indicated, shown at the super-enhancers (SEs) associated with mir290, Esrrb, and Klf4. For each set, the position of the SE (red) and associated gene (black) are indicated beneath the set. The x-axis represents genomic position and ChIP-seq signal enrichment is displayed along the y-axis as reads per million per base pair (rpm/bp). (FIG. 12B) Representative images of Co-localization between BRD4 or MED1 and nascent RNAs of SE-associated genes mir290, Esrrb, or Klf4 by immunofluorescence (IF) and fluorescent in situ hybridization (FISH) in fixed mESC, as indicated. Samples were imaged using spinning disk confocal microscopy. A single z-slice (500 nm) is presented individually for indicated IF and FISH and then as a merge of the two channels (overlap in white). The blue line highlights the nuclear periphery as designated by DAPI staining (not shown). The region of IF and FISH co-localization is highlighted by a yellow box in the “Merge” column and blown-up in the “Merge (zoom)” column to display detail. Scale bar, 5 μm for IF, FISH and Merge and 0.5 μm for Merge (zoom).

FIGS. 13A-13F—show BRD4 and MED1 condensates exhibit liquid-like FRAP kinetics. (FIG. 13A) Representative images of a BRD4-GFP-expressing mESC before and at indicated times after photobleaching of a BRD4-GFP condensate. The yellow box highlights the region being photobleached. The blue box highlights a control region for comparison. Time relative to photobleaching (0″) is indicated in the lower left of each image. Scale bars, 5 μm. (FIG. 13B) Time-lapse, close-up view of regions shown in (A). The photobleached region from panel A (yellow box in panel A) is shown on the top row. Times relative to photobleaching are shown above each view. The control region from panel A (blue box in panel A) is shown on the bottom row. Scale bar, 1 μm. (FIG. 13C) Recovery of fluorescence quantified and averaged. Signal intensity relative to time prior to photobleaching is shown on the y-axis. Time relative to photobleaching is shown on the x-axis. Data are shown for untreated cells (black) and for cells treated with oligomycin to deplete ATP (ATP-depleted, red). Data are shown as average relative intensity±SEM with n=9 for untreated cells and n=3 for ATP-depleted cells. (FIG. 13D) Same as (A), but with MED1-GFP expressing mESCs. Scale bar, 5 μm. (FIG. 13E) Same as (B), but with MED1-GFP expressing mESCs. Scale bar, 1 μm. (FIG. 13F) Same as (FIG. 13C), but with MED1-GFP expressing mESCs. Data are shown as average relative intensity±SEM with n=5 for untreated cells and n=5 for ATP-depleted cells.

FIGS. 14A-14F—show intrinsically disordered regions (IDRs) of BRD4 and MED1 phase separate in vitro. (FIG. 14A) Graphs plotting a score of intrinsic disorder (PONDR VSL2) for stretches of amino acids in BRD4 (top graph) and MED1 (bottom graph). PONDR VSL2 score is shown on the y-axis. Amino acid position is shown on the x-axis. Purple bar indicates intrinsically disordered C-terminal domain of each protein. Amino acid positions of the start and end of each intrinsically disordered domain are noted. (FIG. 14B) Schematic of recombinant GFP fusion proteins used in This manuscript. Purple boxes indicate intrinsically disordered domains of BRD4 (BRD4-IDR) and MED1 (MED1-IDR) that were shown in (FIG. 14C). Visualization of increase in turbidity associated with droplet formation. Tubes containing BRD4-IDR (left pair), MED1-IDR (middle pair) or GFP (right pair) are shown. For each pair, the presence (+) or absence (−) of PEG-8000 (a molecular crowding agent) in the buffer is shown. Blank tubes are included between pairs for contrast. (FIG. 14D) Representative images of droplet formation at different protein concentrations. BRD4-IDR (top row), MED1-IDR (middle row) or GFP (bottom row) were added to droplet formation buffer to a final concentration as indicated. Solutions were loaded onto a homemade chamber and imaged by spinning disk confocal microscopy, focused on the glass coverslip. Scale bar, 5 μm. (FIG. 14E) Representative images of droplet formation at different salt concentrations. BRD4-IDR (top row of images) or MED1-IDR (bottom row of images) was added to droplet formation buffer to achieve 10 μM concentration with a final NaCl concentration of 50 mM, 125 mM, 200 mM or 350 mM as indicated. Droplets were visualized as in (FIG. 14D). Scale bar, 5 μm. (FIG. 14F) Representative images of droplet reversibility experiment. The top row shows droplets of BRD4-IDR that were allowed to form in droplet formation buffer (20 μM protein, 75 mM NaCl) and then subjected to dilution or dilution plus changes in salt concentration. The left column shows representative droplets from the one third of the original volume. The middle column shows droplets representative of a second third of the volume that was diluted 1:1 with an isotonic solution. The right column shows droplets representative of the final third of the volume that was diluted 1:1 with high salt solution to a final concentration of 425 mM NaCl. Droplets were visualized as in (FIG. 14D). Scale bar, 5 μm.

FIGS. 15A-15H—show that the IDR of MED1 participates in phase separation in cells. (FIG. 15A) Schematic of optoIDR assay, depicting recombinant protein with a selected intrinsically disordered domain (purple), mCherry (red) and Cry2 (orange) expressed in cells that are then exposed to blue light. (FIG. 15B) Representative images of NIH3T3 cells expressing mCherry-Cry2 recombinant protein and subjected to 488 nm laser excitation every 2 seconds for 0 (left panel) or 200 seconds (right panel). Scale bar, 10 μm. (FIG. 15C) Representative images of NIH3T3 cells expressing a portion of the MED1 IDR (amino acids 948-1157 of MED1) fused to mCherry-Cry2 (MED1-optoIDR) and subjected to 488 nm laser excitation every 2 seconds for 0 (left panel), 60 seconds (middle panel) or 200 seconds (right panel). 10 μm. (FIG. 15D) Time-lapse images focusing on the nucleus of an NIH3T3 cell expressing MED1-optoIDR subjected to 488 nm laser excitation every 2 seconds for the indicated times. Scale bar, 5 μm. Yellow box highlights one of several regions where fusion events occur. (FIG. 15E) Time-lapse and close-up view of droplet fusion. Region of image highlighted by the yellow box in panel D is shown for extended time frames. Frames are taken at the times indicated in the lower left corner of each frame. Scale bar, 1 μm. (FIG. 15F) Representative images of a MED1-optoIDR optoDroplets before (left panel), during (middle panel) and after (right panel) photobleaching of an optoDroplets in the absence of blue light excitation. The yellow box highlights the region being photobleached. The blue box highlights a control region for comparison. Time relative to photobleaching (0″) is indicated in the lower left of each image. Scale bar, 5 μm. (FIG. 15G) Recovery of fluorescence quantified and averaged. Signal intensity relative to time prior to photobleaching is shown on the y-axis. Time relative to photobleaching is shown on the x-axis. Data are shown as average relative intensity±SD with n=15. (FIG. 15H) Time-lapse and close-up view of droplet recovery shown for regions highlighted in (FIG. 15F). Times relative to photobleaching are shown above views. Scale bar, 1 μm.

FIGS. 16A-16C—show visualizations of BRD4 and MED1 nuclear condensates. (FIG. 16A) ChIP-seq binding profiles for BRD4 and MED1 as indicated, at two loci. For each panel, chromosome coordinates are indicated at the bottom and a scale bar is included in the upper left. X-axes represents genomic position and ChIP-seq signal enrichment is displayed along the y-axis as reads per million (rpm). (FIG. 16B) Heat map showing occupancy of BRD4 (left panel) and MED1 (right panel) at BRD4- or MED1-bound sites in mESCs. Each panel shows the 4 kb window, centered on the peak of BRD4- or MED-1 bound regions, for each BRD4- or MED1-bound region (rows). Red indicates presence of ChIP-seq signal. Black indicates background. (FIG. 16C) Detection by immunofluorescence with secondary IgG antibody in mouse embryonic stem cells (mESCs) using structured illumination microscopy (SIM). Staining with IgG (left panel), DAPI (middle panel) and a merged view (right panel) are shown. Scale bar, 5 μm.

FIG. 17A-17D—show BRD4 and MED1 condensates occur at sites of super-enhancer-associated transcription. (FIG. 17A) ChIP-seq binding profiles for BRD4, MED1, and RNA polymerase II (RNAPII), as indicated, shown at the Nanog locus. X-axes represents genomic position and ChIP-seq signal enrichment is displayed along the y-axis as reads per million per base pair (rpm/bp). (FIG. 17B) Representative image of co-localization between BRD4 or MED1 and nascent RNAs of SE-associated gene Nanog by immunofluorescence (IF) and fluorescent in situ hybridization (FISH) in fixed mESC, as indicated. Samples were imaged using spinning disk confocal microscopy. The top row represents a comparison for BRD4. The bottom row represents a comparison for MED1. For each row, a single z-slice (500 nm) is presented individually for IF (left panel) and FISH (middle panel) and then as a merge of the two channels (right panel). The blue line highlights the nuclear periphery as designated by DAPI staining (not shown). The region of IF and FISH co-localization is highlighted by a yellow box and a close-up view of the highlighted region is shown in the far right panel. Scale bar, 5 μm for IF, FISH and Merge and 0.5 μm for Merge (zoom). (FIG. 17C) Schematic for quantitation of distance between IF and FISH foci. For the nearest focus analysis (top panel), the distance between the FISH signal and the nearest IF feature was selected. For the stochastic focus analysis (bottom panel), the distance between the FISH signal and a random IF feature within a 5 μm radius was selected. (FIG. 17D) Boxplots of the distances between IF foci for BRD4 (top row) or MED1 (bottom row) to the FISH signal for nearest or stochastic as defined in (FIG. 17C) for the genes indicated at the top of each set of boxplots. In the upper left of each set, the p-value (t-test) comparing nearest and stochas-tic, the number of RNA-FISH foci analyzed, and the number of independent replicates is reported.

FIGS. 18A-18C—show BRD4 and MED1 condensates exhibit liquid-like FRAP kinetics. (FIG. 18A) Table showing the half-life of recovery from photobleaching (T half) and the apparent diffusion rate for BRD4 and MED1 in these studies. For comparison, previously published information on DDX4 and NICD are shown. (FIG. 18B) Recovery of fluorescence quantified and averaged. Signal intensity relative to time prior to photobleaching is shown on the y-axis. Time relative to photobleaching is shown on the x-axis. Data are shown for BRD-GFP-expressing (blue) and MED1-GFP-expressing (red) cells treated with PFA to fix the cells and restrict diffusion of proteins post-photo-bleaching. Data are shown as average relative intensity±SEM. (FIG. 18C) Quantitation of ATP depletion as a function of glucose depletion and treatment with oligomycin.

FIGS. 19A-19D—show intrinsically disordered regions (IDRs) of BRD4 and MED1 phase separate in vitro. (FIG. 19A) Box plots showing the distribution of aspect ratios for droplets of BRD4-IDR and MED1-IDR. The number of droplets examined and the mean aspect ratio are shown. Box plot represents 10-90th percentile. (FIG. 19B) Dot plot showing relationship between protein concentration and droplet size for BRD4-IDR (left panel) or MED1-IDR (right panel). Protein concentration (μM) is shown on the x-axis and droplet size as a function of area in a 2-D image is shown on the y-axis. (FIG. 19C) Image showing the presence of small droplets at low protein concentrations. (FIG. 19D) Dot plot showing relationship between salt concentration and droplet size for BRD4-IDR (left panel) or MED1-IDR (right panel). Salt concentration (mM) is shown on the x-axis and droplet size as a function of area in a 2-D image is shown on the y-axis.

FIG. 20 shows OCT4 and Mediator occupy super-enhancers in vivo. ChIP-seq tracks of OCT4 and MED1 in ESCs at SEs (left column) and OCT4 IF with concurrent RNA-FISH demonstrating occupancy of OCT4 at Esrrb, Nanog, Trim28 and Mir290. Hoechst staining was used to determine the nuclear periphery, highlighted with a blue line. The two rightmost columns show average RNA FISH signal and average OCT4 IF signal centered on the RNA-FISH focus from at least 11 images. Average OCT4 IF signal at random randomly selected nuclear position is displayed in FIG. 27.

FIGS. 21A-21I show MED1 condensates are dependent on OCT4 binding in vivo. (FIG. 21A) Schematic of OCT4 degradation. The C-terminus of OCT4 is endogenously biallelically tagged with the FKBP protein; when exposed to the small molecule dTag, OCT4 is ubiquitylated and rapidly degraded. (FIG. 21B) Box plot representation of log 2 fold change in OCT4 and MED1 ChIP-seq reads and RNA-seq reads of Super-enhancer (SE)- or Typical enhancer (TE)-driven genes, in ESCs carrying the OCT4 FKBP tag, treated with DMSO or dTAG for 24 hours. (FIG. 21C) Genome browser view of OCT4 (green) and MED1 (yellow) ChIP-seq data at the Nanog locus. The Nanog SE (red) show a 90% reduction of OCT4 and MED1 binding after OCT4 degradation. (FIG. 21D) Normalized RNA-seq read counts of Nanog mRNA show a 60% reduction upon OCT4 degradation. (FIG. 21E) Confocal microscopy images OCT4 and MED1 IF with DNA FISH to the Nanog locus in ESCs carrying the OCT4 FKBP tag, treated with DMSO or dTAG. Inset represent a zoomed in view of the yellow box. The Merge view displays all three channels (OCT4 IF, MED1 IF and Nanog DNA FISH) together. (FIG. 21F) OCT4 ChIP-qPCR to the Mir290 SE in ESCs and differentiated cells (Diff). Presented as enrichment over control, relative to signal in ESCs. Error bars represents standard error of the mean from two biological replicates. (FIG. 21G) MED1 ChIP-qPCR to the Mir290 SE in ESCs and differentiated cells (Diff). Presented as enrichment over control, relative to signal in ESCs. Error bars represents the SEM from two biological replicates. (FIG. 21H) Normalized RNA-seq read counts of Mir290 miRNA in ESCs or differentiated cells (Diff). Error bars represents the SEM from two biological replicates. (FIG. 21I) Confocal microscopy images of MED1 IF and DNA FISH to the Mir290 genomic locus in ESCs and differentiated cells. Merge (zoom) represent a zoomed in view of the yellow box in the merged channel.

FIGS. 22A-22E show OCT4 forms liquid droplets with MED1 in vitro. (FIG. 22A) Graph of intrinsic disorder of OCT4 as calculated by the VSL2 algorithm (www.pondr.com). The DNA binding domain (DBD) and activation domains (ADs) are indicated above the disorder score graph (Brehm et al., 1997). (FIG. 22B) Representative images of droplet formation of OCT4-GFP (top row) and MED1-IDR-GFP (bottom row) at the indicated concentration in droplet formation buffer with 125 mM NaCl and 10% PEG-8000. (FIG. 22C) Representative images of droplet formation of MED1-IDR-mCherry mixed with GFP or OCT4-GFP at 10 uM each in droplet formation buffer with 125 mM NaCl and 10% PEG-8000. (FIG. 22D) FRAP of heterotypic droplets of OCT4-GFP and MED1-IDR-mCherry. Confocal images were taken at indicated time points relative to photobleaching (0). (FIG. 22E) Representative images of droplet formation of 10 uM MED1-IDR-mCherry and OCT4-GFP in droplet formation buffer with varying concentrations of salt and 10% PEG-8000.

FIG. 23A-23E show OCT4 phase separation with MED1 is dependent on specific interactions. (FIG. 23A) Amino acid enrichment analysis ordered by frequency of amino acid in the ADs (upper panel). Net charge per amino acid residue analysis of OCT4 (lower panel). (FIG. 23B) Representative images of droplet formation showing that Poly-E peptides are incorporated into MED1-IDR droplets. MED1-GFP and a TMR labeled proline or glutamic acid decapeptide (Poly-P and Poly-E respectively) were added to droplet formation buffers at 10 uM each with 125 mM NaCl and 10% PEG-8000. (FIG. 23C) (Upper panel) Schematic of OCT4 protein, horizontal lines in the AD mark acidic D residues (blue) and acidic E residues (red). All 17 acidic residues in the N-AD and 6 acidic residues in the C-AD were mutated to alanine to generate an OCT4-acidic mutant. (Lower panel) Representative confocal images of droplet formation showing that the OCT4 acidic mutant has an attenuated ability to concentrate into MED1-IDR droplets. 10 uM of MED1-IDR-mCherry and OCT4-GFP or OCT4-acidic mutant-GFP were added to droplet formation buffers with 125 mM NaCl and 10% PEG-8000. (FIG. 23D) (Upper panel) Representative images of droplet formation showing that OCT4 but not the OCT4 acidic mutant is incorporated into Mediator complex droplets. Purified Mediator complex was mixed with 10 uM GFP, OCT4-GFP or OCT4-acidic mutant-GFP in droplet formation buffers with 140 mM NaCl and 10% PEG-8000. (Lower panel) Enrichment ratio of GFP, OCT4-GFP or OCT4-acidic mutant-GFP in Mediator complex droplets. N>20, error bars represent the distribution between the 10th and 90th percentiles. (FIG. 23E) (Top panel) GAL4 activation assay schematic. The GAL4 luciferase reporter plasmid was transfected into mouse ES cells with an expression vector for the GAL4-DBD fusion protein. (Bottom panel) The AD activity was measured by luciferase activity of mouse ES cells transfected with GAL4-DBD, GAL-OCT4-CAD or GAL-OCT4-CAD-acidic mutant.

FIGS. 24A-24C show multiple TFs phase separate with Mediator droplets. (FIG. 24A) (Left graph) Percent disorder of various protein classes (x axis) plotted against the cumulative fraction of disordered proteins of that class (y axis). (Right graph) Disorder content of transcription factor (TF) DNA-binding domains (DBD) and putative activation domains (ADs). (FIG. 24B) Representative images of droplet formation assaying homotypic droplet formation of indicated TFs. Recombinant MYC-GFP (12 uM), p53-GFP (40 uM), NANOG-GFP (10 uM), SOX2-GFP (40 uM), RARa-GFP (40 uM), GATA-2-GFP (40 uM), and ER-GFP (40 uM) was added to droplet formation buffers with 125 mM NaCl and 10% PEG-8000. (FIG. 24C) Representative images of droplet formation showing that all tested TFs were incorporated into MED1-IDR droplets. 10 uM of MED1-IDRmCherry and 10 uM of either MYC-GFP, p53-GFP, NANOG-GFP, SOX2-GFP, RARa-GFP, GATA-2-GFP, or ER-GFP was added to droplet formation buffers with 125 mM NaCl and 10% PEG-8000.

FIGS. 25A-25E show Estrogen stimulates phase separation of the Estrogen Receptor with MED1. (FIG. 25A) Schematic of estrogen stimulated gene activation. Estrogen facilitates the interaction of ER with Mediator and RNAPII by binding the ligand binding domain (LBD) of ER, which exposes a binding pocket for LXXLL motifs within the MED1-IDR. (FIG. 25B) Schematic view of the MED1-IDRXL, and MED1-IDR used for recombinant protein production. (FIG. 25C) Representative images of droplet formation, assaying homotypic droplet formation of ER-GFP and MED1-IDRXL-mCherry. Performed with the indicated protein concentration in droplet formation buffers with 125 mM NaCl and 10% PEG-8000. (FIG. 25D) Representative confocal images of droplet formation showing that ER is incorporated into MED1-IDRXL droplets and the addition of estrogen considerably enhanced heterotypic droplet formation. ER-GFP, ER-GFP in the presence of estrogen, or GFP is mixed with MED1-IDRXL. 10 uM of each indicated protein was added to droplet formation buffers with 125 mM NaCl and 10% PEG-8000. (FIG. 25E) Enrichment ratio in MED1-IDRXL droplets of ER-GFP, ER-GFP in the presence of estrogen, or GFP. N>20, error bars represent the distribution between the 10th and 90th percentiles.

FIGS. 26A-26G show TF-Coactivator phase separation is dependent on residues required for transactivation. (FIG. 26A) Representative confocal images of droplet formation of GCN4-GFP or MED15-mCherry were added to droplet formation buffers with 125 mM NaCl and 10% PEG-8000. (FIG. 26B) Representative images of droplet formation showing that GCN4 forms droplets with MED15. GCN4-GFP and mCherry or GCN4-GFP and MED15-mCherry were added to droplet formation buffers at 10 uM with 125 mM NaCl and 10% PEG-8000 and imaged on a fluorescent microscope with the indicated filters. (FIG. 26C) (Top row) Schematic of GCN4 protein composed of an activation domain (AD) and DNA-binding domain (DBD). Aromatic residues in the hydrophobic patches of the AD are marked by blue lines. All 11 aromatic residues in the hydrophobic patches were mutated to alanine (A) to generate an GCN4-aromatic mutant. (Bottom row) Representative images of droplet formation showing that the ability of GCN4 aromatic mutant to form droplets with MED15 is attenuated. GCN4-GFP or GCN4-Aromatic-mutant-GFP and MED15-mCherry were added to droplet formation at 10 uM each with 125 mM NaCl and 10% PEG-8000. (FIG. 26D) (Upper panel) Representative images of droplet formation showing that GCN4 wild type but not GCN4 aromatic mutant are incorporated into Mediator complex droplets. 10 uM of GCN4-GFP or GCN4-Aromatic-mutant-GFP was mixed with purified Mediator complex in droplet formation buffer with 125 mM NaCl and 10% PEG-8000. (FIG. 26E) (Left panel) Schematic of the Lac assay. A U2OS cell bearing 50,000 repeats of the Lac operon is transfected with a Lac binding domain-CFP-AD fusion protein. (Right panel) IF of MED1 in Lac-U2OS cells transfected with the indicated Lac binding protein construct. (FIG. 26F) GAL4 activation assay. Transcriptional output as measured by luciferase activity in 293T cells, of the indicated activation domain fused to the GAL4 DBD. (FIG. 26G) Model showing transcription factors and coactivators forming phase-separated condensates at super-enhancers to drive gene activation. In this model, transcriptional condensates incorporate both dynamic and structured interactions.

FIG. 27 shows a random focus analysis. Average fluorescence centered at the indicated RNA FISH focus (top panels) versus a randomly distributed IF foci+/−1.5 microns in X and Y (bottom panels). Color scale bars present arbitrary units of fluorescence intensity.

FIGS. 28A-28F show OCT4 degradation and ES cell differentiation. (FIG. 28A) Schematic of the Oct4-FKBP cell-engineering strategy. V6.5 mouse ES cells were transfected with a repair vector and Cas9 expressing plasmid to generate knock-in loci with either BFP or RFP for selection (Left). WT or untreated OCT4-dTAG ES cells blotted for OCT4 showing expected shift in size, HA (on FKBP), and ACTIN (Right). (FIG. 28B) Western blot against OCT4 (left panels), MED1 (right panels), and BETA-ACTIN in the OCT4 degron line (dTAG), either treated with dTag47 or vehicle (DMSO). (FIG. 28C) Mean intensity of the MED1 immunofluorescence signal within the Nanog DNA FISH focus in DMSO treated, vs dTAG treated OCT4-degron cells. N=5 images, error bars are distribution between the 10th and 90th percentile. (FIG. 28D) Schematic showing the position of primers used for OCT4 (P1) and MED1 (P2) ChIP-qPCR in differentiated and ES cells at the MiR290 locus. (FIG. 28E) Western blot against MED1 and BETA-ACTIN in ES cells or cells differentiated by LIF withdrawal. (FIG. 28F) Mean intensity of MED1 immunofluorescence signal within MiR290 DNA FISH focus in ES cells versus cells differentiated by LIF withdrawal. N=5 images, error bars are distribution between the 10th and 90th percentile.

FIGS. 29A-29F show MED1 and OCT4 droplet formation. (FIG. 29A) Enrichment ratio of OCT4-GFP versus GFP in MED1-IDR-mCherry droplets formed in droplet formation buffer with 10% PEG-8000 at 125 mM NaCl. N>20, error bars represent the distribution between the 10^{th} and 90^{th} percentile. (FIG. 29B) Area in micrometers-squared of MED1-IDR-OCT4 droplets formed in 10% PEG-8000 at 125 mM salt with 10 uM of each protein. (FIG. 29C) Aspect ratio of MED1-IDR-OCT4 droplets formed in 10% PEG-8000 at 125 mM with 10 uM of each protein. N>20, error bars represent the distribution between the 10^{th} and 90^{th} percentile. (FIG. 29D) Area in micrometers-squared of MED1-IDR-OCT4 droplets formed in 10% PEG-8000 at 125 mM, 225 uM, or 300 uM salt, with 10 uM of each protein. (FIG. 29E) Fluorescence microscopy of droplet formation without crowding agents at 50 mM NaCl for the indicated protein or combination of proteins (at 10 uM each), imaged in the channel indicated at the top of the panel. (FIG. 29F) Enrichment ratio of OCT4-GFP versus GFP in MED1-IDR-mCherry droplets formed in droplet formation buffer without crowding agent at 50 mM NaCl. N>20, error bars represent the distribution between the 10^{th} and 90^{th} percentile.

FIGS. 30A-30E show phase separation of mutant OCT4. (FIG. 30A) Fluorescent microscopy of the indicated TMR-labeled polypeptide, at the indicated concentration in droplet formation buffers with 10% PEG-8000 and 125 mM NaCl. (FIG. 30B) Enrichment ratios of the indicated polypeptide within MED1-IDR-mCherry droplets. N>20, error bars represent the distribution between the 10^{th} and 90^{th} percentile. (FIG. 30C) Enrichment ratios of the indicated protein within MED1-IDR-mCherry droplets. N>20, error bars represent the distribution between the 10^{th} and 90^{th} percentile. (FIG. 30D) (Upper panel) Schematic of OCT4 protein, aromatic residues in the activation domains (ADs) are marked by blue horizontal lines. All 9 aromatic residues in the N-terminal Activation Domain (N-AD) and 10 aromatic residues in the C-terminal Activation Domain (C-AD) were mutated to alanine to generate an OCT4-aromatic mutant. (Lower panel) Representative confocal images of droplet formation showing that the OCT4 aromatic mutant is still incorporated into MED1-IDR droplets. MED1-IDR-mCherry and OCT4-GFP or MED1-IDR-mCherry and OCT4-aromatic mutant-GFP were added to droplet formation buffers with 125 mM NaCl at 10 uM each with 10% PEG-8000 and visualized on a fluorescent microscope with the indicated filters. (FIG. 30E) Droplets of intact Mediator complex were collected by pelleting and equal volumes of input, supernatant, and pellet were run on an SDS-PAGE gel and stained with sypro ruby. Mediator subunits present in the pellet are annotated on the rightmost column.

FIGS. 31A-31B show diverse TFs phase separate with Mediator. (FIG. 31A) Enrichment ratios of the indicated GFP-fused TF in MED1-IDR-mCherry droplets. N>20, error bars represent the distribution between the 10^{th} and 90^{th} percentile. (FIG. 31B) FRAP of heterotypic p53-GFP/MED1-IDR-mCherry droplets formed in droplet formation buffers with 10% PEG-8000 and 125 mM NaCL, imaged every second over 30 seconds.

FIG. 32A shows Estrogen receptor phase separates with MED1. Enrichment ratio of ER-GFP in MED1-IDR-mCherry droplets in the presence or absence of 10 uM estrogen. Droplets were formed in 10% PEG-8000 with 125 mM NaCl. N>20, error bars represent the distribution between the 10^{th} and 90^{th} percentile.

FIGS. 33A-33G show GCN4 and MED15 form phase separated droplets. (FIG. 33A) Enrichment ratio of mCherry or MED15-mCherry in GCN4-GFP droplets, in droplet formation buffer with 10% PEG-8000 and 125 mM NaCl. N>20, error bars represent the distribution between the 10^{th} and 90^{th} percentile. (FIG. 33B) FRAP of heterotypic GCN4-GFP/MED15-IDR-mCherry droplets formed in droplet formation buffers with 10% PEG-8000 and 125 mM NaCl, imaged every second over 30 seconds. (FIG. 33C) Phase diagram of GCN4-GFP and MED15-mCherry added at the indicated concentrations to droplet formation buffers with 10% PEG-8000 and 125 mM salt. (FIG. 33D) Enrichment ratio of GCN4 droplets from FIG. 33C. N>20, error bars represent the distribution between the 10^{th} and 90^{th} percentile. (FIG. 33E) Fluorescent imaging of GCN4-GFP or the aromatic mutant of GCN4-GFP at the indicated concentration in 10% PEG-8000 and 125 mM NaCl. Shown are images from GFP channel. (FIG. 33F) Enrichment ratio of GCN4-GFP or the aromatic mutant of GCN4-GFP in MED15-mCherry droplets, formed in droplet formation buffer with 10% PEG-8000 and 125 mM salt. N>20, error bars represent the distribution between the 10^{th} and 90^{th} percentile. (FIG. 33G) Enrichment ratio of GFP, GCN4-GFP or GCN4-aromatic mutant-GFP in Mediator complex droplets. N>20, error bars represent the distribution between the 10th and 90th percentiles.

FIG. 34 shows tamoxifen inhibits ER mediated gene activation and phase separation of ER and MED1. Top left shows that Tamoxifen binds to the ligand binding domain (LBD) of estrogen receptor (ER). Bottom right shows that in a GAL4 transactivation assay, transcriptional output of ER mediated gene activation is dependent upon estrogen and is blocked by tamoxifen. Left side are confocal microscopy images of GFP labeled ER and mCherry labeled MED1-IDR containing the LXXL binding pocket (MED1-IDRXL) form condensates in the presence of estrogen, but this estrogen dependent condensate formation is blocked by tamoxifen.

FIG. 35 shows that ER is known to establish super-enhancers upon estrogen stimulation and that MED1 is overexpressed in ER+ breast cancer (top right graph). MED1 is required for ER function and ER+ breast cancer oncogenesis.

FIG. 36 shows that ligand bound NHRs (Nuclear Hormone Receptors (e.g., nuclear receptors)) establish transcriptional condensates (TCs) at inducible super-enhancers. Alteration of these TCs is a mechanism of oncogenesis. Evolving oncogenic condensates is a mechanism by which cells develop drug resistance in cancer and existing anti-neoplastic drugs may target oncogenic transcriptional condensates. In view of this, TCs are a rational target for oncogenic-transcription-factor-mediated disease.

FIG. 37 shows confocal microscopy images of ER condensates (left column-green), MED1-IDRXL condensates (middle column-red), and MED1-IDRXL/ER condensates (right column-orange). Bottom right panel shows that estrogen (10 uM) stimulates ER incorporation into MED1-IDRXL condensates. This incorporation is dependent upon the presence of the LXXL pocket in the MED-IDR.

FIG. 38 shows confocal microscopy images of ER condensates (left column-green), MED1-IDRXL condensates (middle column-red), and MED1-IDRXL/ER condensates (right column-orange). Middle right panel shows that estrogen stimulates ER incorporation into MED1-IDRXL condensates. Bottom right panel shows that tamoxifen (100 uM) attenuates ER incorporation into MED1-IDRXL condensates in the presence of estrogen (10 uM).

FIG. 39 shows wild-type Estrogen Receptor LBD-mediated Med1 condensation and gene activation are stimulated by Estrogen and attenuated by Tamoxifen. A Lac binding domain-CFP-ER activation domain fusion protein was introduced into a U2OS cell bearing the Lac operon array. The upper set of confocal microscopy images show images of the CFP signal indicating the fusion protein and the lower set of panels shows immunofluorescence for Mediator. Introduction of 10 nM estrogen (+E) for 45 minutes increases LBD-mediated Med1 condensation, while introduction of 1 uM tamoxifen (+T) for 45 minutes attenuates LBD-mediated Med1 condensation. Bar graph at bottom shows transcriptional output as measured by luciferase activity of the indicated activation domain fused to the GAL4 DBD. Introduction of 10 nM estrogen (+E) increases reporter transcriptional output while introduction of 10 nM tamoxifen (+T) does not increase reporter transcriptional output. In the assay, cells were deprived of estrogen for 2 days and then treated with estrogen or tamoxifen for 24 hours.

FIG. 40 shows endocrine-resistant patient mutations are capable of both Estrogen-independent Med1 condensation and gene activation. A Lac binding domain-CFP-ER activation domain (ER) fusion protein, Lac binding domain-CFP-mutant (Y537S) ER activation domain fusion protein, or Lac binding domain-CFP-ER mutant (D538G) activation domain fusion protein was introduced into U2OS cells bearing the Lac operon array. The upper set of confocal microscopy images show CFP signal indicating the presence of fusion protein in the presence (E+) or absence (E−) of estrogen. Estrogen significantly increased condensate formation for the wild-type ER, but did not significantly affect condensate formation for either mutant. The lower set of confocal microscopy images show mediator immunofluorescence in the presence (E+) or absence (E−) of estrogen. Estrogen significantly increased condensate formation for the wild-type ER, but did not significantly affect condensate formation for either mutant. The bottom bar graph shows transcriptional output as measured by luciferase activity of the indicated activation domain fused to the GAL4 DBD in the presence (E+) or absence (E−) of estrogen. Estrogen caused a must larger increase in transcriptional output for the WT ER activation domain than either mutant. Same experimental conditions as FIG. 39.

FIG. 41 shows endocrine resistant ER patient mutations exhibit ligand-independent condensate formation. Top two rows of confocal microscopy images show MED1/ER condensate formation in the presence of estrogen. This condensate formation is attenuated by the further addition of tamoxifen. Bottom two rows show MED1/mutant ER (Y537S) condensate formation is unaffected by the addition of tamoxifen.

FIG. 42 shows estrogen stimulates MED1 condensate formation at the MYC oncogene. Top row of confocal microscopy images show that MED1 and Myc do not co-locate in the absence of estrogen. Bottom row of photomicrographs show MED1 condensate formation at MYC in the presence of estrogen.

FIG. 43A-43I shows MeCP2 and HP1α reside in liquid-like heterochromatin condensates. (FIG. 43A) Live-cell confocal microscopy of endogenous tagged MeCP2-GFP and Hoechst DNA staining in murine ESCs. (FIG. 43B) Live-cell confocal microscopy of endogenous tagged HP1α-mCherry and Hoechst DNA staining in murine ESCs. (FIG. 43C) Live-cell imaging of double-endogenous tagged MeCP2-GFP and HP1α-mCherry in murine ESCs. (FIG. 43D) Confocal microscopy images of FRAP experiments with endogenously tagged MeCP2-GFP murine ESCs. Post-bleach image shows recovery 12 seconds after photobleaching event. (FIG. 43E) Quantitation of FRAP data for MeCP2-GFP heterochromatin condensates. Photobleaching event occurs at t=0 s. Mean and standard error for 7 events are displayed. (FIG. 43F) Confocal microscopy images of FRAP experiments with endogenously tagged HP1α-mCherry murine ESCs. Post-bleach image shows recovery 12 seconds after photobleaching event. (FIG. 43G) Quantitation of FRAP data for HP1α-mCherry heterochromatin condensates. Photobleaching event occurs at t=0 s. Mean and standard error for 7 events are displayed. (FIG. 43H) Graph displays half-time of photobleaching recovery for MeCP2 and HP1α heterochromatin condensates. Mean and standard error for 7 events are displayed. (FIG. 43I) Graph displays mobile fractions of MeCP2 and HP1α within heterochromatin condensates. Mean and standard error for 7 events are displayed.

FIGS. 44A-44J shows MeCP2 form phase-separated liquid droplets in vitro. (FIG. 44A) Schematic of human MeCP2 protein. Structured methyl-binding domain (MBD) and intrinsically disordered regions (IDR-1 and IDR-2) are indicated. Predicted disorder score along the protein was computed using PONDR VSL2 algorithm. Net charge per residue was computed using a 5 amino acid sliding window. (FIG. 44B) Confocal microscopy of droplet formation assays with increasing concentrations of MeCP2-GFP. (FIG. 44C) Dot plot displaying the distribution of droplet areas over increasing concentrations of MeCP2-GFP. For each condition, 400 droplets were analyzed. (FIG. 44D) Bar plot displaying the condensed protein fraction of MeCP2-GFP in droplets over increasing protein concentration. Mean and standard deviation for 10 images are displayed. (FIG. 44E) Time lapse imaging of MeCP2-GFP droplet fusion in vitro. (FIG. 44F) Imaging of MeCP2-GFP droplet FRAP in vitro. (FIG. 44G) Confocal microscopy of droplet formation assays with MeCP2-GFP performed in the presence of increasing salt concentrations in droplet formation reactions. (FIG. 44H) Dot plot displaying the distribution of droplet areas over increasing concentrations of NaCl in droplet formation reactions. For each condition, 400 droplets were analyzed. (FIG. 44I) Bar plot displaying the condensed protein fraction of MeCP2-GFP in droplets over increasing salt concentrations. Mean and standard deviation for 10 images are displayed. (FIG. 44J) Phase diagram of MeCP2-GFP droplet formation as a function of protein and salt concentrations. Positive conditions are indicated by filled in circles.

FIGS. 45A-45E shows MeCP2 condensate formation depends upon the C-terminal IDR. (FIG. 45A) Schematic of MeCP2 protein indicating the MBD, IDR-1, IDR-2 and displaying the full length (FL) and two different truncation proteins used for in vitro droplet formation and live-cell imaging assays. Bar chart displaying the number of MECP2 coding mutations in female Rett syndrome patients found in RettBASE database for each amino acid position along MeCP2. Positions of nonsense, frameshift, and missense mutations are shown below with a schematic of MeCP2 protein domains. (FIG. 45B) Confocal microscopy of droplet formation assays with MeCP2-GFP full length (FL) and IDR truncation mutants (AIDR-1 and AIDR-2). (FIG. 45C) Live-cell confocal microscopy of three different endogenously tagged MeCP2-GFP lines made in murine ESCs. FL: full length MeCP2-GFP, AIDR-1: IDR-1 deletion, and AIDR-2: IDR-2 deletion. (FIG. 45D) Quantitation of MeCP2-GFP partition coefficient at heterochromatin bodies relative to nucleoplasm for different endogenously tagged lines. Mean and standard deviation for 10 cells are displayed. (FIG. 45E) RT-qPCR of major satellite repeat expression in murine ESCs with full length (FL), AIDR-1, and AIDR-2. Expression normalized to FL and Gapdh. Mean and standard deviation of 3 replicates are displayed.

FIGS. 46A-46D show MeCP2 condensates can compartmentalize heterochromatin factors. (FIG. 46A) Schematic of nuclear extract droplet formation assay. (FIG. 46B) Confocal microscopy images of nuclear extract droplet formation assays containing MeCP2-mCherry and MeCP2-AIDR-2-mCherry. Droplet formation was initiated by reducing the salt concentration of the extract to 150 mM NaCl. (FIG. 46C) Immunoblots for indicated proteins displaying relative protein amounts found in 10% of the input material and the pellet fraction of nuclear extract droplet formation assays after centrifugation at 2700×g. (FIG. 46D) Quantification of immunoblots in FIG. 46C. Bar chart shows for each protein examined the percent of input in each droplet formation reaction that was found in the pellet fraction.

FIGS. 47A-47D show MeCP2-IDR-2 partitions preferentially into heterochromatin condensates. (FIG. 47A) Cartoon of MeCP2 IDR partitioning experiment. Cells were transfected with expression constructs for mCherry-MeCP2-IDR-2 or mCherry alone. Ability to address to heterochromatin condensates was assessed by capacity to selectively partition into heterochromatin condensates relative to nucleoplasm. (FIG. 47B) Live-cell confocal microscopy images of murine ESCs with over-expression of MeCP2-IDR-2 or an mCherry control. Box indicates a heterochromatin condensate. (FIG. 47C) Additional zoom-in examples of heterochromatin condensates in murine ESCs with over-expression of MeCP2-IDR-2 or an mCherry control. Scale bar represents 1 (FIG. 47D) Quantitation of partition coefficients at heterochromatin condensates relative to nucleoplasm. Mean and standard deviation of 5 replicates are displayed.

FIGS. 48A-48F show MeCP2 is concentrated in heterochromatin of neurons of mouse brain. (FIG. 48A) Fixed-cell confocal microscopy of endogenously tagged MeCP2-GFP brain sections from high grade chimeric MeCP2-GFP mice. Immunostaining for MAP2 and PU.1 was used to identify neurons and microglia, respectively. Brain sections of 10 μm thickness were harvested from 2-month-old mice. (FIG. 48B) Quantitation of MeCP2-GFP condensate number per cell in neurons and microglia. Data are represented as mean±standard deviation of 3 cells. (FIG. 48C) Quantitation of MeCP2-GFP condensate number per cell in neurons and microglia. Data are represented as mean±standard deviation of 18 condensates for neurons and 28 condensates for microglia. (FIG. 48D) Live-cell confocal microscopy images of FRAP experiments performed on acute brain slices taken from 2-month-old, endogenously tagged MeCP2-GFP chimeric mice. Post-bleach image displays recovery 12 seconds after photobleaching event. (FIG. 48E) Quantitation of FRAP data for MeCP2-GFP heterochromatin condensates in live brain. Photobleaching event occurs at t=0 s. Mean and standard error for 3 events are displayed. (FIG. 48F) Fixed-cell confocal microscopy of endogenously tagged MED-GFP in brain sections from high grade chimeric MED1-GFP mice. Brain sections of 10 μm thickness were harvested from 2-month-old mice.

FIGS. 49A-49B show MeCP2-GFP and HP1α-mCherry condensate number and volume. (FIG. 49A) Quantification of MeCP2-GFP and HP1α-mCherry condensate number/cell. n=5 cells. (FIG. 49B) Quantification of MeCP2-GFP and HP1α-mCherry condensate volume. MeCP2, n=45 condensates.

FIGS. 50A-50D show MeCP2 forms phase-separated liquid droplets in vitro. (FIG. 50A) Expanded schematic of human MeCP2 protein with line plot showing evolutionary conservation of human MeCP2 protein sequence per residue chart display amino acid composition of MeCP2. Conservation was calculated as Jensen-Shannon divergence with higher values indicating greater sequence conservation. (FIG. 50B) Confocal microscopy image of droplet formation assay with 160 nM MeCP2-GFP. (FIG. 50C) Confocal microscopy image of droplet formation assay with 10 μM HP1α-mCherry. (FIG. 50D) Images for phase diagram of MeCP2-GFP droplet formation as a function of protein and salt concentrations.

FIG. 51 illustrates signaling factors and transcriptional condensate interactions in the nucleus.

FIGS. 52A-52D show signaling factors form signaling dependent condensates at super-enhancers in vivo. (FIG. 52A) Immunofluorescence for β-catenin, STAT3, SMAD3 and MED1 with concurrent RNA-FISH for Nanog nascent RNA demonstrating the presence of condensed nuclear foci of the signaling factors at the Nanog super-enhancer in mES cells. Cells were grown for 24 hours in the presence of CHIR99021, LIF and Activin A to activate the WNT, JAK/STAT and TGF-β signaling pathways respectively 24 hours prior to fixation. Hoechst staining was used to determine the nuclear periphery, highlighted with a dotted line. 100× objective was used for imaging on a spinning disk confocal microscope. Average RNA-FISH signal and average IF signal centered on the RNA-FISH focus for each signaling factor from at least 10 images is shown. Average signaling factor IF signal around randomly selected nuclear positions is displayed in the right most panel. Scale bars indicate 5 μm. (FIG. 52B) ChIP-seq tracks displaying occupancy of β-catenin, STAT3, SMAD3 and MED1 in mES at the super-enhancer associated with the Nanog gene. Reads densities are displayed in reads per million per bin (rpm/bin) and the super-enhancer is indicated with a red bar. (FIG. 52C) Immunofluorescence of mES cells for the signaling factors β-catenin, STAT3 and SMAD3 in unstimulated or stimulated conditions. Cells were stimulated for 24 hours with either CHIR99021, LIF, or Activin A to activate the WNT, JAK/STAT and TGF-β signaling pathways respectively 24 hours prior to fixation. Hoechst staining was used to determine the nuclear periphery, highlighted with a dotted line. 100× objective was used for imaging on a spinning disk confocal microscope. Scale bars indicate 5 μm. (FIG. 52D) Left: Representative images of FRAP experiment of mEGFP-β-catenin engineered HCT116 cells. Yellow box highlights the punctum undergoing targeted bleaching. Right: Quantification of FRAP data for mEGFP-β-catenin puncta. Bleaching event occurs at t=0 s. For both bleached area and unbleached control, background-subtracted fluorescence intensities are plotted relative to a pre-bleach time point (t=−4 s). Data are plotted as mean+/−SEM (N=9). Images were taken using the Zeiss LSM 880 confocal microscope with Airyscan detector with a 63× objective. Scale bar indicates 2 μm.

FIGS. 53A-53C show purified signaling factors can form condensates in vitro. (FIG. 53A) Domain structures of the signaling factors used in this manuscript. DBD: DNA binding domain, PID: protein interaction domain, CC: coiled coil domain, DD: dimerization domain, SH2: Src homology domain 2. The predicted intrinsically disordered regions (IDR) are indicated with red brackets. (FIG. 53B) Representative confocal images of concentration series of droplet formation assay testing homotypic droplet formation of mEGFP-β-catenin, mEGFP-STAT5 and mEGFP-SMAD3. mEGFP alone is included as a control (left panels). Quantification of the partition ratio for the signaling factors (right panels). Partition ratio was calculated by dividing the average fluorescence signal inside the droplets by the average fluorescence signal outside the droplets for at least 10 acquired images at all concentrations tested. All assays were performed in the presence of 125 mM NaCl and 10% PEG-8000 was used as a crowding agent. Scale bars indicate 2 μm. (FIG. 53C) Dilution droplet assay for the signaling factors. Initial droplets were formed at 1.2504 and imaged. The remaining reaction mixture was then diluted 2-fold with reaction buffer containing 4M NaCl to obtain a final salt concentration of 2M NaCl. Representative images of droplets before and after dilution are displayed.

FIGS. 54A-54D show purified signaling factors are incorporated into Mediator condensates in vitro. (FIG. 54A) Schematic representation of addition of signaling factor to pre-existing MED1-IDR droplets. mCherry-MED1-IDR droplets were formed and placed in a glass dish and imaged before and after addition of mEGFP-tagged signaling factors. (FIG. 54B) Representative images of signaling factor incorporation into MED-IDR droplets. Preformed mCherry-MED1-IDR droplets were imaged pre and post addition of mEGFP-tagged signaling factor solution for a total of 10 mins. Signaling factor was added 30 sec after imaging acquisition started. Last image displayed corresponds to the imaging end point. 10 μM of MED1-IDR-mCherry in the presence of PEG-8000 was used for droplet formation and 10 uM of either mEGFP-β-catenin, mEGFP-SMAD3 or mEGFP-STAT3 in the absence of PEG-8000 was added. Scale bars indicate 2 μm. (FIG. 54C) Partition ratio was calculated for pre-formed MED1-IDR-mCherry droplets that were mixed with dilute GFP-tagged signaling factor using the same conditions as in B. At least 10 images were used for quantification. Droplets were called on merged channels and signal intensity for the GFP-tagged factor in the area within the droplet compared to the intensity of the area outside of the droplet. Star indicates p-value obtained by a t-test <0.05. (FIG. 54D) Limited dilution droplet assay with near physiological concentrations of β-catenin, STAT3 and SMAD3. Indicated concentrations of the signaling factors were either added to droplet formation buffer alone (125 mM NaCL and 10% PEG-8000) or in combination with 10 μM MED1-IDR. Scale bars indicate 2 μm.

FIGS. 55A-55E show phase separation of β-catenin is dependent on aromatic amino acids. (FIG. 55A) Diagram of the different mEGFP-β-catenin truncated proteins that were tested. (FIG. 55B) Representative confocal images of a concentration series of droplet formation assays testing homotypic droplet formation for mEGFP-β-catenin, mEGFP-N-terminal-IDR, mEGFP-Armadillo and GFP-C-terminal-IDR. Droplet assays were performed in 125 mM NaCL and 10% PEG-8000. (FIG. 55C) Representative confocal images of concentration series of droplet formation assay testing homotypic droplet formation ability of wild type mEGFP-β-catenin, aromatic mutant mEGFP-β-catenin and mEGFP. Droplet assays were performed in 125 mM NaCl and 10% PEG-8000. Scale bar indicates 1 μm. Schematic of domain structure of wild type mEGFP-β-catenin and the aromatic to alanine mutant used in the described experiments shown above. (FIG. 55D) Representative confocal images of heterotypic droplet formation assays mixing 10 μM MED1-IDR-mCherry with 10 μM of wild type mEGFP-β-catenin or aromatic mutant mEGFP-β-catenin. Scale bar indicates 1 μm. (FIG. 55E) Partition ratio of factors was quantified for at least 10 images each. Droplets were called on merged channels and signal intensity for the factor in the area within the droplet compared to the intensity of the area outside the droplet.

FIGS. 56A-56C show that addressing of β-catenin and activation of target genes is dependent on aromatic amino acids. (FIG. 56A) Schematic of the ChIP experiment. TdTomato-tagged wild type or aromatic mutant β-catenin were stably integrated in mES cells under a doxycycline-inducible promoter. Doxycycline was added to the media 24 hours prior to crosslinking. ChIP was preformed using antibodies against TdTomato. TRE=Tetracycline responsive element. (FIG. 56B) (Top) ChIP-qPCR of ectopically-expressed wild type or aromatic mutant β-catenin at Myc, Sp5, and Klf4 enhancers. Error bars indicate standard deviation of three replicates. Stars indicate p-values obtained by a t-test <0.05. (Bottom) RT-qPCR of mRNA levels after ectopic expression of wild type or aromatic mutant β-catenin of Myc, Sp5, and Klf4. Error bars indicate standard deviation of three replicates. Stars indicate p-values obtained by a t-test <0.05. (FIG. 56C) Luciferase assay using a synthetic WNT-reporter containing 10 copies of the consensus TCF/LEF motif were wild type or aromatic mutant β-catenin was overexpressed in HEK293T cells. Average of 3 biological replicates is shown. Error bars show the standard deviation. Star indicates p-value obtained by a t-test <0.05.

FIGS. 57A-57E show β-catenin-condensate interaction can occur independent of TCF factors. (FIG. 57A) Immunofluorescence of β-catenin in Lac-U2OS cells transfected with a Lac binding domain-CFP or a Lac binding domain-CFP-MED1-IDR construct, imaged with a 100× objective on a spinning disk confocal microscope. Hoechst staining was used to determine the nuclear periphery, highlighted with a dotted line. Quantification shows the relative intensity of β-catenin in CFP foci. Scale bar indicates 5 μm. (FIG. 57B) IF of TCF4 in Lac-U2OS cells transfected with a Lac binding domain-CFP-MED1-IDR construct. Images were obtained using a 100× objective on a spinning disk confocal microscope. Scale bars indicate 5 μm. (FIG. 57C) Fluorescence imaging of overexpressed TdTomato-tagged wild type or aromatic mutant β-catenin in U2OS 2-6-3 cells co-transfected with a Lac binding domain-CFP or a Lac binding domain-CFP-MED1-IDR construct, imaged with a 100× objective on a spinning disk confocal microscope. Hoechst staining was used to determine the nuclear periphery, highlighted with a dotted line. Quantification shows the relative intensity of over-expressed β-catenin forms in called CFP foci. Scale bar indicates 5 μm. (FIG. 57D) ChIP-qPCR for β-catenin-GFP-chimera at the enhancers of SOX9, SMAD7, KLF9 or GATA3 in HEK293T cells. Error bars show the standard deviation of the mean. Stars indicate p-values obtained by a t-test <0.05. (FIG. 57E) Luciferase assay of cells over-expressing β-catenin-mEGFP-chimera in combination with a synthetic WNT-reporter containing 10 copies of the consensus TCF/LEF motif. Average of 3 biological replicates is shown. Error bars show the standard deviation. Stars indicate p-values obtained by a t-test <0.05.

FIGS. 58A-58D show show signaling factors form signaling dependent condensates at super-enhancers in vivo. (FIG. 58A) ChIP-seq tracks displaying occupancy of β-catenin, STAT3, SMAD3 and MED1 at the super-enhancer of the miR290 gene. Reads densities are displayed in reads per million per bin (rpm/bin) and the super-enhancer is indicated with a red bar. (FIG. 58B) Immunofluorescence for β-catenin, STAT3, SMAD3 and MED1 with concurrent RNA-FISH for miR290 nascent RNA demonstrating the presence of condensed nuclear foci of the signaling factors at the miR290 super-enhancer in mES cells. Cells were grown for 24 hours in the presence of CHIR99021, LIF or Activin A prior to fixation. Hoechst staining was used to determine the nuclear periphery, highlighted with a dotted line. 100× objective was used for imaging on a spinning disk confocal microscope. Average RNA-FISH signal and average IF signal centered on the RNA-FISH focus for each signaling factor from at least 10 images is shown. Average signaling factor IF signal at randomly selected nuclear positions is displayed in the right most panel. Scale bars indicate 5 μm. (FIG. 58C) Immunofluorescence for β-catenin with concurrent DNA-FISH for Nanog demonstrating the absence of nuclear foci of the signaling factors at the Nanog super-enhancer in C2C12 cells. Cells were grown for 24 hours in the presence of CHIR99021 prior to fixation. Hoechst staining was used to determine the nuclear periphery, highlighted with a dotted line. 100× objective was used for imaging on a spinning disk confocal microscope. Average DNA-FISH signal and average IF signal centered on the DNA-FISH focus for each signaling factor from at least 10 images is shown. Average signaling factor IF signal at randomly selected nuclear positions is displayed in the right most panel. Scale bar indicates 5 μm. (FIG. 58D) Western blot showing levels of endogenously tagged mEGFP-β-catenin in comparison to endogenous β-catenin in HCT116 cells.

FIG. 59 shows the domain structures of β-catenin, STAT3 and SMAD3. DBD: DNA binding domain, PID: protein interaction domain, CC: coiled coil domain, DD: dimerization domain, SH2: Src homology domain 2. The predicted intrinsically disordered regions (IDR) are marked in red. PONDR VL3 score per amino acid was used to predict disorder and is plotted below. Barcode plots indicate the location of different amino acids below. Red boxes indicate the top 3 over-represented amino acids in the predicted IDRs of the protein. Lowest panel shows the net charge per residue (NCPR) for the indicated protein.

FIG. 60A is a western blot showing expression levels of wild type and mutant β-catenin that were integrated in mES cells under a doxycycline inducible promoter. Cell were induced with 1 μg/ml doxycycline for 24 hours and FACS sorted for expression of the TdTomato-tagged β-catenin and individual colonies were picked and grown to generate clonal cell lines.

FIGS. 61A-61B show that addressing of β-catenin and activation of target genes is dependent on aromatic amino acids. (FIG. 61A) IF of HP1α in U20S2-6-3 cells transfected with a Lac binding domain-CFP-MED1-IDR construct. Images were obtained using a 100× objective on a spinning disk confocal microscope. Scale bars indicate 5 μm. (FIG. 61BB) Western blot showing the levels of wild type β-catenin or IDR-mEGFP-IDR chimera protein in HEK293T cells. Histone H3 was used as a loading control.

FIG. 62A-62F show that the CTD of Pol II is integrated and concentrated in Mediator condensates. (FIG. 62A) A model depicting the transition from transcription initiation to elongation and the role of Pol II CTD phosphorylation in this transition. During initiation, Pol II with a hypophosphorylated CTD interacts with Mediator. CDK7 phosphorylation of the CTD leads to formation of a paused Pol II approximately 50-100 bp downstream of the initiation site, and subsequent CDK9 phosphorylation leads to pause release and elongation. For simplicity, we show CDK7 and CDK9 phosphorylating the CTD, leading to elongation. During elongation, Pol II with phosphorylated CTD interacts with various RNA processing factors. (FIG. 62B) Representative images of droplet experiments showing recombinant full-length human CTD with 52 heptapeptide repeats fused to GFP (GFP-CTD52) is incorporated into human Mediator complex droplets. Purified human Mediator complex (˜200-300 nM; see methods) was mixed with 10 uM GFP or GFP-CTD52 in droplet formation buffers with 135 mM monovalent salt and 10% PEG-8000 or 16% Ficoll-400 and visualized on a fluorescence microscope with the indicated filters. (FIG. 62C) Representative images of droplet experiments showing GFP-CTD52 is incorporated into MED1-IDR droplets. Purified human MED1-IDR fused to mCherry (mCherry-MED1-IDR) at 10 uM was mixed with 3.3 uM GFP or GFP-CTD52 in droplet formation buffers with 125 mM NaCl and 10% PEG-8000 or 16% Ficoll-400 and visualized on a fluorescence microscope with the indicated filters. (FIG. 62D) The CTD is concentrated into MED1-IDR droplets depending on the CTD repeat length. GFP, GFP-CTD52, or GFP fused to CTD truncation mutants with 26 (GFP-CTD26) or 10 (GFP-CTD10) heptapeptide repeats at 10 uM were mixed with 10 uM mCherry-MED1-IDR in droplet formation buffers with 125 mM NaCl and 16% Ficoll-400 and visualized on a fluorescence microscope with the indicated filters. (FIG. 62E) Images of a fusion event between two full-length CTD/MED1-IDR droplets. Droplet formation condition is the same as in FIG. 62D. (FIG. 62F) FRAP of heterotypic droplets of GFP-CTD52 and MED1-IDR-mCherry. Droplet formation condition is the same as in FIG. 62D.

FIG. 63A-63D show phosphorylation of the CTD reduces CTD incorporation into MED1-IDR condensates in vitro. (FIG. 63A) Representative images showing CDK7-mediated CTD phosphorylation (see methods) causes loss of ability of CTD to be incorporated into MED1-IDR condensates. (Left) mCherry-MED1-IDR at 10 uM was mixed with 3.3 uM GFP, GFP-CTD52 or GFP-phospho-CTD52 in droplet formation buffers with 125 mM NaCl and 16% Ficoll-400 and visualized on a fluorescence microscope with the indicated filters. (Right) Enrichment ratio of GFP-CTD52 with or without CDK7-mediated phosphorylation in MED1-IDR droplets (see methods). Enrichment ratio of GFP is set to 1. The box in the boxplot extends from the 25th to 75th percentiles. The line in the middle of the box is plotted at the median. The whiskers go down to the smallest value and up to the largest value. The p-values are determined by a two-tailed Student's t-test. (FIG. 63B) Representative images showing CDK7-mediated CTD phosphorylation causes loss of ability of CTD to be incorporated into MED1-IDR condensates. (Left) mCherry-MED1-IDR at 10 uM was mixed with 3.3 uM GFP, GFP-CTD52 or GFP-phospho-CTD52 in droplet formation buffers with 125 mM NaCl and 10% PEG-8000 and visualized on a fluorescence microscope with the indicated filters. (Right) Enrichment ratio of GFP-CTD52 with or without CDK7-mediated phosphorylation in MED1-IDR droplets as displayed in 2a. (FIG. 63C) Representative images showing CDK9-mediated CTD phosphorylation (see methods) causes loss of ability of CTD to be incorporated into MED1-IDR condensates. (Left) mCherry-MED1-IDR at 10 uM was mixed with 10 uM GFP, GFP-CTD52 or GFP-phospho-CTD52 in droplet formation buffers with 125 mM NaCl and 16% Ficoll-400 and visualized on a fluorescence microscope with the indicated filters. (Right) Enrichment ratio of GFP-CTD52 with or without CDK9-mediated phosphorylation in MED1-IDR droplets as displayed in FIG. 63A. (FIG. 63D) Representative images showing CDK9-mediated CTD phosphorylation causes loss of ability of CTD to be incorporated into MED1-IDR condensates. (Left) mCherry-MED1-IDR at 10 uM was mixed with 10 uM GFP, GFP-CTD52 or GFP-phospho-CTD52 in droplet formation buffers with 125 mM NaCl and 10% PEG-8000 and visualized on a fluorescence microscope with the indicated filters. (Right) Enrichment ratio of GFP-CTD52 with or without CDK9-mediated phosphorylation in MED1-IDR droplets as displayed in FIG. 63A.

FIGS. 64A-64B show splicing condensates occur at active super-enhancer driven genes. (FIG. 64A) Representative immunofluorescence (IF) imaging of SRSF2 coupled to RNA FISH of nascent RNA of Nanog and Trim28 in fixed mouse embryonic stem cells (mESCs). The first two columns on the right show average RNA FISH signal and average splicing factor IF signal centered on RNA FISH foci (97 Nanog foci, 115 Trim28 foci were used). The rightmost column shows average IF signal for splicing factor centered on randomly selected nuclear positions (see methods). The positions of RNA FISH probes used for Nanog and Trim28 are illustrated on their respective gene models. (FIG. 64B) Representative IF imaging of splicing factors SRRM1 and SRSF1 coupled to RNA FISH of nascent RNA of Nanog and Trim28 in fixed mESCs. The first two columns on the right show average RNA FISH signal and average splicing factor IF signal centered on RNA FISH foci (for SRRM1,137 Nanog foci, 209 Trim28 foci were used; for SRSF1, 109 Nanog foci, 248 Trim28 foci were used). The rightmost column shows average IF signal for splicing factor centered on randomly selected nuclear positions.

FIGS. 65A-65F show phosphorylated CTD colocalizes with SRSF2 in mESCs and is incorporated and concentrated into SRSF2 droplets in vitro. (FIG. 65A) Representative ChIP-seq tracks of MED1, SRSF2 and two different phosphoforms of Pol II (unphosphorylated or serine 2 phosphorylated) in mESCs at Nanog and Trim28 loci. The y-axis represents reads per million. (FIG. 65B) Metagene plots of average ChIP-seq reads per million (RPM) for MED1, SRSF2 and two different phosphoforms of Pol II (unphosphorylated or serine 2 phosphorylated) across gene bodies from transcription start site (TSS) to transcription end site (TES) with 2 kb upstream of TSS and 2 kb downstream of TES at the top 20% most highly expressed genes. (FIG. 65C) Representative images of droplet experiments showing CTD is efficiently incorporated into SRSF2 droplets when the CTD is phosphorylated by CDK7. (Left) Purified human SRSF2 fused to mCherry (mCherry-SRSF2) at 2.4 uM was mixed with 3.3 uM GFP, GFP-CTD52 or GFP-phospho-CTD52 in droplet formation buffers with 100 mM NaCl and 16% Ficoll-400 and visualized on a fluorescence microscope with the indicated filters. (Right) Enrichment ratio of GFP-CTD52 with or without CDK7-mediated phosphorylation in SRSF2 droplets (see methods). Enrichment ratio of GFP is set to 1. The box in the boxplot extends from the 25th to 75th percentiles. The line in the middle of the box is plotted at the median. The whiskers go down to the smallest value and up to the largest value. The p-values are determined by a two-tailed Student's t-test. (FIG. 65D) Representative images of droplet experiments showing CTD is efficiently incorporated into SRSF2 droplets when the CTD is phosphorylated by CDK7. (Left) mCherry-SRSF2 at 2.4 uM was mixed with 3.3 uM GFP, GFP-CTD52 or GFP-phospho-CTD52 in droplet formation buffers with 100 mM NaCl and 10% PEG-8000 and visualized on a fluorescence microscope with the indicated filters. (Right) Enrichment ratio of GFP-CTD52 with or without CDK7-mediated phosphorylation in SRSF2 droplets as displayed in 4c. (FIG. 65E) Representative images of droplet experiments showing CTD is efficiently incorporated into SRSF2 droplets when the CTD is phosphorylated by CDK9. (Left) mCherry-SRSF2 at 2.4 uM was mixed with 10 uM GFP, GFP-CTD52 or GFP-phospho-CTD52 in droplet formation buffers with 120 mM NaCl and 16% Ficoll-400 and visualized on a fluorescence microscope with the indicated filters. (Right) Enrichment ratio of GFP-CTD52 with or without CDK9-mediated phosphorylation in SRSF2 droplets as displayed in FIG. 65C. (FIG. 65F) Representative images of droplet experiments showing CTD is efficiently incorporated into SRSF2 droplets when the CTD is phosphorylated by CDK9. (Left) mCherry-SRSF2 at 2.4 uM was mixed with 10 uM GFP, GFP-CTD52 or GFP-phospho-CTD52 in droplet formation buffers with 120 mM NaCl and 10% PEG-8000 and visualized on a fluorescence microscope with the indicated filters. (Right) Enrichment ratio of GFP-CTD52 with or without CDK9-mediated phosphorylation in SRSF2 droplets as displayed in FIG. 65C.

FIGS. 66A-66C show CDK7 and CDK9-mediated CTD phosphorylation in vitro, and loss of CTD incorporation into MED1-IDR droplets mediated by CDK7 is ATP dependent. (FIG. 66A) Western blot showing phosphorylation of GFP-CTD52 at Ser5 and Ser2 residues by CDK7. Equal amounts of GFP-CTD52 were used in each condition as shown by anti-GFP antibody. (FIG. 66B) Western blot showing phosphorylation of GFP-CTD52 at Ser5 and Ser2 residues by CDK9. Equal amounts of GFP-CTD52 were used in each condition as shown by anti-GFP antibody. (FIG. 66C) Representative images showing that loss of CTD incorporation into MED1-IDR droplets requires CDK7 and ATP. GFP-CTD52 at 10 uM, which has been incubated with recombinant CDK7 and/or ATP (see methods), was mixed with 10 uM mCherry-MED1-IDR in droplet formation buffers with 125 mM NaCl and 16% Ficoll-400 and visualized on a fluorescence microscope with the indicated filters.

FIGS. 67A-67C show SRSF2 is a phospho-CTD interacting factor, and enhanced CTD incorporation into SRSF2 droplets mediated by CDK7 is ATP dependent. (FIG. 67A) Histogram showing the average iBAQ (intensity-based absolute quantification) enrichment score from mass spectrometry for different Mediator subunits, SR family splicing factors, and components of the spliceosome enriched by pull-down using different phosphoforms of the CTD. Mediator subunits from different modules are shown. For the splicing factors, canonical SR proteins that are detected in Ebmeier et al., (Cell Rep 20, 1173-1186 (2017)) and spliceosome components that are thought to interact with Pol II are shown. Briefly, iBAQ scores across all samples were downloaded from Ebmeier et al (2017). Scores from multiple replicates were averaged for pull-downs using unphosphorylated full length CTD (Unphos), TFIIH phosphorylated full length CTD (Phospho CDK7), or p-TEFb phosphorylated full length CTD (Phospho CDK9). Averaged iBAQ score for each protein is plotted on the y-axis. (FIG. 67B) Representative immunofluorescence (IF) imaging of splicing factors SRSF2, SRRM1, and SRSF1 in C2C12 cells transfected with control siRNA (left), or siRNA against the indicated factor (right). (FIG. 67C) Representative images showing enhanced CTD incorporation into SRSF2 condensates requires CDK7 and ATP. GFP-CTD52 at 3.3 uM, which has been incubated with recombinant CDK7 and/or ATP (see methods), was mixed with 1.2 uM mCherry-SRSF2 in droplet formation buffers with 100 mM NaCl and 10% PEG-8000 and visualized on a fluorescence microscope with the indicated filters.

FIGS. 68A-68D show the MYC oncogene is occupied by Mediator condensates in tumor tissue and cancer cells. (FIG. 68A) (Left) Hematoxylin and eosin stained ER+ human invasive ductal carcinoma of the breast. (Right) Confocal microscopy images of MED1 or ER IF and RNA FISH to the MYC locus in ER+ human breast cancer tissue. (FIG. 68B) (Left) Confocal microscopy images of ER or MED1 IF with RNA FISH to the MYC locus in the breast cancer cell line MCF7 grown in the presence of estrogen. (Right) Enrichment analysis and random focus analysis of MED1 (top, n=23) or ER (bottom, n=18) IF at the MYC RNA FISH focus in MCF7 cells. (FIG. 68C) FRAP of mEGFP-tagged MED1 in MCF7 cells. Quantification shown to the right, n=3, average (green line), best fit line (solid black), and 95% confidence intervals (dashed black). (FIG. 68D) Confocal microscopy images of MED1 IF and RNA FISH to the MYC locus in the indicated cancer cell lines.

FIGS. 69A-69F show ER forms estrogen-dependent, tamoxifen-sensitive condensates with Mediator. (FIG. 69A) (Left) Confocal microscopy images of MED1 IF with DNA FISH to the MYC locus in unstimulated, estrogen stimulated, or tamoxifen treated MCF7 cells. (Right) Model showing effects of estrogen and tamoxifen treatment on Mediator condensates at an estrogen responsive oncogene. (FIG. 69B) RT-qPCR of MYC expression in the indicated condition in MCF7 cells. (FIG. 69C) (Left) Schematic of the Lac array in U2OS cells. (Top Right) Confocal microscopy images of a Lac-CFP-ER-LBD fusion protein shown with MED1 IF with the indicated ligand. (Bottom Right) Quantification of MED1 enrichment at the Lac array, n≥8. (FIG. 69D) (Top) Live cell imaging of mEGFP-MED1 endogenously tagged U2OS cells, transfected with LAC-mCherry-ER-LBD, treated with tamoxifen and imaged at 0 and 30 minutes. (Bottom) Quantification of enrichment ratio at the Lac array 30 minutes with the indicated ligand, n=3. (FIG. 69E) (Left) Schematic of the in vitro droplet assay. (Top Right) Confocal images of in vitro droplet assays of ER-GFP and MED1-mCherry with the indicated ligand. (Bottom Right) Schematic of droplet behavior. (FIG. 69F) Phase diagram schematic of ER-MED1 droplet formation.

FIGS. 70A-70G show hormonal therapy-resistant ER mutations constitutively condense with Mediator. (FIG. 70A) Phase diagram schematic of ER-MED1 droplet formation. (FIG. 70B) Schematic of the patient-derived ER point mutations and translocations. (FIG. 70C-FIG. 70D) In vitro droplet assay with the indicated ER mutant fused to GFP and MED1-mCherry with the indicated ligand. (FIG. 70E) Schematic of the GAL4 transactivation assay. (FIG. 70F-FIG. 70G) Transactivation activity of GAL4-DBD ER LBD wildtype or mutant proteins with the indicated ligand, n=9, asterisks represent p<0.01 relative to ER without estrogen.

FIGS. 71A-71G show MED1 overexpression facilitates Mediator condensation. (FIG. 71A) Phase diagram schematic of ER-MED1 droplet formation. (FIG. 71B) Western blot of MED1 in MCF7 cells or an established tamoxifen resistant MCF7 cell line. (FIG. 71C) Droplet formation assays of ER-GFP and MED1-mCherry at low (200 nM) or high (1600 nM) concentrations of MED1 in the presence of the indicated ligand, visualized in the MED1 channel. Quantification shown below, n>20. (FIG. 71D) Confocal microscopy images of a U2OS cell transfected with Lac-ER-LBD fusion protein (top row) followed by MED1 IF (bottom row). Quantification shown below, n≥8. (FIG. 71E) Transactivation assay with GAL4-ER LBD performed in the presence of low or high MED1 levels, in the presence of tamoxifen, n=9. (FIG. 71F) Survival of MCF7 cells with WT or high MED1 levels treated with tamoxifen. Quantification is shown below, n=4. (FIG. 71G) Schematic of estrogen-independent condensate formation and oncogene activation in the presence of high MED1 levels.

FIGS. 72A-72C show the MYC oncogene is occupied by Mediator condensates in tumor tissue and cancer cells. (FIG. 72A) Clinical data from the biopsied breast cancer specimen. (FIG. 72B) Confocal microscopy images of MED1 IF and DAPI staining on the ER+ breast carcinoma biopsy showing MED1 puncta. (FIG. 72C) Western blot of MED1 levels in MCF7 MED1-mEGFP cell line.

FIGS. 73A-73C show ER forms estrogen-dependent, tamoxifen-sensitive condensates with Mediator. (FIG. 73A) Schematic of the knockin strategy for generating mEGFP-MED1 U2OS Lac cells. (FIG. 73B) Western blot demonstrating the presence of mEGFP-tagged MED1 in U20S-Lac cells. (FIG. 73C) Quantification of the in vitro droplet assay shown in FIG. 2E, n>20.

FIGS. 74A-74C show hormonal therapy-resistant ER mutations constitutively condense with Mediator. (FIG. 74A) Frequency of ER mutations with the hotspots 537 and 538, data derived from 220 patients in the cBioPortal database. (FIG. 74B) Quantification of ER mutant protein incorporation into MED1 droplets with the indicated ligand, n>20. (FIG. 74C) Lac assay of ER point mutants with MED1 IF. Quantification of enrichment shown below, n≥8.

FIGS. 75A-75B show MED1 overexpression facilitates Mediator condensation. (FIG. 75A) Droplet formation assays of ER-GFP and MED1-mCherry at increasing concentrations of MED1 with the indicated ligand. (FIG. 75B) Transactivation assay with GAL4-ER LBD performed in the presence of low or high MED1 levels, without ligand.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Modulation of Transcription by Targeting Components of Condensates

Condensate Proteins

Many of the protein components of transcriptional condensates have regions of intrinsic disorder, also termed intrinsic (or intrinsically) disordered regions (IDR) or intrinsic (or intrinsically) disordered domains. Each of these terms is used interchangeably throughout the disclosure. Many components of heterochromatin condensates and condensates physically associated with mRNA initiation or elongation complexes also have IDRs. IDR lack stable secondary and tertiary structure. In some embodiments, an IDR may be identified by the methods disclosed in Ali, M., & Ivarsson, Y. (2018). High-throughput discovery of functional disordered regions. Molecular Systems Biology, 14(5), e8377.

In some embodiments of the compositions and methods described herein, a condensate component is a transcription factor. As used herein, a “transcription factor” (TF) is a protein that regulates transcription by binding to a specific DNA sequence. TFs generally contain a DNA binding domain and activation domain. In some embodiments, the transcription factor has an IDR in an activation domain. In some embodiments, the transcription factor (TF) is OCT4, p53, MYC or GCN4, NANOG, MyoD, KLF4, a SOX family transcription factor, or a GATA family transcription factor. In some embodiments, the TF is regulated by a signaling factor (e.g., transcription is modulated by TF interaction with a signaling factor). In some embodiments, the TF is a nuclear receptor (e.g., a nuclear hormone receptor, Estrogen Receptor, Retinoic Acid Receptor-Alpha). Nuclear receptors are members of a large superfamily of evolutionarily related DNA-binding transcription factors that exhibit a characteristic modular structure consisting of five to six domains of homology (designated A to F, from the N-terminal to the C-terminal end). The activity of NRs is regulated at least in part by the binding of a variety of small molecule ligands to a pocket in the ligand-binding domain. The human genome encodes about 50 NRs. Members of the NR superfamily include glucocorticoid, mineralocorticoid, progesterone, androgen, and estrogen receptors, peroxisome proliferator-activated (PPAR) receptors, thyroid hormone receptors, retinoic acid receptors, retinoid X receptors, NR1H and NR1I receptors, and orphan nuclear receptors (i.e., receptors for which no ligand has been identified as of a particular date). In some embodiments a nuclear receptor (NR) is a nuclear receptor subfamily 0 member, nuclear receptor subfamily 1 member, nuclear receptor subfamily 2 member, nuclear receptor subfamily 3 member, nuclear receptor subfamily 4 member, nuclear receptor subfamily 5 member, or nuclear receptor subfamily 6 member. In some embodiments a nuclear receptor is NR1D1 (nuclear receptor subfamily 1, group D, member 1), NR1D2 (nuclear receptor subfamily 1, group D, member 2), NR1H2 (nuclear receptor subfamily 1, group H, member 2; synonym: liver X receptor beta), NR1H3 (nuclear receptor subfamily 1, group H, member 3; synonym: liver X receptor alpha), NR1H4 (nuclear receptor subfamily 1, group H, member 4), NR1I2 (nuclear receptor subfamily 1, group I, member 2; synonym: pregnane X receptor), NR1I3 (nuclear receptor subfamily 1, group I, member 3; synonym: constitutive androstane receptor), NR1I4 (nuclear receptor subfamily 1, group I, member 4), NR2C1 (nuclear receptor subfamily 2, group C, member 1), NR2C2 (nuclear receptor subfamily 2, group C, member 2), NR2E1 (nuclear receptor subfamily 2, group E, member 1), NR2E3 (nuclear receptor subfamily 2, group E, member 3), NR2F1 (nuclear receptor subfamily 2, group F, member 1), NR2F2 (nuclear receptor subfamily 2, group F, member 2), NR2F6 (nuclear receptor subfamily 2, group F, member 6), NR3C1 (nuclear receptor subfamily 3, group C, member 1; synonym: glucocorticoid receptor), NR3C2 (nuclear receptor subfamily 3, group C, member 2; synonym: aldosterone receptor, mineralocorticoid receptor), NR4A1 (nuclear receptor subfamily 4, group A, member 1), NR4A2 (nuclear receptor subfamily 4, group A, member 2), NR4A3 (nuclear receptor subfamily 4, group A, member 3), NR5A1 (nuclear receptor subfamily 5, group A, member 1), NR5A2 (nuclear receptor subfamily 5, group A, member 2), NR6A1 (nuclear receptor subfamily 6, group A, member 1), NROB1 (nuclear receptor subfamily 0, group B, member 1), NROB2 (nuclear receptor subfamily 0, group B, member 2), RARA (retinoic acid receptor, alpha), RARB (retinoic acid receptor, beta), RARG (retinoic acid receptor, gamma), RXRA (retinoid X receptor, alpha; synonym: nuclear receptor subfamily 2 group B member 1), RXRB (retinoid X receptor, beta; synonym: nuclear receptor subfamily 2 group B member 2), RXRG (retinoid X receptor, gamma; synonym: nuclear receptor subfamily 2 group B member 3), THRA (thyroid hormone receptor, alpha), THRB (thyroid hormone receptor, beta), AR (androgen receptor), ESR1 (estrogen receptor 1), ESR2 (estrogen receptor 2; synonym: ER beta), ESRRA (estrogen-related receptor alpha), ESRRB (estrogen-related receptor beta), ESRRG (estrogen-related receptor gamma), PGR (progesterone receptor), PPARA (peroxisome proliferator-activated receptor alpha), PPARD (peroxisome proliferator-activated receptor delta), PPARG (peroxisome proliferator-activated receptor gamma), VDR (vitamin D (1,25-dihydroxyvitamin D3) receptor).

In some embodiments, the nuclear receptor is a naturally occurring truncated form of a nuclear receptor generated by proteolytic cleavage, such as truncated RXR alpha, or truncated estrogen receptor. In some embodiments a receptor, e.g., a NR, is an HSP70 client. For example, androgen receptor (AR) and glucocorticoid receptor (GR) are HSP70 clients. Extensive information regarding NRs may be found in Germain, P., et al., Pharmacological Reviews, 58:685-704, 2006, which provides a review of nuclear receptor nomenclature and structure, and other articles in the same issue of Pharmacological Reviews for reviews on NR subfamilies). In some embodiments, an HSP90A client is a steroid hormone receptor (e.g., an estrogen, progesterone, glucocorticoid, mineralocorticoid, or androgen receptor), PPAR alpha, or PXR. In some embodiments, the nuclear receptor (NR) is a ligand-dependent NR. A ligand-dependent NR is characterized in that binding of a ligand to the NR modulates activity of the NR. In some embodiments binding of a ligand to ligand-dependent NF causes a conformational change in the NR that results in, e.g., nuclear translocation of the NR, dissociation of one or more proteins from the NR, activatation of the NR, or repressesion of the NR. In some embodiments, the NR is a mutant that lacks one or more activities of the wild-type NR upon ligand binding (e.g., nuclear translocation of the NR, dissociation of one or more proteins from the NR, activatation of the NR, or repressesion of the NR). In some embodiments, the NR is a mutant having a ligand-binding independent activity (e.g., nuclear translocation of the NR, dissociation of one or more proteins from the NR, activation of the NR, or repression of the NR) that is ligand dependent in the wild-type NR. In some embodiments, the nuclear receptor activates transcription when bound to a cognate ligand. In some embodiments, the nuclear receptor is a mutant nuclear receptor that activates transcription in the absence of the cognate ligand.

NRs play important roles in a wide range of biological processes such as development, differentiation, reproduction, immune responses, metabolic regulation, and xenobiotic metabolism, among others, as well as in a variety of pathological conditions. NRs represent an important class of drug targets. Pharmacological modulation of NRs (e.g., by modulation of transcription condensates containing NRs) may be of use in a variety of disorders including cancer, autoimmune, metabolic, and inflammatory/immune system disorders (e.g., arthritis, asthma, allergies) as well as post-transplant immunosuppression in order to reduce the likelihood of rejection. In addition to interacting with endogenous and/or exogenous small molecule ligand(s), NRs interact with a variety of endogenous proteins such as dimerization partners, coactivators, corepressors, ubiquitin ligases, kinases, phosphatases, which can modulate their activity.

Nuclear receptor ligands modulate activity of some NRs. Some ligands stimulate activity of a NR. Such a ligand may be referred to as an “agonist”. Some ligands do not affect activity of a NR or other ligand-dependent TF in the absence of an agonist. However, the ligand, which may be referred to as an “antagonist” is capable of inhibiting the effect of an agonist through, e.g., competitive binding to the same binding site in the protein as does the agonist or by binding to a different site in the protein. Certain NRs promote a low level of gene transcription in the absence of agonists (also referred to as basal or constitutive activity). Ligands that reduce this basal level of activity in nuclear receptors may be referred to as as inverse agonists.

In some embodiments, the transcription factor is a transcription factor listed in Table S3. In some embodiments, the transcription factor is a transcription factor that interacts with a mediator component (e.g., a mediator component listed in Table S3).

In some embodiments, the TF is a TF having activity regulated by a signaling factor. In some embodiments, the signaling factor comprises an IDR. In some embodiments, the signaling factor is TCF7L2, TCF7, TCF7L1, LEF1, Beta-Catenin, SMAD2, SMAD3, SMAD4, STAT1, STAT2, STAT3, STAT4, STAT5A, STAT5B, STAT6, or NF-κB. In some embodiments of the compositions and methods described herein, a signaling factor can be NF-kB, FOXO1, FOXO2, FOXO4, IKKalpha, CREB, Mdm2, YAP, BAD, p65, p50, GLI1, GLI2, GLI3, YAP, TAZ, TEAD1, TEAD2, TEAD3, TEAD4, STAT1, STAT2, STAT3, STAT4, STAT5A, STAT5B, STAT6, AP-1, C-FOS, CREB, MYC, JUN, CREB, ELK1, SRF, NOTCH1, NOTCH2, NOTCH3, NOTCH4, RBPJ, MAML1, SMAD2, SMAD3, SMAD4, IRF3, ERK1, ERK2, MYC, TCF7L2, TCF7, TCF7L1, LEF1, or Beta-Catenin.

In some embodiments of the compositions and methods described herein, a condensate component is a protein listed in Table S1. In some embodiments, a condensate component in any of the compositions or methods described herein comprises an IDR of a protein listed in Table S1. In some embodiments, a condensate component in any of the compositions or methods described herein associates with a protein listed in Table S1. In some embodiments, a condensate component in any of the compositions or methods described herein associates with an IDR of a protein listed in Table S1. In some embodiments, a condensate component is a mediator component listed in Table S3.

TABLE S1
proteins and regions of disorder (IDR):

| | UniProt | UniProt | Whyte_SE_ | | | IDR |
|---|---|---|---|---|---|---|
| | ID | ID | foldOver_T | Length | % | length |
| | (mouse) | (human) | E_Density | (aa) | Disorder | (aa) |
| MED1 | Q925J9 | Q15648 | 5.59 | 1575 | 43.43 | 684 |
| PoIII | P08775 | P24928 | 4.35 | 1970 | 19.49 | 384 |
| MI2B | Q14839 | P19876 | 4.31 | 1915 | 28.56 | 547 |
| (CHD4) | | | | | | |
| SPT5 | O55201 | O00267 | 4.22 | 1082 | 31.98 | 346 |
| AFF4 | Q9ESC8 | Q9UHB7 | 3.49 | 1160 | 72.24 | 838 |
| CTR9 | Q62018 | Q6PD62 | 3.42 | 1173 | 24.04 | 282 |
| MED12 | A2AGH6 | Q93074 | 3.18 | 2190 | 11.78 | 258 |
| P300 | B2RWS6 | Q09472 | 3.06 | 2414 | 36.29 | 876 |
| IN080 | Q6ZPV2 | Q9ULG1 | 3.06 | 1559 | 14.5 | 226 |
| BRD4 | Q9ESU6 | O60885 | 2.95 | 1400 | 72.5 | 1015 |
| SETD7 | Q8VHL1 | Q8WTS6 | 2.87 | 366 | 0 | 0 |
| CDK8 | Q8R3L8 | P49336 | 2.83 | 464 | 23.06 | 107 |
| SMAD3 | Q8BUN5 | P84022 | 2.59 | 425 | 0 | 0 |
| ESRRB | Q61539 | O95718 | 2.47 | 433 | 8.78 | 38 |
| MCEF | Q9ESC8 | Q9UHB7 | 2.46 | 1160 | 72.24 | 838 |
| (AFF4) | | | | | | |
| BRD2 | Q7JJ13 | P25440 | 2.45 | 798 | 40.23 | 321 |
| ZFX | P17012 | P17010 | 2.39 | 799 | 0 | 0 |
| CBP | P45481 | Q92793 | 2.36 | 2441 | 23.43 | 572 |
| NELFA | Q8BG30 | Q9H3P2 | 2.34 | 530 | 12.08 | 64 |
| TAF3 | Q5HZG4 | Q5VWG9 | 2.32 | 932 | 52.04 | 485 |
| TBP_2 | P29037 | P20226 | 2.32 | 316 | 0 | 0 |
| ELL2 | Q3UKU1 | O00472 | 2.3 | 639 | 28.01 | 179 |
| TAF1 | Q80UV9 | P21675 | 2.19 | 1891 | 15.86 | 300 |
| TBP_1 | P29037 | P20226 | 2.19 | 316 | 0 | 0 |
| ZMYND8 | Q80Y82 | Q9ULU4 | 2.11 | 1255 | 49.8 | 625 |
| SMAD2_3 | Q62432 | Q15796 | 2.11 | 467 | 0 | 0 |
| E2F4 | Q8R0K9 | Q16254 | 2.02 | 410 | 16.1 | 66 |
| cMYC | P01108 | P01106 | 2.01 | 439 | 36.67 | 161 |
| TCFCP2L1 | Q3UNW5 | | 2.01 | 479 | 6.26 | 30 |
| STAT3 | P42227 | P40763 | 2 | 770 | 0 | 0 |
| NPAT | Q8BMA5 | Q14207 | 1.99 | 1420 | 27.96 | 397 |
| NIPBL | Q6KCD5 | 06KC79 | 1.98 | 2798 | 29.16 | 816 |
| KLF4 | Q60793 | O43474 | 1.94 | 483 | 15.11 | 73 |
| CDK7 | Q03147 | P50613 | 1.94 | 346 | 0 | 0 |
| CDK9 | Q99J95 | P50750 | 1.9 | 372 | 8.06 | 30 |
| CDX2 | P43241 | Q99626 | 1.89 | 311 | 23.47 | 73 |
| CAPD3 | Q6ZQKO | P42695 | 1.89 | 1506 | 9.1 | 137 |
| LSD1 | Q6Z088 | O60341 | 1.88 | 853 | 20.75 | 177 |
| SA2 | O35638 | Q8N3U4 | 1.88 | 1231 | 7.72 | 95 |
| SA1 | Q9D3E6 | Q8WVM7 | 1.86 | 1258 | 12.16 | 153 |
| ELL3 | Q80VR2 | Q9HB65 | 1.85 | 395 | 28.86 | 114 |
| RAD21 | Q61550 | Q60216 | 1.84 | 635 | 22.83 | 145 |
| HCFC1 | Q61191 | P51610 | 1.83 | 2045 | 9.54 | 195 |
| SMC1 | Q9CU62 | Q14683 | 1.82 | 1233 | 5.6 | 69 |
| BioUTF1 | Q6J1H4 | Q5T230 | 1.77 | 339 | 50.15 | 170 |
| CAPH | Q8C156 | Q15003 | 1.77 | 731 | 16.55 | 121 |
| REX1 | P22227 | Q96MM3 | 1.74 | 288 | 16.67 | 48 |
| TETI_ | Q3URK3 | Q8NFU7 | 1.73 | 2007 | 21.33 | 428 |
| ATM | Q62388 | Q13315 | 1.73 | 3066 | 3.49 | 107 |
| HP1g | P23198 | Q13185 | 1.71 | 183 | 41.53 | 76 |
| (CBX3) | | | | | | |
| SMC3 | Q9CW03 | Q9U0E7 | 1.69 | 1217 | 4.85 | 59 |
| YY1 | Q00899 | P25490 | 1.68 | 414 | 18.36 | 76 |
| RONIN | Q9JJDO | B5APZ3 | 1.66 | 305 | 16.72 | 51 |
| ESCO2 | Q8CIB9 | Q56NI9 | 1.66 | 592 | 4.73 | 28 |
| SETDB1 | O88974 | Q15047 | 1.64 | 1307 | 33.59 | 439 |
| KAP1 | Q62318 | Q13263 | 1.62 | 834 | 7.91 | 66 |
| (TRIM28) | | | | | | |
| NCOA3 | O09000 | Q9Y609 | 1.61 | 1398 | 21.17 | 296 |
| CAPH2 | Q8BSP2 | Q6IBW4 | 1.6 | 607 | 12.36 | 75 |
| MCAF1 | Q7TT18 | Q6VM06 | 1.58 | 1306 | 53.29 | 696 |
| MYOD | P10085 | P15172 | 1.58 | 318 | 33.02 | 105 |
| SETD8 | Q2YDW7 | Q9NQR1 | 1.57 | 349 | 49.28 | 172 |
| TET2 | Q4JK59 | Q6N021 | 1.56 | 1912 | 27.46 | 525 |
| MED15 | Q924H2 | Q96RN5 | 1.55 | 792 | 20.2 | 160 |
| H2AX | P27661 | P16104 | 1.54 | 143 | 31.47 | 45 |
| CDK11 | P24788 | P21127 | 1.51 | 784 | 55.61 | 436 |
| BRG1 | Q3TKT4 | P51532 | 1.5 | 1613 | 34.22 | 552 |
| PTTG1 | Q9C0J7 | Q95997 | 1.5 | 199 | 29.65 | 59 |
| H3 | P84244 | P84243 | 1.49 | 136 | 31.62 | 43 |
| CDK19 | Q8BWD8 | Q9BWU1 | 1.48 | 501 | 27.94 | 140 |
| HDAC2 | P70288 | Q92769 | 1.48 | 488 | 20.49 | 100 |
| MBD3 | Q9Z2D8 | O95983 | 1.47 | 285 | 10.88 | 31 |
| SOX17 | Q61473 | Q9H612 | 1.45 | 419 | 18.62 | 78 |
| PBRM1 | Q8BS09 | Q86U86 | 1.44 | 1634 | 12.42 | 203 |
| ZFP143 | O70230 | P52747 | 1.44 | 638 | 0 | 0 |
| REST | Q8VIG1 | Q13127 | 1.43 | 1082 | 55.36 | 599 |
| CTCF | Q61164 | P49711 | 1.43 | 736 | 22.28 | 164 |
| SMC2 | Q8CG48 | O95347 | 1.43 | 1191 | 0 | 0 |
| RING1B | Q9C0J4 | Q99496 | 1.42 | 336 | 14.58 | 49 |
| CAPG | P24452 | P40121 | 1.42 | 352 | 0 | 0 |
| CDK1 | P11440 | P06493 | 1.41 | 297 | 0 | 0 |
| pSMC1 | Q9CU62 | Q14683 | 1.4 | 1233 | 5.6 | 69 |
| LaminB | P14733 | P20700 | 1.39 | 588 | 13.1 | 77 |
| HDAC1 | O09106 | Q13547 | 1.35 | 482 | 19.29 | 93 |
| SUV39H2 | Q9E000 | Q9H511 | 1.34 | 477 | 12.37 | 59 |
| ADAM10 | O35598 | O14672 | 1.34 | 749 | 5.61 | 42 |
| IKBKAP | Q7TT37 | O95163 | 1.34 | 1333 | 2.48 | 33 |
| PRDM14 | E903T6 | Q9GZV8 | 1.32 | 561 | 0 | 0 |
| SMAD1 | P70340 | Q15797 | 1.3 | 465 | 8.17 | 38 |
| SUV39H1 | O54864 | O43463 | 1.29 | 412 | 0 | 0 |
| BRN2 | P31360 | P20265 | 1.28 | 445 | 47.19 | 210 |
| SUZ12 | Q80U70 | Q15022 | 1.25 | 741 | 9.99 | 74 |
| TFE3 | Q64092 | P19532 | 1.18 | 572 | 20.63 | 118 |
| ZFP57 | Q8C6P8 | Q9NU63 | 1.16 | 421 | 19.48 | 82 |
| GATA6 | Q61169 | Q92908 | 1.14 | 589 | 28.18 | 166 |
| RAD21_GFP | Q61550 | O60216 | 1.14 | 635 | 22.83 | 145 |
| H2AZ | P0C0S6 | P0C0S5 | 1.06 | 128 | 19.53 | 25 |
| TCF3_1 | P15806 | P15923 | 1.02 | 651 | 35.33 | 230 |
| TCF3_2 | P15806 | P15923 | 0.99 | 651 | 35.33 | 230 |
| OCT_4 | P20263 | Q01860 | 0.99 | 352 | 7.1 | 25 |
| NANOG | Q80Z64 | Q9H9S0 | 0.97 | 305 | 26.23 | 80 |
| SOX2 | P48432 | P48431 | 0.88 | 319 | 13.17 | 42 |
| OLIG2 | Q9EQW6 | Q13516 | 0.8 | 323 | 33.13 | 107 |

In Table S1, “IDR length (aa)” was calculated by multiplying the % Disorder by the total length of the protein. The methods set forth in Potenza, et al., “MobiDB 2.0: an improved database of intrinsically disordered and mobile proteins,” Nucleic Acids Res. 2015 January; 43 (Database issue):D315-20 can be used to obtain % Disorder for a given protein, which is incorporated herein in its entirety.

A number of amino acid sequence motifs or biases in these disordered regions have been identified.

TABLE S2
list of motifs:

| Motif_ID | Motif | Width |
|---|---|---|
| motif_1 | SYSPTSP (SEQ ID NO: 1) | 7 |
| motif_2 | QQQQQ (SEQ ID NO: 2) | 5 |
| motif_3 | PCETHETGTTHTATT (SEQ ID NO: 3) | 15 |
| motif_4 | EEEGEEEEEEE (SEQ ID NO: 4) | 11 |
| motif_5 | MEPAQMEVAQIEPAP (SEQ ID NO: 5) | 15 |
| motif_6 | DKRISICASDKRIAC (SEQ ID NO: 6) | 15 |
| motif_7 | HHHHH (SEQ ID NO: 7) | 5 |
| motif_8 | GRPETPKQK (SEQ ID NO: 8) | 9 |
| motif_9 | FFPQRQF (SEQ ID NO: 9) | 7 |
| motif_10 | QHRLQQAQLLRRRMA (SEQ ID NO: 10) | 15 |
| motif_11 | RKKEKKEKKKKRKKE (SEQ ID NO: 11) | 15 |
| motif_12 | RTPMYGSQTPLHD (SEQ ID NO: 12) | 13 |

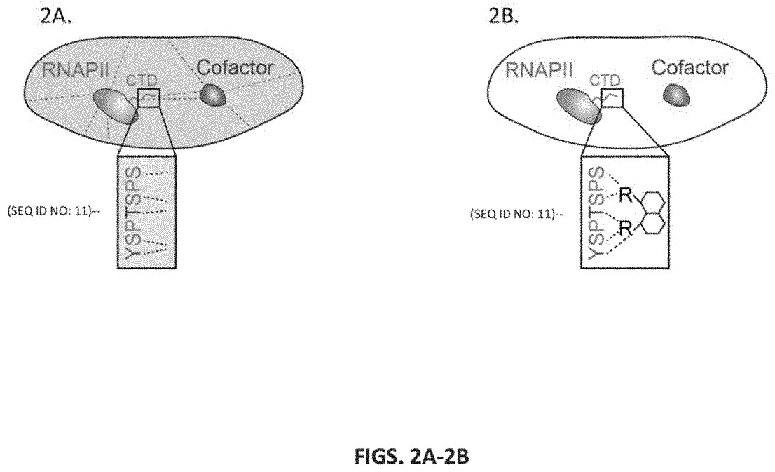

It is proposed that these motifs participate in condensate formation, maintenance, dissolution or regulation. (FIG. 2A). A peptide, nucleic acid or a small chemical molecule that interacts specifically with any one type of protein motif would be expected to influence condensate formation, composition, maintenance, dissolution or regulation and thereby result in altering the transcription output of condensates that employ such a motif (FIG. 2B). Thus, expression of one or more genes can be influenced by modulating a transcriptional condensate.

For instance, in some embodiments, modulating a transcriptional condensate can modulate expression of genes controlled by an enhancer or super-enhancer (SE). As used herein, a “super-enhancer” is a cluster of enhancers that are occupied by exceptionally high densities of transcription apparatus, certain SEs regulate genes with especially important roles in cell identity (e.g., cell growth, cell differentiation). The disclosure contemplates the modulation of any enhancer or super-enhancer. Exemplary super-enhancers are disclosed in PCT International Application No. PCT/US2013/066957 (attorney docket no. WIBR-137-WO1), filed Oct. 25, 2013, the entirety of which is incorporated by reference herein.

As used herein, the phrase “super-enhancer component” refers to a component, such as a protein, that has a higher local concentration, or exhibits a higher occupancy, at a super-enhancer, as opposed to a normal enhancer or an enhancer outside a super-enhancer, and in embodiments, contributes to increased expression of the associated gene. In an embodiment, the super-enhancer component is a nucleic acid (e.g., RNA, e.g., eRNA transcribed from the super-enhancer, i.e., an eRNA). In an embodiment, the nucleic acid is not chromosomal nucleic acid. In an embodiment, the super-enhancer component is involved in the activation or regulation of transcription. In some embodiments, the super-enhancer component comprises RNA polymerase II, Mediator, cohesin, Nipbl, p300, CBP, Chd7, Brd4, and components of the esBAF (Brg1) or a Lsd1-Nurd complex (e.g., RNA polymerase II).

In some embodiments, the super-enhancer component is a transcription factor. In some embodiments, the transcription factor is OCT4, p53, MYC, or GCN4. In some embodiments, the transcription factor has an IDR (e.g., an IDR in an activation domain of the transcription factor). In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3). As used herein, the term “transcription factor” refers to a protein that binds to specific parts of DNA using DNA binding domains and is part of the system that controls the transfer (or transcription) of genetic information from DNA to RNA. As used herein, transcription activator domains (AD) are regions of a transcription factor which in conjunction with a DNA binding domain can activate transcription from a promoter. In some embodiments, the AD does not comprise the transcription factor DNA-Binding Domain. In some embodiments, the AD is from a human transcription factor as defined in Violaine Saint-André et al., Gen Res, 2015. In some embodiments, the AD comprises an IDR. In some embodiments, the IDR is at least about 5, 10, 15, 20, 30, 40, 50, 60, 75, 100, 150, or more disordered amino acids (e.g., contiguous disordered amino acids). In some embodiments, an amino acid is considered a disordered amino acid if at least 75% of the algorithms employed by D2P2 (Oates et al., 2013) predict the residue to be disordered. In some embodiments a fragment of an identified AD that, for example, retains at least 30%, 40%, 50%, 60%, 70%, 80%, 90%, or more, of the activation capacity of the full length AD, may be selected.

As used herein, “enhancer” refers to a short region of DNA to which proteins (e.g., transcription factors) bind to enhance transcription of a gene. As used herein, “transcriptional coactivator” refers to a protein or complex of proteins that interacts with transcription factors to stimulate transcription of a gene. In some embodiments, the transcriptional coactivator is Mediator. In some embodiments, the transcriptional coactivator is Med1 (Gene ID: 5469) or MED15. In some embodiments, the transcriptional coactivator is a Mediator component. As used herein, “Mediator component” comprises or consists of a polypeptide whose amino acid sequence is identical to the amino acid sequence of a naturally occurring Mediator complex polypeptide. The naturally occurring Mediator complex polypeptide can be, e.g., any of the approximately 30 polypeptides found in a Mediator complex that occurs in a cell or is purified from a cell (see, e.g., Conaway et al., 2005; Kornberg, 2005; Malik and Roeder, 2005). In some embodiments a naturally occurring Mediator component is any of Med1-Med 31 or any naturally occurring Mediator polypeptide known in the art. For example, a naturally occurring Mediator complex polypeptide can be Med6, Med7, Med10, Med12, Med14, Med15, Med17, Med21, Med24, Med27, Med28 or Med30. In some embodiments a Mediator polypeptide is a subunit found in a Med11, Med17, Med20, Med22, Med 8, Med 18, Med 19, Med 6, Med 30, Med 21, Med 4, Med 7, Med 31, Med 10, Med 1, Med 27, Med 26, Med14, Med15 complex. In some embodiments a Mediator polypeptide is a subunit found in a Med12/Med13/CDK8/cyclin complex. Mediator is described in further detail in PCT International Application No. WO 2011/100374, the teachings of which are incorporated herein by reference in their entirety.

A peptide, nucleic acid or a small chemical molecule (e.g., a compound, a small molecule, an agent described herein) that interacts specifically with any one type of motif in a protein that participates in condensate formation may cause preferential accumulation of the compound in the condensate, which may act to preferentially influence the behaviors of condensate associated functions. For example, the compound might stabilize or dissolve the condensate and thus modulate transcription. In some embodiments, the compound may stabilize or dissolve the condensate and thus modulate gene silencing. In some embodiments, the compound may stabilize or dissolve the condensate and thus modulate mRNA initiation or elongation (e.g., splicing). In some aspects, a method comprises identifying a compound that physically associates with a motif listed in Table S2. In some aspects, a method comprises identifying a compound that physically associates with an IDR of a nuclear receptor AD. In some embodiments, the nuclear receptor is a mutant nuclear receptor associated with a disease. In some embodiments, the mutant nuclear receptor is associated with breast cancer. In some embodiments of the methods and compounds disclosed herein, the nuclear receptor is a mutant estrogen receptor (e.g., estrogen receptor alpha) (e.g., Y537S ESR1, D538G ESR1). In some embodiments, the method comprises identifying a compound that interacts with a component of a heterochromatin or gene silencing condensate (e.g., a compound that interacts with methylated DNA, a methyl-DNA binding protein, a suppressor, or methylated DNA in a super-enhancer). In some embodiments, the method comprises identifying a compound that preferentially interacts with condensate physically associated with an initiation or elongation complex.

Thus, some aspects of the invention are directed to a method of modulating transcription of one or more genes in a cell, comprising modulating formation, composition, maintenance, dissolution and/or regulation of a condensate (e.g., transcriptional condensate) associated with the one or more genes. Some aspects of the invention are directed to a method of modulating gene silencing (e.g., suppression of transcription of one or more genes, suppression of transcription of one or more genes in heterochromatin), comprising modulating formation, composition, maintenance, dissolution and/or regulation of a condensate associated with the one or more genes. Some aspects of the disclosure are directed to modulating mRNA initiation or elongation, comprising modulating formation, composition, maintenance, dissolution and/or regulation of a condensate physically associated with an initiation or elongation complex.

As used herein “modulating” (and verb forms thereof, such as “modulates”) means causing or facilitating a qualitative or quantitative change, alteration, or modification. Without limitation, such change may be an increase or decrease in a qualitative or quantitative aspect.

The terms “increased,” “increase” or “enhance” may be, for example, increase or enhancement by a statically significant amount. In some instances, for example, an element can be increased or enhanced by at least about 10% as compared to a reference level (e.g., a control), at least about 20%, at least about 30%, at least about 40%, at least about 50%, at least about 60%, at least about 70%, at least about 80%, at least about 90%, or at least about 100%, and these ranges will be understood to include any integer amount therein (e.g., 2%, 14%, 28%, etc.) which are not exhaustively listed for brevity. In other instances an element can be increased or enhanced by at least about 2-fold, at least about 3-fold, at least about 4-fold, at least about 5-fold at least about 10-fold or more as compared to a reference level.

The terms “decrease,” “reduce,” “reduced,” “reduction,” and “inhibit” may be, for example, a decrease or reduction by a statistically significant amount relative to a reference (e.g., a control). In some instances an element can be, for example, decreased or reduced by at least 10% as compared to a reference level, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the element as compared to a reference level. These ranges will be understood to include any integer amount therein (e.g., 6%, 18%, 26%, etc.) which are not exhaustively listed for brevity.

For example, modulating transcription of a gene includes increasing or decreasing the rate or frequency of gene transcription; modulating the formation of a condensate includes increasing or decreasing the rate of formation or whether or not formation occurs; modulating the composition of a condensate includes increasing or decreasing the level of a component associated with the condensate; modulating the maintenance of a condensate includes increasing or decreasing the rate of condensate maintenance; modulating the dissolution of the condensate includes increasing or decreasing the rate of condensate dissolution and preventing or suppressing condensate dissolution; modulating condensate regulation includes modifying cell regulation of condensates. Modulating gene silencing includes increasing or reducing inhibition of transcription of the gene. Modulating mRNA initiation or transcription includes increasing or decreasing mRNA transcription initiation, mRNA elongation, and mRNA splicing activity. As used herein, modulating a condensate includes one, two, three, four or all five of modulating formation, composition, maintenance, dissolution and/or regulation of a condensate. In some embodiments, modulating a condensate includes changing the morphology or shape of the condensate.

As used herein, “gene silencing” (also sometimes referred to as gene transcription repression) refers to reducing or eliminating transcription of a gene. Transcription of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 98%, 99%, 99.5%, 99.9%, or more as compared to a reference level (e.g., an untreated control cell or condensate). In some embodiments, gene silencing is associated with heterochromatin or methylated genomic DNA. In some embodiments, gene silencing comprises the binding of methyl-DNA binding proteins to methylated DNA. In some embodiments, gene silencing comprises modifying chromatin. As used herein, “heterochromatin” refers to chromosome material of different density from normal (usually greater), in which the activity of the genes is modified or suppressed. In some embodiments of the methods and compositions herein, heterochromatin refers to facultative heterochromatin which, under specific developmental or environmental signaling cues, loses its condensed structure and becomes transcriptionally active.

In some embodiments, the one or more genes modulated comprise an oncogene. Exemplary oncogenes include MYC, SRC, FOS, JUN, MYB, RAS, ABL, HOXI1, HOXI1 1L2, TAL1/SCL, LMO1, LMO2, EGFR, MYCN, MDM2, CDK4, GLI1, IGF2, activated EGFR, mutated genes, such as FLT3-ITD, mutated of TP53, PAX3, PAX7, BCR/ABL, HER2/NEU, FLT3R, FLT6-ITD, SRC, ABL, TAN1, PTC, B-RAF, PML-RAR-alpha, E2A-PRX1, and NPM-ALK, as well as fusion of members of the PAX and FKHR gene families. Other exemplary oncogenes are well known in the art. In some embodiments the oncogene is selected from the group consisting of c-MYC and IRF4. In some embodiments the gene encodes an oncogenic fusion protein, e.g., an MLL rearrangement, EWS-FLI, ETS fusion, BRD4-NUT, NUP98 fusion.

In some embodiments, the one or more genes are associated with a hallmark of a disease such as cancer (e.g., breast cancer). In some embodiments, the one or more genes are associated with a disease associated DNA sequence variation such as a SNP. In some embodiments, the disease is Alzheimer's disease, and the genes comprises BIN1 (e.g., having a disease associated DNA sequence variation such as a SNP). In some embodiments, the disease is type 1 diabetes, and the one or more genes are associated with a primary Th cell (e.g., having a disease associated DNA sequence variation such as a SNP). In some embodiments, the disease is systemic lupus erythematosus, and the one or more genes play a key role in B cell biology (e.g., having a disease associated DNA sequence variation such as a SNP). In some embodiments, the one or more genes are associated with a disease or condition associated with a mutation in a gene encoding a nuclear receptor (e.g., a nuclear hormone receptor, a ligand dependent nuclear receptor). In some embodiments, the one or more genes are associated with a hallmark characteristic of the cell. In some embodiments, the one or more genes are aberrantly expressed or are associated with a DNA variation such as a SNP. “Aberrantly expressed” is used to indicate that the gene expression in one or more cells or in vitro condensates of interest is detectably different from a control level that is typical of that found in normal cells (e.g., normal cells of the same cell type or, for cultured cells, cultured cells under comparable conditions) or condensates not subject to a test treatment or condition (e.g., for condensates isolated from cells, isolated condensates from normal cells of the same cell type or, for cultured cells, cultured cells under comparable conditions). In some embodiments, the one or more genes are associated with aberrant signaling in a cell (e.g. aberrant signaling associated with the WNT, TGF-β or JAK/STAT pathways). In some embodiments, the one or more genes comprise genes with aberrant mRNA initiation or elongation (e.g., aberrant splicing). As used herein, “aberrant mRNA initiation or elongation” is detectably or significantly different than mRNA initiation or elongation in a control cell or subject (e.g., higher than or lower than in (increased or decreased as compared to) a healthy cell or subject, or cell or subject without a disease or condition characterized by atypical mRNA initiation or elongation). In some embodiments, the one or more genes are associated with splicing variants characteristic of a disease or condition (e.g., splicing variants comprising more or less mRNA sequence than mRNA sequence in a control subject without the disease or condition). In some embodiments, the one or more genes are associated with a disease or disorder associated with aberrant gene silencing (e.g., increased or decreased gene silencing as compared to gene silencing in a healthy cell or healthy subject (e.g., control cell or subject)). In some embodiments, the disease or disorder associated with aberrant gene silencing is Rett syndrome, MeCP2 over-expression syndrome or MeCP2 under-expression or activity. MeCP2 refers to methyl CpG binding protein 2 (Human UniProt ID: P51608). In some embodiments, the one or more genes are found in a mammalian cell, e.g., human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, e.g., cancerous blood cell, fetal blood cell, monocyte; B cell, e.g., Pro-B cell; brain, e.g., astrocyte cell, angular gyrus of the brain, anterior caudate of the brain, cingulate gyms of the brain, hippocampus of the brain, inferior temporal lobe of the brain, middle frontal lobe of the brain, brain cancer cell; T cell, e.g., naïve T cell, memory T cell; CD4 positive cell; CD25 positive cell; CD45RA positive cell; CD45RO positive cell; IL-17 positive cell; a cell that is stimulated with PMA; Th cell; Th17 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate, e.g., prostate cancer; colon, e.g., colorectal cancer cell; crypt cell, e.g., colon crypt cell; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cell; skin, e.g., fibroblast cell; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MM1S cell; NHLF cell; NHDF-Ad cell; RPMI-8402 cell; U87 cell; VACO 9M cell; VACO 400 cell; or VACO 503 cell.

In some embodiments, the one or more genes are disease-associated variations related to rheumatoid arthritis, multiple sclerosis, systemic scleroderma, primary biliary cirrhosis, Crohn's disease, Graves disease, vitiligo and atrial fibrillation. In some embodiments, the one or more genes are associated with a developmental disorder. In some embodiments, the one or more genes are associated with a neurological disorder or developmental neurological disorder.

In some embodiments, the one or more genes are considered cell type specific. A cell type specific gene need not be expressed only in a single cell type but may be expressed in one or several, e.g., up to about 5, or about 10 different cell types out of the approximately 200 commonly recognized (e.g., in standard histology textbooks) and/or most abundant cell types in an adult vertebrate, e.g., mammal, e.g., human. In some embodiments, a cell type specific gene is one whose expression level can be used to distinguish a cell, e.g., a cell as disclosed herein, such as a cell of one of the following types from cells of the other cell types: adipocyte (e.g., white fat cell or brown fat cell), cardiac myocyte, chondrocyte, endothelial cell, exocrine gland cell, fibroblast, glial cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell. In some embodiments a cell type specific gene is lineage specific, e.g., it is specific to a particular lineage (e.g., hematopoietic, neural, muscle, etc.) In some embodiments, a cell-type specific gene is a gene that is more highly expressed in a given cell type than in most (e.g., at least 80%, at least 90%) or all other cell types. Thus specificity may relate to level of expression, e.g., a gene that is widely expressed at low levels but is highly expressed in certain cell types could be considered cell type specific to those cell types in which it is highly expressed. In some embodiments, a cell-type specific gene is a gene that is less expressed, or not expressed, in a given cell type than in most (e.g., at least 80%, at least 90%) or all other cell types. Thus specificity may relate to level of expression, e.g., a gene that is widely expressed but is much less expressed in certain cell types could be considered cell type specific to those cell types in which it is less, or not at all, expressed. It will be understood that expression can be normalized based on total mRNA expression (optionally including miRNA transcripts, long non-coding RNA transcripts, and/or other RNA transcripts) and/or based on expression of a housekeeping gene in a cell. In some embodiments, a gene is considered cell type specific for a particular cell type if it is expressed at levels at least 2, 5, or at least 10-fold greater or less than in that cell than it is, on average, in at least 25%, at least 50%, at least 75%, at least 90% or more of the cell types of an adult of that species, or in a representative set of cell types. One of skill in the art will be aware of databases containing expression data for various cell types, which may be used to select cell type specific genes. In some embodiments a cell type specific gene is a transcription factor. In some embodiments, a cell type specific gene is associated with embryonic, fetal, or post-natal development.

In some embodiments, the transcriptional condensate is modulated by increasing or decreasing a valency of a component associated with the condensate (i.e. a condensate component). In some embodiments, the heterochromatin condensate or condensate physically associated with mRNA initiation or elongation complex is modulated by increasing or decreasing a valency of a component associated with the condensate (i.e. a condensate component). As used herein, “valency” refers to both the number of different binding partners for a component and the strength of the binding to one or more binding partners. In some embodiments, “a component associated with a condensate” may be a protein, a nucleic acid, or a small molecule. In some embodiments, the component is a nucleic acid (e.g., RNA, eRNA). In an embodiment, the nucleic acid is not chromosomal nucleic acid. In an embodiment, the component is involved in the activation or regulation of transcription. In some embodiments, the component comprises RNA polymerase II, Mediator, cohesin, Nipbl, p300, CBP, Chd7, Brd4, and/or components of the esBAF (Brg1) or a Lsd1-Nurd complex (e.g., RNA polymerase II). In some embodiments, the component is Mediator or a Mediator subunit (e.g., Med1). In some embodiments, the component is a chromatin regulator (e.g., a BET bromodomain protein, BRD4). In some embodiments, the component is a nuclear receptor ligand (e.g., a hormone). In some embodiments, the component is a signaling factor. In some embodiments, the component is a methyl-DNA binding protein. In some embodiments, the component is a gene silencing factor. In some embodiments, the component is a splicing factor. In some embodiments, the component is a component of an mRNA initiation or elongation complex (i.e., apparatus). In some embodiments, the component is an RNA polymerase. In some embodiments, the component is or comprises an enzyme that, adds, detects or reads, or removes a functional group, e.g., a methyl or acetyl group, from a chromatin component, e.g., DNA or histones. In some embodiments, the component is or comprises an enzyme that alters, reads, or detects the structure of a chromatin component, e.g., DNA or histones, e.g., a DNA methylase or demythylase, a histone methylase or demethylase, or a histone acetylase or de-acetylase that write, read or erase histone marks, e.g., H3K4me1 or H3K27Ac. In some embodiments, the component is or comprises an enzyme that adds, detects or reads, or removes a functional group, e.g., a methyl or acetyl group, from a chromatin component, e.g., DNA or histones. In some embodiments, the component is or comprises a protein needed for development into, or maintenance of, a selected cellular state or property, e.g., a state of differentiation, development or disease, e.g., a cancerous state, or the propensity to proliferate or the propensity or the propensity to undergo apoptosis. In some embodiments the disease state is a proliferative disease, an inflammatory disease, a cardiovascular disease, a neurological disease or an infectious disease. In some embodiments, the component is not an enzyme as described herein. In some embodiments the component is not a DNA methylase or demythylase, a histone methylase or demethylase, and/or a histone acetylase or de-acetylase.

In some embodiments, the component is a transcription factor. In some embodiments, the transcription factor is OCT4, p53, MYC, or GCN4, NANOG, MyoD, KLF4, a SOX family transcription factor (e.g., SRY, SOX1, SOX2, SOX3, SOX14, SOX21, SOX4, SOX11, SOX12, SOX5, SOX6, SOX13, SOX8, SOX9, SOX10, SOX7, SOX17, SOX18, SOX15, SOX30), a GATA family transcription factor (e.g., GATA 1-6), or a nuclear receptor (e.g., a nuclear hormone receptor, Estrogen Receptor, Retinoic Acid Receptor-Alpha). In some embodiments, the transcription factor has an IDR (e.g., an IDR in an activation domain of the transcription factor). In some embodiments, the nuclear receptor activates transcription when bound to a cognate ligand. In some embodiments, the nuclear receptor is a mutant nuclear receptor that activates transcription in the absence of the cognate ligand. In some embodiments, the TF is regulated by a signaling factor (e.g., transcription is modulated by TF interaction with a signaling factor).

In some embodiments, the component (e.g., heterochromatin component) is a gene silencing factor or mutant form thereof. In some embodiments, the heterochromatin factor is ATRX, MECP2, WRN, DNMT1, DNMT3B, EZH2, HP1, D4Z4, ICR, Lamin A, WRN, Mutant ICR IGF2-H19, or Mutant ICR IGF2-H19.

In some embodiments, the component is a protein listed in Table S1. In some embodiments, the component is a mediator component listed in Table S3. In some embodiments, the component is a protein having a motif (e.g., having an IDR with a motif) listed in Table S2. In some embodiments, the component has an IDR that interacts with an IDR listed in Table S2. In some embodiments, the component has at least 50%, at least 60%, at least 70%, at least 80%, at least 90%, at least 95% of an IDR (e.g., an IDR having a motif listed in Table S2). In some embodiments, the component has multiple IDRs (e.g., 2, 3, 4, 5, or more IDR regions). In some embodiments, the component has at least one IDR separated into multiple discrete sections. In some embodiments, the component is part of a scaffold of a transcriptional condensate. In some embodiments, the component is a client of the condensate. In some embodiments, the transcriptional condensate is modulated by contacting the condensate with an agent that interacts with one or more intrinsic disorder domains or regions (IDR) of a component associated with the transcriptional condensate. In some embodiments, the component is Mediator, a mediator component, MED1, MED15, GCN4, a nuclear receptor ligand, a signaling factor, or BRD4. In some embodiments, the component is part of a scaffold of a heterochromatin condensate or a condensate associated with an mRNA initiation or elongation complex. In some embodiments, the component is a client of the heterochromatin condensate or condensate associated with an mRNA initiation or elongation complex. In some embodiments, the heterochromatin condensate or condensate associated with an mRNA initiation or elongation complex is modulated by contacting the condensate with an agent that interacts with one or more intrinsic disorder domains or regions (IDR) of a component associated with the condensate. In some embodiments, the component is Mediator, a mediator component, MED1, MED15, GCN4, a nuclear receptor ligand, a gene silencing factor, a splicing factor, or BRD4.

In some embodiments, the IDR has a motif shown in Table S2. In some embodiments, the component having an IDR is listed in Table S1. In some embodiments, the IDR is an IDR of a nuclear receptor AD. In some embodiments, the component is any component described herein. The IDRs useful for the methods disclosed herein are not limited. IDRs can be identified by bioinformatics methods known in the art. See, e.g., Best R B (February 2017). “Computational and theoretical advances in studies of intrinsically disordered proteins”. Current Opinion in Structural Biology. 42: 147-154; See also the http: address //d2p2.pro/about/predictors. In some embodiments, the component having an IDR is BRD4, Mediator, or MED1. In some embodiments, the IDR has a length of at least 5, 7, 10, 15, 20, 25, 30, 35, 40, 45, 50, or 100 amino acids. In some embodiments, the IDR has separate discrete regions. In some embodiments, the IDR is at least about 5, 10, 15, 20, 30, 40, 50, 60, 75, 100, 150, or more disordered amino acids (e.g., contiguous disordered amino acids). In some embodiments, an amino acid is considered a disordered amino acid if at least 75% of the algorithms employed by D2P2 (Oates et al., 2013) predict the residue to be disordered.

In some embodiments, the component is Mediator, a mediator component, MED1, MED15, p300, BRD4, TFIID, TCF7L2, TCF7, TCF7L1, LEF1, Beta-Catenin, SMAD2, SMAD3, SMAD4, STAT1, STAT2, STAT5, STAT4, STAT5A, STAT5B, STAT6, NF-κB, MECP2, MBD1, MBD2, MBD3, MBD4, HP1α, TBL1R, HDAC3, SMRT, RNA polymerase II, SRSF2, SRRM1, SRSF1, a hormone, or a variant, mutant form, or fragment (e.g., functional fragment) thereof.

As used herein, a “functional fragment” of a protein or nucleic acid exhibits at least one bioactivity of the full length protein or nucleic acid. In some embodiments, the level of the bioactivity can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the level of bioactivity of the full length protein or nucleic acid. “Fragment” as used herein is understood to include functional fragments. In some embodiments, the length of the functional fragment is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, or any range therebetween, the length of the full length protein or nucleic acid. In some embodiments, the functional fragment comprises at least one functional domain or at least two functional domains. In some embodiments, the functional fragment comprises a ligand binding domain and a DNA-binding domain. In some embodiments, the functional fragment comprises an activation domain and a DNA-binding domain. In some embodiments, the functional fragment comprises an IDR. In some embodiments the bioactivity may be binding activity (e.g., ligand-binding activity, hormone binding activity, DNA-binding activity, transcriptional co-factor binding activity, gene-silencing factor binding activity, mRNA-binding activity).

In some embodiments, a functional fragment can incorporate into a heterotypic condensate and/or a homotypic condensate. It is understood that incorporation (or incorporate) means under relevant physiological conditions (e.g., conditions the same as or approximating conditions in a cell) or relevant experimental conditions (e.g., suitable conditions for the formation of a condensate in vitro). In some embodiments, a functional fragment is a fragment of a condensate component described below in the Examples section.

In some embodiments, a functional fragment of a signaling factor can bind a transcription factor. In some embodiments, a functional fragment of a signaling factor has the capacity to incorporate into a condensate (e.g., heterotypic condensate, transcriptional condensate).

In some embodiments, a functional fragment of a hypophosphorylated RNA polymerase II C-terminal domain is a fragment that has RNA synthesis bioactivity and/or has the capacity to incorporate into a condensate (e.g., heterotypic condensates, homotypic condensates, condensates comprising mediator). In some embodiments, a functional fragment of a splicing factor is a fragment that has mRNA splicing activity and/or has the capacity to incorporate into a condensate (e.g., heterotypic condensates, homotypic condensates, or condensates comprising phosphorylated RNA polymerase).

In some embodiments, a functional fragment of a methyl-DNA binding protein can bind methylated DNA and/or has the capacity to incorporate into a condensate (e.g., heterotypic condensates, homotypic condensates, or condensates comprising suppressors). In some embodiments, a functional fragment of a suppressor has gene silencing activity and/or has the capacity to incorporate into a condensate (e.g., heterotypic condensates, homotypic condensates, or condensates comprising methyl-DNA binding protein).

In some embodiments, a functional fragment of an estrogen receptor has the capacity to (a) activate transcription when bound to estrogen (e.g., a wild-type ER fragment), (b) activate transcription constitutively (e.g., a mutant ER fragment), (c) bind to estrogen, (d) bind to mediator, (e) form heterotypic condensates, and/or (f) form homotypic condensates. In some embodiments, the estrogen receptor fragment has at least one, two, three, four, five or all five of the bioactivities (a) through (e). In some embodiments, a functional fragment of an ER ligand binding domain has estrogen binding activity.

As used herein, and in some embodiments, a variant of a protein comprises or consists of a polypeptide whose amino acid sequence is at least 70%, 80%, 90%, 95%, 96%, 97%, 98%, 99%, 99.5%, or greater than 99.5% identical to the amino acid sequence of the subject protein (e.g., wild-type protein, defined mutant protein). As used herein, and in some embodiments, a variant of a nucleic acid sequence comprises or consists of a nucleic acid sequence with at least 70%, 80%, 90%, 95%, 96%, 97%, 98%, 99%, 99.5%, or greater than 99.5% identical sequence to the nucleic acid sequence of the subject nucleic acid.

“Agent” is used herein to refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest, e.g., for modulating a condensate. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long.

“Nucleic acid construct” refers to a nucleic acid that is generated by man and is not identical to nucleic acids that occur in nature, i.e., it differs in sequence from naturally occurring nucleic acid molecules and/or comprises a modification that distinguishes it from nucleic acids found in nature. A nucleic acid construct may comprise two or more nucleic acids that are identical to nucleic acids found in nature, or portions thereof, but are not found as part of a single nucleic acid in nature. In some embodiments an agent that modulates a transcriptional condensate is encoded by a nucleic acid construct. In some embodiments the nucleic acid construct is introduced into a cell and expressed therein so as to modulate a transcriptional condensate in said cell. In some embodiments an agent that modulates a heterochromatin condensate or a condensate physically associated with an mRNA initiation or elongation complex is encoded by a nucleic acid construct. In some embodiments the nucleic acid construct is introduced into a cell and expressed therein so as to modulate a heterochromatin condensate or a condensate physically associated with an mRNA initiation or elongation complex in said cell.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics. In some embodiments, the peptide mimetic is a signaling factor mimetic. The signaling factor is not limited and may be any one known in the art and/or described herein. In some embodiments, the peptide mimetic is a nuclear receptor ligand mimetic.

In some embodiments, the agent is a protein, polypeptide, or nucleic acid associated with a condensate (e.g., transcriptional condensate, gene silencing condensate, condensate physically associated with mRNA initiation or elongation complex). In some embodiments, the agent is a variant or mutant of a protein, polypeptide, or nucleic acid associated with a condensate. In some embodiments, the agent is an antagonist or agonist of a nuclear receptor (e.g., nuclear hormone receptor). In some embodiments, the agent preferentially binds to a nuclear receptor having a mutation (e.g., nuclear hormone receptor having a mutation, ligand dependent nuclear receptor having a mutation) over a wild-type nuclear condensate. In some embodiments, the agent preferentially disrupts a transcriptional condensate comprising a nuclear receptor having a mutation (e.g., nuclear hormone receptor having a mutation, ligand dependent nuclear receptor having a mutation) over a condensate comprising a wild-type nuclear receptor.

In some embodiments, the agent is an antagonist or agonist of a signaling factor. The signaling factor is not limited and may be any signaling factor described herein or known in the art. In some embodiments, the signaling factor comprises an IDR. In some embodiments, the agent comprises a phosphorylated or hypophosphorylated RNA polymerase II C-terminal domain (Pol II CTD), or a functional fragment thereof. In some embodiments, the agent preferentially binds phosphorylated or hypophosphorylated Pol II CTD. In some embodiments, the agent binds a splicing factor, an elongation complex component, or a initiation complex component. In some embodiments, the agent preferentially binds methylated DNA. In some embodiments, the agent binds a methyl-DNA binding protein.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. For example, the synthetic RNA can encode an agent that modulates condensate composition, maintenance, dissolution, formation, or regulation. In some embodiments, the synthetic RNA encodes an IDR (e.g., an IDR listed in Table S2), an antibody (single chain, e.g., nanobody) or engineered affinity protein (e.g., affibody) that binds to a transcriptional condensate component, a heterochromatin condensate component, or a component of a condensate physically associated with an mRNA initiation or elongation complex. In some embodiments, the agent is a synthetic RNA.

In some embodiments, the agent is, or is encoded by, a synthetic RNA (e.g., modified mRNAs) conjugated to non-nucleic acid molecules. In some embodiments, the synthetic RNAs are conjugated to (or otherwise physically associated with) a moiety that promotes cellular uptake, nuclear entry, and/or nuclear retention (e.g., peptide transport moieties or the nucleic acids). In some embodiments, the synthetic RNA is conjugated to a peptide transporter moiety, for example a cell-penetrating peptide transport moiety, which is effective to enhance transport of the oligomer into cells. For example, in some embodiments the peptide transporter moiety is an arginine-rich peptide. In further embodiments, the transport moiety is attached to either the 5′ or 3′ terminus of the oligomer. When such peptide is conjugated to either termini, the opposite termini is then available for further conjugation to a modified terminal group as described herein. Peptide transport moieties are generally effective to enhance cell penetration of the nucleic acids. In some embodiments, a glycine (G) or proline (P) amino acid subunit is included between the nucleic acid and the remainder of the peptide transport moiety (e.g., at the carboxy or amino terminus of the carrier peptide) to reduces the toxicity of the conjugate, while maintaining or improving efficacy relative to conjugates with different linkages between the peptide transport moiety and nucleic acid.

In some embodiments, the agent is a phase (e.g., a disruptor of formation of a condensate) disruptor. In some embodiments, the phase disruptor is an ATP depletor (e.g., sodium azide (NaN3) and dinitrophenol (DNP)) or 1,6-hexanediol.

In some embodiments, an agent as described herein targets a transcriptional condensate component for intracellular degradation, e.g., by the ubiquitin-proteasome system (UPS). In some embodiments, such an agent may be used to reduce the level of a transcriptional condensate component and thereby inhibit condensate formation, maintenance, and/or activity. In some embodiments an agent that targets a transcriptional condensate component for intracellular degradation comprises a first domain that binds to a transcriptional condensate component and a second domain that targets an entity with which it is associated for degradation, e.g., by the proteasome. In some embodiments, an agent as described herein targets a condensate (a heterochromatin condensate, or a condensate physically associated with an mRNA initiation or elongation complex) component for intracellular degradation, e.g., by the ubiquitin-proteasome system (UPS). In some embodiments, such an agent may be used to reduce the level of a condensate component and thereby inhibit condensate formation, maintenance, and/or activity. In some embodiments an agent that targets a condensate (a heterochromatin condensate, or a condensate physically associated with an mRNA initiation or elongation complex) component for intracellular degradation comprises a first domain that binds to a condensate component and a second domain that targets an entity with which it is associated for degradation, e.g., by the proteasome. Such an agent may be used to reduce the level of the condensate component to which it binds. In some embodiments a condensate component is targeted for degradation based upon the proteolysis targeting chimera (PROTAC) concept (see, e.g., Protacs: chimeric molecules that target proteins to the Skp1-Cullin-F box complex for ubiquitination and degradation Sakamoto, Kathleen M. et al. Proceedings of the National Academy of Sciences (2001), 98 (15), 8554-8559; Carmony, K C and Kim, K, PROTAC-Induced Proteolytic Targeting, Methods Mol Biol. 2012; 832: Ch. 44). In this approach, a heterobifunctional agent is designed to contain a first domain that binds to a protein of interest (in this case a condensate component (e.g., transcriptional condensate component)), a second domain that binds to an E3 ubiquitin ligase complex, and, typically, a linker to tether these domains together. In some embodiments the first domain, the second domain, or both, comprises a peptide. In some embodiments the first domain, the second domain, or both, comprises a small molecule. For example, the molecule that binds to the ubiquitin ligase complex may be a small molecule that is a ligand for cereblon, a component of the Cullin4A ubiquitin ligase complex. A small molecule that binds to cereblon may be a phthalimide, e.g., thalidomide, lenalidomide, or pomalidomide (see, e.g., Winter, G E, et al. Science 348 (6241), 1376-1381; Pat. Pub. Nos. 20160235731 and 20180009779). In some embodiments a molecule that binds to the von Hippel-Lindau E3 ubiquitin ligase, such as the small molecules (e.g., hydroxyproline analogues) described in Buckley D L, et al. Targeting the von Hippel-Lindau E3 ubiquitin ligase using small molecules to disrupt the VHL/HIF-1α interaction. J Am Chem Soc. 2012; 134(10):4465-4468 or the small molecules described in Galdeano, C. et al. Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J. Med. Chem. 57, 8657-8663 (2014) may be used. In some embodiments the PROTAC may target a bromodomain-containing protein such as BRD1, BRD2, BRD3, and/or BRD4 for degradation. In some embodiments the PROTAC may target a kinase such as CDK7 or CDK9 for degradation. See, e.g., Robb, C M, et al., Chem Commun (Camb). 2017 Jul. 4; 53(54):7577-7580.

In some embodiments, the agent is a small molecule that binds to a component (e.g., a component as described herein) which may be linked to a small molecule that binds to a ubiquitin ligase complex, the resulting complex used to target the protein for degradation. In some embodiments, the small molecule binds to an IDR having a motif listed in Table 51. In some embodiments, a method comprises identifying a small molecule that binds to a component (or IDR) listed in Table 51 and linking said small molecule to a small molecule that binds to a component of an ubiquitin ligase complex.

In some embodiments, contact between the agent and the transcriptional condensate (e.g., a transcriptional condensate component) stabilizes or dissolves the condensate, thereby modulating transcription, splicing, or silencing of the one or more genes. In some embodiments, contact between the agent and the condensate (e.g., a heterochromatin condensate, or a condensate physically associated with an mRNA initiation or elongation complex) stabilizes or dissolves the condensate, thereby modulating transcription, splicing, or silencing of the one or more genes. In some embodiments, the agent increases or the decreases the half-life of the condensate by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the agent increases or the decreases the half-life of the condensate by at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more relative to the half-life of an uncontacted condensate.

In some embodiments, the agent can bind DNA, RNA, or proteins and prevent integration of a component into a transcriptional condensate, a heterochromatin condensate, or a condensate physically associated with an mRNA initiation or elongation complex. In other embodiments, the agent integrates into existing transcriptional condensates. In other embodiments, the agent integrates into existing heterochromatin condensates, or condensates physically associated with an mRNA initiation or elongation complex. In other embodiments, the agent forces integration of another component into existing transcriptional condensates, heterochromatin condensates, or condensates physically associated with an mRNA initiation or elongation complex. In other embodiments, the agent prevents a component from entering a transcriptional condensate, a heterochromatin condensate, or a condensate physically associated with an mRNA initiation or elongation complex.

In some embodiments, the agent binds to, masks, and/or neutralizes an acidic residue in an IDR (e.g., an activation domain of a transcription factor; an IDR of a signaling factor, nuclear receptor, methyl-DNA binding protein, RNA polymerase, or suppressor). This may, in some embodiments, inhibit interaction of the TF with a coactivator, e.g., Mediator, e.g., a Mediator component. This may, in some embodiments, modulate signal factor dependent transcription, gene silencing, or mRNA initiation and/or elongation (e.g., splicing). In some embodiments an agent binds to, or modifies, a non-acidic residue in an activation domain of a transcription factor. This may, in some embodiments, enhance interaction of the transcription factor with a coactivator, e.g., Mediator, e.g., a Mediator component. In some embodiments, the agent may enhance interaction of the transcription factor (e.g., nuclear receptor, ligand independent mutant nuclear receptor) with a gene silencing factor or signaling factor. In some embodiments, the agent may preferentially interact with a mutant transcription factor (e.g., ligand independent mutant nuclear receptor) than a wild-type transcription factor.

In some embodiments, the agent is a polypeptide or protein that has at least 50%, at least 60%, at least 70%, at least 80%, at least 90%, at least 95% of an IDR (e.g., an IDR having a motif listed in Table S2, an IDR of a transcription factor listed in Table S3). In some embodiments, the agent has multiple IDRs (e.g., 2, 3, 4, 5, or more IDR regions). In some embodiments, the component has at least one IDR separated into multiple discrete sections (e.g., 2, 3, 4, 5 or more sections). In some embodiments, the sections are separated by linker sequences or structured amino acids.

In some embodiments, the agent is a modified transcriptional condensate component (e.g., a transcription factor, a transcriptional co-activator, a nuclear receptor ligand). In some embodiments, the agent is a modified heterochromatin condensate component (e.g., methyl-DNA binding protein, gene silencing factor). In some embodiments, the agent is a modified condensate physically associated with mRNA initiation or elongation complex component (e.g., splicing factor, RNA polymerase II). In some embodiments, the component has a modified IDR region. In some embodiments, the IDR is located in or is derived from the activation domain of a transcription factor. In some embodiments, the modified IDR has an increased or reduced number of serines than the wild-type sequence. In some embodiments, the IDR has a reduced or increased number of aromatic acids as compared to the wild type sequence. In some embodiments, the IDR has a reduced or increased number of acidic residues as compared to the wild type sequence. In some embodiments, the IDR has a reduced or increased positive or negative net charge as compared to the wild type sequence.

In some embodiments, the IDR has a reduced or increased number of proline residues as compared to the wild type sequence. In some embodiments, the IDR has a reduced or increased number of serine and/or threonine residues as compared to the wild type sequence. In some embodiments, the IDR has a reduced or increased number of glutamine residues as compared to the wild type sequence. In some embodiments, residue or residues of the IDR ((e.g., serine, threonine, proline, acidic residues, glutamic acid, aromatic residues) may be increased or decreased relative to the wild type sequence by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 75, 100, or more. In some embodiments, residue or residues of the IDR ((e.g., serine, threonine, proline, acidic residues, glutamic acid, aromatic residues) may be increased or decreased relative to the wild type sequence by a factor of about 1.2, 1.5, 2, 2.5, 3, 3.5, 4, 4.5, 5, 6, 7, 8, 9, 10, or more. In some embodiments, residue or residues of the IDR ((e.g., serine, threonine, proline, acidic residues, glutamic acid, aromatic residues) may be increased or decreased relative to the wild type sequence by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, all acidic residues of the IDR may be replaced by non-acidic residues (e.g., non-charged residues, basic residues). In some embodiments, all proline residues of the IDR may be replaced by non-proline residues (e.g., hydrophilic residues, polar residues). In some embodiments, all serine and/or threonine residues of the IDR may be replaced by non-serine and/or threonine residues (e.g., hydrophobic residues, acidic residues). In some embodiments, the modified component has a reduced or increased valency for other components of a condensate (e.g., transcriptional condensate). In some embodiments, the modified transcriptional condensate component suppresses or prevents condensate formation. In some embodiments, the modified heterochromatin condensate component or modified component of a condensate physically associated with mRNA initiation or elongation complex suppresses or prevents condensate formation or condensate activity.

Transcription Factor Activity

Master transcription factors (TFs) are known to regulate key cell identity genes by establishing cell type specific enhancers (e.g., super-enhancers). Further, nuclear receptors are TFs associated with numerous diseases and conditions, including cancers. TFs activate transcription of their target genes by recruiting coactivators. The binding between TFs and coactivators has been described as “fuzzy” since their interaction interface cannot be described by a single conformation. These dynamic interactions are also typical of the IDR-IDR interactions that compose phase-separated condensates. TFs with diverse types of low complexity activation domains are thought to interact with the same small set of multisubunit coactivator complexes, which include Mediator, p300 and general transcription factor II D (TFIID). We propose that the mechanism of action by which TFs interact with coactivators and thereby activate transcription is by nucleating coactivator condensates. Thus, altering TF activation domains will disrupt the interaction with the coactivator complexes and thereby alter the transcriptional output.

Thus, in some embodiments, a transcriptional condensate is modulated by modulating the binding of a transcription factor (TF) associated with the transcriptional condensate to a component of the transcriptional condensate. In some embodiments, the affinity of TF activation domains for one or more condensate components is modulated. In some embodiments, the affinity of a component for a TF (e.g., a TF activation domain) is modulated. In some embodiments, formation of the transcriptional condensate is modulated by modulating the binding of a transcription factor (TF) associated with the transcriptional condensate to a component of the transcriptional condensate. In some embodiments, binding of the TF to a component associated with a transcriptional condensate is modulated by modulating a level of the TF or the component. In other embodiments, a heterochromatin condensate, or a condensate physically associated with an mRNA initiation or elongation complex is modulated by modulating the binding of a transcription factor (TF) associated with the condensate to a component of the condensate. In some embodiments, the affinity of TF activation domains for one or more condensate components (e.g., a heterochromatin condensate component, or a component of a condensate physically associated with an mRNA initiation or elongation complex) is modulated. In some embodiments, the affinity of a component for a TF (e.g., a TF activation domain) is modulated. In some embodiments, formation of the heterochromatin condensate, or a condensate physically associated with an mRNA initiation or elongation complex is modulated by modulating the binding of a transcription factor (TF) associated with the condensate to a component of the condensate. In some embodiments, binding of the TF to a component associated with a heterochromatin condensate, or a condensate physically associated with an mRNA initiation or elongation complex e is modulated by modulating a level of the TF or the component.

The component is not limited and may be any component described herein. In some embodiments, the component is a coactivator, cofactor, or nuclear receptor ligand. In some embodiments, the component is Mediator, a mediator component, MED1, MED15, GCN4, p300, BRD4, a hormone (e.g. estrogen) or TFIID. In some embodiments, the component is a transcription factor. In some embodiments, the transcription factor has an IDR in an activation domain. In some embodiments, the transcription factor is OCT4, p53, MYC or GCN4, NANOG, MyoD, KLF4, a SOX family transcription factor, a GATA family transcription factor, or a nuclear receptor (e.g., a nuclear hormone receptor, Estrogen Receptor, Retinoic Acid Receptor-Alpha). In some embodiments, the nuclear receptor activates transcription when bound to a cognate ligand. In some embodiments, the nuclear receptor is a mutant nuclear receptor that activates transcription in the absence of the cognate ligand. The mutant nuclear receptor maybe any mutant nuclear receptor described herein. In some embodiments, the transcription factor is a transcription factor associated with a super-enhancer. In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3).

In some embodiments, the binding of the transcription factor to a component of the transcriptional condensate (e.g., a non-transcription factor component) is modulated by contacting the transcription factor or transcriptional condensate with an agent described herein. In some embodiments, the binding of the transcription factor to a component of the heterochromatin condensate, or a condensate physically associated with an mRNA initiation or elongation complex is modulated by contacting the transcription factor or heterochromatin condensate, or a condensate physically associated with an mRNA initiation or elongation complex, with an agent described herein. In some embodiments, the agent is a peptide, nucleic acid, or small molecule. In some aspects, a peptide having a negative charge may bind to an IDR having a positive charge. In some aspects, a peptide having a positive charge may bind to an IDR having a negative charge.

In some embodiments, the agent may be any small molecule described herein. Small molecules may be designed to prevent the association of the transcription factor activation domain (e.g., an IDR in the transcription factor activation domain) with the intrinsically disordered region on cognate coactivators. This may be especially relevant in cancers that harbor oncogenic fusion proteins that involve IDRs (MLL-rearrangements, EWS-FLI, ETS fusions, BRD4-NUT, NUP98 fusions, oncogenic transcription factor fusions, etc.). Perturbing such an interaction may be utilized to enhance, diminish or otherwise alter the transcriptional output associated with either a specific transcription factor or a specific locus. Small molecules may also be designed to preferentially bind to a mutant transcription factor (e.g., mutant nuclear receptor) over a wild-type transcription factor.

Altering Client Interactions with Scaffolds

Molecular condensates have been described to have multiple types of components that can be divided in “scaffolds” and “clients” (Banani, S. F., Rice, A. M., Peeples, W. B., Lin, Y., Jain, S., Parker, R., and Rosen, M. K. (2016). Compositional Control of Phase-Separated Cellular Bodies. Cell 166, 651-663). Scaffold components phase separate and form condensates in which they are highly concentrated. While phase separated, these scaffold components can interact with client components that, by themselves, are not phase separated, but reach high local concentrations through client scaffold interactions (Banani et al., 2016). We propose that transcriptional condensates consist of scaffold and client components and that the introduction of peptide mimetics and other biomolecules that target the interacting domains of these client components, i.e. intrinsically disordered domains or regions, will exclude these clients from the transcriptional condensate. These clients can be transcriptional co-factors so that exclusion from the transcriptional condensate alters transcription. These clients can also be signaling transcriptions factors so that exclusion from the transcriptional condensate specifically renders over-activated signaling pathways transcriptionally inactive. In some aspects, the scaffold is a component that can assemble to form a condensate in a cell, or in vitro, then the component can be considered a scaffold component.

In some embodiments, the transcriptional condensate is modulated by modulating the amount or level of a component (e.g., client component) associated with the transcriptional condensate. The component (e.g., client component) is not limited and may be any condensate component described herein. In some embodiments, the component (e.g., client component) is one or more transcriptional co-factors and/or signaling transcriptions factors and/or nuclear receptor ligands (e.g., hormones). In some embodiments, the component (e.g., client component) is Mediator, MED1, MED15, GCN4, p300, BRD4, a hormone, or TFIID.

In some embodiments, the amount or level of the component (e.g., client component) associated with the transcriptional condensate is modulated by contact with an agent that reduces or eliminates interactions between the component (e.g., client component) and the transcriptional condensate. The agent is not limited and may be any agent described herein. In some embodiments, the agent is a peptide mimetic or analogous biomolecule.

In some embodiments, the agent targets an interacting domain of the component (e.g., client component). In some embodiments, the interacting domain is an intrinsically disordered domain or region (IDR). The IDR is not limited. In some embodiments, the IDR is an IDR having a motif listed in Table S2.

Signaling

The examples described here show that the cell type-dependent specificity of signaling may be achieved, at least in part, by addressing signaling factors to transcriptional condensates through phase separation at super-enhancers. In this manner, multiple signaling factor molecules could be concentrated in such condensates and occupy appropriate sites on the genome.

Thus, in some embodiments, a condensate (e.g., transcriptional condensates) may be modulated to increase or decrease affinity for a signaling factor (e.g., with an agent). In some embodiments, the condensate (e.g., transcriptional condensates) may be contacted with an agent that increases or decreases affinity for the signaling factor. For example, the agent may associate with the signaling factor and another component of the condensate (e.g., transcriptional condensates). Alternatively, the agent may reduce or block association of the agent with a component of the transcription factor. In some embodiments, the affinity of the signaling factor for the condensate (e.g., transcriptional condensates) may be modulated (e.g., with an agent). In some embodiments, the agent may modulate transcription activation by the signaling factor (e.g., by modulating formation, composition, maintenance, dissolution, activity and/or regulation of a transcriptional condensate associated with the signaling factor). In some embodiments, the agent's modulation of condensate/signaling factor affinity or activity is cell-type or enhancer (e.g. super-enhancer) specific. In some embodiments, the agent modulates affinity between the signaling factor and a co-factor (e.g., mediator or a mediator component).

In some embodiments, the condensate (e.g., transcriptional condensates) is associated with an enhancer (e.g., a super-enhancer). The enhancer may be associated with one or more genes described herein or known in the art. In some embodiments, the enhancer is associated with one or more genes involved in cell identity. In some embodiments, the enhancer is associated with genes associated with a disease or condition described herein (e.g., cancer). The condensate may be associated with any TF described herein or known in the art. In some embodiments, the TF comprises one or more IDRs. In some embodiments, the condensate is associated with a master TF. In some embodiments, the TF associated with the condensate is MyoD, Oct4, Nanog, Klf4 or Myc.

The condensates (e.g., transcriptional condensates) may be associated with (e.g. control transcription of) any gene or group of genes. In some embodiments, the gene or genes are involved in cell identity. In some embodiments, the genes are associated with a disease or condition described herein (e.g., cancer). The condensate (e.g., transcriptional condensates) may comprise a co-factor. The co-factor is not limited. In some embodiments, the co-factor and signaling factor preferentially associate in a condensate. In some embodiments, the co-factor is Mediator, a mediator component, MED1, MED15, p300, BRD4, TFIID.

The condensate (e.g., transcriptional condensates) may be associated with a signal response element (e.g., short sequences of DNA within a gene promoter region that are able to bind specific signaling factors and regulate transcription). In some embodiments, the signal response element is associated with a super-enhancer. In some embodiments, the signal response element is present in both regions of the genome associated with super-enhancers and regions of the genome not associated with super-enhancers.

The signaling factor is not limited and may be any signaling factor described herein or known in the art. In some embodiments, the signaling factor comprises one or more IDRs. In some embodiments, the signaling factor is selected from the group consisting of NF-kB, FOXO1, FOXO2, FOXO4, IKKalpha, CREB, Mdm2, YAP, BAD, p65, p50, GLI1, GLI2, GLI3, YAP, TAZ, TEAD1, TEAD2, TEAD3, TEAD4, STAT1, STAT2, STAT5, STAT4, STAT5A, STAT5B, STAT6, AP-1, C-FOS, CREB, MYC, JUN, CREB, ELK1, SRF, NOTCH1, NOTCH2, NOTCH3, NOTCH4, RBPJ, MAML1, SMAD2, SMAD3, SMAD4, IRF3, ERK1, ERK2, MYC, TCF7L2, TCF7, TCF7L1, LEF1, or Beta-Catenin. In some embodiments, the signaling factor preferentially binds to one or more signal response elements or mediator associated with the condensate. In some embodiments, the condensate comprises a master transcription factor.

Signaling factors and cofactors may interact specifically with transcriptional condensates, and some signaling pathways are altered in disease. The signaling pathways are not limited. In some embodiments, the signaling pathway is the Akt/PKB signaling pathway, AMPK signaling pathway, cAMP-dependent pathway, EGF receptor signaling pathway, Hedgehog signaling pathway, Hippo signaling pathway, hypoxia inducible factor (HIF) signaling pathway, insulin signaling pathway, IGF signaling pathway, JAK-STAT signaling pathway, MAPK/ERK signaling pathway, mTOR signaling pathway, NF-kB pathway, Notch signaling pathway, PI3K/AKT signaling pathway, PDGF receptor pathway, T cell receptor signaling pathway, TGF beta signaling pathway, TLR signaling pathway, VEGF receptor signaling pathway, or Wnt signaling pathway. In some embodiments, the signaling pathway is a nuclear receptor associated signaling pathway. The nuclear receptor is not limited and may be any nuclear receptor identified herein. Altering condensate formation, composition, maintenance, dissolution, morphology and/or regulation may provide therapeutic benefit when signaling pathways contribute to disease pathogenesis.

In some embodiments, modulating the transcriptional condensate modulates one or more signaling pathways. In some embodiments, the signaling pathway contributes to disease pathogenesis. In some embodiments, the disease is a proliferative disease, an inflammatory disease, a cardiovascular disease, a neurological disease or an infectious disease. In some embodiments, the disease is cancer (e.g., breast cancer).

The type of cancer is not limited. “Cancer” is generally used to refer to a disease characterized by one or more tumors, e.g., one or more malignant or potentially malignant tumors. The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclear to cytoplasmic ratio, atypical mitoses, etc.); invasion of adjacent tissues (e.g., breaching a basement membrane); and/or metastasis. Malignant tumors have a tendency for sustained growth and an ability to spread, e.g., to invade locally and/or metastasize regionally and/or to distant locations, whereas benign tumors often remain localized at the site of origin and are often self-limiting in terms of growth. The term “tumor” includes malignant solid tumors, e.g., carcinomas (cancers arising from epithelial cells), sarcomas (cancers arising from cells of mesenchymal origin), and malignant growths in which there may be no detectable solid tumor mass (e.g., certain hematologic malignancies). Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., the WHO Classification of Tumours series, 4th ed, or 3rd ed (Pathology and Genetics of Tumours series), by the International Agency for Research on Cancer (IARC), WHO Press, Geneva, Switzerland, all volumes of which are incorporated herein by reference. In some embodiments, the cancer is lung cancer, breast cancer, cervical cancer, colon cancer, gastric cancer, kidney cancer, leukemia, liver cancer, lymphoma, (e.g., a Non-Hodgkin lymphoma, e.g., diffuse large B-cell lymphoma, Burkitts lymphoma) ovarian cancer, pancreatic cancer, prostate cancer, rectal cancer, sarcoma, skin cancer, testicular cancer, or uterine cancer. The type of cancer is not limited. In some embodiments, the cancer exhibits aberrant gene expression. In some embodiments, the cancer exhibits aberrant gene product activity. In some embodiments, the cancer expresses a gene product at a normal level but harbor a mutation that alters its activity. In the case of an oncogene that has an aberrantly increased activity, the methods of the invention can be used to reduce expression of the oncogene. In the case of a tumor suppressor gene that has aberrantly reduced activity (e.g., due to a mutation), the methods of the invention can be used to increase expression of the tumor suppressor gene by modulating the regulatory landscape.

Nuclear Pore Association

Transcriptional condensates can interact with nuclear pore proteins allowing preferential access to incoming signals and preferential export of newly transcribed mRNA. The stabilization or disruption of the interaction between the condensate and the nuclear pore may alter the transcriptional output of the condensate. It may also favor export and translation of the mRNAs from the genes associated with the condensate.

In some embodiments, modulating the transcriptional condensate modulates interactions between the transcriptional condensate and one or more nuclear pore proteins. In some embodiments, modulation of the interactions between the transcriptional condensate and the one or more nuclear pore proteins modulates nuclear signaling, mRNA export, and/or mRNA translation. In some embodiments, the nuclear signaling, mRNA export, and/or mRNA translation is associated with a disease.

Inflammation

The inflammatory response to bacterial or viral infection is dependent on the activation of key cytokines and chemokines. Reduction in transcription of these inflammatory response genes is known to reduce the deleterious effects of bacterial or viral infection. Robust expression of key inflammatory genes could be dependent on condensate formation, which might be especially dependent on specific proteins, RNA or DNA motifs that can be targeted by a peptide, nucleic acid or small molecule.

In some embodiments, modulating the transcriptional condensate (or, in some embodiments, heterochromatin condensate, or a condensate physically associated with an mRNA initiation or elongation complex) modulates an inflammatory response. In some embodiments, the inflammatory response is an inflammatory response to a virus or bacteria. In some embodiments, the inflammatory response is an inappropriate, misregulated, or overactive inflammatory response. In certain embodiments, methods of the disclosure are used to decrease inflammation, to decrease expression of one or more inflammatory cytokines, and/or to decrease an overactive inflammatory response in a subject having an inflammatory condition. In some embodiments, an inflammatory response is modulated by modulating a condensate and thereby modulating transcription, mRNA initiation and/or elongation, or gene silencing of one or more genes involved in inflammation or reducing an inflammation response. In some embodiments, the activity of a signaling pathway involved in inflammation or reducing an inflammation response is modulated via a method disclosed herein (e.g, my modulating affinity of a signaling factor with a condensate).

Modulating Condensates with DNA

Alteration of DNA sequences or modification by DNA methylation/demethylation or other DNA modification such as acetylation/deacetylation may influence condensate formation, composition, maintenance, dissolution, morphology and/or regulation. In addition, components (DNA, RNA, or protein) may be tethered to the genomic DNA in a site-specific manner by utilizing a fusion to dCas9 (or other catalytically inactive site-specific nuclease) and using specific guide RNAs. A similar approach may be used to localize specific components to an existing condensate, which may alter its composition, maintenance, dissolution or regulation.

In some embodiments, the condensate (e.g., transcriptional condensate) is modulated by altering a nucleotide sequence (e.g., genomic DNA sequence) associated with the condensate. For instance, an enhancer (e.g., super-enhancer) associated with a transcriptional condensate may be altered. A transcription factor binding site may also be altered. In some embodiments, a hormone response element or a signal response element may be altered. Furthermore, a gene encoding a component associated with a condensate (e.g., encoding a transcription factor, a co-factor, a co-activator, a repressive factor, a methyl-DNA associated binding protein) may be altered. The alteration could be in coding or noncoding region. In some embodiments, the alteration comprises adding or deleting nucleotides. In some embodiments, nucleotides are added to trigger or enhance condensate formation or modulate condensate stability. In some embodiments, nucleotides are deleted to prevent condensate formation or modulate condensate stability. In some embodiments, addition or deletion of nucleotides influences condensate formation, composition, maintenance, dissolution, morphology and/or regulation.

In some embodiments, the DNA associated with the condensate is localized in heterochromatin (e.g., facultative heterochromatin). In some embodiments, the DNA associated with the condensate is methylated. In some embodiments, genomic DNA is methylated or demethylated to modulate condensate formation. In some embodiments, the DNA is methylated or demethylated to modulate condensate formation or stability and thereby modulate gene silencing. In some embodiments, site-specific catalytically inactive endonucleases are used to methylate or demethylate heterochromatin to modulate condensate formation or stability and thereby modulate gene silencing.

In some embodiments, the alteration comprises an epigenetic modification. In some embodiments, the epigenetic modification comprises DNA methylation. In some embodiments, the alteration of the nucleotide sequence comprises the tethering of a DNA, RNA, or protein to the nucleotide sequence. In some embodiments, the DNA, RNA, or protein is a transcriptional condensate component or fragment thereof (e.g., an IDR containing fragment) as described herein. In some embodiments, the DNA, RNA, or protein is a heterochromatin condensate component or fragment thereof (e.g., an IDR containing fragment) as described herein. In some embodiments, the DNA, RNA, or protein is an agent as described herein. In some embodiments, the DNA, RNA, or protein promotes or enhances formation of a condensate. In some embodiments, the DNA, RNA, or protein suppresses or prevents formation of a condensate. In some embodiments, a cofactor (e.g., mediator) or fragment thereof (e.g., an IDR containing fragment) is tethered to the nucleotide sequence. In some embodiments, a methyl-DNA binding protein or fragment thereof (e.g., an IDR containing fragment) is tethered to the nucleotide sequence. In some embodiments, a cyclin dependent kinase or fragment thereof is tethered to the nucleotide sequence. In some embodiments, a splicing factor or fragment thereof (e.g., an IDR containing fragment) is tethered to the nucleotide sequence.

In some embodiments, a catalytically inactive site specific nuclease and an effector domain capable of attaching a DNA, RNA, or protein to the nucleotide sequence is used. In some embodiments, the catalytically inactive site specific nuclease dCas (e.g., dCas9 or Cpf1) is used.

A variety of CRISPR associated (Cas) genes or proteins which are known in the art can be modified to make a catalytically inactive site specific nuclease, the choice of Cas protein will depend upon the particular conditions of the method (e.g., ncbi.nlm.nih.gov/gene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Crptococcus, a Corynebacterium, a Haemophilus, a Eubacterium, a Pasteurella, a Prevotella, a VeiUonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs.

In some embodiments, the Cas protein is Cpf1 protein or a functional portion thereof. In some embodiments, the Cas protein is Cpf1 from any bacterial species or functional portion thereof. In certain embodiments, a Cpf1 protein is a Francisella novicida U112 protein or a functional portion thereof, a Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain.

In some embodiments a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al, Science, 339:819-823). Other amino acids mutations that create a catalytically inactive Cas9 protein includes mutating at residue 10 and/or residue 840. Mutations at both residue 10 and residue 840 can create a catalytically inactive Cas9 protein, sometimes referred herein as dCas9. For example, a D10A and a H840A Cas9 mutant is catalytically inactive.

As used herein an “effector domain” is a molecule (e.g., protein) that modulates the expression and/or activation of a genomic sequence (e.g., gene). The effector domain may have methylation activity or demethylation activity (e.g., DNA methylation or DNA demethylation activity). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas (e.g., dCas) nucleic acid sequence or variant thereof and an effector domain nucleic acid sequence are introduced into a cell having a condensate as a chimeric sequence. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

In some embodiments, the catalytically inactive site specific nuclease can be guided to specific DNA sites by one or more RNA sequences (sgRNA) to modulate activity and/or expression of one or more genomic sequences (e.g., exert certain effects on transcription or chromatin organization, or bring specific kind of molecules into specific DNA loci, or act as sensor of local histone or DNA state). In specific aspects, fusions of a dCas9 tethered with all or a portion of an effector domain create chimeric proteins that can be guided to specific DNA sites by one or more RNA sequences to modulate or modify methylation or demethylation of one or more genomic sequences. As used herein, a “biologically active portion of an effector domain” is a portion that maintains the function (e.g. completely, partially, minimally) of an effector domain (e.g., a “minimal” or “core” domain). The fusion of the Cas9 (e.g., dCas9) with all or a portion of one or more effector domains created a chimeric protein.

Examples of effector domains include a chromatin organizer domain, a remodeler domain, a histone modifier domain, a DNA modification domain, a RNA binding domain, a protein interaction input devices domain (Grunberg and Serrano, Nucleic Acids Research, 3 ′8 (8): ′2663-267 ′5 (2010)), and a protein interaction output device domain (Grunberg and Serrano, Nucleic Acids Research, 3 ′8 (8): ′2663-267 ′5 (2010)). In some aspects, the effector domain is a DNA modifier. Specific examples of DNA modifiers include 5hmc conversion from 5mC such as Tet1 (Tet1CD); DNA demethylation by Tet1, ACID A, MBD4, Apobec1, Apobec2, Apobec3, Tdg, Gadd45a, Gadd45b, ROS1; DNA methylation by Dnmt1, Dnmt3a, Dnmt3b, CpG Methyltransferase M.SssI, and/or M.EcoHK31I. In specific aspects, an effector domain is Tet1. In other specific aspects, as effector domain is Dmnt3a. In some embodiments, dCas9 is fused to Tet1. In other embodiments, dCas9 is fused to Dnmt3a. Other examples of effector domains are described in PCT Application No. PCT/US2014/034387 and U.S. application Ser. No. 14/785,031, which are incorporated herein by reference in their entirety. Methods of using catalytically inactive site specific nuclease, effector domains for modifying a nucleotide sequence (e.g., genomic sequence), and sgRNA are taught in PCT/US2017/065918 filed 12 Dec. 2017, which is incorporated herein by reference.

Modulating Condensates with RNA

It is further noted that addition of exogenous RNAs, stabilization of RNAs, or removal of certain RNAs, can modulate condensates. Thus, in some embodiments, the transcriptional condensate is modulated by contacting the condensate with exogenously added RNA. In some embodiments, a heterochromatin condensate is modulated by contacting the condensate with exogenously added RNA. In some embodiments, a condensate associated with an mRNA initiation or elongation complex is modulated by contacting the condensate with exogenously added RNA.

In some embodiments, the exogenous RNA is a naturally occurring RNA sequence, a modified RNA sequence (e.g., a RNA sequence comprising one or more modified bases), a synthetic RNA sequence, or a combination thereof. As used herein a “modified RNA” is an RNA comprising one or more modifications (e.g., RNA comprising one or more non-standard and/or non-naturally occurring bases) to the RNA sequence (e.g., modifications to the backbone and or sugar). Methods of modifying bases of RNA are well known in the art. Examples of such modified bases include those contained in the nucleosides 5-methylcytidine (5mC), pseudouridine (Ψ), 5-methyluridine, 2′O-methyluridine, 2-thiouridine, N-6 methyladenosine, hypoxanthine, dihydrouridine (D), inosine (I), and 7-methylguanosine (m7G). It should be noted that any number of bases in a RNA sequence can be substituted in various embodiments. It should further be understood that combinations of different modifications may be used.

In some aspects, the exogenous RNA sequence is a morpholino. Morpholinos are typically synthetic molecules, of about 25 bases in length and bind to complementary sequences of RNA by standard nucleic acid base-pairing. Morpholinos have standard nucleic acid bases, but those bases are bound to morpholine rings instead of deoxyribose rings and are linked through phosphorodiamidate groups instead of phosphates. Morpholinos do not degrade their target RNA molecules, unlike many antisense structural types (e.g., phosphorothioates, siRNA). Instead, morpholinos act by steric blocking and bind to a target sequence within a RNA and block molecules that might otherwise interact with the RNA. In some embodiments, the synthetic RNA is as described in WO 2017075406.

In some embodiments an RNA sequence can vary in length from about 8 base pairs (bp) to about 200 bp, about 500 bp, or about 1000 bp. In some embodiments, the RNA sequence can be about 9 to about 190 bp; about 10 to about 150 bp; about 15 to about 120 bp; about 20 to about 100 bp; about 30 to about 90 bp; about 40 to about 80 bp; about 50 to about 70 bp in length.

In some embodiments, the exogenous RNA stabilizes or enhances the formation or stability of the condensate. In some embodiments, the exogenous RNA accelerates dissolution or prevents/suppresses formation of the condensate.

In some embodiments, removal of certain (i.e., specific) RNAs is performed using interference RNA (RNAi). As used herein, the term “RNA interference” (“RNAi”) (also referred to in the art as “gene silencing” and/or “target silencing”, e.g., “target mRNA silencing”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. In some aspects, removal of specific RNA is via transcriptional repression of the specific RNA.

In some embodiments, RNA is stabilized by protecting (capping) one or both ends of the RNA by methods known in the art. In some embodiments, RNA is stabilized by associating the RNA with a molecule (i.e., antisense nucleic acid or small molecule) that does not interfere with binding to a component of the condensate.

Modulation of RNA Processing by Targeting Components of Condensates

Some diseases are associated with abnormal processing of RNA species. In some embodiments, transcriptional condensates may fuse with condensates formed by the RNA processing apparatus. The stabilization or disruption of these condensates may alter RNA processing in a manner that is therapeutically beneficial. In some embodiments, the methods described herein may be used to modulate a condensate to enhance or stabilize fusion of a transcriptional condensate and a condensate formed by the RNA processing apparatus. In some embodiments, the methods described herein may be used to modulate a condensate to suppress or destabilize fusion of a transcriptional condensate and a condensate formed by the RNA processing apparatus. In some embodiments, a condensate physically associated with mRNA an initiation or elongation complex may be modulated by a method disclosed herein thereby modulating RNA processing. In some embodiments, a condensate physically associated with mRNA an initiation or elongation complex is modulated in a manner that is therapeutically beneficial. In some embodiments, condensates associated with mRNA elongation are modulated, thereby modulating mRNA splicing in a manner that is therapeutically beneficial (e.g., reduction in aberrant splicing variants, an increase in beneficial splicing variants).

Modulation of Translation by Modulation of mRNA Export

Transcriptional condensates can interact with nuclear pore proteins allowing preferential export of newly transcribed mRNA. The stabilization or disruption of the interaction between the condensate and the nuclear pore may thus alter translation of the mRNAs from the genes associated with the condensate. Such alteration may be therapeutically useful when diseases cause pathological levels of specific proteins. In some embodiments, the methods described herein may be used to modulate a condensate to enhance preferential export of newly transcribed mRNA. In some embodiments, the methods described herein may be used to modulate a condensate to suppress preferential export of newly transcribed mRNA. In some embodiments, modulating mRNA is therapeutic for treating a disease. In some embodiments, modulating mRNA returns a pathological level of a protein to a non-pathological level.

Utilizing Multivalent Molecules to Target Condensates

Condensates (e.g., transcriptional condensates, heterochromatin condensates, or condensates associated with mRNA initiation or elongation complexes) may be formed by multiple weak interactions between proteins having IDRs. Given that such disordered regions may not have any defined secondary or tertiary structure, small molecules or peptidomimetics that bind to these regions may do so with weak affinities. In order to concentrate such molecules into condensates (e.g., transcriptional condensates, heterochromatin condensates, or condensates associated with mRNA initiation or elongation complexes) to disturb weak IDR-IDR interactions, a bivalent molecule composed of an “anchor” and a “disruptor” may be utilized. The “disruptor” is a molecule that weakly binds interacting components of the condensate to disrupt or alter the nature of the interaction. The anchor component is a molecule which has strong affinity for a more structured region of a protein that is in or near the condensate, thus serving to concentrate the disruptor molecule in or near the condensate (e.g., transcriptional condensates, heterochromatin condensates, or condensates associated with mRNA initiation or elongation complexes).

In some embodiments, the transcriptional condensate is modulated by contacting the condensate with an agent that binds to an intrinsically disordered domain of a condensate component. In some embodiments, a heterochromatin condensate is modulated by contacting the condensate with an agent that binds to an intrinsically disordered domain of a condensate component. In some embodiments, a condensate associated with an mRNA initiation or elongation complex is modulated by contacting the condensate with an agent that binds to an intrinsically disordered domain of a condensate component. The component is not limited and may be any component described herein. In some embodiments, the component is Mediator, MED1, MED15, GCN4, p300, BRD4, a nuclear receptor ligand, or TFIID. In some embodiments, the component is a mediator component listed in Table S3. In some embodiments, the component is a transcription factor. In some embodiments, the transcription factor has an IDR in an activation domain. In some embodiments, the transcription factor is OCT4, p53, MYC, GCN4, NANOG, MyoD, KLF4, a SOX family transcription factor, a GATA family transcription factor, a nuclear receptor, or a a fusion oncogenic transcription factor. In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3).

The agent is also not limited and may be any suitable agent described herein. In some embodiments, the agent is multivalent (e.g., bivalent, trivalent, tetravalent, etc.). In some embodiments, the agent binds to an intrinsically disordered domain of a component and further binds to a non-intrinsically disordered domain of the same component. In some embodiments, the agent binds to an intrinsically disordered domain of a component and further binds to a second component associated with the transcriptional condensate. In some embodiments, the agent is multivalent and binds to an activation domain (e.g., IDR of an activation domain) and further binds to a non-activation domain (e.g., DNA binding domain), or a non-intrinsically disordered region of a transcription factor. In some embodiments, the agent specifically binds to a mutant transcription factor (e.g., a mutant transcription factor associated with a disease or condition) non-activation domain or a non-intrinsically disordered region of a transcription factor. In some embodiments, the agent does not bind to a wild-type transcription factor non-activation domain or a non-intrinsically disordered region of the wild-type transcription factor. In some embodiments, the multivalent agent binds to a nuclear receptor. In some embodiments, the multivalent agent preferentially binds to a mutant form of a nuclear receptor (e.g. a mutant form associated with a disease or condition). In some embodiments, the multivalent agent binds to a signaling factor, a co-factor, a methyl-DNA binding protein, a splicing factor, or an RNA polymerase.

In some embodiments, the agent alters or disrupts interactions between components of the transcriptional condensates. In some embodiments, the agent enhances or stabilizes the transcriptional condensate. In some embodiments, the agent suppresses or destabilizes the transcriptional condensate.

Tethering Components to DNA to Initiate Formation of a New Condensate or Alteration of an Existing Condensate

Transcriptional condensates and heterochromatin condensates can form on DNA. Thus, in order to form a new condensate, components (DNA, RNA, or protein) may be tethered to the genomic DNA in a site-specific manner by utilizing a catalytically inactive site specific nuclease and effector domain by methods disclosed herein. In some embodiments, the components are tethered to DNA (e.g., genomic DNA) using a dCas (e.g., dCas9) as described herein.

In some embodiments, formation of the transcriptional condensate is caused, enhanced, or stabilized by tethering one or more transcriptional condensate components to genomic DNA. In some embodiments, formation of the heterochromatin condensate is caused, enhanced, or stabilized by tethering one or more heterochromatin condensate components to genomic DNA. The components are not limited and may comprise any component described herein. In some embodiments, the components comprise DNA, RNA, and/or protein. In some embodiments, the components comprise Mediator, MED1, MED15, GCN4, p300, BRD4, β-catenin, STAT3, SMAD3, NF-kB, MECP2, MBD1, MBD2, MBD3, MBD4, HP1α, TBL1R, HDAC3, SMRT, RNA polymerase II, SRSF2, SRRM1, SRSF1, a nuclear receptor ligand, or TFIID. In some embodiments, the component is a mediator component listed in Table S3. In some embodiments, the component has an IDR disclosed herein. In some embodiments, the component is a transcription factor. In some embodiments, the transcription factor has an IDR in an activation domain. In some embodiments, the transcription factor is OCT4, p53, MYC, GCN4, NANOG, MyoD, KLF4, a SOX family transcription factor, a GATA family transcription factor, a nuclear receptor, or a fusion oncogenic transcription factor. In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3).

Using Principles in Phase Separation to Sequester Disease Related Proteins

Many diseases, including cancer, can be dependent on specific proteins involved in transcription. For example, the Myc transcription factor is overexpressed in a majority of all cancers and its perturbation leads to cancer cell death and differentiation. Myc has been shown to be preferentially incorporated into synthetic MED1 condensates. Thus, condensate formation induced by exogenous peptides, nucleic acids, or a small chemical molecules could be used sequester Myc away from its normal location at the promoters of active genes. Similar strategies could be used for any disease related protein that has the ability to be incorporated into a condensate. Disease related proteins that undergo mutation or fusion events could be especially vulnerable to this approach if the mutated version can be specifically incorporated into the synthetic condensate while the wildtype version is left alone.

In some embodiments, the methods described herein can be used to form or stabilize a condensate in order to sequester a protein, DNA, RNA or other condensate component as described herein. For example, a condensate may be induced to form by tethering a component to DNA and nucleating condensate formation. A condensate may also be induced to form by adding a suitable agent (e.g., exogenously added protein, DNA or RNA) or suitable component to a cell as described herein. In some embodiments, the sequestration of a component in a condensate modulates a second condensate by restricting access to the component. In some embodiments, the sequestered component is Myc. In some embodiments, the sequestered component is a mutant version of a wild-type protein. In some embodiments, the wild-type protein is not sequestered. In some embodiments, the sequestered component is a component over-expressed in a disease state. In some embodiments, sequestration of the component treats a disease state. The sequestration component is not limited and may be any component of a condensate described herein (e.g., Mediator, MED1, MED15, GCN4, p300, BRD4, a nuclear receptor ligand, and TFIID). In some embodiments, the sequestration component is a transcription factor or portion thereof, e.g., an activation domain. In some embodiments, the transcription factor has an IDR in an activation domain. In some embodiments, the transcription factor is OCT4, p53, MYC GCN4, NANOG, MyoD, KLF4, a SOX family transcription factor, a GATA family transcription factor, a nuclear receptor, or a fusion oncogenic transcription factor. In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3).

Non-Coding RNA is an Important Component of at Least Some Transcriptional Condensates

Many condensates have RNA components (Banani, S. F., Lee, H. O., Hyman, A. A., and Rosen, M. K. (2017). Biomolecular condensates: organizers of cellular biochemistry. Nat. Rev. Mol. Cell Biol. 18, 285-298). Gene regulatory elements produce exceptionally high levels of noncoding RNAs (Li, W., Notani, D., and Rosenfeld, M. G. (2016). Enhancers as non-coding RNA transcription units: recent insights and future perspectives. Nat. Rev. Genet. 17, 207-223). Yet the biological function of these RNAs are not understood. In addition, many transcription factors and co-factors can interact with RNA (Li et al., 2016). We propose that the formation and maintenance of some transcriptional condensates depend on noncoding RNAs. Anti-sense oligonucleotides, RNase (enzyme that degrades RNAs), or chemical compounds that directly target these noncoding RNA components within transcriptional condensates may cause the dissolution of transcriptional condensates in healthy and disease cells.

In some embodiments, a transcriptional condensate is modulated by modulating a level or activity of ncRNA associated with the transcriptional condensate. Modulating a level or activity of an ncRNA can be performed by any suitable method. In some embodiments, modulating a level or activity of an ncRNA may be performed by a method described herein (e.g., using RNAi). In some embodiments, the level or activity of the ncRNA is modulated by contacting the ncRNA with an anti-sense oligonucleotide, an RNase, or a small molecule that binds the ncRNA.

Methods of Screening

Some aspects of the disclosure are directed to methods of screening for agents as defined herein that are capable of modifying condensates (e.g., transcriptional condensates, heterochromatin condensates, condensates associated with mRNA initiation or elongation complexes).

In Vivo Assays to Screen for Condensate-Modifying Therapeutics

Some aspects of the disclosure are directed to methods of identifying an agent that modulates formation, stability, or morphology of a condensate (e.g., transcriptional condensate), comprising providing a cell having a condensate, contacting the cell with a test agent, and determining if contact with the test agent modulates formation, stability, or morphology of the condensate. In some embodiments, the condensate has a detectable tag and the detectable tag is used to determine if contact with the test agent modulates formation, stability, or morphology of the condensate. In some embodiments, the cell is a genetically engineered to express the detectable tag. The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label.

In some aspects, the method comprises a cell having condensate components, contacting the cell with a test agent, and determining if contact with the test agent modulates formation or activity of a condensate comprising the components (e.g., forms a heterotypic condensate, forms a homotypic condensate). In some embodiments, the one or more condensate components comprise a detectable label. In some embodiments, the condensate components will form a condensate and the test agent will be screened for modulating condensate formation (e.g., increasing or decreasing condensate formation or the rate of condensate formation). In some embodiments, the condensate components will not form a condensate and the test agent will be screened to see if it causes the formation of a condensate. In some embodiments, the condensate components comprise MED1 (or a fragment thereof) and ER or a fragment thereof, e.g., mutant ER (e.g., as described herein), e.g., mutant ER that is able to incorporate into a condensate comprising MED1 in the presence of tamoxifen.

In some embodiments, “determining” comprises measuring a physical property as compared to a control or reference. For example, determining if the stability of a condensate is modulated may comprise measuring the period of time a condensate exists as compared to a control condensate not subject to a test condition or agent. Determining if the shape of a condensate is modulated can comprise comparing the shape of a condensate as compared to a control condensate not subject to a test condition or agent. In some embodiments, one or more properties of a condensate may be “determined” to be modulated if they are changed by a statistically significant amount (e.g., at least 10%, at least 20%, at least 30%, at least 50%, at least 75%, or more).

In some embodiments, the detectable tag is a fluorescent tag (e.g., tdTomato). In some embodiments, the detectable tag is attached to a condensate component as described herein. In some embodiments, the component is selected from OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, a nuclear receptor ligand, a fusion oncogenic transcription factor, TFIID, a signaling factor, methyl-DNA binding protein, splicing factor, gene silencing factor, RNA polymerase, β-catenin, STAT3, SMAD3, NF-KB, MECP2, MBD1, MBD2, MBD3, MBD4, HP1α, TBL1R, HDAC3, SMRT, RNA polymerase II, SRSF2, SRRM1, SRSF1, and fragments thereof comprising an intrinsically disordered region (IDR).

In some embodiments, an antibody selectively binding to the condensate is used to determine if contact with the test agent modulates formation, stability, or morphology of the condensate. In some embodiments, the antibody binds to a condensate component as described herein. In some embodiments, the component is selected from Mediator, MED1, MED15, GCN4, p300, BRD4, a nuclear receptor ligand and TFIID, or a mediator component or transcription factor shown in Table S3 or described herein. In some embodiments, the component is a nuclear receptor or fragment thereof as described herein. In some embodiments, the component is selected from OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, a nuclear receptor ligand, a fusion oncogenic transcription factor, TFIID, a signaling factor, methyl-DNA binding protein, splicing factor, gene silencing factor, RNA polymerase, β-catenin, STAT3, SMAD3, NF-KB, MECP2, MBD1, MBD2, MBD3, MBD4, HP1α, TBL1R, HDAC3, SMRT, RNA polymerase II, SRSF2, SRRM1, SRSF1, and fragments thereof comprising an intrinsically disordered region (IDR).

Any suitable method of detecting modulation of the condensate by the test agent may be used, including methods known in the art and taught herein. In some embodiments, the step of determining if contact with the test agent modulates formation, stability, or morphology of the condensate is performed using microscopy, which is not limited. In some embodiments, the microscopy is deconvolution microscopy, structured illumination microscopy, or interference microscopy. In some embodiments, the step of determining if contact with the test agent modulates formation, stability, or morphology of the condensate is performed using DNA-FISH, RNA-FISH, or a combination thereof.

The type of cell having a condensate is not limited and may be any cell type disclosed herein. In some embodiments, the cell is affected by a disease (e.g., a cancer cell). In some embodiments, the cell having a condensate is a primary cell, a member of a cell line, cell isolated from a subject suffering from a disease, or a cell derived from a cell isolated from a subject suffering from a disease (e.g., a progenitor of an induced pluripotent cell isolated from a subject suffering from a disease).

In some embodiments, the cell is responsive to estrogen mediated gene activation. In some embodiments, the cell is responsive to nuclear receptor ligand mediated gene activation. In some embodiments, the cell comprises a mutant nuclear receptor. In some embodiments, the cell is a transgenic cell expressing a nuclear receptor (e.g., mutant nuclear receptor). In some embodiments, the cell is a cancer cell (e.g., breast cancer cell). In some embodiments, the cell is contacted with a test agent in the presence of estrogen and estrogen mediated gene activation is assessed. In some embodiments, the cell comprises estrogen receptor having a label and condensate incorporation of estrogen receptor in the presence of the test agent is assessed.

In some embodiments, the cell is responsive to estrogen mediated gene activation in the presence of tamoxifen. In some embodiments, the cell is a cancer cell (e.g., breast cancer cell). In some embodiments, the cell is contacted with a test agent in the presence of estrogen and tamoxifen and estrogen mediated gene activation is assessed. In some embodiments, the cell comprises estrogen receptor having a label and condensate incorporation of estrogen receptor in the presence of the test agent is assessed.

In some embodiments, the test agent is a tamoxifen analog. In some embodiments, the test agent is not a tamoxifen analog.

In some embodiments, the condensate comprises a signaling factor. In some embodiments, the in vitro condensate comprises a signaling factor or a fragment thereof comprising an IDR necessary for the activation of transcription of a gene. In some embodiments, the signaling factor is associated with an oncogenic signaling pathway.

In some embodiments, the condensate comprises a methyl-DNA binding protein or a fragment thereof comprising a C-terminal IDR, or a suppressor or fragment thereof comprising an IDR. In some embodiments, the condensate is associated with methylated DNA or heterochromatin. In some embodiments, the condensate comprises an aberrant level or activity of methyl-DNA binding protein (e.g., an increased or decreased level as compared to a reference level). In some embodiments, silencing of genes associated with the condensate by the agent are assessed. In some embodiments, the condensate comprises a splicing factor or a fragment thereof comprising an IDR, or an RNA polymerase or fragment thereof comprising an IDR.

In some embodiments, the condensate is associated with a transcription initiation complex or elongation complex. In some embodiments, the condensate is contacted with a cyclin dependent kinase. In some embodiments, the RNA polymerase is RNA polymerase II (Pol II). In some embodiments, changes in RNA transcription initiation activity associated with the condensate caused by contact with the agent are assessed In some embodiments, changes in RNA elongation or splicing activity associated with the condensate caused by contact with the agent are assessed.

In Vitro Assays to Screen for Condensate-Modifying Agents, e.g., Therapeutics

Condensates can form liquid droplets in vitro composed of RNA, DNA, and protein. Transcriptional condensate components can also form liquid droplets in vitro comprising one or more proteins, e.g., a TF and one or more coactivators or cofactors. Such droplets may further comprise RNA and/or DNA. Such liquid droplets are in vitro condensates and can correspond to and/or serve as models of condensates (e.g., transcriptional condensates, heterochromatin condensates, condensates associated with mRNA an initiation or elongation complex, condensates comprising splicing factors) that exist in vivo. These liquid droplets have measurable physical properties (i.e. size, concentration, permeability, and viscosity). These physical properties can correlate with the condensate's ability to activate a reporter gene in vivo. The effect of libraries of small molecules, peptides, RNA or DNA oligos on any physical property of the liquid droplet can be measured. Additionally, molecules that modulate droplet properties can be assayed for effects on gene expression using cell-based reporters. When individual components are absent from this condensate, it may be rendered non-functional (i.e., incapable of productive transcription). Additionally, incorporating novel components into existing condensates may modify, attenuate, or amplify their output. As such, it may be desirable to add or remove components from a preexisting condensate. Thus, in some embodiments, screening may be performed to isolate small molecules that bind DNA, RNA, or proteins and drive components into a transcriptional condensate, a heterochromatin condensate, or a condensate physically associated with mRNA initiation or elongation complexes. In other embodiments, screening may be performed to isolate small molecules that bind DNA, RNA, or proteins and prevent integration of a component into a condensate. In other embodiments, screening may be performed to isolate small molecules, proteins, RNA, proteins or DNAs that are designed, expressed or introduced that integrate into existing condensates. In other embodiments, screening may be performed to isolate small molecules, proteins, RNA, protein or DNAs that are designed, expressed or introduced that force integration of another component into existing condensates. In other embodiments, screening may be performed to isolate small molecules, proteins, RNA, or DNAs that are designed, expressed or introduced that prevent a component from entering a transcriptional condensate, a heterochromatin condensate, or a condensate physically associated with an mRNA initiation or elongation complex. In other embodiments, screening may be performed to isolate small molecules, proteins, RNA, or DNAs that are designed, expressed or introduced that prevent or decrease the likelihood of one or more components from forming a condensate.

Some aspects of the disclosure are directed to methods of identifying an agent that modulates formation, stability, or morphology of a condensate, comprising providing an in vitro condensate and assessing one or more physical properties of the in vitro condensate, contacting the in vitro condensate with a test agent, and assessing whether the test agent causes a change in the one or more physical properties of the in vitro condensate. In some embodiments, the one or more physical properties correlate with the in vitro condensate's ability to cause expression of a gene in a cell. In some embodiments, the one or more physical properties comprise size, concentration, permeability, morphology, or viscosity of the in vitro condensate. Any suitable method known in the art may be used to measure the one or more physical properties.

Some aspects of the disclosure are directed to methods of identifying an agent that modulates condensate formation. In some embodiments, the method comprises providing a composition comprising one or more condensate component or fragment thereof (e.g., any condensate component described herein, any condensate component having an IDR, mediator or a subunit thereof (e.g., MED1), a transcription factor), contacting the composition with a test agent, and determines whether the test agent modulates formation of a condensate comprising the condensate component(s) or modulates one or more properties of a condensate formed by the condensate component(s) (e.g., increases or decreases in stability, function, activity, morphology). In some embodiments, the one or more condensate components comprise a detectable label. One can provide the components, combine them in a vessel, and observe what happens in terms of condensate formation and/or measure the propert(ies) (e.g., increases or decreases in stability, function, activity, morphology) of resulting condensates. In some embodiments, the provided composition will form a condensate and the test agent will be screened for modulating formation (e.g., increasing or decreasing condensate formation or the rate of condensate formation). In some embodiments, the provided composition will not form a condensate and the test agent will be screened to see if it causes the formation of a condensate. In some embodiments, the condensate components comprise one or more co-factors (e.g., MED1 or a functional fragment thereof) and a nuclear receptor (e.g., wild-type nuclear receptor, mutant nuclear receptor, mutant nuclear receptor associated with a disease or condition) or a functional fragment thereof. In some embodiments, the condensate components comprise MED1 (or a fragment thereof) and ER or a fragment thereof, e.g., mutant ER (e.g., as described herein), e.g., mutant ER that is able to incorporate into a condensate comprising MED1 in the presence of tamoxifen.

In some embodiments, the in vitro condensate is responsive to nuclear receptor ligand mediated gene activation. In some embodiments, the in vitro condensate has constitutive mutant nuclear receptor mediated gene activation. In some embodiments, the in vitro condensate is responsive to estrogen mediated gene activation. In some embodiments, the in vitro condensate is contacted with a test agent in the presence of estrogen and estrogen mediated gene activation is assessed. In some embodiments, if estrogen mediated gene activation is decreased or eliminated in the presence of the test agent, then the test agent is identified as a candidate anti-cancer agent for treatment of an ER+ cancer. In some embodiments, the in vitro condensate comprises estrogen receptor having a label and condensate incorporation of estrogen receptor in the presence of the test agent is assessed. In some embodiments, if ER incorporation is decreased or eliminated in the presence of the test agent, then the test agent is identified as a candidate anti-cancer agent for treatment of an ER+ cancer.

In some embodiments, the in vitro condensate is responsive to estrogen mediated gene activation in the presence of tamoxifen (e.g., the in vitro condensate is isolated from a tamoxifen resistance breast cancer cell, the condensate comprises a mutant ER (e.g., as described herein) having constitutive activity. In some embodiments, the in vitro condensate is contacted with a test agent in the presence of estrogen and tamoxifen and estrogen mediated gene activation is assessed. In some embodiments, if estrogen mediated gene activation is decreased or eliminated in the presence of the test agent, then the test agent is identified as a candidate anti-cancer agent for treatment of tamoxifen resistant cancer. In some embodiments, the in vitro condensate comprises estrogen receptor having a label and condensate incorporation of estrogen receptor in the presence of the test agent is assessed. In some embodiments, if ER incorporation is decreased or eliminated in the presence of the test agent, then the test agent is identified as a candidate anti-cancer agent for treatment of tamoxifen resistant cancer.

In some embodiments, the test agent is a tamoxifen analog. In some embodiments, the test agent is not a tamoxifen analog.

The test agent is not limited and includes any agent disclosed herein. In some embodiments, the test agent is a small molecule, a peptide, an RNA or a DNA.

In some embodiments, the in vitro condensate comprises one or more components as described herein. In some embodiments, the in vitro condensate comprises one, two, or all three of DNA, RNA and/or protein as components. In some embodiments, the in vitro condensate comprises DNA, RNA and protein as components. In some embodiments, the in vitro condensate comprises Mediator, MED1, MED15, GCN4, p300, BRD4, a nuclear receptor ligand, or TFIID. In some embodiments, the in vitro condensate comprises OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, a nuclear receptor ligand, a fusion oncogenic transcription factor, TFIID, a signaling factor, methyl-DNA binding protein, splicing factor, gene silencing factor, RNA polymerase, β-catenin, STAT5, SMAD3, NF-KB, MECP2, MBD1, MBD2, MBD3, MBD4, HP1α, TBL1R, HDAC3, SMRT, RNA polymerase II, SRSF2, SRRM1, SRSF1, and fragments thereof comprising an intrinsically disordered region (IDR). In some embodiments, the condensate comprises a single component (i.e., homotypic). In some embodiments, the in vitro condensate is heterotypic and comprises 2, 3, 4, 5, or more client or scaffold components. In some embodiments, the in vitro condensate comprises MED15 and GCN4. In some embodiments, the in vitro condensate comprises a nuclear receptor or fragment thereof as described herein. In some embodiments, the in vitro condensate comprises MED1 and ER. In some embodiments the ER is a mutant ER (e.g., a mutant ER described herein, a mutant ER having constitutive activity, a mutant ER having a mutation conferring tamoxifen resistance). In some embodiments, the condensate comprises a splicing factor and RNA polymerase. In some embodiments, the condensate comprises a methyl-DNA binding protein (e.g., MeCP2). In some embodiments, the condensate comprises a signaling factor.

In some embodiments, the in vitro condensate comprises a plurality of detectable tags as described herein. In some embodiments, the detectable tag comprises different fluorescent tags on different components (e.g., MED15 labeled with one fluorescent tag and GCN4 or a nuclear receptor or fragment thereof labeled with a different fluorescent tag). In some embodiments, one or more components of the condensate have a quencher.

The in vitro condensate can also comprise intrinsically disordered regions or domains or proteins having intrinsically disordered regions or domains. The IDR may be any described herein or obtained by methods in the art (e.g., in the article and website referred to herein). In some embodiments, the IDR is an IDR having a motif set forth in Table S2. In some embodiments, the component is set forth in Table S1. In some embodiments, the intrinsically disordered regions or domains are MED1, MED15, GCN4 or BRD4 intrinsically disordered regions or domains. In some embodiments, the IDR comprises an IDR, or a portion thereof, from OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, a nuclear receptor ligand, a fusion oncogenic transcription factor, TFIID, a signaling factor, methyl-DNA binding protein, splicing factor, gene silencing factor, RNA polymerase, β-catenin, STAT3, SMAD3, NF-KB, MECP2, MBD1, MBD2, MBD3, MBD4, HP1α, TBL1R, HDAC3, SMRT, RNA polymerase II, SRSF2, SRRM1, or SRSF1 IDR. In some embodiments, the in vitro condensate can comprise a portion of an IDR. For example, the condensate can comprise at least 20%, 30%, 40%, 50%, 60%, 70%, 80%, 90% or more of an IDR of a protein (e.g. a protein associated with an in vivo transcriptional condensate). In some embodiments, the in vitro condensate can comprise an at least about 20, 30, 40, 50, 60, 75, 100, 150, 200, 250, or 300 amino acid portion of an IDR.

In some embodiments, the in vitro condensate comprises a signaling factor or a fragment thereof. In some embodiments, the in vitro condensate comprises a signaling factor or a fragment thereof comprising an IDR necessary for the activation of transcription of a gene. In some embodiments, the signaling factor is associated with an oncogenic signaling pathway.

In some embodiments, the condensate comprises a methyl-DNA binding protein or a fragment thereof comprising a C-terminal IDR, or a suppressor or fragment thereof comprising an IDR. In some embodiments, the condensate is associated with methylated DNA or heterochromatin. In some embodiments, the condensate comprises an aberrant level or activity of methyl-DNA binding protein. In some embodiments, the silencing of genes associated with the condensate by the agent are assessed. In some embodiments, the condensate comprises a splicing factor or a fragment thereof comprising an IDR, or an RNA polymerase or fragment thereof comprising an IDR.

In some embodiments, the condensate is associated with a transcription initiation complex or elongation complex. In some embodiments, the condensate is contacted with a cyclin dependent kinase. In some embodiments, the RNA polymerase is RNA polymerase II (Pol II). In some embodiments, changes in RNA transcription initiation activity associated with the condensate caused by contact with the agent are assessed In some embodiments, changes in RNA elongation or splicing activity associated with the condensate caused by contact with the agent are assessed.

In some embodiments, the in vitro condensate is formed by weak protein-protein interactions. In some embodiments, the weak protein-protein interactions comprise interactions between IDRs or portions of IDRs.

In some embodiments, the in vitro condensate comprises (intrinsically disordered domain)-(inducible oligomerization domain) fusion proteins. The inducible oligomerization domain is also not limited. In some embodiments, the inducible oligomerization domain oligomerizes in response to electromagnetic radiation (e.g., visible light) or an agent (e.g., a small molecule). Example of inducible oligomerization domains include FK506 and cyclosporin binding domains of FK506 binding proteins and cyclophilins, and the rapamycin binding domain of FRAP. In some, embodiments, the inducible oligomerization domain is a Cry protein (e.g., Cry2). In some embodiments, the fusion protein is an intrinsically disordered domain-Cry2 fusion protein. “CRY” is used in this document refers to a crypto-chromium (chryptochrome) protein, it is typically a CRY2 (GenBank No.: NM_100320) of Arabidopsis thaliana. Methods of using of Cry2 for light induced oligomerization is taught in Che, et al, “The Dual Characteristics of Light-Induced Cryptochrome 2, Homo-oligomerization and Heterodimerization, for Optogenetic Manipulation in Mammalian Cells,” ACS Synth Biol. 2015 Oct. 16; 4(10): 1124-1135 and Duan, et al., “Understanding CRY2 interactions for optical control of intracellular signaling,” Nature Communications, vol. 8:547(2017), herein incorporated by reference. In some embodiments, the inducible oligomerization domain is induced by a small molecule, protein, or nucleic acid. In some embodiments, the inducible oligomerization domain is induced by visible light (e.g., blue light).

The IDR is not limited and may be any one described or referred to herein. In some embodiments, the IDR has a motif set forth in Table S2. In some embodiments, the intrinsically disordered domain is MED1, MED15, GCN4, or BRD4 intrinsically disordered domain. In some embodiments, the IDR is an IDR of a transcription factor listed in Table S3. In some embodiments, the IDR is an IDR of a nuclear receptor activation domain. In some embodiments, the IDR is an IDR of a nuclear receptor activation domain, wherein the nuclear receptor has a mutation associated with a disease.

In some embodiments, the in vitro condensate simulates a transcriptional condensate found in a cell.

In some embodiments, an in vitro transcriptional condensate, heterochromatin condensate, or condensate physically associated with mRNA initiation or elongation complex, is isolated. Any suitable means of isolation is encompassed herein. In some embodiments, the in vitro condensate is chemically or immunologically precipitated. In some embodiments, the in vitro condensate is isolated by centrifugation (e.g., at about 5,000×g, 10,000×g, 15,000×g for about 5-15 minutes; about 10.000×g for about 10 min).

In some embodiments, the in vitro condensate is a transcriptional condensate, heterochromatin condensate, or condensate physically associated with mRNA initiation or elongation complex isolated from a cell. Any suitable methods may be used in the art to isolate the condensate. For instance, the condensate may be isolated by lysis of the nucleus of a cell with a homogenizer (i.e., dounce homogenizer) under suitable buffer conditions, followed by centrifugation and/or filtration to separate the condensate.

Some aspects of the disclosure are directed to a method of identifying an agent that modulates condensate formation, stability, function, or morphology of a condensate, comprising providing a cell with transcriptional condensate dependent expression of a reporter gene, contacting the cell with a test agent, and assessing expression of the reporter gene. In some embodiments, the cell does not express the reporter gene prior to contact with a test agent and expresses the reporter gene after contact with an agent that enhances condensate formation, stability, function, or morphology. In some embodiments, the cell does express the reporter gene prior to contact with a test agent and stops or reduces expression of the reporter gene after contact with an agent that suppresses, degrades, or prevents condensate formation, stability, function, or morphology.

In some embodiments, a method of identifying an agent that modulates condensate formation, stability, function, or morphology, comprises providing a cell or an in vitro transcription assay (or providing both an in vitro assay and a cell) expressing a reporter gene under the control of a transcription factor, contacting the cell or assay with a test agent, and assessing expression of the reporter gene. In some embodiments, the TF comprises a heterologous DNA-binding domain (DBD) and activation domain. In some embodiments, the TF may comprise the activation domain of a mammalian TF, a TF described herein, or a mutant mammalian TF, or a mutant TF of a TF described herein. In some embodiments, the TF is a nuclear receptor (e.g., a mutant nuclear receptor, a mutant nuclear receptor with constitutive activity independent of cognate ligand binding, a mutant estrogen receptor causing estrogen mediated gene activation in the presence of tamoxifen, a mutant estrogen receptor causing gene activation without the presence of estrogen). In some embodiments, the mutant TF activation domain may be associated with a disease or condition (e.g., a disease or condition described herein). The DBD is not limited and may be any suitable DBD. In some embodiments, the DBD is a GAL4 DBD. The in vitro assay is not limited and may be any disclosed in the art. In some embodiments, the in vitro assay is the in vitro transcription assay disclosed in Sabari et al. Science. 2018 Jul. 27; 361(6400).

In some embodiments of the methods of identifying an agent disclosed herein, the condensate comprises a nuclear receptor (e.g., wild-type nuclear receptor, mutant nuclear receptor, mutant nuclear receptor associated with a disease or condition, a nuclear hormone receptor, a mutant nuclear hormone receptor having constitutive activity not dependent upon cognate ligand binding) or fragment thereof comprising an activation domain IDR. Any nuclear receptor or fragment described herein may be used. In some embodiments, the nuclear receptor activates transcription when bound to a cognate ligand. In some embodiments, the nuclear receptor activates transcription independent of ligand binding (e.g., a nuclear receptor having a mutation making it ligand independent, a mutant estrogen receptor causing estrogen mediated gene activation in the presence of tamoxifen, a mutant estrogen receptor causing gene activation without the presence of estrogen). In some embodiments, the nuclear receptor is a nuclear hormone receptor. In some embodiments, the nuclear receptor has a mutation. In some embodiments, the mutation is associated with a disease or condition. In some embodiments, the disease or condition is cancer (e.g., breast cancer). In some embodiments of the methods of identifying an agent disclosed herein, an agent is screened against both a condensate comprising a wild-type nuclear receptor and a nuclear receptor having a mutation associated with a disease. In some embodiments, the identified agent preferentially binds to a nuclear receptor having a mutation (e.g., nuclear hormone receptor having a mutation, ligand dependent nuclear receptor having a mutation, a mutant estrogen receptor causing estrogen mediated gene activation in the presence of tamoxifen, a mutant estrogen receptor causing gene activation without the presence of estrogen) over a wild-type nuclear condensate. In some embodiments, the identified agent preferentially disrupts a transcriptional condensate comprising a nuclear receptor having a mutation (e.g., nuclear hormone receptor having a mutation, ligand dependent nuclear receptor having a mutation, a mutant estrogen receptor causing estrogen mediated gene activation in the presence of tamoxifen, a mutant estrogen receptor causing gene activation without the presence of estrogen) over a condensate comprising a wild-type nuclear receptor.

In some embodiments, an agent identified by the methods disclosed herein of modulating condensate formation, stability, function, or morphology is further, or alternatively, tested to assess its effect on one or more functional properties of a condensate, e.g., ability to modulate transcription of one or more genes associated with the condensate. In some embodiments, an agent identified by the methods disclosed herein of modulating condensate formation, stability, function, or morphology is further tested for its ability to modulate one or more features of a disease. The disease is not limited and may be any disease disclosed herein. For example, if the agent inhibits condensate formation by an oncogenic mutant TF, could test the ability of the agent to inhibit proliferation of cancer cells that comprise that TF (e.g., cancer cells that depend on that TF for continued viability and/or proliferation).

In some embodiments, an agent identified as modulating one or more structural property of a condensate (e.g., formation, stability, or morphology) or functional properties of a condensate (e.g. modulation of transcription) by the methods disclosed herein may be administered to a subject, e.g., a non-human animal that serves as a model for a disease, or a subject in need of treatment for the disease. In some embodiments, a subject in need of treatment with an agent identified as modulating one or more structural property of a condensate may be identified by a method disclosed herein.

In some embodiments, an analog of an agent identified as modulating one or more structural property of a condensate (e.g., formation, stability, function, or morphology) or functional properties of a condensate (e.g. modulation of transcription) by the methods disclosed herein may be generated. Methods of generating analogs are known in the art and include methods described herein. In some embodiments, generated analogs can be tested for a property of interest, such as increased stability (e.g., in an aqueous medium, in human blood, in the GI tract, etc.), increased bioavailability, increased half-life upon administration to a subject, increased cell uptake, increased activity to modulate a condensate property including structural property of a condensate (e.g., formation, stability, function, or morphology) or functional properties of a condensate (e.g. modulation of transcription), increased specificity for a condensate containing a wild-type or mutant component (e.g., mutant TF, mutant NR), increased specificity for a cell type disclosed herein.

In some embodiments, a high throughput screen (HTS) is performed. A high throughput screen can utilize cell-free or cell-based assays (e.g., a condensate containing cell as described herein, an in vitro condensate, an isolated in vitro condensate). High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg H\ddot{υ}ser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. For example, a candidate anticancer agent may be tested against multiple different cancer cell lines or in an in vivo tumor model to determine its effect on cancer cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cancer cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells. The test agent may be any agent as described herein.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

Screening Assays Involving Nuclear Receptors

Some aspects of the disclosure are related to a method of identifying an test agent that modulates formation, stability, or morphology of a condensate, comprising providing a cell, contacting the cell with a test agent, and determining if contact with the test agent modulates formation, stability, or morphology of a condensate, wherein the condensate comprises an nuclear receptor (NR), or a fragment thereof, as a condensate component. The nuclear receptor is not limited and may be any nuclear receptor described herein. In some embodiments, the nuclear receptor is a mutant nuclear receptor (e.g., a mutant nuclear receptor associated with a disease, a mutant nuclear receptor with constitutive activity (e.g., transcriptional activity) independent of cognate ligand binding). In some embodiments, the nuclear receptor is a nuclear hormone receptor, an Estrogen Receptor, or a Retinoic Acid Receptor-Alpha. In some embodiments, the condensate further comprises a co-factor (e.g., Mediator, MED1) as a condensate component. The components of the condensate may be any suitable condensate component described herein. In some embodiments, the cell comprises the condensate. In some embodiments, the agent causes the formation of the condensate in the cell.

In some embodiments of the methods of identifying a test agent, an agent that modulate formation, stability, or morphology of the condensate, (e.g., if it decreases formation or stability of the condensate) is identified as a candidate therapeutic agent (e.g., a therapeutic agent to a disease characterized by a mutant nuclear receptor, cancer, or a disease characterized by a signaling pathway comprising the nuclear receptor). In some embodiments, the identified agent may be a candidate for therapy of any corresponding disease or condition described herein. In some embodiments of the methods of identifying a test agent described herein, an agent that decreases formation or stability of a condensate comprising mutant nuclear receptor is identified as a candidate agent for treating a disease or condition characterized by the mutant NR. In some embodiments of the methods of identifying a test agent described herein, an agent that decreases formation or stability of a condensate comprising a nuclear receptor (e.g., mutant nuclear receptor) or fragment thereof is identified a candidate modulator of activity of the nuclear receptor.

In some embodiments of the methods of identifying a test agent, modulation of the condensate reduces or eliminates transcription of a target gene (e.g., MYC oncogene or other gene described herein or involved in cancer growth or viability). In some embodiments, transcription of the target gene (e.g., MYC oncogene) is reduced by at least about 5%, 10%, 15%, 20%, 25%, 30%, 35%, 40%, 45%, 50%, 55%, 60%, 65%, 70%, 75%, 80%, 85%, 90%, 95%, 99% or more.

In some embodiments, the condensate comprises a detectable label. The label is not limited and may be any label described herein. In some embodiments, a component of the condensate comprises the detectable label. In some embodiments, the nuclear receptor or a fragment thereof comprises the detectable label.

Some aspects of the invention are related to a method of identifying an agent that modulates formation, stability, or morphology of a condensate, comprising providing an in vitro condensate, contacting the condensate with a test agent, and determining if contact with the test agent modulates formation, stability, or morphology of the condensate, wherein the condensate comprises an nuclear receptor (NR), or a fragment thereof, as a condensate component. The nuclear receptor is not limited and may be any nuclear receptor described herein. In some embodiments, the nuclear receptor is a mutant nuclear receptor (e.g., a mutant nuclear receptor associated with a disease, a mutant nuclear receptor with constitutive activity (e.g., transcriptional activity) independent of cognate ligand binding). In some embodiments, the nuclear receptor is a nuclear hormone receptor, an Estrogen Receptor, or a Retinoic Acid Receptor-Alpha. In some embodiments, the condensate further comprises a co-factor (e.g., Mediator, MED1) as a condensate component. The components of the condensate may be any suitable condensate component described herein. In some embodiments, the condensate is isolated from a cell. The cell from which the condensate is isolated may be any suitable cell. In some embodiments, the agent causes the formation of the condensate in vitro.

In some embodiments of the methods of identifying a test agent, an agent that modulate formation, stability, or morphology of the in vitro condensate, (e.g., if it decreases formation or stability of the condensate) is identified as a candidate therapeutic agent (e.g., a therapeutic agent to a disease characterized by a mutant nuclear receptor, cancer, or a disease characterized by a signaling pathway comprising the nuclear receptor). In some embodiments, the identified agent may be a candidate for therapy of any corresponding disease or condition described herein. In some embodiments of the methods of identifying a test agent described herein, an agent that decreases formation or stability of an in vitro condensate comprising mutant nuclear receptor is identified as a candidate agent for treating a disease or condition characterized by the mutant NR. In some embodiments of the methods of identifying a test agent described herein, an agent that decreases formation or stability of an in vitro condensate comprising a nuclear receptor (e.g., mutant nuclear receptor) or fragment thereof is identified a candidate modulator of activity of the nuclear receptor.

In some embodiments, the in vitro condensate comprises a detectable label. The label is not limited and may be any label described herein. In some embodiments, a component of the condensate comprises the detectable label. In some embodiments, the nuclear receptor or a fragment thereof comprises the detectable label.

Diseases and Disease Dependencies

Cancer cells can become highly dependent on transcription of certain genes, as in transcriptional addiction, and this transcription can be dependent upon specific condensates. For example, a transcriptional condensate might be formed at an oncogene on which the tumor is dependent and this condensate might be especially dependent on a specific protein, RNA or DNA motif that can be targeted by an agent described herein (e.g., a peptide, nucleic acid or a small molecule). Some embodiments of the disclosure are directed to using the methods described herein to screen for anti-cancer agents that suppress, eliminate or degrade transcriptional condensates in cancer cells. Some embodiments of the disclosure are directed to using the methods described herein to screen for anti-cancer agents that modulate heterochromatin condensates in cancer cells. In some embodiments, methods described herein are used to identify an agent that decreases formation or stability of transcriptional condensates comprising nuclear receptors (e.g., mutant nuclear receptors, mutant hormone receptors).

For example, in some embodiments, methods described herein are used to identify an agent that decreases formation or stability of transcriptional condensates comprising MED1 and ER. In some embodiments, methods described herein are used to identify an agent that decreases formation or stability of transcriptional condensates comprising MED1 and a mutant ER that is resistant to tamoxifen. In some embodiments, methods described herein are used to identify an agent that decreases formation or stability of transcriptional condensates comprising MED1 and ER (e.g., agents having SERM activity as described herein, e.g., candidate agents effective against ER+ breast cancer). In some embodiments, methods described herein are used to identify an agent that decreases formation or stability of transcriptional condensates comprising increased levels of MED1 (e.g., at least 4-fold more MED1 than in a condensate from an ER+ breast cancer cell that is not tamoxifen resistant). In some embodiments, methods described herein are used to identify an agent that decreases formation or stability of transcriptional condensates comprising mutant ER (e.g., as described herein) and MED1. In some embodiments, the identified agent is a candidate agent for preventing the development of, or overcoming SERM (tamoxifen) resistant cancer (e.g., breast cancer).

Cells that harbor mutations or epigenetic alterations that cause diseases suffer altered transcription that is dependent on specific condensates. For example, a disease may be caused by, and dependent on, condensate formation, composition, maintenance, dissolution or regulation at one or more disease genes. Some embodiments of the disclosure are directed to modulating condensates associated with disease using the methods described herein. Some embodiments of the disclosure are directed to screening for agents that can modulate condensates associated with disease by the methods described herein.

In some embodiments, the diseases or conditions described herein are associated with a nuclear receptor. In some embodiments, the diseases or conditions described herein are associated with a mutation in a nuclear receptor or aberrant expression of a nuclear receptor (e.g., an increased or decreased level as compared to a reference level).

### Condensate and Condensate Component Compositions

Some aspects of the disclosure are directed to isolated synthetic condensates comprising one, two, or all three of DNA, RNA and protein. The synthetic condensates may comprise any of the components described herein. In some embodiments, the synthetic condensates may comprise IDR-inducible oligomerization domains as described herein. In some embodiments, the synthetic condensates may comprise Mediator, MED1, MED15, p300, BRD4, a nuclear receptor ligand, or TFIID. In some aspects, the synthetic transcriptional condensates may comprise a transcription factor (e.g., OCT4, p53, MYC, NANOG, MyoD, KLF4, a SOX family transcription factor, a GATA family transcription factor, a nuclear receptor, a fusion oncogenic transcription factor, or GCN4). In some embodiments, the synthetic condensate may comprise OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, signaling factor, methyl-DNA binding protein, splicing factor, gene silencing factor, RNA polymerase, β-catenin, STAT5, SMAD3, NF-KB, MECP2, MBD1, MBD2, MBD3, MBD4, HP1α, TBL1R, HDAC3, SMRT, RNA polymerase II, SRSF2, SRRM1, SRSF1, or TFIID, or a fragment or intrinsically disordered domain thereof. In some embodiments, the transcription factor has an activation domain of a transcription factor listed in Table S3. In some embodiments, the transcription factor has an IDR of a transcription factor listed in Table S3. In some embodiments, the transcription factor is listed in Table S3. In some embodiments, the transcription factor is a transcription factor that interacts with a mediator component (e.g., a mediator component listed in Table S3). Some aspects of the disclosure are directed to a liquid droplet comprising one or more synthetic transcriptional condensates. Some aspects of the disclosure are directed to a composition comprising the components needed for a screening assay as described herein.

Some aspects of the disclosure are directed to a fusion protein comprising a transcriptional condensate component as described herein and a domain that confers inducible oligomerization as described herein. In some embodiments, the domain that confers inducible oligomerization is Cry2. In some embodiments, the fusion protein further comprises a detectable tag as described herein. In some aspects, the detectable tag is a fluorescent tag. In some embodiments, the domain that confers inducible oligomerization is inducible with a small molecule, protein, or nucleic acid.

Some aspects of the disclosure provide methods of making synthetic transcriptional condensates, heterochromatin condensates, and condensates physically associated with mRNA initiation or elongation complex. In some embodiments the method comprises combining two or more condensate components in vitro under conditions suitable for formation of transcriptional condensates, heterochromatin condensates, and condensates physically associated with mRNA initiation or elongation complex. The conditions can include appropriate concentrations of components, salt concentration, pH, etc. In some embodiments, the conditions include a salt concentration (e.g., NaCl) of about 25 mM, 40 mM, 50 mM, 125 mM, 200 mM, 350 mM, or 425 mM; or in the range of about 10-250 mM, 25-150 mM, or 40-100 mM. In some embodiments, the conditions include a pH of about 7-8, 7.2-7.8, 7.3-7.7, 7.4-7.6, or about 7.5. In some embodiments, the transcriptional condensate components comprise MED1, BRD4, the intrinsically disordered domain of BRD4 (BRD4-IDR), and/or the intrinsically disordered domain of MED1 (MED1-IDR). In some embodiments, the transcriptional condensate components comprise BRD4-IDR and MED1-IDR. In some embodiments, the transcriptional condensate components comprise an IDR of an activation domain of a transcription factor (e.g., OCT4, p53, MYC, NANOG, MyoD, KLF4, a SOX family transcription factor, a GATA family transcription factor, a nuclear receptor, a fusion oncogenic transcription factor, or GCN4). In some embodiments, the IDR is an IDR of a transcription factor listed in Table S3. In some embodiments, the transcriptional condensate components comprise a nuclear receptor (e.g., ER) activation domain. In some embodiments, the IDR is and IDR of OCT4, p53, MYC, GCN4, Mediator, a mediator component, MED1, MED15, p300, BRD4, NANOG, MyoD, KLF4, a SOX family transcription factor, a GATA family transcription factor, a nuclear receptor, signaling factor, methyl-DNA binding protein, splicing factor, gene silencing factor, RNA polymerase, β-catenin, STAT5, SMAD3, NF-KB, MECP2, MBD1, MBD2, MBD3, MBD4, HP1α, TBL1R, HDAC3, SMRT, RNA polymerase II, SRSF2, SRRM1, SRSF1, or TFIID.

mRNA Initiation or Elongation Complex Associated Condensates

As shown below, Pol II CTD phosphorylation alters its condensate partitioning behavior and may thus drive an exchange of Pol II from condensates involved in transcription initiation to those involved in RNA splicing. This model is consistent with evidence from previous studies that large clusters of Pol II can fuse with Mediator condensates in cells, that phosphorylation dissolves CTD-mediated Pol II clusters, that CDK9/Cyclin T can interact with the CTD through a phase separation mechanism, that Pol II is no longer associated with Mediator during transcription elongation, and that nuclear speckles containing splicing factors can be observed at loci with high transcriptional activity.

Some aspects of the disclosure are directed to a method of modulating mRNA initiation, comprising modulating formation, composition, maintenance, dissolution and/or regulation of a condensate physically associated with mRNA initiation. In some embodiments, modulating mRNA initiation also modulates mRNA elongation, splicing or capping. In some embodiments, modulating formation, composition, maintenance, dissolution and/or regulation of the condensate physically associated with mRNA initiation modulates an mRNA transcription rate. In some embodiments, modulating formation, composition, maintenance, dissolution and/or regulation of the condensate physically associated with mRNA initiation modulates a level of a gene product.

In some embodiments, formation, composition, maintenance, dissolution and/or regulation of the condensate physically associated with mRNA initiation is modulated with an agent. The agent is not limited and may be any agent described herein. In some embodiments, the agent comprises a phosphorylated or hypophosphorylated RNA polymerase II C-terminal domain (Pol II CTD) or a functional fragment thereof. In some embodiments, the agent preferentially binds phosphorylated or hypophosphorylated Pol II CTD. In some embodiments, the agent phosphorylates or dephosphorylates Pol CTD. In some embodiments, the agent modulates phosphorylation activity of a cyclin dependent kinase (CDK). In some embodiments, the agent enhances or inhibits phosphorylated RNA polymerase association with splicing factors. The splicing factors may be any splicing factor described herein and is not limited.

Some aspects of the disclosure are directed to a method of modulating mRNA elongation, comprising modulating formation, composition, maintenance, dissolution and/or regulation of a condensate physically associated with mRNA elongation. In some embodiments, modulating mRNA elongation also modulates mRNA initiation. In some embodiments, modulating formation, composition, maintenance, dissolution and/or regulation of the condensate physically associated with mRNA elongation modulates co-transcriptional processing of an mRNA. In some embodiments, modulating formation, composition, maintenance, dissolution and/or regulation of the condensate physically associated with mRNA elongation modulates the number or relative proportion of mRNA splice variants. In some embodiments, formation, composition, maintenance, dissolution and/or regulation of the condensate physically associated with mRNA elongation is modulated with an agent. The agent is not limited and may be any agent disclosed herein. In some embodiments, the agent comprises a phosphorylated or hypophosphorylated RNA polymerase II C-terminal domain (Pol II CTD) or a functional fragment thereof. In some embodiments, the agent preferentially binds a phosphorylated or hypophosphorylated Pol II CTD. In some embodiments, the agent preferentially binds phosphorylated or hypophosphorylated Pol II CTD. In some embodiments, the agent phosphorylates or dephosphorylates Pol CTD. In some embodiments, the agent modulates phosphorylation activity of a cyclin dependent kinase (CDK). In some embodiments, the agent enhances or inhibits phosphorylated RNA polymerase association with splicing factors. The splicing factors may be any splicing factor described herein and is not limited.

Some aspects of the disclosure are related to a method of modulating formation, composition, maintenance, dissolution and/or regulation of a condensate comprising modulating the phosphorylation or dephosphorylation of a condensate component. In some embodiments, the component is RNA polymerase II or an RNA polymerase II C-terminal region. In some embodiments, an agent is used to modulate the phosphorylation or dephosphorylation of a condensate component. The agent is not limited and may be any agent disclosed herein. In some embodiments, the agent modulates phosphorylation activity of a cyclin dependent kinase (CDK).

Some aspects of the disclosure are related to a method of treating or reducing the likelihood of a disease or condition associated with aberrant mRNA processing comprising modulating formation, composition, maintenance, dissolution and/or regulation of a condensate physically associated with mRNA elongation. The method of modulating a condensate is not limited and may be any method described herein for modulating a condensate. In some embodiments, the condensate is modulated with an agent described herein. In some embodiments, the disease or condition associated with aberrant mRNA processing is characterized by aberrant splicing variants. In some embodiments, the disease or condition associated with aberrant mRNA processing is characterized by aberrant mRNA initiation.

Some aspects of the disclosure are related to a method of identifying an agent that modulates formation, stability, or morphology of a condensate physically associated with mRNA initiation or elongation complex. The method of identifying an agent may be any method of identifying an agent or screening for an agent described herein.

In some embodiments, the method comprises providing a cell having a condensate, contacting the cell with a test agent, and determining if contact with the test agent modulates formation, stability, or morphology of the condensate, wherein the condensate comprises a hypophosphorylated RNA polymerase II C-terminal domain (Pol II CTD), a phosphorylated RNA polymerase II C-terminal domain (Pol II CTD), a splicing factor, or a functional fragment thereof. Some aspects of the disclosure are related to a method of identifying an agent that modulates formation, stability, or morphology of a condensate, comprising providing an in vitro condensate and assessing one or more physical properties of the in vitro condensate, contacting the in vitro condensate with a test agent, and assessing whether the test agent causes a change in the one or more physical properties of the in vitro condensate, wherein the condensate comprises a hypophosphorylated RNA polymerase II C-terminal domain (Pol II CTD), a phosphorylated RNA polymerase II C-terminal domain (Pol II CTD), a splicing factor, or a functional fragment thereof.

Some aspects of the disclosure are related to methods of identifying amino acid residues in cellular proteins whose phosphorylation status regulates condensate formation, stability, localization, partitioning, activity, or other properties. Identified residues could be targets for modification to modulate condensate formation, stability, localization, partitioning, activity, or other properties in a subject or in vitro. In some embodiments, the method entails physically or computationally identifying one or more phosphorylation sites or potential phosphorylation sites in a condensate component (e.g., a serine, threonine, or tyrosine), mutating one or more such residue e.g., changing the residue to alanine), and determining whether the mutation alters a property (e.g., formation, stability, localization, partitioning, activity) of the condensate comprising the mutant condensate component (e.g., as compared with a condensate component that did not contain the mutation). If the mutation alters the condensate property, then that phosphorylation site is identified as a target for modification to modulate the formation, stability, localization, partitioning, or activity of the condensate. In some embodiments of the invention, the kinase that is responsible for phosphorylation of the identified residue is identified (e.g., using in vitro kinase assays in which the condensate is a substrate, using cells that have reduced expression of individual kinases (e.g., performing a kinome-wide siRNA screen), using known kinase inhibitors that are known to inhibit particular kinases) Alternately or additionally, in some embodiments, a library of known kinase inhibitors is screened to identify one or more kinases that affect the phosphorylation status of the identified residue. In some embodiments of the invention, the phosphatase that is responsible for dephosphorylation of the identified residue is identified (e.g., using in vitro phosphatase assays in which the condensate is a substrate, using cells that have reduced expression of individual phosphatases (e.g., performing a siRNA screen of known phosphatases), using known phosphatase inhibitors that are known to inhibit particular phosphatases) Alternately or additionally, in some embodiments, a library of known phosphatase inhibitors is screened to identify one or more phosphatases that affect the phosphorylation status of the identified residue. These assays could be performed in vitro, in a cell-free system, or in cells in various embodiments.

Some aspects of the disclosure are related to an isolated synthetic condensate comprising hypophosphorylated RNA polymerase II C-terminal domain (Pol II CTD) or a functional fragment thereof. Some aspects of the disclosure are related to an isolated synthetic condensate comprising phosphorylated RNA polymerase II C-terminal domain (Pol II CTD) or a functional fragment thereof. Some aspects of the disclosure are related to an isolated synthetic condensate comprising a splicing factor or a functional fragment thereof.

Heterochromatin Condensates

Heterochromatin plays important roles in chromosome maintenance and gene silencing. It is shown below that MeCP2, a methyl-DNA binding protein that is ubiquitously expressed in cells and essential for normal development, is a key component of dynamic liquid heterochromatin condensates. MeCP2 containing condensates can compartmentalize repressive heterochromatin factors that contribute to gene silencing. The ability of MeCP2 to form condensates, to incorporate into heterochromatin in cells, and to compartmentalize gene silencing factors is dependent on its C-terminal intrinsically disordered region (IDR).

Some aspects of the disclosure are related to a method of modulating transcription of one or more genes, comprising modulating formation, composition, maintenance, dissolution and/or regulation of a condensate associated with heterochromatin (i.e., heterochromatin condensate). The method of modulating the heterochromatin condensate is not limited and may be any method for modulating a condensate described herein. In some embodiments, modulating the heterochromatin condensate increases or stabilizes repression of transcription (i.e., gene silencing) of the one or more genes. In some embodiments, modulating the heterochromatin condensate decreases repression of transcription (i.e., gene silencing) of the one or more genes. In some embodiments, a plurality of condensates associated with heterochromatin are modulated. In some embodiments, formation, composition, maintenance, dissolution and/or regulation of the heterochromatin condensate is modulated with an agent. The agent is not limited and may be any agent described herein. In some embodiments, the agent comprises, or consists of, a peptide, nucleic acid, or small molecule. In some embodiments, the agent binds methylated DNA, a methyl-DNA binding protein, or a gene silencing factor.

Some aspects of the disclosure are related to a method of modulating gene silencing, comprising modulating formation, composition, maintenance, dissolution and/or regulation of a heterochromatin condensate. In some embodiments, gene silencing is stabilized or increased. In some embodiments, gene silencing is decreased. In some embodiments, gene silencing is modulated with an agent. The agent is not limited and may be any agent described herein.

Some aspects of the disclosure are related to a method of treating or reducing the likelihood of a disease or condition associated with aberrant gene silencing (e.g., an increased or decreased level as compared to a reference or control level) comprising modulating formation, composition, maintenance, dissolution and/or regulation of a heterochromatin condensate. In some embodiments, the disease or condition associated with aberrant gene silencing is associated with aberrant expression or activity of a methyl-DNA binding protein. In some embodiments, the disease or condition associated with aberrant gene silencing is ATR-X syndrome, Juberg-Marsidi syndrome, Sutherland-Haan syndrome, Smith-Finemers syndrome, Breast cancer, MECP2 duplication syndrome, Rett syndrome, Autism, Down syndrome, ADHD/ADD, Alzheimer's, Huntington's, Parkinson's, Epilepsy, Bipolar mood disorder, Depression, Fetal alcohol syndrome, Werner syndrome, Colon cancer, Lymphoma, Pancreatic cancer, ICF syndrome, Bladder cancer, Breast cancer, Colon cancer, Hepatocellular carcinoma, Lung cancer, Barrett's esophagus, Bladder cancer, Breast cancer, Colorectal cancer, Melanoma, Myeloma/lymphoma, Hepatocellular carcinoma, Prostate cancer, Wilms tumor, Breast cancer, Medulloblastoma, Papillary thyroid carcinoma, Facioscappulohumeral muscular dystrophy, Friedreich's ataxia, Fragile X syndrome, Angelman syndrome, Prader-Willi syndrome, Hutchinson-Gilford progeria syndrome, Werner syndrome, Beckwith-Weidemann syndrome, Silver-Russel syndrome, Spinocerebellar ataxias, or Cocaine substance abuse. In some embodiments, the disease or condition associated with aberrant gene silencing is Rett syndrome or MeCP2 overexpression syndrome.

Some aspects of the disclosure are related to a method of identifying an agent that modulates condensate formation, stability, or morphology of a heterochromatin condensate. The method of identifying an agent may be any method of identifying an agent or screening for an agent described herein. In some embodiments, the method comprises providing a cell having a condensate, contacting the cell with a test agent, and determining if contact with the test agent modulates formation, stability, or morphology of the heterochromatin condensate, wherein the condensate comprises a methyl-DNA binding protein (e.g., MeCP2) or a fragment thereof (e.g., a C-terminal intrinsically disordered region of MeCP2), or a suppressor or functional fragment thereof. In some embodiments, the condensate is associated with methylated DNA. In some embodiments, the method comprises providing an in vitro condensate and assessing one or more physical properties of the in vitro condensate, contacting the in vitro condensate with a test agent, and assessing whether the test agent causes a change in the one or more physical properties of the in vitro condensate, wherein the condensate comprises methyl-DNA binding protein (e.g., MeCP2) or a fragment thereof (e.g., a C-terminal intrinsically disordered region of MeCP2), or a suppressor or functional fragment thereof.

Some aspects of the disclosure are related to an isolated synthetic condensate comprising a methyl-DNA binding protein (e.g., MeCP2) or a fragment thereof (e.g., a C-terminal intrinsically disordered region of MeCP2), or a suppressor or functional fragment thereof.

Diagnostic Methods

Some aspects of the disclosure are related to diagnostic methods and methods of identifying a subject who is a candidate for treatment with a condensate-targeted therapeutic agent. In some embodiments, methods of identifying a subject who is a candidate for treatment with a condensate-targeted therapeutic agent comprises obtaining a sample isolated from the subject, determining the level (or a property selected from stability, dissolution, or maintenance) of one or more condensates in the sample, and identifying the subject as a candidate for treatment with a condensate-targeted therapeutic agent if an aberrant level (e.g., an increased or decreased level as compared to a reference level), or a aberrant property selected from stability, dissolution, or maintenance, of the condensate is detected. The method may further include administering a condensate-targeted therapeutic agent to the subject, wherein the agent at least partly normalizes the aberrant level (or a property selected from stability, dissolution, or maintenance) of the condensate. A “condensate-targeted therapeutic agent” is defined herein as an agent that modulates the formation, stability, composition, maintenance, dissolution, or regulation of a condensate in a therapeutically beneficial manner, e.g., by physically associating with a condensate component, modifying a condensate component, or inhibiting or activating a modifier/demodifier of a condensate component. In some embodiments, the subject suffers from cancer. In some embodiments, the condensate comprises an oncogene or drives transcription of an oncogene. In some embodiments, the condensate is a transcriptional condensate. In some embodiments, the condensate is a heterochromatin-associated condensate.

In some aspects, a method comprises providing a sample obtained from a subject, e.g., a mammalian subject, e.g., a human subject, and detecting a transcriptional condensate in the sample. In some embodiments the sample comprises at least one cell, e.g., at least one cancer cell. In some embodiments the method comprises detecting an aberrant level (e.g., an increased or decreased level as compared to a reference level), aberrant composition, or aberrant localization of a transcriptional condensate in a cell or sample, as compared with a control cell or sample (e.g., healthy cell or sample from a healthy subject). In some embodiments, detection of aberrant level, composition, or localization of a transcriptional condensate may be used to diagnose a disease.

In some aspects, a method comprises providing a sample obtained from a subject, e.g., a mammalian subject, e.g., a human subject, and detecting a mutation or aberrant level or activity of a component of a transcriptional condensate in the sample, as compared with a control cell or sample (e.g., healthy cell or sample from a healthy subject). In some embodiments the sample comprises at least one cell, e.g., at least one cancer cell. In some embodiments the mutation or alteration in level or activity of a component of a transcriptional condensate affects the formation, stability, localization, activity, or morphology of a transcriptional condensate. In some embodiments, detection of mutation or aberrant level or activity of a component of a transcriptional condensate in the sample may be used to diagnose a disease.

Transgenic Non-Human Animals

Some aspects of the disclosure are related to transgenic non-human animals (e.g., non-human mammal, non-human primate, rodent (e.g., mouse, rat, rabbit, hamster), canine, feline, bovine, or other mammal), cells of which comprise a transgene encoding a polypeptide comprising a condensate component fused to a detectable label. In some embodiments the method may comprise administering a test agent to such an animal, obtaining a sample comprising one or more cells isolated from the animal, and determining the effect of the test agent on formation, stability, or activity of a condensate comprising the polypeptide. In some embodiments, the sample is a tissue sample.

Some aspects of the disclosure are related to a transgenic animal as an animal model for a disease or condition. The disease or condition is not limited and may be any disease or condition disclosed herein. In some embodiments, the transgenic animal is used to test candidate agents for the disease. In some embodiments, the transgenic animals are a source of primary cells for performing methods disclosed herein (e.g., methods of screening for or identifying agents).

Breast Cancer

Breast cancer is one of the most common cancers and a leading cause of cancer mortality. Approximately 70% of human breast cancers are hormone-dependent and estrogen receptor positive (ER+) (e.g., dependent upon estrogen for growth). Selective estrogen receptor modulator (SERM), such as tamoxifen, raloxifene, or toremifene are often used to treat ER+ breast cancers. It will be appreciated that SERMs can act as ER inhibitors (antagonists) in breast tissue but, depending on the agent, may act as activators (e.g., partial agonists) of the ER in certain other tissues (e.g., bone). It will also be understood that tamoxifen itself is a prodrug that has relatively little affinity for the ER but is metabolized into active metabolites such as 4-hydroxytamoxifen (afimoxifene) and N-desmethyl-4-hydroxytamoxifen (endoxifen). As used herein, the term “tamoxifen” will be interpreted in context to mean tamoxifen or an active metabolite thereof. For example, tamoxifen is usually the form administered to patients. However, active metabolites such as 4-hydroxytamoxifen (afimoxifene) and/or N-desmethyl-4-hydroxytamoxifen (endoxifen) may be more suitable for in vitro uses.

Tamoxifen is the most commonly used chemotherapeutic agent for patients with ER-positive breast cancer. It is believed that tamoxifen competes with estrogen for binding to ER and tamoxifen bound ER has reduced or eliminated transcription factor activity. However, many patients taking tamoxifen eventually develop tamoxifen resistant breast cancers. Upon estrogen stimulation, ER establishes super-enhancers (Bojcsuk et al, Nucleic Acids Res 2017). Furthermore, as shown below, MED1 is over-expressed in ER+ breast cancer and is required for ER function and ER+ oncogenesis. Also as shown below, estrogen stimulates ER incorporation into MED1 condensates. This incorporation is dependent upon the presence of the LXXL motif in MED1.

The results herein show that MED1-IDR and ER form condensates dependent upon estrogen in vitro and in cells. Condensate formation is attenuated by tamoxifen. However, some tamoxifen resistant ER+ breast cancers comprise a mutant ER that is active independent of estrogen (e.g., Y537S and D538G mutants). Other tamoxifen resistant ER+ breast cancers comprise an ER fusion protein (e.g., ER-YAP1, ER-PCDH11X) that is active independent of estrogen. These ER form condensates with MED1 independent of the presence of estrogen. Further results shown herein demonstrate that ER+ breast cancer cells overexpressing MED1 (e.g., more than four-fold more than non-tamoxifen resistant ER+ breast cancer cells) incorporate ER into MED1 containing condensates independent of estrogen binding to the ER.

Some aspects of the disclosure are related to a method of modulating transcription of one or more genes in a cell, comprising modulating composition, maintenance, dissolution and/or regulation of a condensate associated with the one or more genes, wherein the condensate comprises an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding (e.g., Y537S and D538G mutants). In some embodiments, the mutant estrogen receptor is a fusion protein. In some embodiments, the fusion protein has constitutive activity not dependent upon estrogen binding (e.g., ER-YAP1, ER-PCDH11X). In some embodiments, the estrogen receptor fragment comprises a ligand binding domain or a functional fragment thereof. In some embodiments, the ER fragment comprises 2 ligand binding domains or functional fragments thereof. In some embodiments, the ER fragment comprises a DNA binding domain. In some embodiments, the MED1 fragment comprises an IDR, an LXXLL motif, or both. In some embodiments, the ER or MED1 is human ER or MED1. In some embodiments of the methods and compositions described herein, the ER or MED1 is a non-human mammal (e.g., rat, mouse, rabbit) ER or MED1.

In some embodiments, the condensate is contacted with estrogen or a functional fragment thereof (e.g., the estrogen or fragment thereof is physically associated with the condensate or is in a solution comprising the condensate). In some embodiments, the condensate is contacted with a selective estrogen selective modulator (SERM) (e.g., the SERM is physically associated with the condensate or is in a solution comprising the condensate). In some embodiments, the SERM is tamoxifen or an active metabolite thereof (4-hydroxytamoxifen and/or N-desmethyl-4-hydroxytamoxifen). In some embodiments, modulation of the condensate reduces or eliminates transcription of MYC oncogene. In some embodiments, transcription of the MYC oncogene is reduced by at least about 5%, 10%, 15%, 20%, 25%, 30%, 35%, 40%, 45%, 50%, 55%, 60%, 65%, 70%, 75%, 80%, 85%, 90%, 95%, 99% or more.

The cell may be any suitable cell. In some embodiments, the cell is a breast cancer cell (e.g., a breast cancer cell isolated from a patient, a breast cancer cell from a cell line (e.g., 600MPE, AU565, BT-20, BT-474, BT483, BT-549, Evsa-T, Hs578T, MCF-7, MDA-MB-231, SkBr3, T-47D)). In some embodiments, the cell is a transgenic cell expressing MED1 and estrogen receptor (e.g. human MED1 and/or estrogen receptor). In some embodiments, the cell is a transgenic cell expressing MED1, or functional fragment thereof, and estrogen receptor (e.g., mutant estrogen receptor) or functional fragment thereof (e.g. human MED1 and/or estrogen receptor). In some embodiments, the cell over-expresses MED1. As used herein, “over-expresses MED1” means that the cell expresses MED1 at a level that is at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more relative to a control cell or reference level. In some embodiments, the cell is a tamoxifen resistant ER+ breast cancer cell and the control cell is a non-tamoxifen resistant ER+ breast cancer cell. In some embodiments, the cell (e.g, a tamoxifen resistant ER+ breast cancer cell) overexpresses MED1 at a level of about 4-fold or more (e.g., about 4-fold to 4.5-fold) as compared to a control cell (e.g., non-tamoxifen resistant ER+ breast cancer cell).

In some embodiments, the transcriptional condensate is modulated by contacting the transcriptional condensate with an agent. In some embodiments, the agent reduces or eliminates physical interactions between the ER and MED1. In some embodiments, the agent reduces physical interactions between the ER and MED1 by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the agent reduces or eliminates interactions between ER and estrogen. In some embodiments, the agent reduces physical interactions between the ER and estrogen by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the condensate comprises a mutant ER or fragment thereof and the agent reduces transcription of the one or more genes.

Some aspects of the disclosure are related to a method of identifying an agent that modulates formation, stability, or morphology of a condensate, comprising providing a cell, contacting the cell with a test agent, and determining if contact with the test agent modulates formation, stability, or morphology of a condensate, wherein the condensate comprises an estrogen receptor (ER) or a fragment thereof, and MED1 or a fragment thereof, as condensate components. In some embodiments, the cell comprises the condensate. In some embodiments, the agent causes the formation of the condensate.

In some embodiments of the methods of identifying a test agent described herein, an agent that modulate formation, stability, or morphology of the condensate, (e.g., if it decreases formation or stability of the condensate) is identified as a candidate therapeutic agent (e.g., anti-cancer agent). In some embodiments, the agent is identified as an anti-ER+ cancer agent (e.g., ER+ breast cancer agent, anti-tamoxifen resistant breast cancer agent). In some embodiments of the methods of identifying a test agent described herein, an agent that decreases formation or stability of a condensate comprising mutant ER (or fragment thereof) and MED1 (or fragment thereof) is identified as a candidate agent for treating ER+ cancer, (e.g., tamoxifen-resistant ER+ cancer). In some embodiments of the methods of identifying a test agent described herein, an agent that decreases formation or stability of a condensate comprising ER (or fragment thereof) is identified a candidate modulator of ER activity (e.g., ER-mediated transcription).

In some embodiments, the estrogen receptor is a mutant estrogen receptor. In some embodiments, the mutant estrogen receptor has constitutive activity not dependent upon estrogen binding (e.g., Y537S and D538G mutants). In some embodiments, the mutant estrogen receptor is a fusion protein. In some embodiments, the fusion protein has constitutive activity not dependent upon estrogen binding (e.g., ER-YAP1, ER-PCDH11X). In some embodiments, the estrogen receptor fragment comprises a ligand binding domain or a functional fragment thereof. In some embodiments, the ER fragment comprises 2 ligand binding domains or functional fragments thereof. In some embodiments, the ER fragment comprises a DNA binding domain. In some embodiments, the MED1 fragment comprises an IDR, an LXXLL motif, or both. In some embodiments, the ER or MED1 is human ER or MED1. In some embodiments, the ER or MED1 is a non-human mammal (e.g., rat, mouse, rabbit) ER or MED1.

In some embodiments, the condensate is contacted with estrogen or a functional fragment thereof. In some embodiments, the condensate is contacted with a selective estrogen selective modulator (SERM). The SERM is not limited and may be any described herein our known in the art. In some embodiments, the SERM is tamoxifen or an active metabolite thereof (e.g., as described herein). In some embodiments of the methods described herein, modulation of the condensate reduces or eliminates transcription of a target gene (e.g., MYC oncogene or other gene described herein or involved in cancer growth or viability). In some embodiments, transcription of the target gene (e.g., MYC oncogene) is reduced by at least about 5%, 10%, 15%, 20%, 25%, 30%, 35%, 40%, 45%, 50%, 55%, 60%, 65%, 70%, 75%, 80%, 85%, 90%, 95%, 99% or more.

In some embodiments, the cell is a breast cancer cell (e.g., as described herein). In some embodiments, the cell over-expresses MED1 (e.g., as described herein). In some embodiments, the cell (e.g, a tamoxifen resistant ER+ breast cancer cell) overexpresses MED1 at a level of about 4-fold or more (e.g., about 4-fold to 4.5-fold) as compared to a control cell (e.g., non-tamoxifen resistant ER+ breast cancer cell). In some embodiments, the cell is an ER+ breast cancer cell. In some embodiments, the ER+ breast cancer cell is resistant to tamoxifen treatment. In some embodiments, the condensate comprises a detectable label. The label is not limited and may be any label described herein. In some embodiments, a component of the condensate comprises the detectable label. In some embodiments, the ER or a fragment thereof, and/or the MED1 or a fragment thereof comprises the detectable label. In some embodiments, the one or more genes comprise a reporter gene. The reporter gene is not limited and may be any reporter gene described herein.

Some aspects of the invention are related to a method of identifying an agent that modulates formation, stability, or morphology of a condensate, comprising providing an in vitro condensate, contacting the condensate with a test agent, and determining if contact with the test agent modulates formation, stability, or morphology of the condensate, wherein the condensate comprises an estrogen receptor (ER) or a fragment thereof, and MED1 or a fragment thereof, as condensate components. In some embodiments, the estrogen receptor is a mutant estrogen receptor (e.g., any mutant estrogen receptor described herein). In some embodiments, the mutant estrogen receptor has constitutive activity not dependent upon estrogen binding (e.g., Y537S and D538G mutants). In some embodiments, the mutant estrogen receptor is a fusion protein. In some embodiments, the fusion protein has constitutive activity not dependent upon estrogen binding (e.g., ER-YAP1, ER-PCDH11X). In some embodiments, the estrogen receptor fragment comprises a ligand binding domain or a functional fragment thereof. In some embodiments, the MED1 fragment comprises an IDR, an LXXLL motif, or both.

In some embodiments, the condensate is contacted with estrogen or a functional fragment thereof (e.g., the estrogen or fragment thereof is physically associated with the condensate or is in a solution comprising the condensate). In some embodiments, the condensate is contacted with a selective estrogen selective modulator (SERM) (e.g., the SERM is physically associated with the condensate or is in a solution comprising the condensate). In some embodiments, the SERM is tamoxifen or an active metabolite thereof (4-hydroxytamoxifen and/or N-desmethyl-4-hydroxytamoxifen).

In some embodiments, the condensate is isolated from a cell. The cell from which the condensate is isolated may be any suitable cell. In some embodiments, the cell is a breast cancer cell (e.g., a breast cancer cell isolated from a patient, a breast cancer cell from a cell line (e.g., 600MPE, AU565, BT-20, BT-474, BT483, BT-549, Evsa-T, Hs578T, MCF-7, MDA-MB-231, SkBr3, T-47D)). In some embodiments, the cell is a transgenic cell expressing MED1 and estrogen receptor (e.g. human MED1 and/or estrogen receptor). In some embodiments, the cell is a transgenic cell expressing MED1, or functional fragment thereof, and estrogen receptor (e.g., mutant estrogen receptor) or functional fragment thereof (e.g. human MED1 and/or estrogen receptor).

In some embodiments, the condensate comprises a detectable label. The detectable label is not limited and may be any label described herein or known in the art. In some embodiments, a component of the condensate comprises the detectable label. In some embodiments, the ER or a fragment thereof, and/or the MED1 or a fragment thereof comprises the detectable label.

Some aspects of the disclosure are related to an isolated synthetic transcriptional condensate comprising an estrogen receptor (ER) or a fragment thereof, and MED1 or a fragment thereof, as condensate components. In some embodiments, the estrogen receptor is a mutant estrogen receptor. In some embodiments, the mutant estrogen receptor has constitutive activity not dependent upon estrogen binding. In some embodiments, the estrogen receptor fragment comprises a ligand binding domain or a functional fragment thereof. In some embodiments, the MED1 fragment comprises an IDR, an LXXLL motif, or both. In some embodiments, the condensate comprises estrogen or a functional fragment thereof. In some embodiments, the condensate comprises a selective estrogen selective modulator (SERM).

Compositions

Some aspects of the invention are directed to compositions comprising agents identified by the methods disclosed herein. In some embodiments, the composition is a pharmaceutical composition.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue. Direct tissue administration may be achieved by direct injection. The agents may be administered once, or alternatively they may be administered in a plurality of administrations. If administered multiple times, the peptides may be administered via different routes. For example, the first (or the first few) administrations may be made directly into the affected tissue while later administrations may be systemic.

For oral administration, compositions can be formulated readily by combining the agent with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated in some embodiments to achieve appropriate systemic levels of compounds.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

A key feature of existing models of transcriptional control is that the underlying regulatory interactions occur in a step-wise manner dictated by biochemical rules that are probabilistic in nature. These models have limitations when called upon to explain recent observations involving super-enhancers or the ability of an enhancer to cause synchronous transcriptional bursts at two different genes. Phase-separated multi-molecular assemblies provide an essential regulatory mechanism to compartmentalize biochemical reactions within cells. We propose that a phase separation model more readily explains known features of transcriptional control, including the formation of super-enhancers, the sensitivity of super-enhancers to perturbation, their transcriptional bursting patterns and the ability of an enhancer to produce simultaneous effects at multiple genes. This model provides a conceptual framework to further explore principles of gene control in mammals.

Introduction

Recent studies of transcriptional regulation have revealed several puzzling observations that have heretofore lacked quantitative description, but whose further understanding would likely afford new and valuable insights into gene control during development and disease. For example, although thousands of enhancer elements control the activity of thousands of genes in any given human cell type, several hundred clusters of enhancers, called super-enhancers (SEs), control genes that have especially prominent roles in cell-type-specific processes (ENCODE Project Consortium et al., 2012; Hnisz et al., 2013; Loven et al., 2013; Parker et al., 2013; Roadmap Epigenomics et al., 2015; Whyte et al., 2013). Cancer cells acquire super-enhancers to drive expression of prominent oncogenes, so SEs play key roles in both development and disease (Chapuy et al., 2013; Loven et al., 2013). Super-enhancers are occupied by an unusually high density of interacting factors, are able to drive higher levels of transcription than typical enhancers, and are exceptionally vulnerable to perturbation of components that are commonly associated with most enhancers (Chapuy et al., 2013; Hnisz et al., 2013; Loven et al., 2013; Whyte et al., 2013).

Another puzzling observation that has emerged from recent studies is that a single enhancer is able to simultaneously activate multiple proximal genes (Fukaya et al., 2016). Enhancers physically contact the promoters of the genes they activate, and early studies using chromatin contact mapping techniques (e.g. at the β-globin locus) found that at any given time, enhancers activate only one of the several globin genes within the locus (Palstra et al., 2003; Tolhuis et al., 2002). However, more recent work using quantitative imaging at a high temporal resolution revealed that enhancers typically activate genes in bursts, and that two gene promoters can exhibit synchronous bursting when activated by the same enhancer (Fukaya et al., 2016).

Previous models of transcriptional control have provided important insights into principles of gene regulation. A key feature of most previous transcriptional control models is that the underlying regulatory interactions occur in a step-wise manner dictated by biochemical rules that are probabilistic in nature (Chen and Larson, 2016; Elowitz et al., 2002; Levine et al., 2014; Orphanides and Reinberg, 2002; Raser and O'Shea, 2004; Spitz and Furlong, 2012; Suter et al., 2011; Zoller et al., 2015). Such kinetic models predict that gene activation on a single gene level is a stochastic, noisy process, and also provide insights into how multi-step regulatory processes can suppress intrinsic noise and result in bursting. These models do not shed light on the mechanisms underlying the formation, function, and properties of SEs or explain puzzles such as how two gene promoters exhibit synchronous bursting when activated by the same enhancer.

We propose and explore herein a model that may explain the puzzles described above. This model is based on principles involving phase separation of multi-molecular assemblies.

Co-Operativity in Transcriptional Control

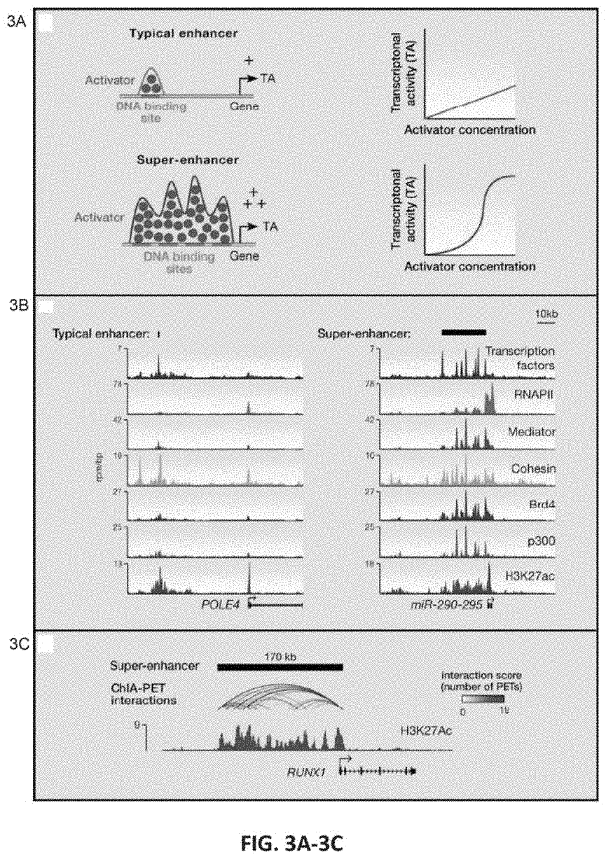

Since the discovery of enhancers over 30 years ago, studies have attempted to describe functional properties of enhancers in a quantitative manner, and these efforts have mostly relied on the concept of co-operative interactions between enhancer components. Classically, enhancers have been defined as elements that can increase transcription from a target gene promoter when inserted in either orientation at various distances upstream or downstream of the promoter (Banerji et al., 1981; Benoist and Chambon, 1981; Gruss et al., 1981). Enhancers typically consist of hundreds of base-pairs of DNA and are bound by multiple transcription factor (TF) molecules in a co-operative manner (Bulger and Groudine, 2011; Levine et al., 2014; Malik and Roeder, 2010; Ong and Corces, 2011; Spitz and Furlong, 2012). Classically, co-operative binding describes the phenomenon that the binding of one TF molecule to DNA impacts the binding of another TF molecule (FIG. 3A) (Carey, 1998; Kim and Maniatis, 1997; Thanos and Maniatis, 1995; Tjian and Maniatis, 1994). Co-operative binding of transcription factors at enhancers has been proposed to be due to the effects of TFs on DNA bending (Falvo et al., 1995), interactions between TFs (Johnson et al., 1979) and combinatorial recruitment of large cofactor complexes by TFs (Merika et al., 1998).

Super-Enhancers Exhibit Highly Co-Operative Properties

Several hundred clusters of enhancers, called super-enhancers (SEs), control genes that have especially prominent roles in cell-type-specific processes (Hnisz et al., 2013; Whyte et al., 2013). Three key features of SEs indicate that co-operative properties are especially important for their formation and function: 1) SEs are occupied by an unusually high density of interacting factors; 2) SEs can be formed by a single nucleation event; and 3) SEs are exceptionally vulnerable to perturbation of some components (i.e., super-enhancer components) that are commonly associated with most enhancers.

SEs are occupied by an unusually high density of enhancer-associated factors, including transcription factors, co-factors, chromatin regulators, RNA polymerase II, and non-coding RNA (Hnisz et al., 2013). The non-coding RNA (enhancer RNA or eRNA), produced by divergent transcription at transcription factor binding sites within SEs (Hah et al., 2015; Sigova et al., 2013), can contribute to enhancer activity and the expression of the nearby gene in cis (Dimitrova et al., 2014; Engreitz et al., 2016; Lai et al., 2013; Pefanis et al., 2015). The density of the protein factors and eRNAs at SEs has been estimated to be approximately 10-fold the density of the same set of components at typical enhancers in the genome (FIG. 3B) (Hnisz et al., 2013; Loven et al., 2013; Whyte et al., 2013). Chromatin contact mapping methods indicate that the clusters of enhancers within SEs are in close physical contact with one another and with the promoter region of the gene they activate (FIG. 3C) (Dowen et al., 2014; Hnisz et al., 2016; Ji et al., 2016; Kieffer-Kwon et al., 2013).

SEs can be formed as a consequence of introducing a single transcription factor binding site into a region of DNA that has the potential to bind additional factors. In T cell leukemias, a small (2-12 bp) mono-allelic insertion nucleates the formation of an entire SE by creating a binding site for the master transcription factor MYB, leading to the recruitment of additional transcriptional regulators to adjacent binding sites and assembly of a host of factors spread over an 8 kb domain whose features are typical of a SE (Mansour et al., 2014) Inflammatory stimulation also leads to rapid formation of SEs in endothelial cells; here again, the formation of a SE is apparently nucleated by a single binding event of a transcription factor responsive to inflammatory stimulation (Brown et al., 2014).

Entire super-enhancers spanning tens of thousands of base-pairs can collapse as a unit when their co-factors are perturbed, and genetic deletion of constituent enhancers within an SE can compromise the function of other constituents. For example, the co-activator BRD4 binds acetylated chromatin at SEs, typical enhancers and promoters, but SEs are far more sensitive to drugs blocking the binding of BRD4 to acetylated chromatin (Chapuy et al., 2013; Loven et al., 2013). A similar hypersensitivity of SEs to inhibition of the cyclin-dependent kinase CDK7 has also been observed in multiple studies (Chipumuro et al., 2014; Kwiatkowski et al., 2014; Wang et al., 2015). This kinase is critical for initiation of transcription by RNA Polymerase II (RNAPII) and phosphorylates its repetitive C-terminal domain (CTD) (Larochelle et al., 2012). Furthermore, genetic deletion of constituent enhancers within SEs can compromise the activities of other constituents within the super-enhancer (Hnisz et al., 2015; Jiang et al., 2016; Proudhon et al., 2016; Shin et al., 2016), and can lead to the collapse of an entire super-enhancer (Mansour et al., 2014), although this interdependence of constituent enhancers is less apparent for some developmentally regulated super-enhancers (Hay et al., 2016).

In summary, several lines of evidence indicate that the formation and function of SEs involves co-operative processes that bring many constituent enhancers and their bound factors into close spatial proximity. High densities of proteins and nucleic acids—and co-operative interactions among these molecules—have been implicated in the formation of membraneless organelles, called cellular bodies, in eukaryotic cells (Banjade et al., 2015; Bergeron-Sandoval et al., 2016; Brangwynne et al., 2009). Below, we first describe features of the formation of cellular bodies, and then develop a model of super-enhancer formation and function that exploits related concepts.

Formation of Membraneless Organelles by Phase Separation

Eukaryotic cells contain membraneless organelles, called cellular bodies, which play essential roles in compartmentalizing essential biochemical reactions within cells. These bodies are formed by phase separation mediated by co-operative interactions between multivalent molecules (Banjade et al., 2015; Bergeron-Sandoval et al., 2016; Brangwynne et al., 2009). Examples of such organelles in the nucleus include nucleoli, which are sites of rRNA biogenesis; Cajal bodies, which serve as an assembly site for small nuclear RNPs; and nuclear speckles, which are storage compartments for mRNA splicing factors (Mao et al., 2011; Zhu and Brangwynne, 2015). These organelles exhibit properties of liquid droplets; for example, they can undergo fission and fusion, and hence their formation has been described as mediated by liquid-liquid phase separation. Mixtures of purified RNA and RNA-binding proteins form these types of phase-separated bodies in vitro (Berry et al., 2015; Feric et al., 2016; Kato et al., 2012; Kwon et al., 2013; Li et al., 2012; Wheeler et al., 2016). Consistent with these observations, past theoretical work indicates that the formation of a gel is usually accompanied by phase separation (Semenov and Rubinstein, 1998). Thus, a number of studies show that high densities of proteins and nucleic acids—and co-operative interactions among these molecules—are implicated in the formation of phase separated cellular bodies.

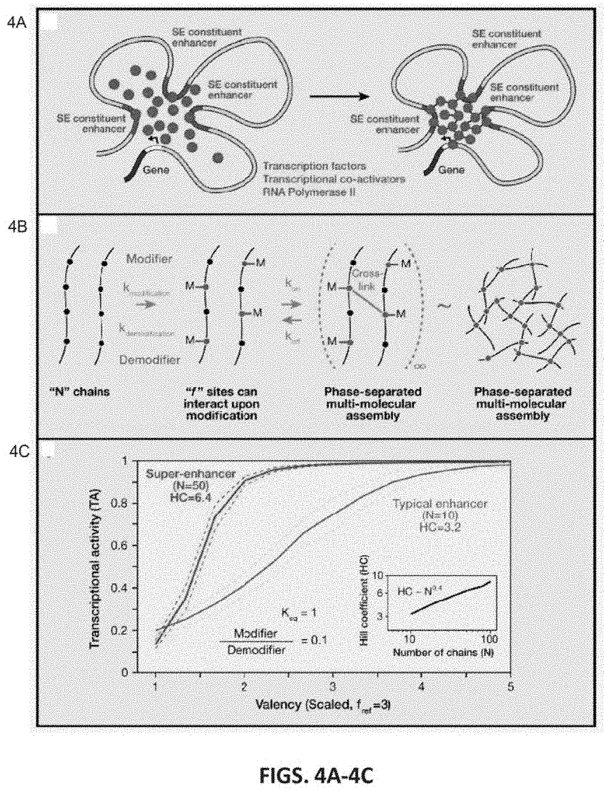

As described above, super-enhancers can be in essence considered to be co-operative assemblies of high densities of transcription factors, transcriptional co-factors, chromatin regulators, non-coding RNA and RNA Polymerase II (RNAPII). Furthermore, some transcription factors with low complexity domains have been proposed to create gel-like structures in vitro (Han et al., 2012; Kato et al., 2012; Kwon et al., 2013). We thus hypothesize that phase-separation with formation of a phase separated multi-molecular assembly likely occurs during the formation of SEs and less frequently with typical enhancers (FIG. 4A).

We propose a simple model that emphasizes co-operativity in the context of the number and valency of the interacting components, and affinity of interactions between these transcriptional regulators and nucleic acids, to explore the role of a phase separation for SE assembly and function. Computer simulations of this model show that phase separation can explain critical features of SEs, including aspects of their formation, function, and vulnerability. The simulations are also consistent with observed differences between transcriptional bursting patterns driven by weak and strong enhancers, and the simultaneous bursting of genes controlled by a shared single enhancer. We conclude by noting several implications and predictions of the phase separation model that could guide further exploration of this concept of transcriptional control in vertebrates.

A Phase Separation Model of Enhancer Assembly and Function

Many molecules bound at enhancers and SEs, such as transcription factors, transcriptional co-activators (e.g., BRD4), RNAPII and RNA can undergo reversible chemical modifications (e.g., acetylation, phosphorylation) at multiple sites. Upon such modifications, these multivalent molecules are able to interact with multiple other components, thus forming “cross-links” (FIG. 4A). Here, a cross-link can be defined as any reversible feature, including reversible chemical modification, or any other feature involved in dynamic binding and unbinding interactions. In considering whether phase separation may underlie certain observed features of transcriptional control, a simple model is needed to describe the dependence of phase separation on changes in valences and affinities of the interacting molecules, parameters biologists measure. Below we describe such a model, and explain how the parameters of this model represent characteristics of typical enhancers and super-enhancers.

In the model, the protein and nucleic acid components of enhancers are represented as chain-like molecules, each of which contains a set of residues that can potentially engage in interactions with other chains (FIG. 4B). These residues are represented as sites that can undergo reversible chemical modifications, and modification of the residues is associated with their ability to form non-covalent cross-linking interactions between the chains (FIG. 4B). Numerous enhancer-components, including transcription factors, co-factors, and the heptapeptide repeats of the C-terminal domain (CTD) of RNA polymerase II are subject to phosphorylation, and are known to bind other proteins based on their phosphorylation status (Phatnani and Greenleaf, 2006). Our model encompasses such phosphorylation or dephosphorylation that can result in binding interactions, as well as interactions of histones and other proteins found at enhancers and transcriptional regulators that are modulated by acetylation, methylation or other types of chemical modifications. For simplicity, we refer to all types of chemical modifications and de-modifications generically as “modification” and “demodification” mediated by “modifiers” and “demodifiers”, respectively.

In its simplest form, the model has three parameters: 1) “N”=the number of macromolecules (also referred to as “chains”) in the system; this parameter sets the concentration of interacting components—the larger the value of N, the greater the concentration—SEs are considered to have a larger value of N while typical enhancers are modeled as having fewer components. 2) “f”=valency, which corresponds to the number of residues in each molecule that can potentially be modified and engage in a cross-link with other chains. Note that in our simplified model, the modification of a residue is required to allow the residue to create a cross-link with another chain. Conceptually, the model works in a similar way if the demodified state of a residue is required for cross-link formation, except the enzymatic activities that allow or inhibit cross-link formation are reversed. 3) K_{eq}=(kₒₙ/k_{off}) the equilibrium constant, defined by the on and off-rates describing the cross-link reaction or interaction (FIG. 4B).

With a few assumptions, such as large chain length and not allowing intramolecular cross-links or multiple bonds between the same two chains, the equilibrium properties of this model can be obtained analytically (Cohen and Benedek, 1982; Semenov and Rubinstein, 1998). Above a critical concentration of the interacting chains, C*, phase separation occurs creating a multi-molecular assembly. Under these conditions, C* varies as 1/K_{eq}f². Thus the critical concentration for formation of the assembly depends sensitively on valency and less so on the binding constant.

We carried out computer simulations of the model (relaxing some of the assumptions in the equilibrium theories noted above) to explore its dynamic, rather than equilibrium, properties. In dynamic computer simulations of the model, the valency changes between 0 and “f” as the residues are modified and de-modified; the rates of the modification and de-modification reactions are not varied in our studies. The modifier to demodifier ratio (e.g., kinase to phosphatase ratio) in the system determines the number of sites on each component that are modified and can be cross-linked, and is varied in our studies.

The model was simulated with N chains in a fixed volume representing the region where various components of the enhancer or SE are concentrated. We considered various values of N. During the simulation, the chains can undergo modifications and de-modifications with kinetic constants, k_{mod}=0.05, k_{demod}=0.05. The modifier and demodifier levels (N_{mod}, N_{demod}) are varied. Cross-link formation and disassociation is simulated with kinetic constants, kₒₙ=0.5 and

$${k}_{off} = 0.5 ( {K}_{e q} = \frac{{k}_{o n}}{{k}_{o f f}} = 1 ) .$$

Only modified residues on different chains were allowed to cross-link—i.e., intra-chain cross-linking reactions are disallowed, but multiple bonds can form between two chains. The simulations were carried out in the limit where every site on every chain is permitted to cross-link with all other sites on other chains (Cohen and Benedek, 1982; Semenov and Rubinstein, 1998)—i.e., while there is an average concentration of interacting sites (determined by N and the number of modified sites); variations in local concentrations within the simulation volume are not considered.

The simulations were carried out using the Gillespie algorithm (Gillespie, 1977), which generates stochastic trajectories of the temporal evolution of the considered dynamic processes (i.e., modifications and cross-linking reactions). Any single trajectory describes the time-evolution of the state of interacting chains, including how they are distributed amongst clusters of varying sizes. All trajectories are initialized with demodified, non-crosslinked chains—i.e., each chain is in a “separate cluster”. Simulations are run until steady state is reached, where properties of the system (e.g. average cluster size) are time-invariant. Multiple trajectories (50 replicates) are performed for all calculations to obtain statistically averaged properties when desired.

The proxy for transcriptional activity (TA) in the simulations was defined as the size of the largest cluster of cross-linked chains, scaled by the total number of chains [TA=(size of Clusterₘₐₓ)/N]. When all chains in the system form a single cross-linked cluster (TA≈1), the phase-separated assembly results. This assembly is thought to encompass binding of factors at the enhancer/SE and also at the promoter, which leads to the concentration of components important for enhanced transcription of the gene. We recorded the transcriptional activity generated by the enhancers and SEs as a function of time.

Transcriptional Regulation with Changes in Valency

Modeling transcriptional activity as a function of valency revealed that the formation of SEs involved more pronounced co-operativity than the formation of typical enhancers (FIG. 4C). In these simulations, SEs were modeled as a system consisting of N=50 molecules, and typical enhancers as a system consisting of N=10 molecules, consistent with an approximately one order of magnitude difference in the density of components at these elements (Hnisz et al., 2013). We then graphed the transcriptional activity (TA) for different valences, while all other parameters remained constant. SEs reached ˜90% of the maximum transcriptional activity at a normalized valency value of 2 (i.e. twice the reference value of f=3), while for typical enhancers 90% of the maximum transcriptional activity is attained at a normalized valency value of 5. At a normalized valency value of 2, typical enhancers reached ˜40% of the maximum transcriptional activity (FIG. 4C). These results suggest that, under identical conditions, SEs consisting of a larger number of components form larger connected clusters (i.e. undergo phase separation) at a lower level of valency than typical enhancers consisting of a smaller number of components. Furthermore, we observed a sharp increase of transcriptional activity at a normalized valency value of −1.5 for SEs, while increases in valency leads to a more moderate, smooth increase of transcriptional activity for typical enhancers (FIG. 4C), in agreement with previous considerations (FIG. 3A) (Loven et al., 2013).

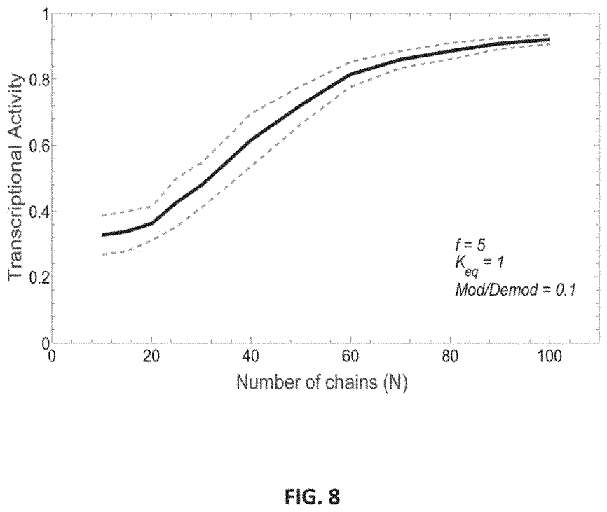

The sharper change in transcriptional activity of SEs upon changing the valency of the interacting components (i.e., super-enhancer components) due to enhanced co-operativity can be quantified by the Hill coefficient. The behavior of SEs is characterized by a larger value of the Hill coefficient, indicating greater co-operativity and ultrasensitivity to valency changes (FIG. 4C). Indeed, as the inset in FIG. 4C shows, the Hill coefficient increases with the number of components involved in the enhancer as ˜N^{0.4}, over a large range of values of N. Also, as expected, the difference between the transcriptional activity of typical enhancers and SEs correlated with the difference in values of “N” that are used to model them; for a sufficiently large difference in N, the behavior reported in FIG. 4C is recapitulated (FIG. 8).

Super-Enhancer Formation and Vulnerability

These predictions of the phase separation model are qualitatively consistent with previously published experimental data. For example, stimulation of endothelial cells by TNFα leads to the formation of SEs at inflammatory genes (Brown et al., 2014). In This manuscript, SE formation was monitored by the genomic occupancy of the transcriptional co-factor BRD4, which is a key component of SEs and typical enhancers. The inflammatory stimulation in these cells resulted in a more pronounced recruitment of BRD4 at the SEs of inflammatory genes as compared to typical enhancers at other genes (Brown et al., 2014). Our phase separation model suggests that this is because stimulation by TNFα led to modifications that change the valency of interacting components, and for SEs, phase separation occurs sharply above a lower value of valency compared to typical enhancers, thus resulting in enhanced recruitment of interacting components such as BRD4 (FIG. 4C).

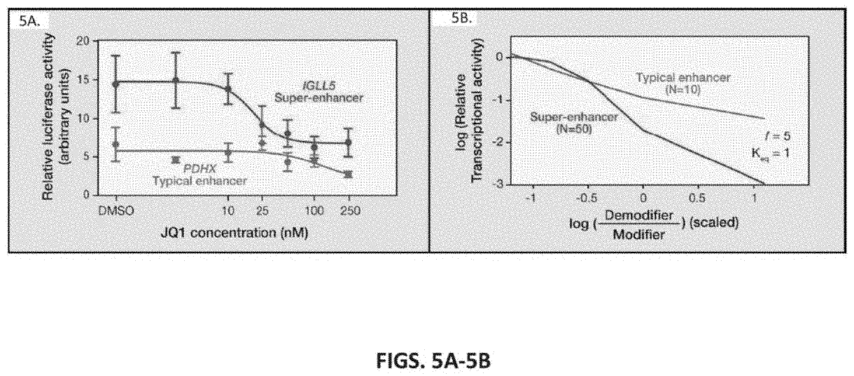

We next investigated whether the phase separation model explains the unusual vulnerability of SEs to perturbation by inhibitors of common transcriptional co-factors. BRD4 and CDK7 are components of both typical enhancers and SEs, but SEs and their associated genes are much more sensitive to chemical inhibition of BRD4 and CDK7 than typical enhancers (FIG. 5A) (Chipumuro et al., 2014; Christensen et al., 2014; Kwiatkowski et al., 2014; Loven et al., 2013). We modeled the effect of BRD4 and CDK7 inhibitors as reducing valency by changing the ratio of Demodifier/Modifier activity in our system, which shifts the balance of modified sites within the interacting molecules. This is because CDK7 is a kinase which acts as a modifier, and BRD4 has a large valency as it can interact with many components, and so inhibiting BRD4 reduces the average valency of the interacting components disproportionately. As shown in FIG. 5B, SEs (N=50) lose more of their activity sharply at a lower Demodifier/Modifier ratio than typical enhancers (N=10). These results are consistent with the notion that SE activity is very sensitive to variations in valency because phase separation is a co-operative phenomenon that occurs suddenly when a key variable exceeds a threshold value.

Transcriptional Bursting

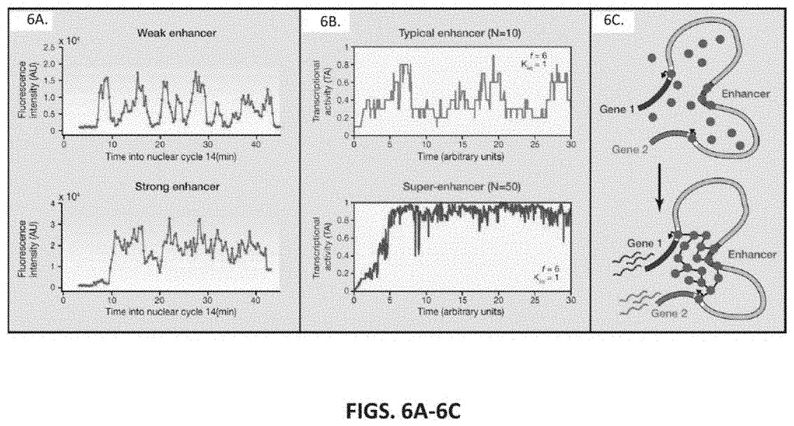

Gene expression in eukaryotes is generally episodic, consisting of transcriptional bursts, and we investigated whether the phase-separation model can predict transcriptional bursting. A recent study using quantitative imaging of transcriptional bursting in live cells suggested that the level of gene expression driven by an enhancer correlates with the frequency of transcriptional bursting (Fukaya et al., 2016). Strong enhancers were found to drive higher frequency bursting than weak enhancers, and above a certain level of strength the bursts were not resolved anymore and resulted in a relatively constant high transcriptional activity (FIG. 6A). The phase separation model shows that SEs recapitulate the high frequency with low variation (around a relatively constant high transcriptional activity) bursting pattern exhibited by strong enhancers while typical enhancers exhibit more variable bursts with a lower frequency (FIG. 6B). Once sustained phase separation occurs (TA saturates), fluctuations are quenched, which results in lower variation in TA for SEs. This difference in bursting patterns can be quantified by translating our results to a power spectrum. We expect that strong enhancers, in spite of having fewer components (N) than SEs will form stable phase separated multi-molecular assemblies more readily than typical enhancers because of higher valency cross-links. Therefore, a prediction of our model is that strong enhancers, like SE, should display a different transcriptional bursting pattern compared to weak or typical enhancers.

The phase separation model is also consistent with the intriguing observation that two promoters can exhibit synchronous bursting when activated by the same enhancer (Fukaya et al., 2016); in this case the phase-separated assembly incorporates the enhancer and both promoters (FIG. 6C).

Candidate Transcriptional Regulators Forming the Phase-Separated Assembly In Vivo

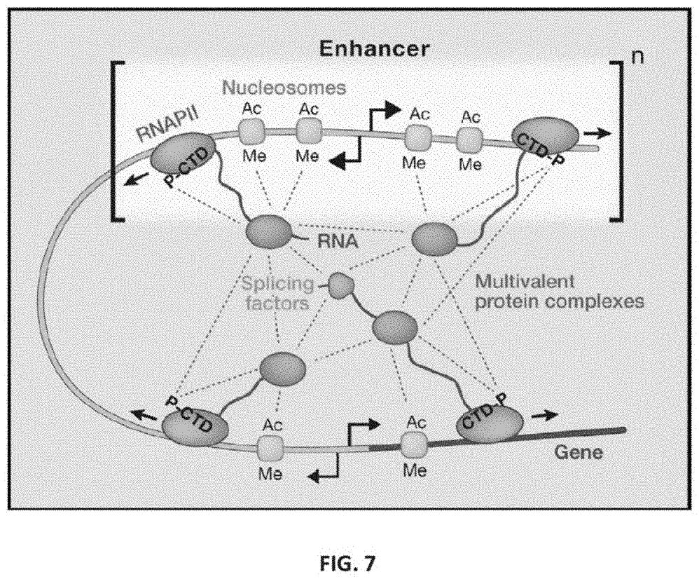

In our simplified model, phase separation is mediated by changes in the extent to which residues on the interacting components (i.e., super-enhancer components) are modified (or valency), with resulting intermolecular-interactions. In reality, however, enhancers are composed of many diverse factors that could account for such interactions, most of which are subject to reversible chemical modifications (FIG. 7). These components include transcription factors, transcriptional co-activators such as the Mediator complex and BRD4, chromatin regulators (e.g. readers, writers and erasers of histone modifications), cyclin-dependent kinases (e.g. CDK7, CDK8, CDK9, CDK12), non-coding RNAs with RNA-binding proteins and RNA polymerase II (Lai and Shiekhattar, 2014; Lee and Young, 2013; Levine et al., 2014; Malik and Roeder, 2010). Many of these molecules are multivalent, i.e. contain multiple modular domains or interaction motifs, and are thus able to interact with multiple other enhancer components. For example, the large subunit of RNA polymerase II contains 52 repeats of a heptapeptide sequence at its C-terminal domain (CTD) in human cells, and several transcription factors contain repeats of low-complexity domains or repeats of the same amino-acid stretch prone to polymerization (Gemayel et al., 2015; Kwon et al., 2013). The DNA portion of enhancers and many promoters contain binding sites for multiple transcription factors, some of which can bind simultaneously to both DNA and RNA (Sigova et al., 2015). Histone proteins at enhancers are enriched for modifications that can be recognized by chromatin readers, and thus adjacent nucleosomes can be considered as a platform able to interact with multiple chromatin readers. RNA itself can be chemically modified and physically interact with multiple RNA-binding molecules and splicing factors. Many of the residues involved in these interactions can create a “cross-link” (FIG. 7).

Possible Implications and Predictions of the Phase Separation Model

Our simple phase separation model provides a conceptual framework for further exploration of principles of gene control in development and disease. Below we discuss a few examples of phenomena possibly related to assemblies of phase separated multi-molecular complexes in transcriptional control and some testable predictions of the model.

Visualization of Phase Separated Multi-Molecular Assemblies of Transcriptional Regulators

A critical test of the model is whether phase separation of multi-molecular assemblies of transcriptional regulators can be directly observed in vivo, with the demonstration that phase separation of those complexes is associated with gene activity. Several lines of recent work provide initial insights into these questions. For example, recent studies using high resolution microscopy indicate that signal stimulation leads to the formation of large clusters of RNA polymerase II in living mammalian cells (Cisse et al., 2013) and concordant activation of transcription at a subset of genes (Cho et al., 2016). This, as well as other single molecule technologies (Chen and Larson, 2016; Shin et al., 2017), may thus enable visualization and testing of whether phase separated multi-molecular complexes form in the vicinity of genes regulated by SEs, and whether the simple model we describe here predicts features of transcriptional control. As an example, we hypothesize that the RNAPII C-terminal domain, which consists of 52 heptapeptide repeats, is a key contributor to the valency within this assembly, and in cells that express an RNAPII with a truncated CTD, the clusters would exhibit significantly lower half-lives.

Signal-Dependent Gene Control

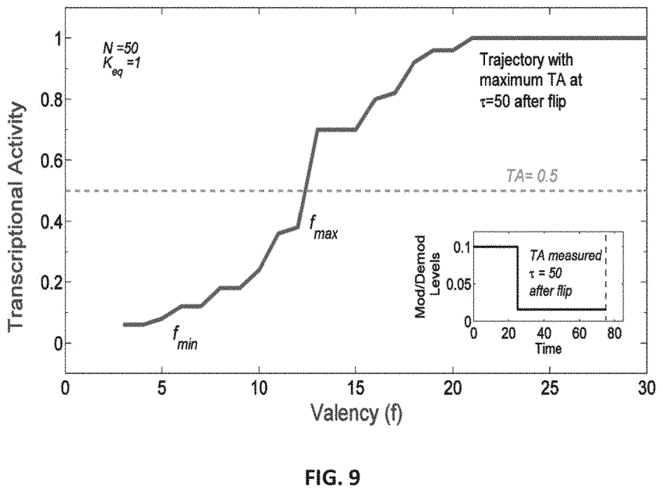

Cells sense and respond to their environment through signal transduction pathways that relay information to genes, but genes responding to a particular signaling pathway may exhibit different amplitudes of activation to the same signal. We have carried out calculations with the hypothesis that once phase separation occurs, the assembly recruits components that are de-modifiers. Under these conditions, transition to and resolution of phase separation, i.e. transcriptional activity, are more distinct for SEs compared to typical enhancers. Interestingly, such simulations suggest that there is a maximum valency and a maximum number of SE components, which if exceeded, does not allow disassembly in a realistic time scale (FIG. 9). This is because the molecules are so heavily cross-linked that it remains in a metastable state for long periods of time. The prediction of the model is that pathological hyperactivation of cellular signaling could underlie disease states through locking cells in an expression program that—at least transiently—becomes unresponsive to signals that would counteract them under normal physiological conditions. We speculate that such states can be artificially induced by increasing the valency or number of interacting components.

Fidelity of Transcriptional Control

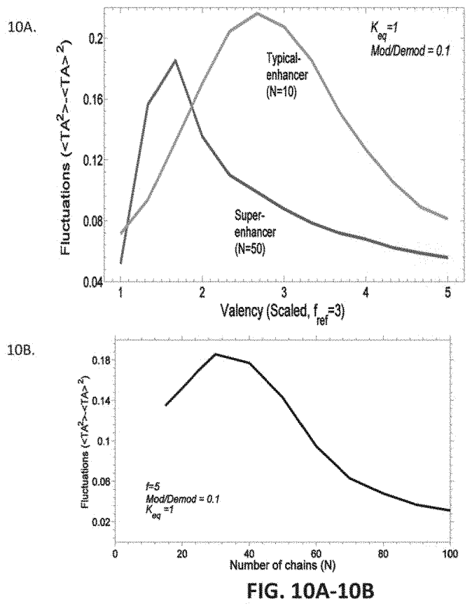

Variability in the transcript levels of genes within isogenic population of cells exposed to the same environmental signals—referred to as transcriptional noise—can have a profound impact on cellular phenotypes (Raj and van Oudenaarden, 2008). The phase separation model indicates that because of the high co-operativity involved in the formation of SEs, transcription occurs when the valency (modulated by the modifier/demodifier ratio, which is in fact similar to the developmental signals being transduced through activation cascades) exceeds a sharply defined threshold (FIG. 4C). For the smaller number of components in a typical enhancer, the variation of transcription with the environmental signal is more continuous, potentially leading to “noisier” or more error-prone transcription over a wider range of signal strength. In the vicinity of a phase separation point, there are fluctuations between the two phases (low TA and robust TA in our case). Our model shows that these fluctuations (or noise) are confined to a narrow range of environmental signals for SEs compared to the broad range over which this occurs for a typical enhancer (FIG. 10). The normalized amplitude of these fluctuations is also smaller for SEs. These results suggest that one reason why SEs have evolved is to enable relatively error free and robust transcription of genes necessary to maintain cell identity. This form of transcriptional fidelity through co-operativity, and not chemical specificity mediated by evolving specific molecules for controlling each gene, may however be co-opted to drive aberrant gene expression in disease states (e.g., SEs in cancer cells).

Resistance to Transcriptional Inhibition

Small molecule inhibitors of super-enhancer components such as BRD4 are currently being tested as anticancer therapeutics in the clinic, where a ubiquitous challenge has been the emergence of tumor cells resistant to the targeted therapeutic agent (Stathis et al., 2016). Interestingly, recent studies revealed that resistance to JQ1, a drug that inhibits BRD4, develops without any genetic changes in various tumor cells (Fong et al., 2015; Rathert et al., 2015; Shu et al., 2016). While JQ1 inhibits the interaction of BRD4 with acetylated histones, BRD4 is still recruited to super-enhancers due to its hyper-phosphorylation in JQ1-resistant cells (Shu et al., 2016). This is consistent with a prediction of our model that BRD4 is a high valency component of SEs, and inhibition of its interaction with acetylated histones (i.e. decrease of its valency) may be compensated for by increasing its valency through the activation of kinase pathways targeting BRD4 itself. In our model, super-enhancers are characterized by a high Hill coefficient, i.e. high co-operativity (FIG. 4C), which suggests that inhibition of multiple properly chosen SE components might have a synergistic effect SE-driven oncogenes in tumor cells. If this prediction is true, resistance to BRD4 inhibitors may be prevented through combined treatment with additional inhibitors of transcriptional regulators.

Concluding Remarks

The essential feature of this phase separation model of transcriptional control is that it considers co-operativity between the interacting components in the context of changes in valency and number of components. This single conceptual framework consistently describes diverse recently observed features of transcriptional control, such as clustering of factors, dynamic changes, hyper-sensitivity of SEs to transcriptional inhibitors, and simultaneous activation of multiple genes by the same enhancer. Cellular signaling pathways could modulate transcription over short time periods by alterations of valency. Selection of cell growth and survival would expand or contract the number of interactions or size of the enhancer over longer times. The model also makes a number of predictions (some noted above) that could be explored in many cellular contexts. Also, attractively, this model sets enhancer, and especially super-enhancer-type gene regulation into the broad family of membraneless organelles such as the nucleolus, Cajal bodies and splicing-speckles in the nucleus, and stress granules and P bodies in the cytoplasm, as results of phase-separated multi-molecular assemblies.

### REFERENCES

Banerji, J., Rusconi, S., and Schaffner, W. (1981). Expression of a beta-globin gene is enhanced by remote SV40 DNA sequences. Cell 27, 299-308.
Banjade, S., Wu, Q., Mittal, A., Peeples, W. B., Pappu, R. V., and Rosen, M. K. (2015). Conserved interdomain linker promotes phase separation of the multivalent adaptor protein Nck. Proceedings of the National Academy of Sciences of the United States of America 112, E6426-6435.
Benoist, C., and Chambon, P. (1981). In vivo sequence requirements of the SV40 early promotor region. Nature 290, 304-310.
Bergeron-Sandoval, L. P., Safaee, N., and Michnick, S. W. (2016). Mechanisms and Consequences of Macromolecular Phase Separation. Cell 165, 1067-1079.
Berry, J., Weber, S. C., Vaidya, N., Haataja, M., and Brangwynne, C. P. (2015). RNA transcription modulates phase transition-driven nuclear body assembly. Proceedings of the National Academy of Sciences of the United States of America 112, E5237-5245.
Brangwynne, C. P., Eckmann, C. R., Courson, D. S., Rybarska, A., Hoege, C., Gharakhani, J., Julicher, F., and Hyman, A. A. (2009). Germline P granules are liquid droplets that localize by controlled dissolution/condensation. Science 324, 1729-1732.
Brown, J. D., Lin, C. Y., Duan, Q., Griffin, G., Federation, A. J., Paranal, R. M., Bair, S., Newton, G., Lichtman, A. H., Kung, A. L., et al. (2014). NF-kappaB Directs Dynamic Super Enhancer Formation in Inflammation and Atherogenesis. Molecular cell.
Bulger, M., and Groudine, M. (2011). Functional and mechanistic diversity of distal transcription enhancers. Cell 144, 327-339.
Carey, M. (1998). The enhanceosome and transcriptional synergy. Cell 92, 5-8.
Chapuy, B., McKeown, M. R., Lin, C. Y., Monti, S., Roemer, M. G., Qi, J., Rahl, P. B., Sun, H. H., Yeda, K. T., Doench, J. G., et al. (2013). Discovery and characterization of super-enhancer-associated dependencies in diffuse large B cell lymphoma. Cancer cell 24, 777-790.
Chen, H., and Larson, D. R. (2016). What have single-molecule studies taught us about gene expression? Genes & development 30, 1796-1810.
Chipumuro, E., Marco, E., Christensen, C. L., Kwiatkowski, N., Zhang, T., Hatheway, C. M., Abraham, B. J., Sharma, B., Yeung, C., Altabef, A., et al. (2014). CDK7 Inhibition Suppresses Super-Enhancer-Linked Oncogenic Transcription in MYCN-Driven Cancer. Cell 159, 1126-1139.
Cho, W. K., Jayanth, N., English, B. P., Inoue, T., Andrews, J. O., Conway, W., Grimm, J. B., Spille, J. H., Lavis, L. D., Lionnet, T., et al. (2016). RNA Polymerase II cluster dynamics predict mRNA output in living cells. eLife 5.
Christensen, C. L., Kwiatkowski, N., Abraham, B. J., Carretero, J., Al-Shahrour, F., Zhang, T., Chipumuro, E., Herter-Sprie, G. S., Akbay, E. A., Altabef, A., et al. (2014). Targeting Transcriptional Addictions in Small Cell Lung Cancer with a Covalent CDK7 Inhibitor. Cancer cell 26, 909-922.
Cisse, II, Izeddin, I., Causse, S. Z., Boudarene, L., Senecal, A., Muresan, L., Dugast-Darzacq, C., Hajj, B., Dahan, M., and Darzacq, X. (2013). Real-time dynamics of RNA polymerase II clustering in live human cells. Science 341, 664-667.
Cohen, R. J., and Benedek, G. B. (1982). Equilibrium and kinetic theory of polymerization and the sol-gel transition. The Journal of Physical Chemistry 86, 3696-3714.
Dimitrova, N., Zamudio, J. R., Jong, R. M., Soukup, D., Resnick, R., Sarma, K., Ward, A. J., Raj, A., Lee, J. T., Sharp, P. A., et al. (2014). LincRNA-p21 activates p21 in cis to promote Polycomb target gene expression and to enforce the G1/S checkpoint. Molecular cell 54, 777-790.
Dowen, J. M., Fan, Z. P., Hnisz, D., Ren, G., Abraham, B. J., Zhang, L. N., Weintraub, A. S., Schuijers, J., Lee, T. I., Zhao, K., et al. (2014). Control of cell identity genes occurs in insulated neighborhoods in Mammalian chromosomes. Cell 159, 374-387.
Elowitz, M. B., Levine, A. J., Siggia, E. D., and Swain, P. S. (2002). Stochastic gene expression in a single cell. Science 297, 1183-1186.
ENCODE Project Consortium, Bernstein, B. E., Birney, E., Dunham, I., Green, E. D., Gunter, C., and Snyder, M. (2012). An integrated encyclopedia of DNA elements in the human genome. Nature 489, 57-74.
Engreitz, J. M., Haines, J. E., Perez, E. M., Munson, G., Chen, J., Kane, M., McDonel, P. E., Guttman, M., and Lander, E. S. (2016). Local regulation of gene expression by lncRNA promoters, transcription and splicing. Nature 539, 452-455.
Falvo, J. V., Thanos, D., and Maniatis, T. (1995). Reversal of intrinsic DNA bends in the IFN beta gene enhancer by transcription factors and the architectural protein HMG I(Y). Cell 83, 1101-1111.
Feric, M., Vaidya, N., Harmon, T. S., Mitrea, D. M., Zhu, L., Richardson, T. M., Kriwacki, R. W., Pappu, R. V., and Brangwynne, C. P. (2016). Coexisting Liquid Phases Underlie Nucleolar Subcompartments. Cell 165, 1686-1697.
Fong, C. Y., Gilan, O., Lam, E. Y., Rubin, A. F., Ftouni, S., Tyler, D., Stanley, K., Sinha, D., Yeh, P., Morison, J., et al. (2015). BET inhibitor resistance emerges from leukaemia stem cells. Nature 525, 538-542.
Fukaya, T., Lim, B., and Levine, M. (2016). Enhancer Control of Transcriptional Bursting. Cell 166, 358-368.
Gemayel, R., Chavali, S., Pougach, K., Legendre, M., Zhu, B., Boeynaems, S., van der Zande, E., Gevaert, K., Rousseau, F., Schymkowitz, J., et al. (2015). Variable Glutamine-Rich Repeats Modulate Transcription Factor Activity. Molecular cell 59, 615-627.
Gillespie, D. T. (1977). Exact stochastic simulation of coupled chemical reactions. The Journal of Physical Chemistry 81, 2340-2361.
Gruss, P., Dhar, R., and Khoury, G. (1981). Simian virus 40 tandem repeated sequences as an element of the early promoter. Proceedings of the National Academy of Sciences of the United States of America 78, 943-947.
Hah, N., Benner, C., Chong, L. W., Yu, R. T., Downes, M., and Evans, R. M. (2015) Inflammation-sensitive super enhancers form domains of coordinately regulated enhancer RNAs. Proceedings of the National Academy of Sciences of the United States of America 112, E297-302.
Han, T. W., Kato, M., Xie, S., Wu, L. C., Mirzaei, H., Pei, J., Chen, M., Xie, Y., Allen, J., Xiao, G., et al. (2012). Cell-free formation of RNA granules: bound RNAs identify features and components of cellular assemblies. Cell 149, 768-779.
Hay, D., Hughes, J. R., Babbs, C., Davies, J. O., Graham, B. J., Hanssen, L. L., Kassouf, M. T., Oudelaar, A. M., Sharpe, J. A., Suciu, M. C., et al. (2016). Genetic dissection of the alpha-globin super-enhancer in vivo. Nature genetics 48, 895-903.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Hnisz, D., Schuijers, J., Lin, C. Y., Weintraub, A. S., Abraham, B. J., Lee, T. I., Bradner, J. E., and Young, R. A. (2015). Convergence of Developmental and Oncogenic Signaling Pathways at Transcriptional Super-Enhancers. Molecular cell.
Hnisz, D., Weintraub, A. S., Day, D. S., Valton, A. L., Bak, R. O., Li, C. H., Goldmann, J., Lajoie, B. R., Fan, Z. P., Sigova, A. A., et al. (2016). Activation of proto-oncogenes by disruption of chromosome neighborhoods. Science 351, 1454-1458.
Ji, X., Dadon, D. B., Powell, B. E., Fan, Z. P., Borges-Rivera, D., Shachar, S., Weintraub, A. S., Hnisz, D., Pegoraro, G., Lee, T. I., et al. (2016). 3D Chromosome Regulatory Landscape of Human Pluripotent Cells. Cell stem cell 18, 262-275.
Jiang, T., Raviram, R., Snetkova, V., Rocha, P. P., Proudhon, C., Badri, S., Bonneau, R., Skok, J. A., and Kluger, Y. (2016). Identification of multi-loci hubs from 4C-seq demonstrates the functional importance of simultaneous interactions. Nucleic acids research.
Johnson, A. D., Meyer, B. J., and Ptashne, M. (1979). Interactions between DNA-bound repressors govern regulation by the lambda phage repressor. Proceedings of the National Academy of Sciences of the United States of America 76, 5061-5065.
Kato, M., Han, T. W., Xie, S., Shi, K., Du, X., Wu, L. C., Mirzaei, H., Goldsmith, E. J., Longgood, J., Pei, J., et al. (2012). Cell-free formation of RNA granules: low complexity sequence domains form dynamic fibers within hydrogels. Cell 149, 753-767.
Kieffer-Kwon, K. R., Tang, Z., Mathe, E., Qian, J., Sung, M. H., Li, G., Resch, W., Baek, S., Pruett, N., Grontved, L., et al. (2013). Interactome maps of mouse gene regulatory domains reveal basic principles of transcriptional regulation. Cell 155, 1507-1520.
Kim, T. K., and Maniatis, T. (1997). The mechanism of transcriptional synergy of an in vitro assembled interferon-beta enhanceosome. Molecular cell 1, 119-129.
Kwiatkowski, N., Zhang, T., Rahl, P. B., Abraham, B. J., Reddy, J., Ficarro, S. B., Dastur, A., Amzallag, A., Ramaswamy, S., Tesar, B., et al. (2014). Targeting transcription regulation in cancer with a covalent CDK7 inhibitor. Nature 511, 616-620.
Kwon, I., Kato, M., Xiang, S., Wu, L., Theodoropoulos, P., Mirzaei, H., Han, T., Xie, S., Corden, J. L., and McKnight, S. L. (2013). Phosphorylation-regulated binding of RNA polymerase II to fibrous polymers of low-complexity domains. Cell 155, 1049-1060.
Lai, F., Orom, U. A., Cesaroni, M., Beringer, M., Taatjes, D. J., Blobel, G. A., and Shiekhattar, R. (2013). Activating RNAs associate with Mediator to enhance chromatin architecture and transcription. Nature 494, 497-501.
Lai, F., and Shiekhattar, R. (2014). Enhancer RNAs: the new molecules of transcription. Curr Opin Genet Dev 25, 38-42.
Larochelle, S., Amat, R., Glover-Cutter, K., Sanso, M., Zhang, C., Allen, J. J., Shokat, K. M., Bentley, D. L., and Fisher, R. P. (2012). Cyclin-dependent kinase control of the initiation-to-elongation switch of RNA polymerase II. Nature structural & molecular biology 19, 1108-1115.
Lee, T. I., and Young, R. A. (2013). Transcriptional regulation and its misregulation in disease. Cell 152, 1237-1251.
Levine, M., Cattoglio, C., and Tjian, R. (2014). Looping back to leap forward: transcription enters a new era. Cell 157, 13-25.
Li, P., Banjade, S., Cheng, H. C., Kim, S., Chen, B., Guo, L., Llaguno, M., Hollingsworth, J. V., King, D. S., Banani, S. F., et al. (2012). Phase transitions in the assembly of multivalent signalling proteins. Nature 483, 336-340.
Loven, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of super-enhancers. Cell 153, 320-334.
Malik, S., and Roeder, R. G. (2010). The metazoan Mediator co-activator complex as an integrative hub for transcriptional regulation. Nature reviews Genetics 11, 761-772.
Mansour, M. R., Abraham, B. J., Anders, L., Berezovskaya, A., Gutierrez, A., Durbin, A. D., Etchin, J., Lawton, L., Sallan, S. E., Silverman, L. B., et al. (2014). An oncogenic super-enhancer formed through somatic mutation of a noncoding intergenic element. Science.
Mao, Y. S., Zhang, B., and Spector, D. L. (2011). Biogenesis and function of nuclear bodies. Trends in genetics: TIG 27, 295-306.
Merika, M., Williams, A. J., Chen, G., Collins, T., and Thanos, D. (1998). Recruitment of CBP/p300 by the IFN beta enhanceosome is required for synergistic activation of transcription. Molecular cell 1, 277-287.
Ong, C. T., and Corces, V. G. (2011). Enhancer function: new insights into the regulation of tissue-specific gene expression. Nature reviews Genetics 12, 283-293.
Orphanides, G., and Reinberg, D. (2002). A unified theory of gene expression. Cell 108, 439-451.
Palstra, R. J., Tolhuis, B., Splinter, E., Nijmeijer, R., Grosveld, F., and de Laat, W. (2003). The beta-globin nuclear compartment in development and erythroid differentiation. Nature genetics 35, 190-194.
Parker, S. C., Stitzel, M. L., Taylor, D. L., Orozco, J. M., Erdos, M. R., Akiyama, J. A., van Bueren, K. L., Chines, P. S., Narisu, N., Program, N. C. S., et al. (2013). Chromatin stretch enhancer states drive cell-specific gene regulation and harbor human disease risk variants. Proceedings of the National Academy of Sciences of the United States of America 110, 17921-17926.
Pefanis, E., Wang, J., Rothschild, G., Lim, J., Kazadi, D., Sun, J., Federation, A., Chao, J., Elliott, O., Liu, Z. P., et al. (2015). RNA exosome-regulated long non-coding RNA transcription controls super-enhancer activity. Cell 161, 774-789.
Phatnani, H. P., and Greenleaf, A. L. (2006). Phosphorylation and functions of the RNA polymerase II CTD. Genes & development 20, 2922-2936.
Proudhon, C., Snetkova, V., Raviram, R., Lobry, C., Badri, S., Jiang, T., Hao, B., Trimarchi, T., Kluger, Y., Aifantis, I., et al. (2016). Active and Inactive Enhancers Cooperate to Exert Localized and Long-Range Control of Gene Regulation. Cell reports 15, 2159-2169.
Raj, A., and van Oudenaarden, A. (2008). Nature, nurture, or chance: stochastic gene expression and its consequences. Cell 135, 216-226.
Raser, J. M., and O'Shea, E. K. (2004). Control of stochasticity in eukaryotic gene expression. Science 304, 1811-1814.
Rathert, P., Roth, M., Neumann, T., Muerdter, F., Roe, J. S., Muhar, M., Deswal, S., Cerny-Reiterer, S., Peter, B., Jude, J., et al. (2015). Transcriptional plasticity promotes primary and acquired resistance to BET inhibition. Nature 525, 543-547.
Roadmap Epigenomics, C., Kundaje, A., Meuleman, W., Ernst, J., Bilenky, M., Yen, A., Heravi-Moussavi, A., Kheradpour, P., Zhang, Z., Wang, J., et al. (2015). Integrative analysis of 111 reference human epigenomes. Nature 518, 317-330.
Semenov, A. N., and Rubinstein, M. (1998). Thermoreversible gelation in solutions of associative polymers. Macromolecules 31, 1373-1385.
Shin, H. Y., Willi, M., Yoo, K. H., Zeng, X., Wang, C., Metser, G., and Hennighausen, L. (2016). Hierarchy within the mammary STAT5-driven Wap super-enhancer. Nature genetics 48, 904-911.
Shin, Y., Berry, J., Pannucci, N., Haataja, M. P., Toettcher, J. E., and Brangwynne, C. P. (2017). Spatiotemporal Control of Intracellular Phase Transitions Using Light-Activated optoDroplets. Cell 168, 159-171 e114.
Shu, S., Lin, C. Y., He, H. H., Witwicki, R. M., Tabassum, D. P., Roberts, J. M., Janiszewska, M., Huh, S. J., Liang, Y., Ryan, J., et al. (2016). Response and resistance to BET bromodomain inhibitors in triple-negative breast cancer. Nature 529, 413-417.
Sigova, A. A., Abraham, B. J., Ji, X., Molinie, B., Hannett, N. M., Guo, Y. E., Jangi, M., Giallourakis, C. C., Sharp, P. A., and Young, R. A. (2015). Transcription factor trapping by RNA in gene regulatory elements. Science 350, 978-981.
Sigova, A. A., Mullen, A. C., Molinie, B., Gupta, S., Orlando, D. A., Guenther, M. G., Almada, A. E., Lin, C., Sharp, P. A., Giallourakis, C. C., et al. (2013). Divergent transcription of long noncoding RNA/mRNA gene pairs in embryonic stem cells. Proceedings of the National Academy of Sciences of the United States of America 110, 2876-2881.
Spitz, F., and Furlong, E. E. (2012). Transcription factors: from enhancer binding to developmental control. Nature reviews Genetics 13, 613-626.
Stathis, A., Zucca, E., Bekradda, M., Gomez-Roca, C., Delord, J. P., de La Motte Rouge, T., Uro-Coste, E., de Braud, F., Pelosi, G., and French, C. A. (2016). Clinical Response of Carcinomas Harboring the BRD4-NUT Oncoprotein to the Targeted Bromodomain Inhibitor OTX015/MK-8628. Cancer discovery 6, 492-500.
Suter, D. M., Molina, N., Gatfield, D., Schneider, K., Schibler, U., and Naef, F. (2011). Mammalian genes are transcribed with widely different bursting kinetics. Science 332, 472-474.
Thanos, D., and Maniatis, T. (1995). Virus induction of human IFN beta gene expression requires the assembly of an enhanceosome. Cell 83, 1091-1100.
Tjian, R., and Maniatis, T. (1994). Transcriptional activation: a complex puzzle with few easy pieces. Cell 77, 5-8.
Tolhuis, B., Palstra, R. J., Splinter, E., Grosveld, F., and de Laat, W. (2002). Looping and interaction between hypersensitive sites in the active beta-globin locus. Molecular cell 10, 1453-1465.
Wang, Y., Zhang, T., Kwiatkowski, N., Abraham, B. J., Lee, T. I., Xie, S., Yuzugullu, H., Von, T., Li, H., Lin, Z., et al. (2015). CDK7-dependent transcriptional addiction in triple-negative breast cancer. Cell 163, 174-186.
Wheeler, J. R., Matheny, T., Jain, S., Abrisch, R., and Parker, R. (2016). Distinct stages in stress granule assembly and disassembly. eLife 5.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Zhu, L., and Brangwynne, C. P. (2015). Nuclear bodies: the emerging biophysics of nucleoplasmic phases. Current opinion in cell biology 34, 23-30.
Zoller, B., Nicolas, D., Molina, N., and Naef, F. (2015). Structure of silent transcription intervals and noise characteristics of mammalian genes. Molecular systems biology 11, 823.

### Example 2

Here, we provide experimental evidence that super-enhancers form liquid-like phase-separated condensates. This establishes a new framework to account for the diverse properties described for these regulatory elements and expands the biochemical processes regulated by LLPS to include gene control.

BRD4 and MED1 are Components of Nuclear Condensates

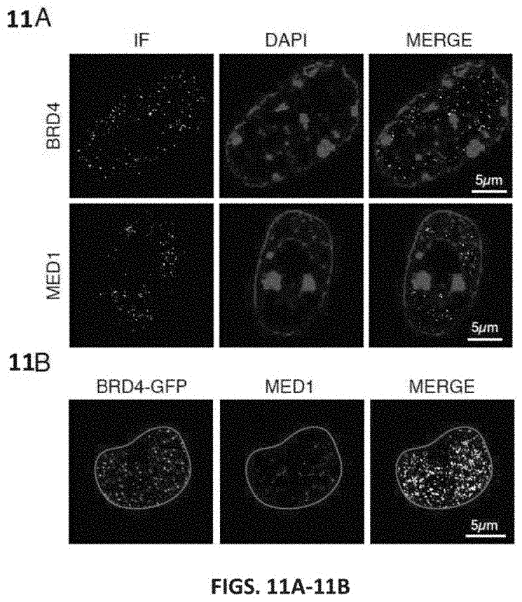

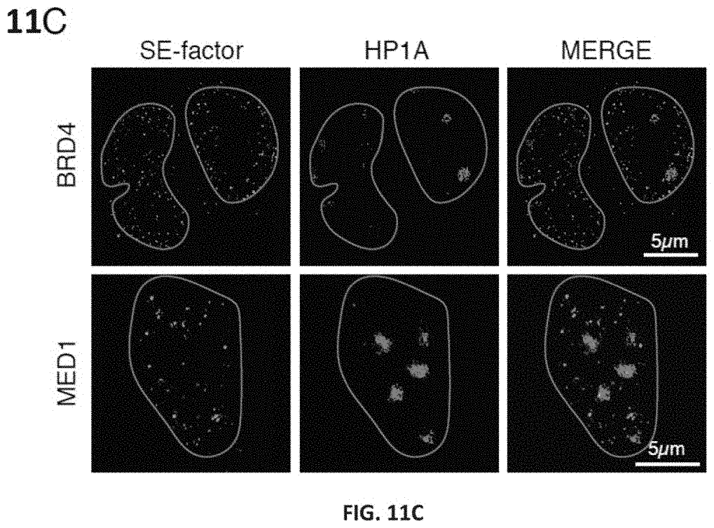

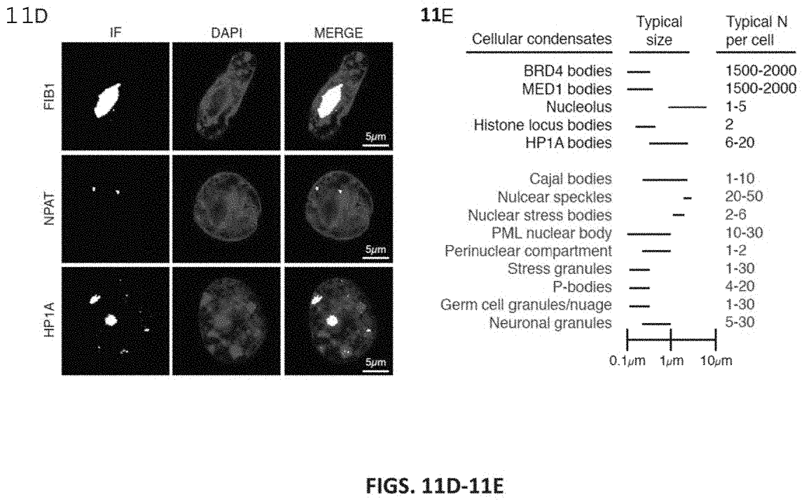

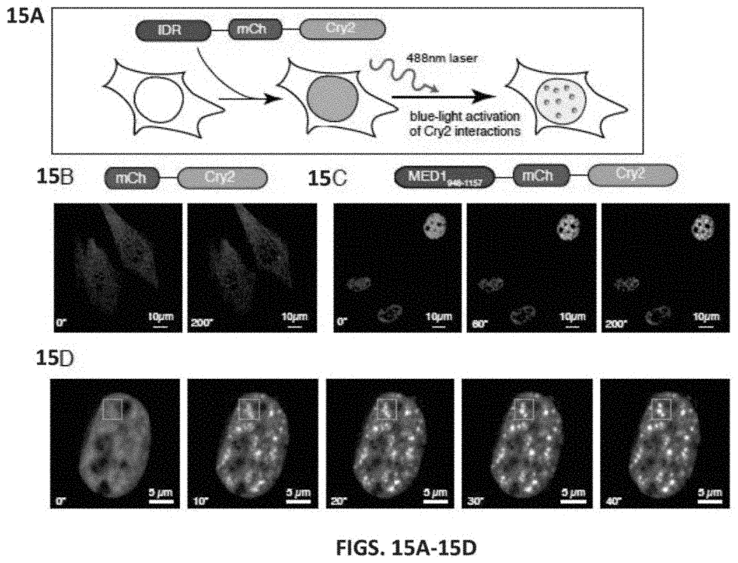

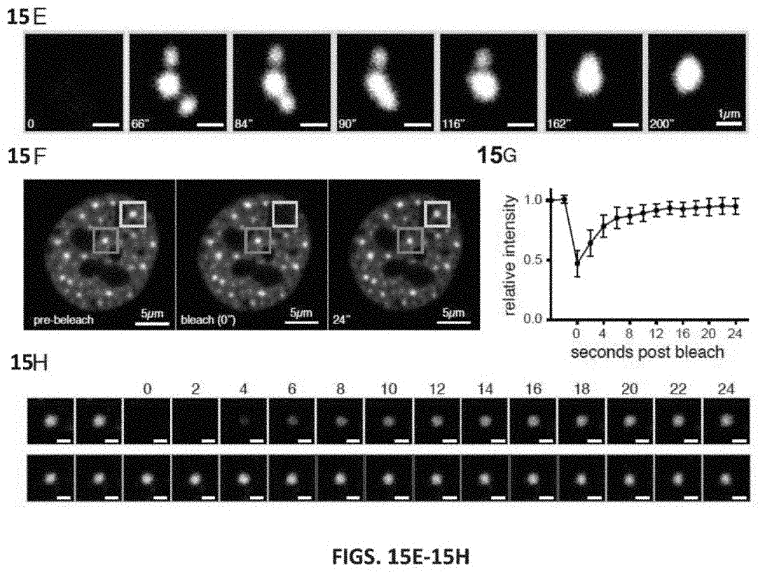

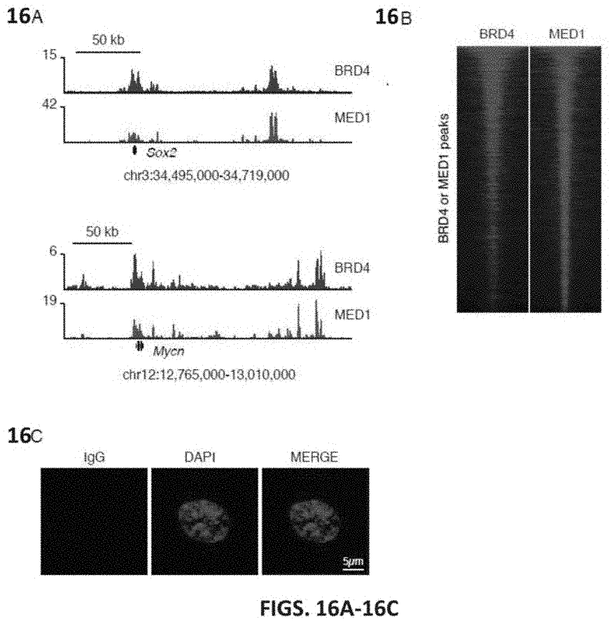

The enhancer clusters comprising SEs are occupied by master transcription factors and unusually high densities of cofactors, such as BRD4 and Mediator, whose presence can be used to define SEs (1, 2, 13). We reasoned that if SEs form nuclear condensates, then these SE-enriched cofactors could be visualized as discrete bodies in the nuclei of cells. Indeed, structured illumination microscopy (SIM) of immunofluorescence (IF) with antibodies against BRD4 and MED1 (a subunit of Mediator) revealed discrete foci in the nuclei of murine embryonic stem cells (mESCs) (FIG. 11A). The BRD4 and MED1 foci showed significant overlap (FIG. 11B), consistent with ChIP-seq data (FIGS. 16A and 15B), suggesting that the two proteins typically co-occupy these condensates. The BRD4 and MED1 foci showed poor overlap with HP1α (FIG. 11C) or other DAPI dense regions of the nucleus (FIG. 11A), indicating that BRD4 and MED1 condensates tend to occur outside heterochromatic regions of the nucleus. We also visualized previously described nuclear condensates by either deconvolution microscopy or SIM, including nucleoli (FIB1) (14), histone bodies (NPAT) (15), constitutive heterochromatin (HP1α) (16, 17) (FIG. 11D). While there is a diversity of size and number of nuclear condensates, those for BRD4 and MED1 are within the size range of previously described condensates (FIG. 11E). These results indicate that BRD4 and MED1 are not diffuse within the nucleus but occupy discrete regions, which we will refer to as BRD4 and MED1 condensates.

BRD4 and MED1 Condensates Occur at Actively Transcribed SEs

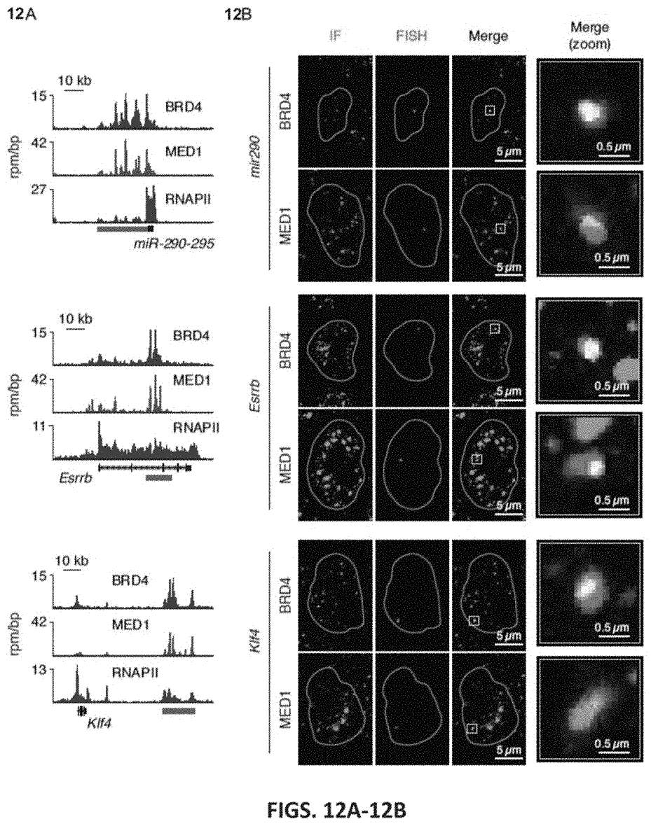

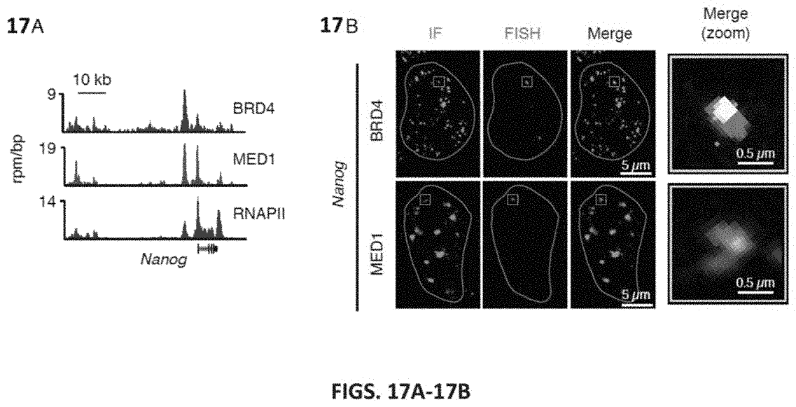

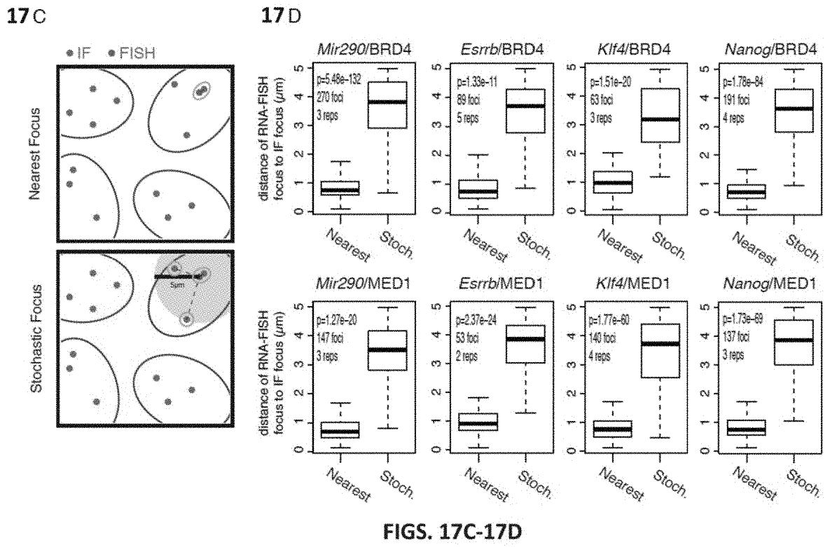

Global analysis of BRD4 and MED1 binding at enhancers by ChIP-seq suggest that there are several hundred SEs and many additional enhancers with relatively high levels of these cofactors in mESCs (1). To determine whether BRD4 and MED1 condensates are coincident with active SEs (sites of SE-driven RNA synthesis), we identified condensates using IF of BRD4 or MED1 and identified active SEs by using RNA-FISH of SE-driven nascent transcripts (probing intron RNAs) (FIG. 12 and FIG. 17). Four different active SEs were examined, and in each case, the sites of active SE-driven transcripts overlapped, or were in close proximity, to BRD4 or MED1 condensates (FIG. 12B and FIG. 17B). The frequency with which the FISH and IF signals overlapped or were in close proximity were far higher than expected by chance (FIG. 17C-17D, see materials and methods). These results indicate that actively transcribed SE-driven genes are associated with condensates containing BRD4 or MED1.

BRD4 and MED1 Condensates Exhibit Liquid-Like Fluorescence Recovery after Photobleaching Kinetics

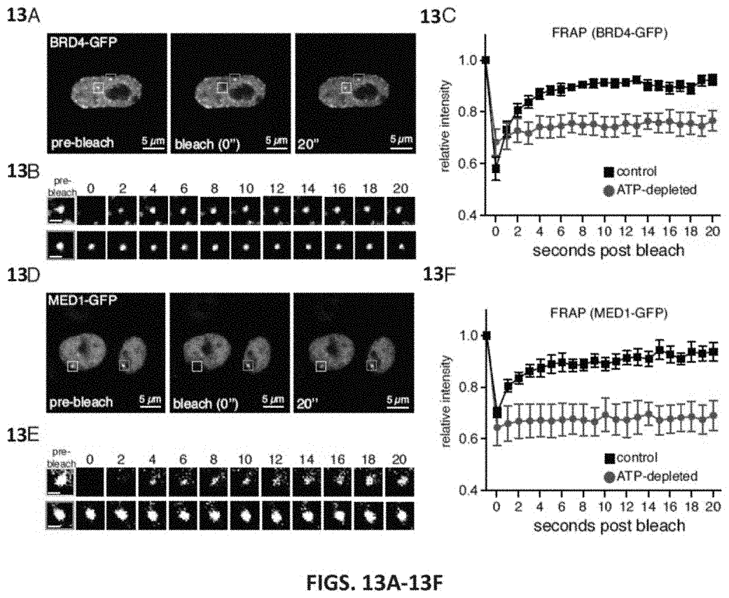

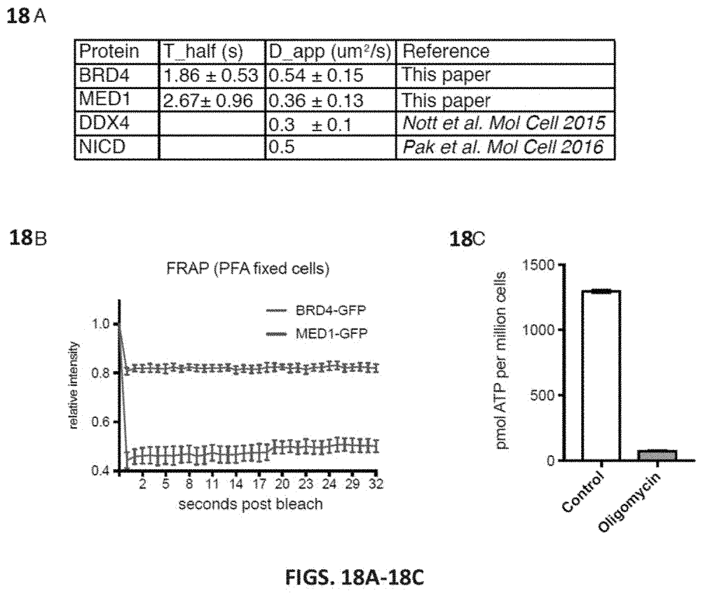

We sought to examine whether BRD4 and MED1 condensates exhibit features characteristic of liquid-like condensates. A hallmark of liquid-like condensates is internal dynamical reorganization and rapid exchange kinetics (10-12), which can be interrogated by measuring the rate of fluorescence recovery after photobleaching (FRAP). To study the dynamics of BRD4 and MED1 bodies in live cells, we ectopically expressed either BRD4-GFP or MED1-GFP in mESCs and performed FRAP experiments. After photobleaching, BRD4-GFP and MED1-GFP condensates recovered fluorescence on a time-scale of seconds (FIGS. 13 and 18A), with an apparent diffusion coefficient of 0.54±0.15 μm2/s and 0.36±0.13 μm2/s, respectively. These values are similar to previously described components of liquid-like condensates (18, 19) (FIG. 18A). Interestingly, recovery of fluorescence occurred within the same boundaries, demonstrating that the fluorescence signal represents a dynamic dense phase that rapidly exchanges components with the dilute phase (FIGS. 13B and 13E). With paraformaldehyde fixation, BRD4-GFP or MED1-GFP condensates were still present, but they exhibited no recovery after photobleaching, demonstrating that crosslinking maintains the overall condensate structure but disrupts exchange with the dilute phase (FIG. 18B). ATP has been implicated in promoting condensate fluidity by driving energy-dependent processes and/or through its intrinsic hydrotrope activity (20, 21). Depletion of cellular ATP by glucose deprivation and oligomycin treatment (FIG. 18C) abrogated fluorescence recovery after photobleaching for both BRD4-GFP and MED1-GFP bodies (FIGS. 13C and 13F). These results indicate that bodies containing BRD4 and MED1 have liquid-like properties in cells, consistent with previously described phase-separated condensates.

Intrinsically Disordered Regions of BRD4 and MED1 Phase Separate In Vitro

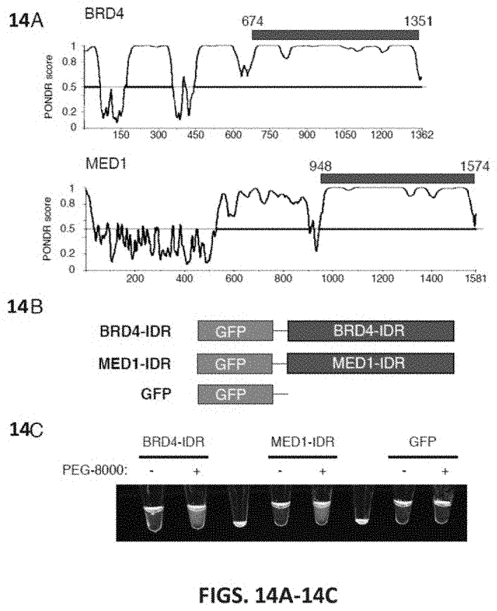

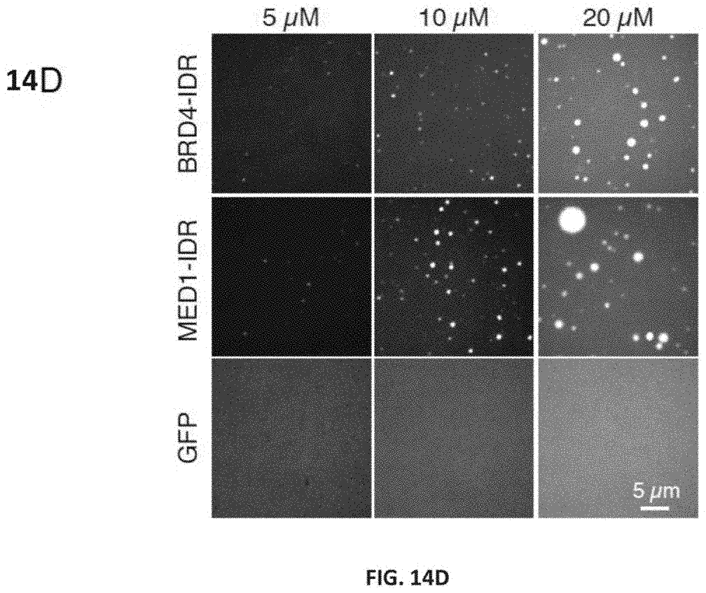

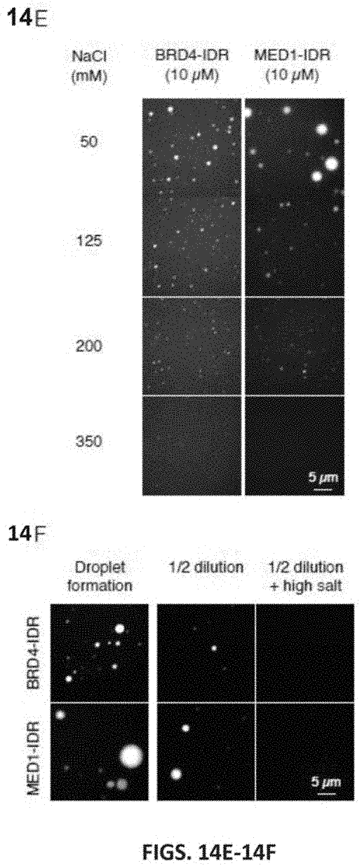

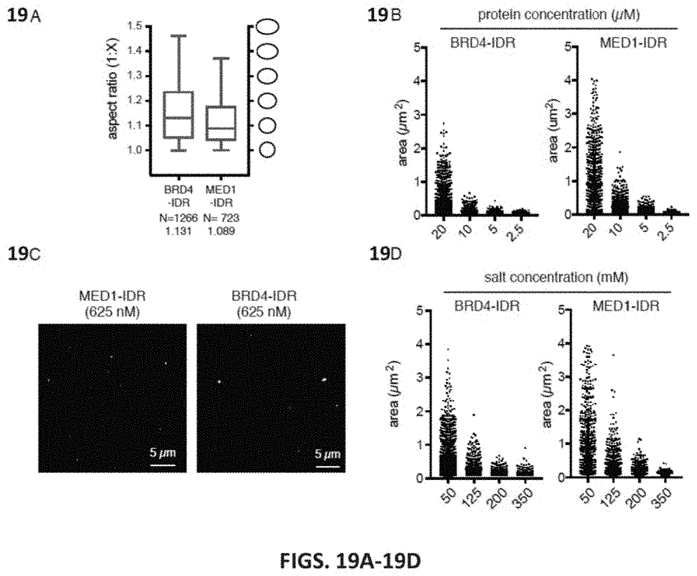

Proteins with intrinsically disordered regions (IDRs) have been implicated in facilitating condensate formation (10, 12). BRD4 and MED1 contain large IDRs (FIG. 14A). The purified IDRs of several proteins involved in condensate formation form phase-separated droplets in vitro (18, 22, 23). Therefore, we investigated whether the IDRs of BRD4 or MED1 form phase-separated droplets in vitro. Purified recombinant GFP-IDR fusion proteins (BRD4-IDR and MED1-IDR) (FIG. 14B) were added to droplet formation buffers (see materials and methods), turning the solution opaque, while equivalent solutions with only GFP remained clear (FIG. 14C). Fluorescence microscopy of the opaque MED1-IDR and BRD4-IDR solutions revealed GFP-positive, micron-sized spherical droplets freely moving in solution and falling onto and wetting the surface of the glass coverslip, where the droplets remained stationary. As determined by aspect ratio analysis, the MED1-IDR and BRD4-IDR droplets were highly spherical (FIG. 19A), a property expected for liquid-like droplets (10-12).

Phase-separated droplets typically scale in size according to the concentration of components in the system (24). We performed the droplet formation assay with varying concentrations of BRD4-IDR, MED1-IDR, and GFP ranging from 0.6 μM to 20 μM. BRD4-IDR and MED1-IDR formed droplets with concentration-dependent size distributions, whereas GFP remained diffuse in all conditions tested (FIGS. 14D and 19B). The droplets become smaller at lower concentrations, but we observed BRD4-IDR and MED1-IDR droplets at the lowest concentration tested (0.6 μM) (FIG. 19C).

Droplets consisting of purified IDRs can be sensitive to increasing salt concentrations (25). The size distributions of both BRD4-IDR and MED1-IDR shifted toward smaller droplets with increasing NaCl concentration (from 50 mM to 350 mM), consistent with droplet formation being driven by networks of weak salt-sensitive protein-protein interactions (FIGS. 14E and 19D).

To test whether the droplets are irreversible aggregates or reversible phase-separated condensates, BRD4-IDR and MED1-IDR were allowed to form droplets and then the protein concentration was diluted by half in equimolar salt or in a high salt solution (FIG. 14F). The pre-formed droplets of both BRD4-IDR and MED1-IDR were reduced in size and number with dilution and with elevated salt concentration (FIG. 14F). These results show that the BRD4-IDR and MED1-IDR droplets form a distribution of sizes dependent on the conditions of the system and, once formed, are responsive to changes in the system, with rapid adjustments in size distributions. These features are characteristic of phase-separated condensates formed by networks of weak protein-protein interactions.

MED1 IDR Participates in Liquid-Liquid Phase Separation in Cells

To investigate whether the IDR of MED1 plays a role in facilitating phase separation in cells, we used a previously developed assay that allows direct observation of droplet formation in vivo (26). Briefly, the photo-activatable, self-associating Cry2 protein is labeled with mCherry and fused to an IDR of interest, which allows for blue light-inducible increases in local concentration of selected IDRs within the cell (FIG. 15A)(26). In this assay, IDRs known to promote phase separation enhance the photo-responsive clustering properties of cry2 (27, 28), causing rapid formation of liquid-like spherical droplets (optoDroplets) upon blue light stimulation (FIG. 15A)(26). Fusion of a portion of the MED1 IDR to Cry2-mCherry facilitated the rapid formation of micron-sized spherical optoDroplets upon blue light stimulation (FIGS. 15B and 15C). During blue light stimulation, proximal optoDroplets fuse together (FIG. 5D). Furthermore, fusions exhibited characteristic liquid-like fusion properties of necking and relaxation to spherical shape (FIG. 5E).

We next tested whether the MED1-IDR optoDroplets exhibit liquid-like FRAP recovery rates (FIG. 15F-H). OptoDroplets formation was induced with blue light followed by photobleaching and recovery in the absence of blue light. Fluorescence recovered within seconds and retained the borders of the optoDroplets (FIGS. 15F and 15H). The rapid FRAP kinetics in the absence of blue light activation of Cry2 interactions suggests that the MED1-IDR optoDroplets established by blue light are dynamic assemblies exchanging with the dilute phase in the absence of the original signal. These data show that the IDR of MED1 can participate in liquid-liquid phase separation at critical local concentrations within the nucleus of live cells.

Discussion

Super-enhancers (SEs) regulate genes with prominent roles in healthy and diseased cellular states, hence improved understanding of these elements could provide new insights into the regulatory mechanisms involved in transcriptional control of these cellular states (1, 2, 29). SEs and their components have been proposed to form phase-separated condensates (3), but there has been little experimental evidence for this hypothesis. Here, we demonstrate that two key components of SEs, BRD4 and MED1, form nuclear condensates at sites of SE-driven transcription. Within these SE condensates, BRD4 and MED1 exhibit apparent diffusion coefficients similar to those previously reported for other proteins that drive in vivo phase separation (18, 19). The IDRs of both BRD4 and MED1 are sufficient to phase separate in vitro and a portion of the MED1-IDR facilitates liquid-liquid phase separation in living cells. These results indicate that SEs form phase-separated condensates that compartmentalize and concentrate the transcription apparatus at key genes and identify SE components that likely play a role in phase separation. This model has implications for the mechanisms involved in control of key cell identity genes and the functional organization of the nucleus.

SEs are established by the binding of master transcription factors (TFs) to enhancer clusters (1, 2), and these master TFs are sufficient to establish control of the gene expression programs that define cell identity (30-36). These TFs typically consist of a DNA binding domain whose structure can be determined by crystallographic methods, and a transcriptional activation domain that consists of IDRs whose structures have failed to be defined by such methods (37-39). The activation domains of these TFs recruit high densities of cofactors such as Mediator and BRD4 to SEs (2), and the concentrations of these and other components of the transcription apparatus appear to be sufficient for formation of liquid condensates. Relative to most proteins encoded in the human genome, the TFs, cofactors and transcription apparatus are enriched in IDRs (40), which might mediate weak multivalent interactions thereby facilitating condensation in vivo. We propose that condensation of high-valency factors at SEs creates a reaction crucible within the separated dense phase, where high local concentrations of the transcriptional machinery ensure robust gene expression.

The nuclear organization of chromosomes is likely influenced by SE condensates. DNA interaction technologies indicate that the individual enhancers within the SEs have exceptionally high interaction frequencies with one another (3, 41-43), consistent with the idea that condensates draw these elements into close proximity in the dense phase. Several recent studies suggest that SEs can interact with one another and may also contribute in this fashion to chromosome organization (44, 45). Cohesin, a Structural Maintenance of Chromosomes (SMC) protein complex, has been implicated in constraining SE-SE interactions because its loss causes extensive fusion of SEs within the nucleus (45). These SE-SE interactions may be due to a tendency of liquid phase condensates to undergo fusion (10-12).

The model, that SEs form phase-separated condensates that compartmentalize the transcription apparatus at key genes, raises many questions. How does condensation contribute to regulation of transcriptional output? A super-resolution study of RNA polymerase II clusters, which may be phase-separated condensates, suggests a positive correlation between condensate lifetime and transcriptional output (46). What components drive formation and dissolution of transcriptional condensates? Our studies indicate that BRD4 and MED1 likely participate, but the roles of DNA-binding TFs, cofactors, RNA POL II and regulatory RNAs require further study. Tumor cells have exceptionally large SEs at driver oncogenes that do not occur in their cell of origin, and some of these are exceptionally sensitive to drugs that target SE enriched components (29, 47).

Materials and Methods

Cell Culture

V6.5 murine embryonic stem cells (mESCs) were a gift from the Jaenisch lab. Cells were grown on 0.2% gelatinized (Sigma, G1890) tissue culture plates in 2i media, DMEM-F12 (Life Technologies, 11320082), 0.5× B27 supplement (Life Technologies, 17504044), 0.5× N2 supplement (Life Technologies, 17502048), an extra 0.5 mM L-glutamine (Gibco, 25030-081), 0.1 mM b-mercaptoethanol (Sigma, M7522), 1% Penicillin Streptomycin (Life Technologies, 15140163), 0.5× nonessential amino acids (Gibco, 11140-050), 1000 U/ml LIF (Chemico, ESG1107), 1 μM PD0325901 (Stemgent, 04-0006-10), 3 μM CHIR99021 (Stemgent, 04-0004-10). Cells were grown at 37° C. with 5% CO₂ in a humidified incubator. For confocal, deconvolution and super-resolution imaging, cells were grown on glass coverslips (Carolina Biological Supply, 633029), glass bottom dishes (Thomas Scientific, 1217N79) or 8-chambered coverglass (Life Technologies, 155409PK or VWR, 100489-104) coated with 5 μg/ml of poly-L-ornithine (Sigma-Aldrich, P4957) for 30 min at 37 C and with 5 μg/ml of Laminin (Corning, 354232) for 2 hrs-16 hrs at 37 C. For passaging, cells were washed in PBS (Life Technologies, AM9625), 1000 U/ml LIF. TrypLE Express Enzyme (Life Technologies, 12604021) was used to detach cells from plates. TrypLE was quenched with FBS/LIF-media, DMEM K/O (Gibco, 10829-018), 1× nonessential amino acids, 1% Penicillin Streptomycin, 2 mM L-Glutamine, 0.1 mM b-mercaptoethanol and 15% Fetal Bovine Serum, FBS, (Sigma Aldrich, F4135). Cells were spun at 1000 rpm for 3 min at RT, resuspended in 2i media and 5×10⁶ cells were plated in 152 cm².

HEK293T cells (ATCC, CRL-3216) were used for generation of virus used in optoDroplets experiments. HEK293T cells were cultured in DMEM (GIBCO, 11995-073) supplemented with 10% FBS (Sigma Aldrich, F4135), 2 mM L-glutamine (Gibco, 25030) and 100 U/mL penicillin-streptomycin (Gibco, 15140), at 37° C. with 5% CO₂ in a humidified incubator.

NIH 3T3 cells (ATCC, CRL-3216) were use in optoDroplets experiments. NIH 3T3 cells were cultured in DMEM (GIBCO, 11995-073) supplemented with 10% FBS (Sigma Aldrich, F4135), 2 mM L-glutamine (Gibco, 25030) and 100 U/mL penicillin-streptomycin (Gibco, 15140), at 37° C. with 5% CO₂ in a humidified incubator.

Construct Generation

MED1-GFP expression constructs were generated by fusing the full-length human MED1 cDNA to mEGFP by virtue of a 30 bp serine-glycine linker, which was juxtaposed to a PGK promoter in a lentiviral expression vector using the NEB Hi-Fi cloning kit (NEB E5520S).

Cell Treatments and Cell Line Generation

Transfection: cells were transfected with Lipofectamine 3000 (Life Technologies, L3000008) following manufacture's instruction with the following modifications. 1×10⁶ cells in 1 ml of FBS/LIF-media were plated in one gelatin-coated well of a 6-multiwell dish and during plating, Lipofectamine-DNA mix was immediately added on top of the cells. After 12 hrs, FBS/LIF-media was replaced with 2i media. Cells were imaged 24-48 hrs post transfection.

ATP depletion: Cells were cultured for 2 hours in glucose-free DMEM (Gibco, 11966025) supplemented with 0.5× B27 supplement and 0.5× N2 supplement followed by incubation with 5 mM 2-deoxy-glucose (Sigma, D6134) and 126 nM Oligomycin (Sigma, 75351) for 2 hours. Cellular ATP levels were measured using a bioluminescence assay (Invitrogen, A22066) following manufacturer's instructions.

Immunofluorescence

Immunofluorescence was performed as previously described with some modifications (49). Briefly, cells grown on coated glass were fixed in 4% paraformaldehyde, PFA, (VWR, BT140770) in PBS for 10 min at RT. After three washes in PBS for 5 min, cells were stored at 4 C or processed for immunofluorescence. Cells were permeabilized with 0.5% triton X100 (Sigma Aldrich, X100) in PBS for 5 min at RT. Following three washes in PBS for 5 min, cells were blocked with 4% IgG-free Bovine Serum Albumin, BSA, (VWR, 102643-516) for at least 15 min at RT and incubated with primary antibodies (see antibody table) in 4% IgG-free BSA O/N at RT. After three washes in PBS, primary antibody was recognized by secondary antibodies (see antibody table) in the dark. Cells were washed three times with PBS, 20 μm/ml HOESCH (Life Technologies, H3569) was used to stain nuclei for 5 min at RT in the dark. Glass slides were mounted onto slides with Vactashield (VWR, 101098-042). Coverslips were sealed with transparent nail polish (Electron Microscopy Science Nm, 72180) and stored at 4° C. Images were acquired at the RPI Spinning Disk confocal microscope with 100× objective using MetaMorph acquisition software and a Hammamatsu ORCA-ER CCD camera (W.M. Keck Microscopy Facility, MIT), or at the Applied Precision DeltaVision-OMX Super-Resolution Microscope microscope with 60× objective (Microscopy Core Facility, Koch Institute for Integrative Cancer Research) as stated in the figure legend. Structured illumination microscopy was used for nuclear bodies whose diameter was smaller than 200 nm, otherwise deconvolution or confocal microscopy was used as stated in the figure legend. Images were post-processed using Fiji Is Just ImageJ (FIJI) (50) or Imaris v9.0.0 Bitplane Inc (W.M. Keck Microscopy Facility, MIT), software available at //bitplane.com or Softworx processing software (Microscopy Core Facility, Koch Institute for Integrative Cancer Research).

RNA-FISH Combined with Immunofluorescence

Immunofluorescence was performed as previously described with the following modifications. Immunofluorescence was performed in a RNase-free environment, pipettes and bench were treated with RNaseZap (Life Technologies, AM9780). RNase-free PBS was used and antibodies were diluted in RNase-free PBS at all times. After immunofluorescence completion. Cells were post-fixed with 4% PFA in PBS for 10 min at RT. Cells were washed twice with RNase-free PBS. Cells were washed once with 20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) for 5 min at RT. Cells were hybridized with 90% Stellaris RNA FISH Hybridization Buffer (Biosearch Technologies, SMF-HB1-10), 10% Deionized Formamide, 12.5 μM Stellaris RNA FISH probes designed to hybridize introns of the transcripts of SE-associated genes. Hybridation was performed O/N at 37 C. Cells were then washed with Wash Buffer A for 30 min at 37° C. and nuclei were stained with 20 μm/ml HOESCH in Wash Buffer A for 5 min at RT. After one 5-min wash with Stellaris RNA FISH Wash Buffer B (Biosearch Technologies, SMF-WB1-20) at RT. Coverslips were mounted as described for immunofluorescence. Images were taken at the RPI Spinning Disk confocal microscope.

Fluorescence Recovery after Photobleaching (FRAP)

Cells expressing fluorescently tagged proteins were imaged ever 1 s for 20 s at a 100× objective on the Andor Revolution Spinning Disk Confocal, FRAPPA system and Metamorph acquisition software (W.M. Keck Microscopy Facility, MIT). One or two images were pre-bleach and on then approximately 0.5 μm² was bleached with the 488 nm laser of the quantifiable laser module (QLM). FRAP was performed on selecting region of interest with 5 pulses of 20 μs each.

Imaging Analysis

For structured illumination and deconvolution processing, Softworx processing software was used (Microscopy Core Facility, Koch Institute for Integrative Cancer Research).

For data displayed in FIG. 11E, nuclear condensates were counted using FIJI Particle Analysis (51) or FIJI Object Counter 3D Plugin (51). Minimum voxel size was 4 and intensity cutoff was decided based on brightness and contrast analysis.

For analysis of IF/RNA-FISH, size and coordinates of BRD4 and MED1 condensates and RNA-FISH foci were measured with FIJI Object Counter 3D Plugin (51). In accordance with image acquisition parameters, pixel width and length for images were set within FIJI to 0.0572009 microns, and the voxel depth was set to 0.5 microns. A minimum of 4 voxels was required for a body. The 3D distance between each nascent RNA transcript body (FISH) and closest protein body (IF) was measured as follows. After separate focus calling with FIJI Object Counter 3D plugin, the 3D distance between the centroids of each FISH focus and all other IF foci in the same set of images was calculated. The single closest IF focus was retained and used to display the distribution of distances to the nearest foci. A random IF focus within 5 microns of each FISH focus was also retained for a stochastic control.

For FRAP analysis, florescence recovery was measured as fluorescence intensity of photobleached area normalized to the intensity of the unbleached area or the entire nucleus. Fluorescence intensity was measured with FIJI FRAP profiler plugin (code written by Jeff Hardin, adapted from Tony Collins' Macbiophotonics plugins, available here: //worms.zoology.wisc.edu/research/4d/4d.html

ChIP-Seq Analysis

ChIP-Seq data were aligned to the mm9 version of the mouse reference genome using bowtie with parameters -k 1 -m 1 -best and -l set to read length (52). Wiggle files for display of read coverage in bins were created using MACS with parameters -w -S -space=50 -nomodel -shiftsize=200, and read counts per bin were normalized to the millions of mapped reads used to make the wiggle file (53). Reads-per-million-normalized wiggle files were displayed in the UCSC genome browser (54). Peaks of enrichment were identified using MACS with -p 1e-9 -keep-dup=1 and input control for BRD4, MED1, and RNA PolII. Super-enhancers positions in mouse embryonic stem cells were downloaded from a previous publication (55).

Factor co-localization heatmaps were created using the collapsed union of regions called a peak in BRD4 or MED1 which was generated using bedtools merge (56). Read density was calculated in 50 equally sized bins for each collapsed region using bamToGFF (https://github.com/BradnerLab/pipeline) with parameters -m 50 -r -f 1 -e 200. Heatmaps were ordered by the read signal in the BRD4/MED1/PolII signal in a given row across all columns. Presumed PCR duplicates were removed using samtools rmdup, and the density of these non-duplicate reads was used for heatmap construction(57).

Datasets are:

HP1α: GSM1375159 RNAPII: GSM1566094 MED1: GSM560348 BRD4: GSM1659409

Input control: GSM1082343

Protein Purification

For recombinant protein expression in bacteria, 6×HIS-mEGFP-linker-IDR for BRD4-IDR (BRD4₆₇₄₋₁₃₅₁) or MED1-IDR (MED1₉₄₈₋₁₅₇₄) or 6×-HIS-mEGFP-linker was cloned into a T7 pET expression vector (addgene: 29663). The linker sequence is GAPGSAGSAAGGSG (SEQ ID NO: 14). Plasmids were transformed into LOBSTR cells (gift of Cheeseman Lab). A fresh bacterial colony was inoculated into LB media containing kanamycin and chloramphenicol and grown overnight at 37° C. These bacteria were diluted 1:15 in 500 ml pre-warmed LB with freshly added kanamycin and chloramphenicol and grown for 1.5 hours at 37° C. After induction of protein expression with 1 mM IPTG, cells were grown for another 5 hours, collected, and stored frozen at −80° C. until ready to use.

Pellets from 500 ml cells were resuspended in 15 ml of Buffer A (50 mMTris pH7.5, 500 mMNaCl) containing 10 mM imidazole, cOmplete protease inhibitors (Roche, 11873580001) and sonicated (ten cycles of 15 seconds on, 60 sec off). The lysate was cleared by centrifugation at 12,000 g for 30 minutes at 4° C. and added to 1 ml of Ni-NTA agarose (Invitrogen, R901-15) pre-equilibrated with 10× volumes of buffer A. Tubes containing this agarose lysate slurry were rotated at 4 C for 1.5 hours. The slurry was poured into a column, and the packed agarose washed with 15 volumes of Buffer A containing 10 mM imidazole. Protein was eluted with 2×2 ml Buffer A containing 50 mM imidazole, 2×2 ml Buffer A with 100 mM imidazole, followed by 4×2 ml Buffer A with 250 mM imidazole.

Elutions containing protein as judged by coomassie stained gel were combined and dialyzed against Buffer D (50 mM Tris-HCl pH 7.5, 500 mM NaCl, 10% glycerol, 1 mM DTT).

In Vitro Droplet Assay

Recombinant GFP fusion proteins were concentrated and desalted to an appropriate protein concentration and 125 mM NaCl using Amicon Ultra centrifugal filters (30K MWCO, Millipore). Recombinant protein was added to solutions at varying concentrations with indicated final salt in droplet formation buffer (50 mM Trish-HCl pH 7.5, 10% glycerol, 10% PEG-8000 (Sigma 89510), 1 mM DTT). The protein solution was immediately loaded onto a homemade chamber comprising a glass slide with a coverslip attached by two parallel strips of double-sided tape. Slides were then imaged on the Andor Revolution Spinning Disk Confocal using a 100× objective. Unless otherwise indicated, images presented are of droplets settled on the glass coverslip.

OptoDroplet Assay

The optoDroplet assay was adapted from Shin, Y et al Cell 2017 (58). For cloning of IDRs, DNA segments encoding intrinsically disordered domains were amplified using Phusion Flash (ThermoFisher F548S). Segments were cloned into generation II lentiviral backbone containing the mCherry-Cry2 fusion protein (obtained from the Brangwynne laboratory) using Hi-Fi NEBuilder (NEB E2621S). Cloned opto-droplet plasmids were co-transfected with psPAX (Addgene 12260), and pMD2.G (Addgene 12259) viral packaging plasmids using PEI transfection reagent (polysciences 23966-1). Virus was produced in HEK293T cells, and was either used directly or concentrated using Takara Lenti-X Concentrator (631232). For transductions, 3T3 Cells were plated 1 day prior to transduction, seeded at 400,000 cells per 35 mm tissue culture well. Viral media was added to cells for 24 hours, at which point cells were expanded in normal media for either imaging or propagation. For imaging, 35 mm MatTek glass-bottom dishes (MatTek P35G-1.5-20-C) were coated for with 0.1 mg/ml fibronectin (EMD-Millipore FC010) for 20 minutes at 37° C. and washed twice with PBS prior to plating. Cells were plated at 400,000 cells per 35 mm dish one day before imaging. Imaging was performed on Zeiss LSM 710 point scanning microscope. Unless otherwise indicated, droplet formation was induced with 488 nm light pulses every 2 seconds for the duration of imaging, with images also taken every 2 seconds. Duration of imaging as indicated. mCherry fluorescence was stimulated with 561 nm light. For FRAP experiments, droplet formation was induced with 488 nm light for 40 seconds, at which point foci were bleached with 561 nm light and recovery was imaged every 2 seconds in the absence of 488 nm stimulation.

Antibodies

| | Company and | |
|---|---|---|
| | Catalog number | Dilution |
| BRD4 | Abcam ab128874 | 1:500 |
| BRD4-Alxa488 | Abcam ab197606 | 1:100-1:200 |
| MED1 | Applied Biosciences B0556 | 1:500 |
| HP1a-Alexa555 | Abcam ab203432 | 1:500 |
| FIB1 | Abcam ab5821 | 1:500 |
| NPAT | Bethyl A302-772A | 1:500 |
| Anti-rabbit IgG-546 | | 1:500 |
| Goat anti-Rabbit IgG | Life Technologies A11008 | 1:500 |
| Alexa Fluor 488 | | |
| Goat anti-Mouse | Life Technologies A11030 | 1:500 |
| IgG Alexa Fluor 546 | | |

Constructs

| | Company and | |
|---|---|---|
| | Catalog number | Reference |
| BRD4-GFP | Addgene Plasmid #65378 | (59) |
| HP1a-GFP | Cheesman lab | |
| mCherry-Cry2WT | Brangwynne laboratory | |
| MED1-GFP | This disclosure | |
| pET-BRD4-IDR | This disclosure | |
| pET-MED1-IDR | This disclosure | |
| pET-GFP | This disclosure | |
| OptoIDR-MED1-frag1 | This disclosure | |

### REFERENCES

1. W. A. Whyte et al., Master Transcription Factors and Mediator Establish Super-Enhancers at Key Cell Identity Genes. Cell. 153, 307-319 (2013).
2. D. Hnisz et al., Super-enhancers in the control of cell identity and disease. Cell. 155, 934-947 (2013).
3. D. Hnisz, K. Shrinivas, R. A. Young, A. K. Chakraborty, P. A. Sharp, A Phase Separation Model for Transcriptional Control. Cell. 169, 13-23 (2017).
4. K. Adelman, J. T. Lis, Promoter-proximal pausing of RNA polymerase II: emerging roles in metazoans. Nature Reviews Genetics. 13, 720-731 (2012).
5. M. Bulger, M. Groudine, Functional and Mechanistic Diversity of Distal Transcription Enhancers. Cell. 144, 327-339 (2011).
6. E. Cabo, J. Wysocka, Modification of Enhancer Chromatin: What, How, and Why? Molecular Cell. 49, 825-837 (2013).
7. F. Spitz, E. E. M. Furlong, Transcription factors: from enhancer binding to developmental control. Nature Reviews Genetics. 13, 613-626 (2012).
8. W. Xie, B. Ren, Enhancing Pluripotency and Lineage Specification. Science. 341, 245-247 (2013).
9. M. Levine, C. Cattoglio, R. Tjian, Looping Back to Leap Forward: Transcription Enters a New Era. Cell. 157, 13-25 (2014).
10. S. F. Banani, H. O. Lee, A. A. Hyman, M. K. Rosen, Biomolecular condensates: organizers of cellular biochemistry. Nat Rev Mol Cell Biol. 18, 285-298 (2017).
11. A. A. Hyman, C. A. Weber, F. Jülicher, Liquid-Liquid Phase Separation in Biology. Annu. Rev. Cell Dev. Biol. 30, 39-58 (2014).
12. Y. Shin, C. P. Brangwynne, Liquid phase condensation in cell physiology and disease. Science. 357, eaaf4382 (2017).
13. B. Chapuy et al., Discovery and Characterization of Super-Enhancer-Associated Dependencies in Diffuse Large B Cell Lymphoma. Cancer Cell. 24, 777-790 (2013).
14. T. Pederson, The nucleolus. Cold Spring Harbor Perspectives in Biology. 3, a000638-a000638 (2011).
15. Z. Nizami, S. Deryusheva, J. G. Gall, The Cajal body and histone locus body. Cold Spring Harbor Perspectives in Biology. 2, a000653 (2010).
16. A. G. Larson et al., Liquid droplet formation by HP1α suggests a role for phase separation in heterochromatin. Nature. 547, 236-240 (2017).
17. A. R. Strom et al., Phase separation drives heterochromatin domain formation. Nature. 547, 241-245 (2017).
18. T. J. Nott et al., Phase transition of a disordered nuage protein generates environmentally responsive membraneless organelles. Molecular Cell. 57, 936-947 (2015).
19. C. W. Pak et al., Sequence Determinants of Intracellular Phase Separation by Complex Coacervation of a Disordered Protein. Molecular Cell. 63, 72-85 (2016).
20. C. P. Brangwynne, T. J. Mitchison, A. A. Hyman, Active liquid-like behavior of nucleoli determines their size and shape in Xenopus laevis oocytes. Proceedings of the National Academy of Sciences. 108, 4334-4339 (2011).
21. A. Patel et al., ATP as a biological hydrotrope. Science. 356, 753-756 (2017).
22. Y. Lin, D. S. W. Protter, M. K. Rosen, R. Parker, Formation and Maturation of Phase-Separated Liquid Droplets by RNA-Binding Proteins. Molecular Cell. 60, 208-219 (2015).
23. K. A. Burke, A. M. Janke, C. L. Rhine, N. L. Fawzi, Residue-by-Residue View of In Vitro FUS Granules that Bind the C-Terminal Domain of RNA Polymerase II. Molecular Cell. 60, 231-241 (2015).
24. C. P. Brangwynne, Phase transitions and size scaling of membrane-less organelles. J Cell Biol. 203, 875-881 (2013).
25. C. P. Brangwynne, P. Tompa, R. V. Pappu, Polymer physics of intracellular phase transitions. Nat Phys. 11, 899-904 (2015).
26. Y. Shin et al., Spatiotemporal Control of Intracellular Phase Transitions Using Light-Activated optoDroplets. Cell. 168, 159-171.e14 (2017).
27. I. Ozkan-Dagliyan et al., Formation of Arabidopsis Cryptochrome 2 Photobodies in Mammalian Nuclei APPLICATION AS AN OPTOGENETIC DNA DAMAGE CHECKPOINT SWITCH. J. Biol. Chem. 288, 23244-23251 (2013).
28. X. Yu et al., Formation of Nuclear Bodies of Arabidopsis CRY2 in Response to Blue Light Is Associated with Its Blue Light-Dependent Degradation. The Plant Cell. 21, 118-130 (2009).
29. J. Lovén et al., Selective Inhibition of Tumor Oncogenes by Disruption of Super-Enhancers. Cell. 153, 320-334 (2013).
30. Y. Buganim, D. A. Faddah, R. Jaenisch, Mechanisms and models of somatic cell reprogramming. Nature Reviews Genetics. 14, 427-439 (2013).
31. T. Graf, T. Enver, Forcing cells to change lineages. Nature. 462, 587-594 (2009).
32. T. I. Lee, R. A. Young, Transcriptional Regulation and Its Misregulation in Disease. Cell. 152, 1237-1251 (2013).
33. S. A. Morris, G. Q. Daley, A blueprint for engineering cell fate: current technologies to reprogram cell identity. Cell Research. 23, 33-48 (2013).
34. I. Sancho-Martinez, S. H. Baek, J. C. I. Belmonte, Lineage conversion methodologies meet the reprogramming toolbox. Nat Cell Biol. 14, ncb2567-899 (2012).
35. T. Vierbuchen, M. Wernig, Molecular Roadblocks for Cellular Reprogramming. Molecular Cell. 47, 827-838 (2012).
36. S. Yamanaka, Induced Pluripotent Stem Cells: Past, Present, and Future. Stem Cell. 10, 678-684 (2012).
37. M. Ptashne, How eukaryotic transcriptional activators work. Nature. 335, 683-689 (1988).
38. P. J. Mitchell, R. Tjian, Transcriptional regulation in mammalian cells by sequence-specific DNA binding proteins. Science. 245, 371-378 (1989).
39. J. Liu et al., Intrinsic Disorder in Transcription Factors. Biochemistry. 45, 6873-6888 (2006).
40. H. Xie et al., Functional Anthology of Intrinsic Disorder. 1. Biological Processes and Functions of Proteins with Long Disordered Regions. J. Proteome Res. 6, 1882-1898 (2007).
41. J. M. Dowen et al., Control of Cell Identity Genes Occurs in Insulated Neighborhoods in Mammalian Chromosomes. Cell. 159, 374-387 (2014).
42. X. Ji et al., 3D Chromosome Regulatory Landscape of Human Pluripotent Cells. Cell Stem Cell. 18, 262-275 (2016).
43. K.-R. Kieffer-Kwon et al., Interactome Maps of Mouse Gene Regulatory Domains Reveal Basic Principles of Transcriptional Regulation. Cell. 155, 1507-1520 (2013).
44. R. A. Beagrie et al., Complex multi-enhancer contacts captured by genome architecture mapping. Nature. 295, 1306 (2017).
45. S. S. P. Rao et al., Cohesin Loss Eliminates All Loop Domains. Cell. 171, 305-320.e24 (2017).
46. W.-K. Cho et al., RNA Polymerase II cluster dynamics predict mRNA output in living cells. Elife. 5, 1123 (2016).
47. N. Kwiatkowski et al., Targeting transcription regulation in cancer with a covalent CDK7 inhibitor. Nature. 511, 616-620 (2014).
48. M. Dundr, T. Misteli, Biogenesis of Nuclear Bodies. Cold Spring Harbor Perspectives in Biology. 2, a000711-a000711 (2010).
49. S. Albini et al., Brahma is required for cell cycle arrest and late muscle gene expression during skeletal myogenesis. EMBO Rep 16, 1037-1050 (2015).
50. J. Schindelin et al., Fiji: an open-source platform for biological-image analysis. Nat Methods 9, 676-682 (2012).
51. S. Bolte, F. P. Cordelieres, A guided tour into subcellular colocalization analysis in light microscopy. J Microsc 224, 213-232 (2006).
52. B. Langmead, C. Trapnell, M. Pop, S. L. Salzberg, Ultrafast and memory-efficient alignment of short DNA sequences to the human genome. Genome Biol 10, R25 (2009).
53. Y. Zhang et al., Model-based analysis of ChIP-Seq (MACS). Genome Biol 9, R137 (2008).
54. W. J. Kent et al., The human genome browser at UCSC. Genome Res 12, 996-1006 (2002).
55. W. A. Whyte et al., Master transcription factors and mediator establish super-enhancers at key cell identity genes. Cell 153, 307-319 (2013).
56. A. R. Quinlan, I. M. Hall, BEDTools: a flexible suite of utilities for comparing genomic features. Bioinformatics 26, 841-842 (2010).
57. H. Li et al., The Sequence Alignment/Map format and SAMtools. Bioinformatics 25, 2078-2079 (2009).
58. Y. Shin et al., Spatiotemporal Control of Intracellular Phase Transitions Using Light-Activated optoDroplets. Cell 168, 159-171 e114 (2017).
59. F. Gong et al., Screen identifies bromodomain protein ZMYND8 in chromatin recognition of transcription-associated DNA damage that promotes homologous recombination. Genes Dev 29, 197-211 (2015).

### Example 3

Gene expression is controlled by transcription factors (TFs) that consist of DNA-binding domains (DBDs) and activation domains (ADs). The DBDs have been well-characterized, but little is known about the mechanisms by which ADs effect gene activation. Here we report that diverse ADs form phase-separated condensates with the Mediator coactivator. For the OCT4 and GCN4 TFs, we show that the ability to form phase-separated droplets with Mediator in vitro and the ability to activate genes in vivo are dependent on the same amino acid residues. For the estrogen receptor (ER), a ligand-dependent activator, we show that estrogen enhances phase separation with Mediator, again linking phase separation with gene activation. These results suggest that diverse TFs can interact with Mediator through the phase-separating capacity of their ADs and that formation of condensates with Mediator is involved in gene activation.

Recent studies have shown that the AD of the yeast TF GCN4 binds to the Mediator subunit MED15 at multiple sites and in multiple orientations and conformations (Brzovic et al., 2011; Jedidi et al., 2010; Tuttle et al., 2018; Warfield et al., 2014). The products of this type of protein-protein interaction, where the interaction interface cannot be described by a single conformation, have been termed “fuzzy complexes” (Tompa and Fuxreiter, 2008). These dynamic interactions are also typical of the IDR-IDR interactions that facilitate formation of phase-separated biomolecular condensates (Alberti, 2017; Banani et al., 2017; Hyman et al., 2014; Shin and Brangwynne, 2017; Wheeler and Hyman, 2018).

Here, we report that diverse TF ADs phase separate with the Mediator coactivator. We show that the embryonic stem cell (ESC) pluripotency TF OCT4, the estrogen receptor (ER) and the yeast TF GCN4 form phase-separated condensates with Mediator and require the same amino acids or ligands for both activation and phase separation. We show that IDR-mediated phase separation with coactivators is a mechanism by which TF ADs activate genes.

Results

Mediator Condensates at ESC Super-Enhancers Depend on OCT4

OCT4 is a master TF essential for the pluripotent state of ESCs and is a defining TF at ESC SEs (Whyte et al., 2013). The Mediator coactivator, which forms condensates at ESC SEs (Sabari et al., 2018), is thought to interact with OCT4 via the MED1 subunit (Table S3) (Apostolou et al., 2013). If OCT4 contributes to the formation of Mediator condensates, then OCT4 puncta should be present at the SEs where MED1 puncta have been observed. Indeed, immunofluorescence (IF) microscopy with concurrent nascent RNA FISH revealed discrete OCT4 puncta at the SEs of the key pluripotency genes Esrrb, Nanog, Trim28 and Mir290 (FIG. 20). Average image analysis confirmed that OCT4 IF was enriched at center of RNA FISH foci. This enrichment was not seen using a randomly selected nuclear position (FIG. 27). These results confirm that OCT4 occurs puncta at the same SEs where Mediator forms condensates (Sabari et al., 2018) and where ChIP-seq shows co-occupancy of OCT4 and MED1 (FIG. 20).

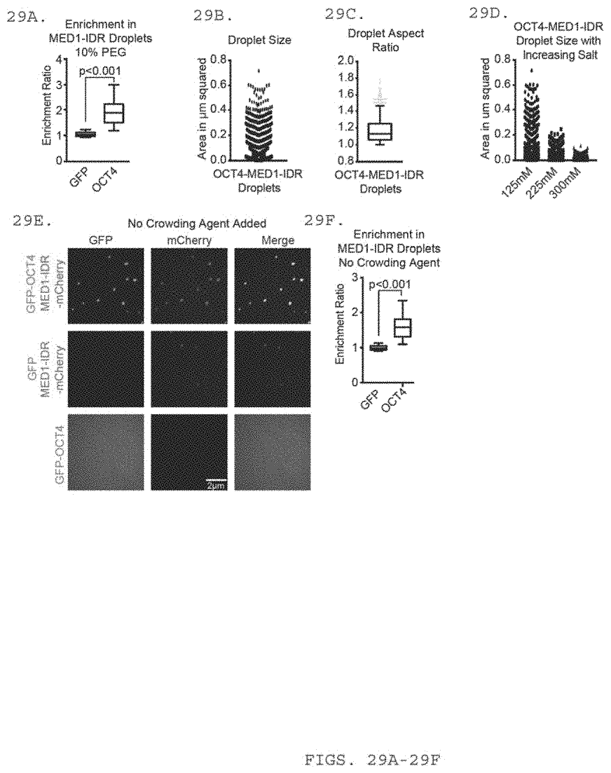

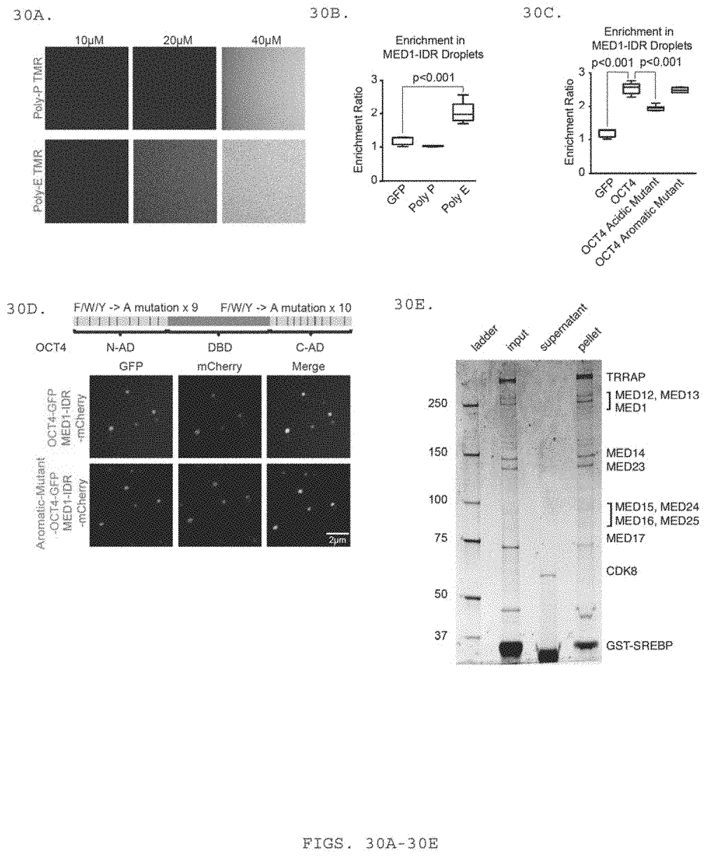

We investigated whether the Mediator condensates present at SEs are dependent on OCT4 using a degradation strategy (Nabet et al., 2018). Degradation of OCT4 in an ESC line bearing endogenous knock-in of DNA encoding the FKBP protein fused to OCT4 was induced by addition of dTag for 24 hours (Weintraub et al., 2017) (FIGS. 21A and 28A). Induction of OCT4 degradation reduced OCT4 protein levels, but did not affect MED1 levels (FIG. 28B). ChIP-seq analysis showed a reduction of OCT4 and MEM occupancy at enhancers, with the most profound effects occurring at SEs, as compared to typical enhancers (TEs). (FIG. 21B). RNA-seq revealed that expression of SE-driven genes was concomitantly decreased (FIG. 21B). For example, OCT4 and MED1 occupancy was reduced by approximately 90% at the Nanog SE (FIG. 21C), associated with a 60% reduction in Nanog mRNA levels (FIG. 21D). Immunofluorescence (IF) microscopy with concurrent DNA FISH showed that OCT4 degradation caused a reduction in MED1 condensates at Nanog (FIGS. 21E and 28C). These results indicate that the presence of Mediator condensates at an ESC SE is dependent on OCT4.

ESC differentiation causes a loss of OCT4 binding at certain ESC SEs, which leads to a loss of these OCT4-dependent SEs, and thus should cause a loss of Mediator condensates at these sites. To test this idea, we differentiated ESCs by LIF withdrawal. In the differentiated cell population, we observed reduced OCT4 and MED1 occupancy at the MiR290 SE (FIGS. 21F, 21G, and 28D) and reduced levels of MiR290 miRNA (FIG. 21H), despite continued expression of MED1 protein (FIG. 28E). Correspondingly, MED1 condensates were reduced at Mir290 (FIGS. 21I and 28F) in the differentiated cell population. These results are consistent with those obtained with the OCT4 degron experiment and support the idea that Mediator condensates at these ESC SEs are dependent on occupancy of the enhancer elements by OCT4.

OCT4 is Incorporated into MED1 Liquid Droplets

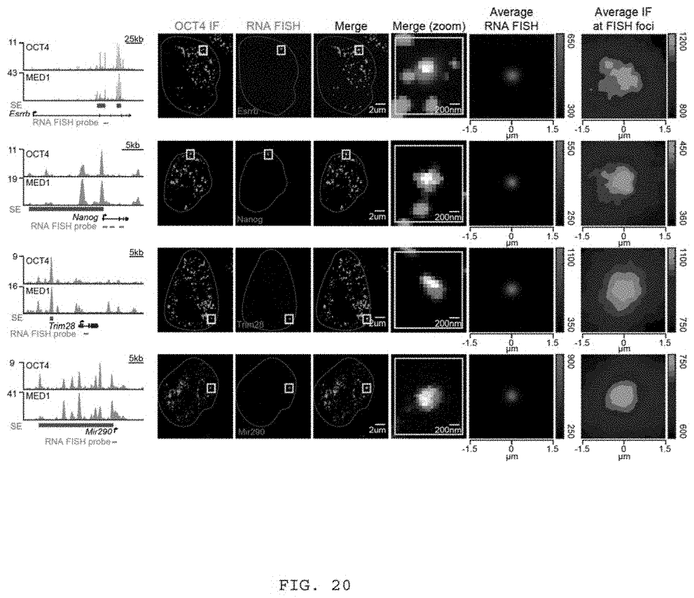

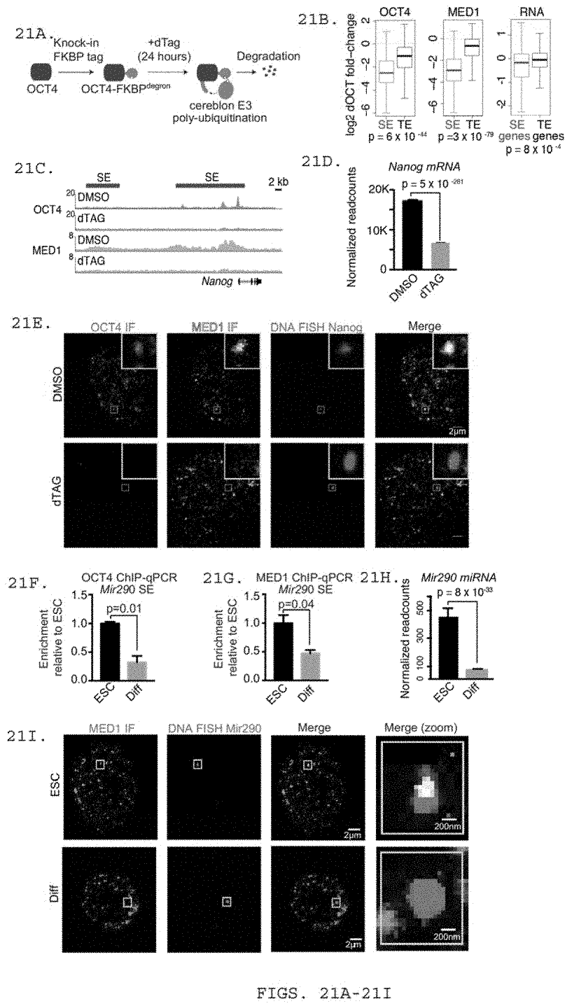

OCT4 has two intrinsically disordered ADs responsible for gene activation, which flank a structured DBD (FIG. 22A) (Brehm et al., 1997). Since IDRs are capable of forming dynamic networks of weak interactions, and the purified IDRs of proteins involved in condensate formation can form phase-separated droplets (Burke et al., 2015; Lin et al., 2015; Nott et al., 2015), we next investigated whether OCT4 is capable of forming droplets in vitro, with and without the IDR of the MED1 subunit of Mediator.

Recombinant OCT4-GFP fusion protein was purified and added to droplet formation buffers containing a crowding agent (10% PEG-8000) to simulate the densely crowded environment of the nucleus. Fluorescent microscopy of the droplet mixture revealed that OCT4 alone did not form droplets throughout the range of concentrations tested (FIG. 22B). In contrast, purified recombinant MED1-IDR-GFP fusion protein exhibited concentration-dependent liquid-liquid phase separation (FIG. 22B), as described previously (Sabari et al., 2018).

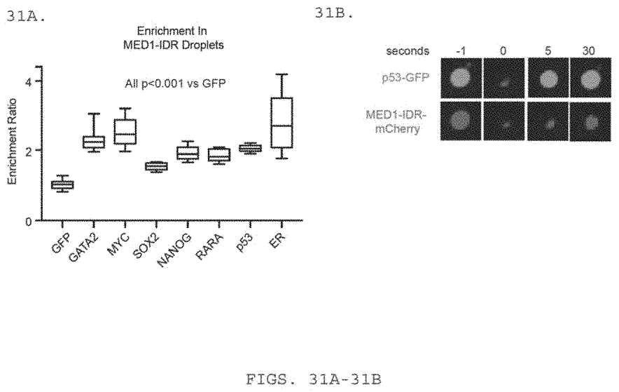

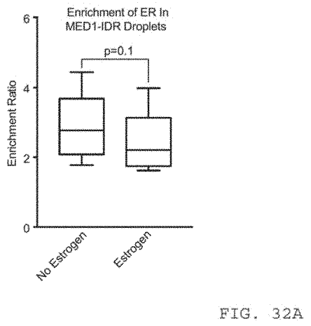

We then mixed the two proteins and found that droplets of MED1-IDR incorporate and concentrate purified OCT4-GFP to form heterotypic droplets (FIG. 22C). In contrast, purified GFP was not concentrated into MED1-IDR droplets (FIG. 22C, 29A). OCT4-MED1-IDR droplets were near-micron-sized (FIG. 29B), exhibited fast recovery after photobleaching (FIG. 22D), spherical shape (FIG. 29C), and were salt sensitive (FIGS. 22E and 29D). Thus, they exhibited characteristics associated with phase-separated liquid condensates (Banani et al 2017; Shin et al 2017). Furthermore, we found that OCT4-MED1-IDR droplets could form in the absence of any crowding agent (FIGS. 29E and 29F).

Residues Required for OCT4-MED1-IDR Droplet Formation and Gene Activation

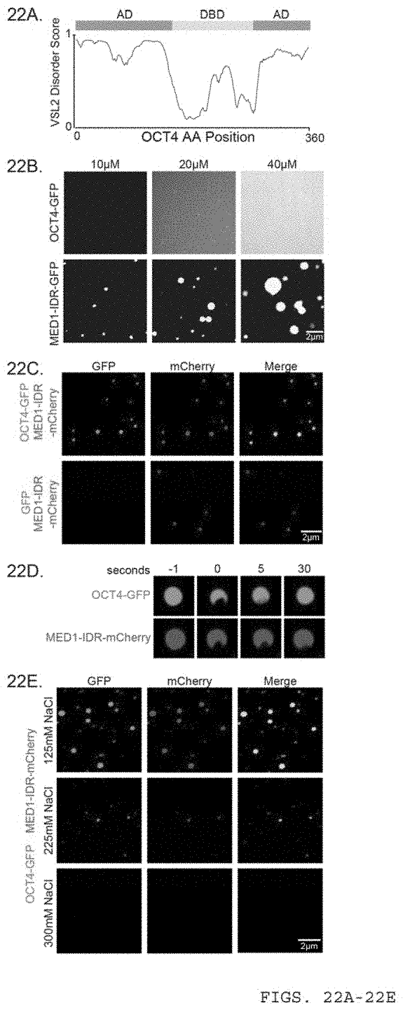

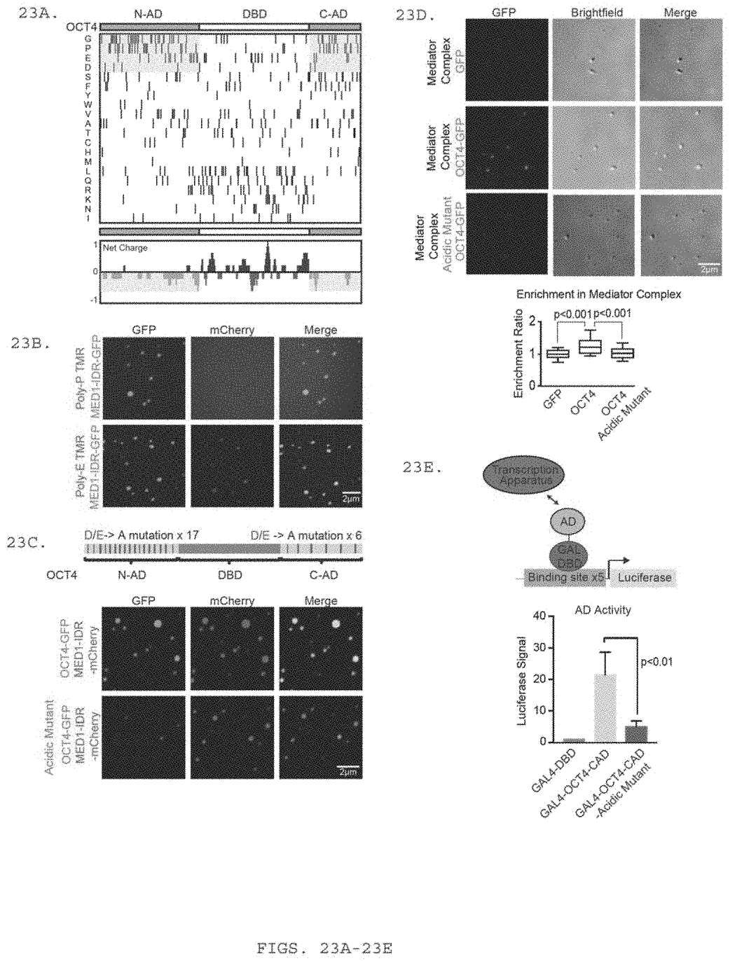

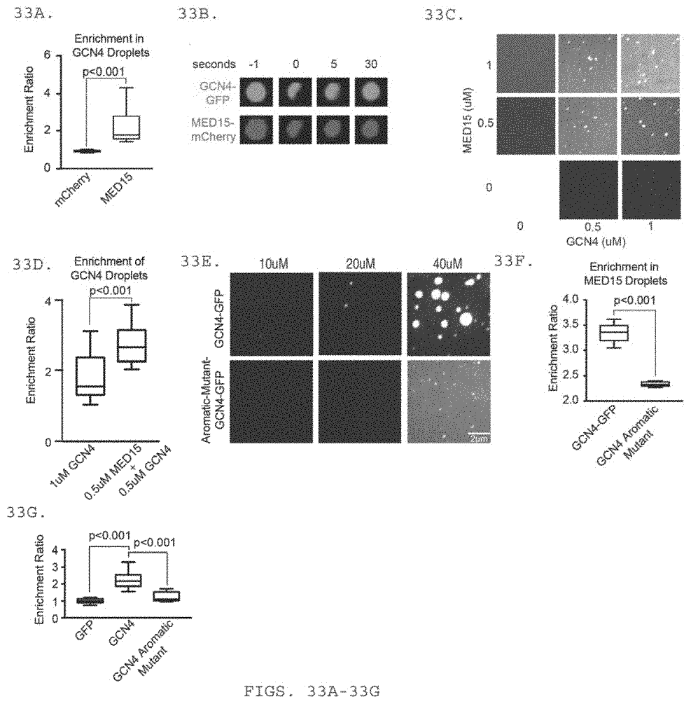

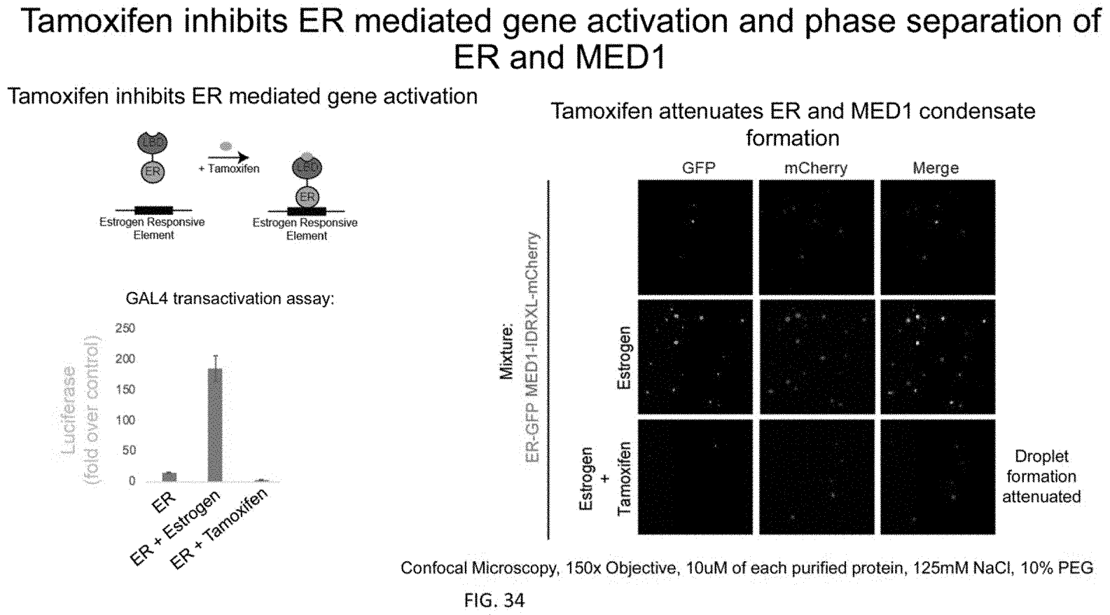

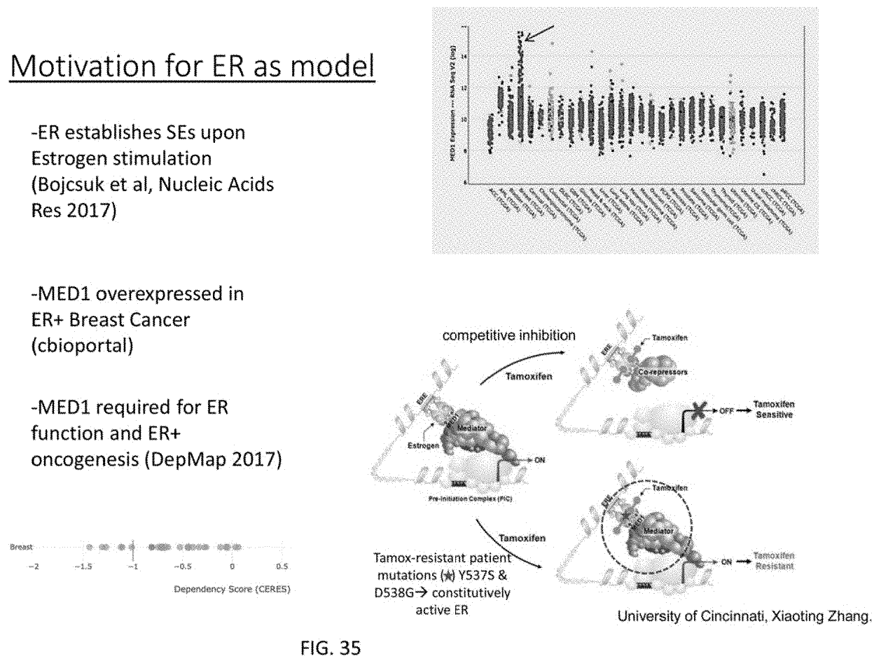

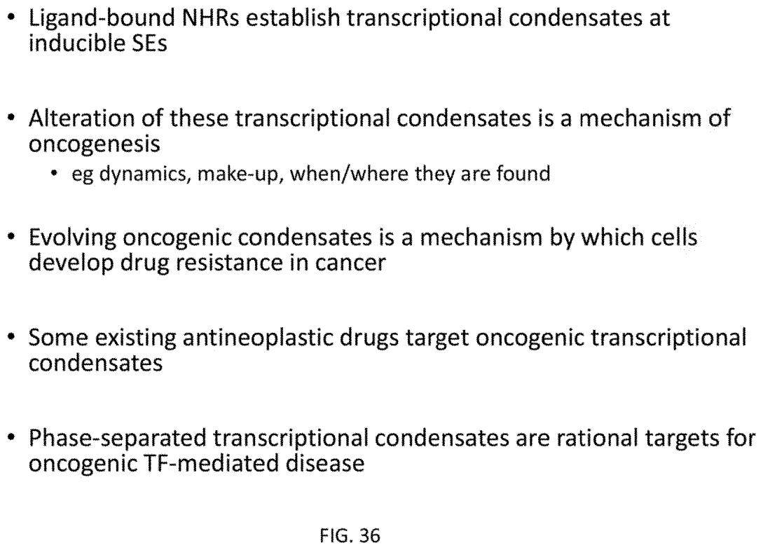

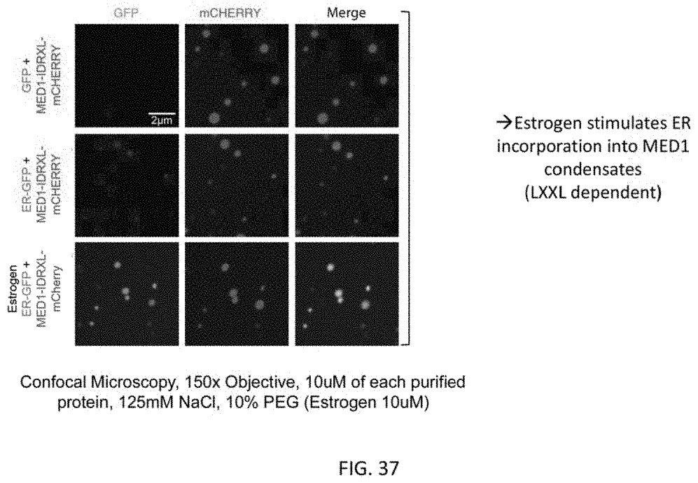

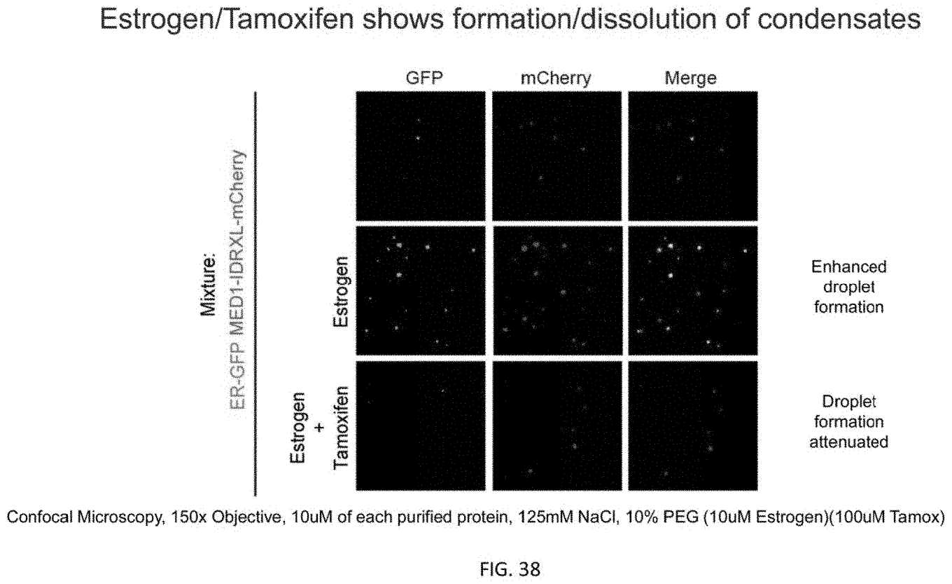

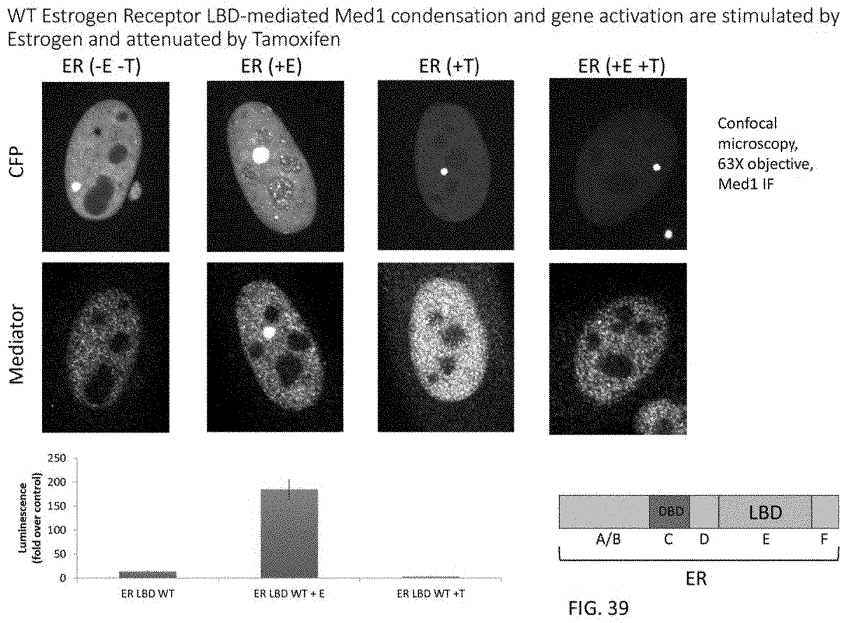

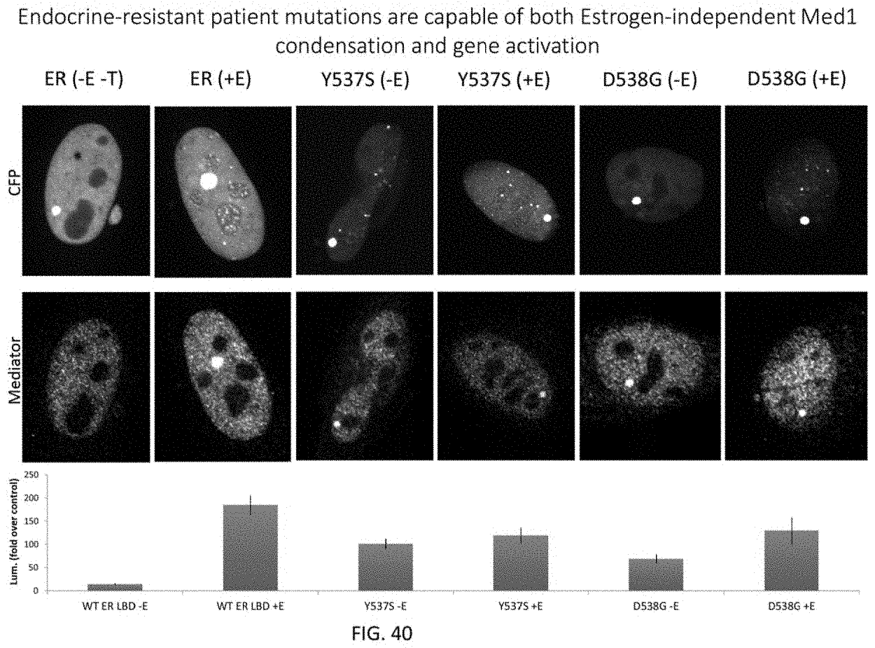

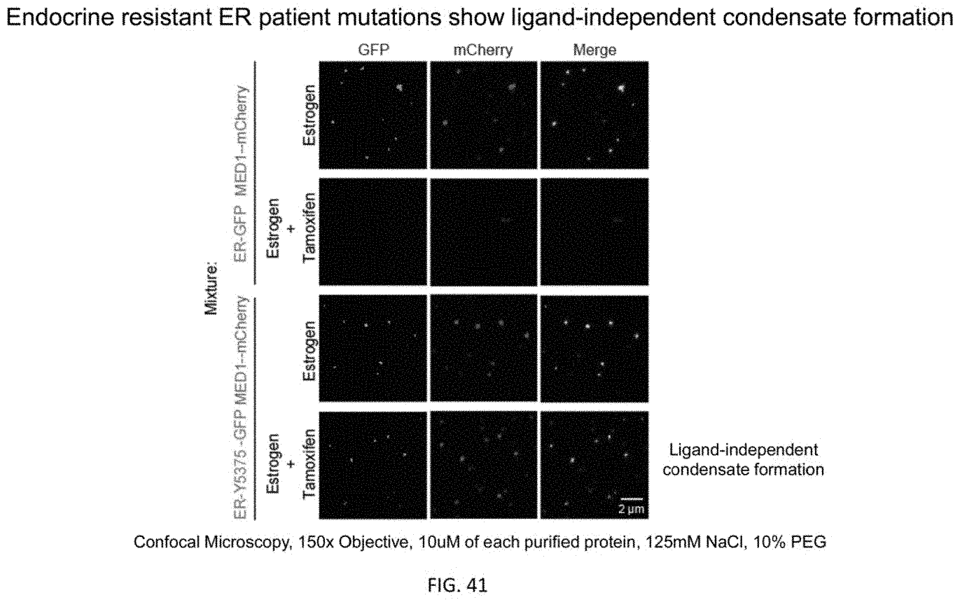

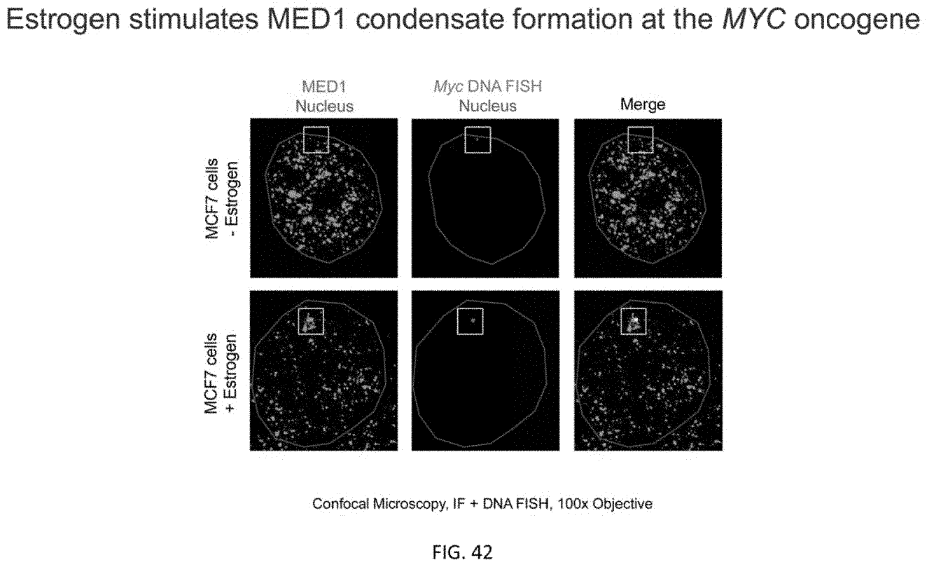

We next investigated whether specific OCT4 amino acid residues are required for the formation of OCT4-MED1-IDR phase-separated droplets, as multiple categories of amino acid interaction have been implicated in forming condensates. For example, serine residues are required for MED1 phase separation (Sabari et al., 2018). We asked whether amino acid enrichments in the OCT4 ADs might point to a mechanism for interaction. An analysis of amino acid frequency and charge bias showed that the OCT4 IDRs are enriched in proline and glycine, and have an overall acidic charge (FIG. 23A). ADs are known to be enriched in acidic amino acids and proline, and have historically been classified on this basis (Frietze and Farnham, 2011), but the mechanism by which these enrichments might cause gene activation is not known. We hypothesized that proline or acidic amino acids in the ADs might facilitate interaction with the phase-separated MED1-IDR droplet. To test this, we designed fluorescently labeled proline and glutamic acid decapeptides and investigated whether these peptides can be concentrated in MED1-IDR droplets. When added to droplet formation buffer alone, these peptides remained in solution (FIG. 30A). When mixed with MED1-IDR-GFP, however, proline peptides were not incorporated into MED1-IDR droplets, while the glutamic acid peptides were concentrated within (FIGS. 23B and 30B). These results show that peptides with acidic residues are amenable to incorporation within MED1 phase-separated droplets.

Based on these results, we deduced that an OCT4 protein lacking acidic amino acids in its ADs might be defective in its ability to phase separate with MED1-IDR. Such a dependence on acidic residues would be consistent with our observation that OCT4-MED1-IDR droplets are highly salt sensitive. To test this idea, we generated a mutant OCT4 in which all acidic residues in the ADs were replaced with alanine (thus changing 17 AAs in the N-terminal AD and 6 in the C-terminal AD) (FIG. 23C). When this GFP-fused OCT4 mutant was mixed with purified MED1-IDR, entry into droplets was highly attenuated (FIGS. 23C and 30C). To test if this effect was specific for acidic residues, we generated a mutant of OCT4 in which all the aromatic amino acids within the ADs were changed to alanine. We found that this mutant was still incorporated into MED1-IDR droplets (30C and 30D). These results indicate that the ability of OCT4 to phase separate with MED1-IDR is dependent on acidic residues in the OCT4 IDRs.

To ensure that these results were not specific to the MED1-IDR we explored whether purified Mediator complexes would form droplets in vitro and incorporate OCT4. The human Mediator complex was purified as previously described (Meyer et al., 2008) and then concentrated for use in the droplet formation assay (FIG. 30E). Because purified endogenous Mediator does not contain a fluorescent tag, we monitored droplet formation by differential interference contrast (DIC) microscopy and found it to form droplets alone at ˜200-400 nM (FIG. 23D). Consistent with the results for MED1-1DR droplets, OCT4 was incorporated within human Mediator complex droplets but incorporation of the OCT4 acidic mutant was attenuated. These results indicate that the MED1-IDR and the complete Mediator complex each exhibit phase-separating behaviors and suggest that they both incorporate OCT4 in a manner that is dependent on electrostatic interactions provided by acidic amino acids.

To test whether the OCT4 AD acidic mutations affect the ability of the factor to activate transcription in vivo, we utilized a GAL4 transactivation assay (FIG. 23E). In this system, ADs or their mutant counterparts are fused to the GAL4 DBD and expressed in cells carrying a luciferase reporter plasmid. We found that the wild-type OCT4-AD fused to the GAL4-DBD was able to activate transcription, while the acidic mutant lost this function (FIG. 23E). These results indicate that the acidic residues of the OCT4 ADs are necessary for both incorporation into MED1 phase-separated droplets in vitro and for gene activation in vivo.

Multiple TFs Phase Separate with Mediator Subunit Droplets

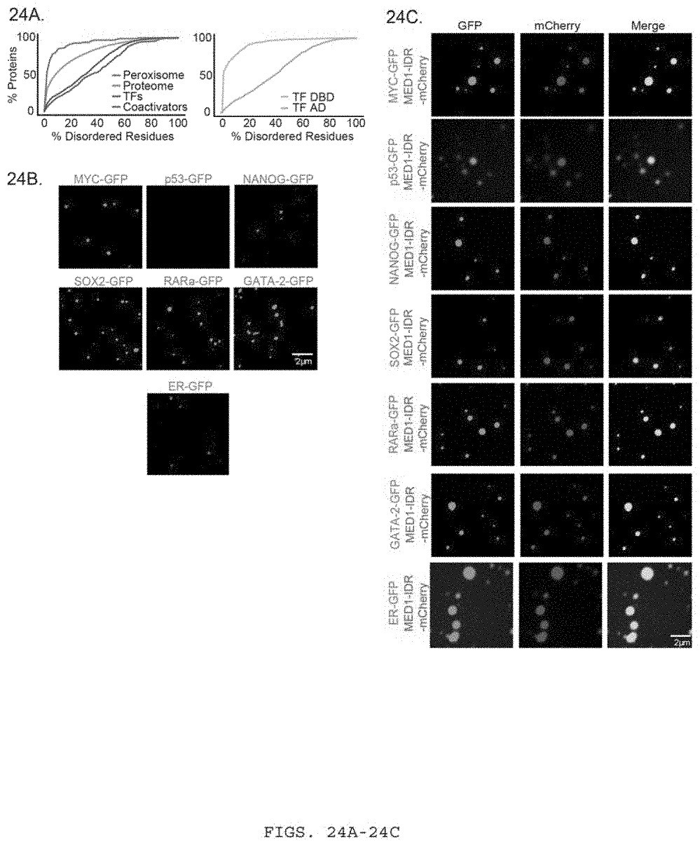

TFs with diverse types of ADs have been shown to interact with Mediator subunits, and MED1 is among the subunits that is most targeted by TFs (Table S3). An analysis of mammalian TFs confirmed that TFs and their putative ADs are enriched in IDRs, as previous analyses have shown (Liu et al., 2006; Staby et al., 2017b) (FIG. 24A). We reasoned that many different TFs might interact with the MED1-IDR to generate liquid droplets and therefore be incorporated into MED1 condensates. To assess whether diverse MED1-interacting transcription factors can phase separate with MED1, we prepared purified recombinant, mEGFP-tagged, full length MYC, p53, NANOG, SOX2, RARa, GATA2, and ER (Table S5). When added to droplet formation buffers, most TFs formed droplets alone (FIG. 24B). When added to droplet formation buffers with MED1-IDR, all 7 of these TFs concentrated into MED1-IDR droplets (FIG. 24C, 31A). We selected p53 droplets for FRAP analysis; they exhibited rapid and dynamic internal reorganization (FIG. 31B), supporting the notion that they are liquid condensates. These results indicate that TFs previously shown to interact with the MED1 subunit of Mediator can do so by forming phase-separated condensates with MED1.

Estrogen Stimulates Phase Separation of the Estrogen Receptor with MED1

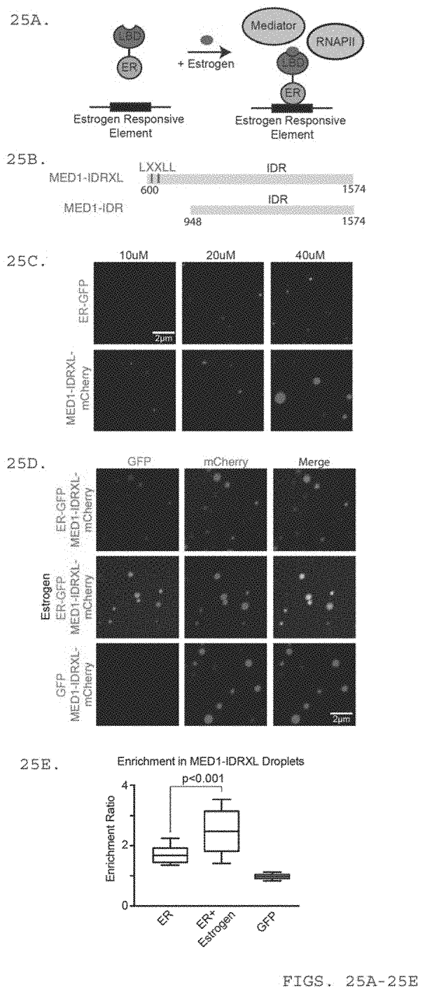

The estrogen receptor (ER) is a well-studied example of a ligand-dependent TF. ER consists of an N-terminal ligand-independent AD, a central DBD, and a C-terminal ligand-dependent AD (also called the ligand binding domain (LBD)) (FIG. 25A). Estrogen facilitates the interaction of ER with MED1 by binding the LBD of ER, which exposes a binding pocket for LXXLL motifs within the MED1-IDR (FIGS. 25A and 25B) (Manavathi et al., 2014). We noted that ER can form heterotypic droplets with the MED1-IDR recombinant protein used thus far in these studies (FIG. 24C), which lacks the LXXLL motifs. This led us to investigate whether ER-MED1 droplet formation is responsive to estrogen and whether this involves the MED1 LXXLL motifs.

We performed droplet formation assays using a MED1-IDR recombinant protein containing LXXLL motifs (MED1-IDRXL-mCherry) and found that, similar to MED1-IDR and complete Mediator, it had the ability to form droplets alone (FIG. 25C). We then tested the ability of ER to phase separate with MED1-IDRXL-mCherry and MED1-IDR-mCherry droplets. Some recombinant ER was incorporated and concentrated into MED1-IDRXL-mCherry droplets, but the addition of estrogen considerably enhanced heterotypic droplet formation (FIGS. 25D and 25E). In contrast, the addition of estrogen had little effect on droplet formation when the experiment was conducted with MED1-IDR-mCherry, which lacks the LXXLL motifs (FIG. 32). These results show that estrogen, which stimulates ER-mediated transcription in vivo, also stimulates incorporation of ER into MED1-IDR droplets in vitro. Thus, OCT4 and ER both require the same amino acids/ligands for both phase separation and activation. Furthermore, since the LBD is a structured domain that undergoes a conformation shift upon estrogen binding to interact with MED1, it appears that structured interactions may contribute to transcriptional condensate formation.

GCN4 and MED15 Phase Separation is Dependent on Residues Required for Activation

Among the best studied TF-coactivator systems is the yeast TF GCN4 and its interaction with the MED15 subunit of Mediator (Brzovic et al., 2011; Herbig et al., 2010; Jedidi et al., 2010). The GCN4 AD has been dissected genetically, the amino acids that contribute to activation have been identified (Drysdale et al., 1995; Staller et al., 2018), and recent studies have shown that the GCN4 AD interacts with MED15 in multiple orientations and conformations to form a “fuzzy complex” (Tuttle et al., 2018). Weak interactions that form fuzzy complexes have features of the IDR-IDR interactions that are thought to produce phase-separated condensates.

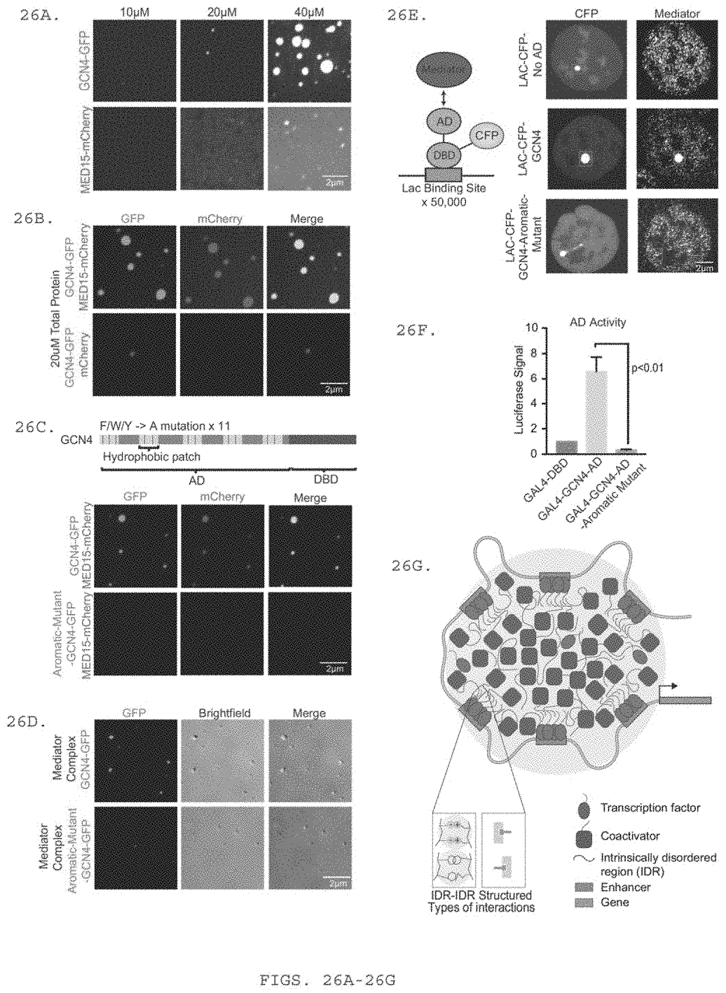

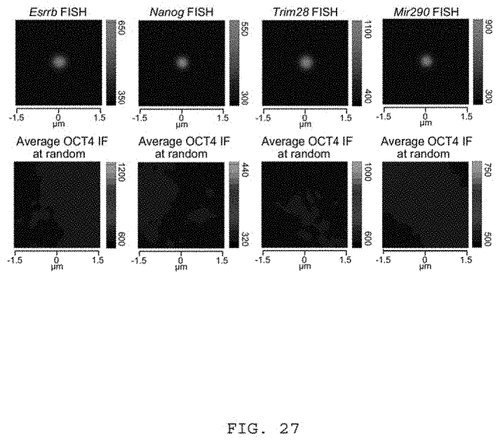

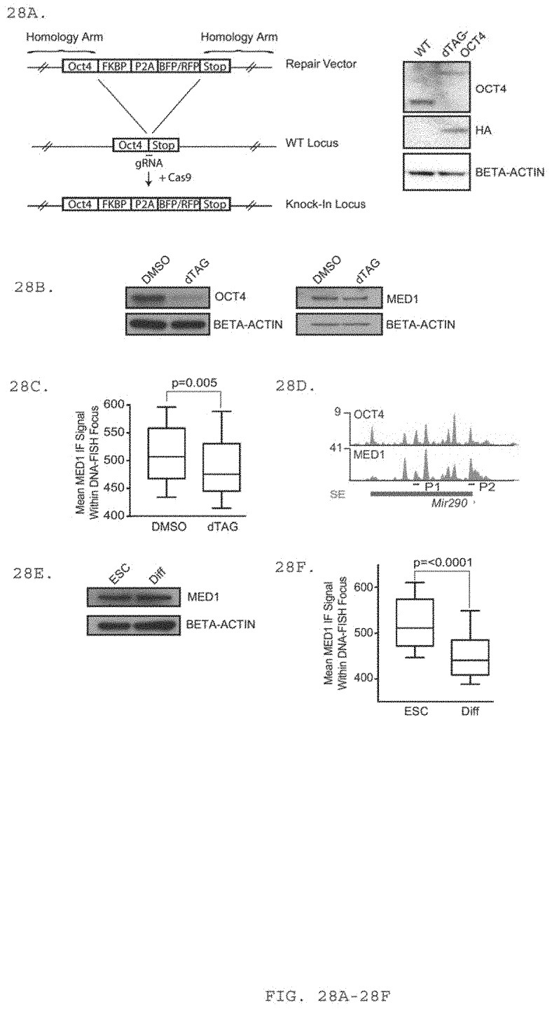

To test whether GCN4 and MED15 can form phase-separated droplets, we purified recombinant yeast GCN4-GFP and the N-terminal portion of yeast MED15-mCherry containing residues 6-651 (hereafter called MED15), which are responsible for the interaction with GCN4. When added separately to droplet formation buffer, GCN4 formed micron-sized droplets only at quite high concentrations (40 uM), and MED15 formed only small droplets at this high concentration (FIG. 26A). When mixed together, however, the GCN4 and MED15 recombinant proteins formed double-positive, micron-sized, spherical droplets at lower concentrations (FIG. 26B, 33A). These GCN4-MED15 droplets exhibited rapid FRAP kinetics (FIG. 33B), consistent with liquid-like behavior. We generated a phase diagram of these two proteins, and found that they formed droplets together at low concentration (FIGS. 33C and 33D). This suggests that interaction between the two is required for phase separation at low concentration.

The ability of GCN4 to interact with MED15 and activate gene expression has been attributed to specific hydrophobic patches and aromatic residues in the GCN4 AD (Drysdale et al., 1995; Staller et al., 2018; Tuttle et al., 2018). We created a mutant of GCN4 in which the 11 aromatic residues contained in these hydrophobic patches were changed to alanine (FIG. 26C). When added to droplet formation buffers, the ability of the mutant protein to form droplets alone was attenuated (FIG. 33E). Next, we tested whether droplet formation with MED15 was affected; indeed, the mutated protein has a compromised ability to form droplets with MED15 (FIGS. 26C and 33F). Similar results were obtained when GCN4 and the aromatic mutant of GCN4 was added to droplet formation buffers with the complete Mediator complex; while GCN4 was incorporated into Mediator droplets, the incorporation of the GCN4 mutant into Mediator droplets was attenuated (FIGS. 26D and 33G). These results demonstrate that multivalent, weak interactions between the AD of GCN4 and MED15 promote phase separation into liquid-like droplets.

The ADs of yeast TFs can function in mammalian cells and can do so by interacting with human Mediator (Oliviero et al., 1992). To investigate whether the aromatic mutant of GCN4 AD is impaired in its ability to recruit Mediator in vivo, the GCN4 AD and the GCN4 mutant AD were tethered to a Lac array in U2OS cells (FIG. 26E) (Janicki et al., 2004). While the tethered GCN4 AD caused robust Mediator recruitment, the GCN4 aromatic mutant did not (FIG. 26E). We used the GAL4 transactivation assay described previously to confirm that the GCN4 AD was capable of transcriptional activation in vivo, whereas the GCN4 aromatic mutant had lost that property (FIG. 26F). These results provide further support for the idea that TF AD amino acids that are essential for phase separation with Mediator are required for gene activation.

Discussion

The results described here support a model whereby TFs interact with Mediator and activate genes by the capacity of their ADs to form phase-separated condensates with this coactivator. For both the mammalian ESC pluripotency TF OCT4 and the yeast TF GCN4, we found that the AD amino acids required for phase separation with Mediator condensates were also required for gene activation in vivo. For the estrogen receptor, we found that estrogen stimulates the formation of phase-separated ER-MED1 droplets. ADs and coactivators generally consist of low-complexity amino acid sequences that have been classified as IDRs, and IDR-IDR interactions have been implicated in facilitating the formation of phase-separated condensates. We propose that IDR-mediated phase separation with Mediator is a general mechanism by which TF ADs effect gene expression, and provide evidence that this occurs in vivo at SEs. We suggest that the ability to phase separate with Mediator, which would employ the features of high valency and low affinity characteristic of liquid-liquid phase-separated condensates, operates alongside an ability of some TFs to form high affinity interactions with Mediator (FIG. 26G) (Taatjes, 2017).

The model that TF ADs function by forming phase-separated condensates with coactivators explains several observations that are difficult to reconcile with classical lock-and-key models of protein-protein interaction. The mammalian genome encodes many hundreds of TFs with diverse ADs that must interact with a very small number of coactivators (Allen and Taatjes, 2015; Arany et al., 1995; Avantaggiati et al., 1996; Dai and Markham, 2001; Eckner et al., 1996; Gelman et al., 1999; Green, 2005; Liu et al., 2009; Merika et al., 1998; Oliner et al., 1996; Yin and Wang, 2014; Yuan et al., 1996), and ADs that share little sequence homology are functionally interchangeable among TFs (Godowski et al., 1988; Hope and Struhl, 1986; Jin et al., 2016; Lech et al., 1988; Ransone et al., 1990; Sadowski et al., 1988; Struhl, 1988; Tora et al., 1989). The common feature of ADs—the possession of low-complexity IDRs—is also a feature that is pronounced in coactivators. The model of coactivator interaction and gene activation by phase-separated condensate formation thus more readily explains how many hundreds of mammalian TFs interact with these coactivators.

Previous studies have provided important insights that prompted us to investigate the possibility that TF ADs function by forming phase-separated condensates. TF ADs have been classified by their amino acid profile as acidic, proline-rich, serine/threonine-rich, glutamine-rich, or by their hypothetical shape as acid blobs, negative noodles, or peptide lassos (Sigler, 1988). Many of these features have been described for IDRs that are capable of forming phase-separated condensates (Babu, 2016; Darling et al., 2018; Das et al., 2015; Dunker et al., 2015; Habchi et al., 2014; van der Lee et al., 2014; Oldfield and Dunker, 2014; Uversky, 2017; Wright and Dyson, 2015). Evidence that the GCN4 AD interacts with MED15 in multiple orientations and conformations to form a “fuzzy complex” (Tuttle et al., 2018) is consistent with the notion of dynamic low-affinity interactions characteristic of phase-separated condensates Likewise, the low complexity domains of the FET (FUS/EWS/TAF15) RNA-binding proteins (Andersson et al., 2008) can form phase-separated hydrogels and interact with the RNA polymerase II C-terminal domain (CTD) in a CTD phosphorylation-dependent manner (Kwon et al., 2013); this may explain the mechanism by which RNA polymerase II is recruited to active genes in its unphosphorylated state and released for elongation following phosphorylation of the CTD.

The model we describe here for TF AD function may explain the function of a class of heretofore poorly understood fusion oncoproteins. Many malignancies bear fusion-protein translocations involving portions of TFs (Bradner et al., 200; Kim et al., 2017; Latysheva et al., 2016). These abnormal gene products often fuse a DNA- or chromatin-binding domain to a wide array of partners, many of which are IDRs. For example, MLL may be fused to 80 different partner genes in AML (Winters and Bernt, 2017), the EWS-FLI rearrangement in Ewing's Sarcoma causes malignant transformation by recruitment of a disordered domain to oncogenes (Boulay et al., 2017; Chong et al., 2017), and the disordered phase-separating protein FUS is found fused to a DBD in certain sarcomas (Crozat et al., 1993; Patel et al, 2015). Phase separation provides a mechanism by which such gene products result in aberrant gene expression programs; by recruiting a disordered protein to the chromatin, diverse coactivators may form phase-separated condensates to drive oncogene expression. Understanding the interactions which compose these aberrant transcriptional condensates, their structures, and behaviors may open new therapeutic avenues.

### REFERENCES

Alberti, S. (2017). The wisdom of crowds: regulating cell function through condensed states of living matter. J. Cell Sci. 130, 2789-2796.
Allen, B. L., and Taatjes, D. J. (2015). The Mediator complex: a central integrator of transcription. Nat. Rev. Mol. Cell Biol. 16, 155-166.
Andersson, M. K., Ståhlberg, A., Arvidsson, Y., Olofsson, A., Semb, H., Stenman, G., Nilsson, O., and Åman, P. (2008). The multifunctional FUS, EWS and TAF15 proto-oncoproteins show cell type-specific expression patterns and involvement in cell spreading and stress response. BMC Cell Biol. 9, 37.
Apostolou, E., Ferrari, F., Walsh, R. M., Bar-Nur, O., Stadtfeld, M., Cheloufi, S., Stuart, H. T., Polo, J. M., Ohsumi, T. K., Borowsky, M. L., et al. (2013). Genome-wide chromatin interactions of the Nanog locus in pluripotency, differentiation, and reprogramming. Cell Stem Cell 12, 699-712.
Arany, Z., Newsome, D., Oldread, E., Livingston, D. M., and Eckner, R. (1995). A family of transcriptional adaptor proteins targeted by the E1A oncoprotein. Nature 374, 81-84.
Avantaggiati, M. L., Carbone, M., Graessmann, A., Nakatani, Y., Howard, B., and Levine, A. S. (1996). The SV40 large T antigen and adenovirus Ela oncoproteins interact with distinct isoforms of the transcriptional co-activator, p300. EMBO J. 15, 2236-2248.
Babu, M. M. (2016). The contribution of intrinsically disordered regions to protein function, cellular complexity, and human disease. Biochem. Soc. Trans. 44, 1185-1200.
Banani, S. F., Lee, H. O., Hyman, A. A., and Rosen, M. K. (2017). Biomolecular condensates: organizers of cellular biochemistry. Nat. Rev. Mol. Cell Biol. 18, 285-298.
Boulay, G., Sandoval, G. J., Riggi, N., Iyer, S., Buisson, R., Naigles, B., Awad, M. E., Rengarajan, S., Volorio, A., McBride, M. J., et al. (2017). Cancer-Specific Retargeting of BAF Complexes by a Prion-like Domain. Cell 171, 163-178.e19.
Bradner, J. E., Hnisz, D., and Young, R. A. (2017). Transcriptional Addiction in Cancer.
Brehm, A., Ohbo, K., and Schöler, H. (1997). The carboxy-terminal transactivation domain of Oct-4 acquires cell specificity through the POU domain. Mol. Cell. Biol. 17, 154-162.
Brent, R., and Ptashne, M. (1985). A eukaryotic transcriptional activator bearing the DNA specificity of a prokaryotic repressor. Cell 43, 729-736.
Brzovic, P. S., Heikaus, C. C., Kisselev, L., Vernon, R., Herbig, E., Pacheco, D., Warfield, L., Littlefield, P., Baker, D., Klevit, R. E., et al. (2011). The acidic transcription activator Gcn4 binds the mediator subunit Gal11/Med15 using a simple protein interface forming a fuzzy complex. Mol. Cell 44, 942-953.
Burke, K. A., Janke, A. M., Rhine, C. L., and Fawzi, N. L. (2015). Residue-by-Residue View of In Vitro FUS Granules that Bind the C-Terminal Domain of RNA Polymerase II. Mol. Cell 60, 231-241.
Chong, S., Dugast-darzacq, C., Liu, Z., Dong, P., and Dailey, G. M. (2017). Dynamic and Selective Low-Complexity Domain Interactions Revealed by Live-Cell Single-Molecule Imaging. Bioarxiv.
Crozat, A., Åman, P., Mandahl, N., and Ron, D. (1993). Fusion of CHOP to a novel RNA-binding protein in human myxoid liposarcoma. Nature 363, 640-644.
Dai, Y. S., and Markham, B. E. (2001). p300 Functions as a coactivator of transcription factor GATA-4. J. Biol. Chem. 276, 37178-37185.
Darling, A. L., Liu, Y., Oldfield, C. J., and Uversky, V. N. (2018). Intrinsically Disordered Proteome of Human Membrane-Less Organelles. Proteomics 18, 1700193.
Das, R. K., Ruff, K. M., and Pappu, R. V (2015). Relating sequence encoded information to form and function of intrinsically disordered proteins. Curr. Opin. Struct. Biol. 32, 102-112.
Drysdale, C. M., Dueñas, E., Jackson, B. M., Reusser, U., Braus, G. H., and Hinnebusch, A. G. (1995). The transcriptional activator GCN4 contains multiple activation domains that are critically dependent on hydrophobic amino acids. Mol. Cell. Biol. 15, 1220-1233.
Dunker, A. K., Bondos, S. E., Huang, F., and Oldfield, C. J. (2015). Intrinsically disordered proteins and multicellular organisms. Semin. Cell Dev. Biol. 37, 44-55.
Eckner, R., Yao, T. P., Oldread, E., and Livingston, D. M. (1996). Interaction and functional collaboration of p300/CBP and bHLH proteins in muscle and B-cell differentiation. Genes Dev. 10, 2478-2490.
Frietze, S., and Farnham, P. J. (2011). Transcription factor effector domains. Subcell. Biochem. 52, 261-277.
Fulton, D. L., Sundararajan, S., Badis, G., Hughes, T. R., Wasserman, W. W., Roach, J. C., and Sladek, R. (2009). TFCat: the curated catalog of mouse and human transcription factors. Genome Biol. 10, R29.
Gelman, L., Zhou, G., Fajas, L., Raspé, E., Fruchart, J. C., and Auwerx, J. (1999). p300 interacts with the N- and C-terminal part of PPARgamma2 in a ligand-independent and -dependent manner, respectively. J. Biol. Chem. 274, 7681-7688.
Godowski, P. J., Picard, D., and Yamamoto, K. R. (1988). Signal transduction and transcriptional regulation by glucocorticoid receptor-LexA fusion proteins. Science 241, 812-816.
Green, M. R. (2005). Eukaryotic Transcription Activation: Right on Target. Mol. Cell 18, 399-402.
Habchi, J., Tompa, P., Longhi, S., and Uversky, V. N. (2014). Introducing Protein Intrinsic Disorder. Chem. Rev. 114, 6561-6588.
Herbig, E., Warfield, L., Fish, L., Fishburn, J., Knutson, B. A., Moorefield, B., Pacheco, D., and Hahn, S. (2010). Mechanism of Mediator Recruitment by Tandem Gcn4 Activation Domains and Three Gal11 Activator-Binding Domains. Mol. Cell. Biol. 30, 2376-2390.
Hnisz, D., Shrinivas, K., Young, R. A., Chakraborty, A. K., and Sharp, P. A. (2017). Perspective A Phase Separation Model for Transcriptional Control. Cell 169, 13-23.
Holehouse, A. S., Das, R. K., Ahad, J. N., Richardson, M. O. G., and Pappu, R. V (2017). CIDER: Resources to Analyze Sequence-Ensemble Relationships of Intrinsically Disordered Proteins. Biophys. J. 112, 16-21.
Hope, I. A., and Struhl, K. (1986). Functional dissection of a eukaryotic transcriptional activator protein, GCN4 of yeast. Cell 46, 885-894.
Hume, M. A., Barrera, L. A., Gisselbrecht, S. S., and Bulyk, M. L. (2015). UniPROBE, update 2015: new tools and content for the online database of protein-binding microarray data on protein-DNA interactions. Nucleic Acids Res. 43, D117-D122.
Hyman, A. A., Weber, C. A., and Jülicher, F. (2014). Liquid-Liquid Phase Separation in Biology. Annu. Rev. Cell Dev. Biol. 30, 39-58.
Janicki, S. M., Tsukamoto, T., Salghetti, S. E., Tansey, W. P., Sachidanandam, R., Prasanth, K. V, Ried, T., Shav-Tal, Y., Bertrand, E., Singer, R. H., et al. (2004). From silencing to gene expression: real-time analysis in single cells. Cell 116, 683-698.
Jedidi, I., Zhang, F., Qiu, H., Stahl, S. J., Palmer, I., Kaufman, J. D., Nadaud, P. S., Mukherjee, S., Wingfield, P. T., Jaroniec, C. P., et al. (2010). Activator Gcn4 employs multiple segments of Med15/Gal11, including the KIX domain, to recruit mediator to target genes in vivo. J. Biol. Chem. 285, 2438-2455.
Jin, W., Wang, L., Zhu, F., Tan, W., Lin, W., Chen, D., Sun, Q., and Xia, Z. (2016). Critical POU domain residues confer Oct4 uniqueness in somatic cell reprogramming. Sci. Rep. 6, 20818.
Jolma, A., Yan, J., Whitington, T., Toivonen, J., Nitta, K. R., Rastas, P., Morgunova, E., Enge, M., Taipale, M., Wei, G., et al. (2013). DNA-Binding Specificities of Human Transcription Factors. Cell 152, 327-339.
Juven-Gershon, T., and Kadonaga, J. T. (2010). Regulation of gene expression via the core promoter and the basal transcriptional machinery. Dev. Biol. 339, 225-229.
Keegan, L., Gill, G., and Ptashne, M. (1986). Separation of DNA binding from the transcription-activating function of a eukaryotic regulatory protein. Science 231, 699-704.
Khan, A., Fornes, O., Stigliani, A., Gheorghe, M., Castro-Mondragon, J. A., van der Lee, R., Bessy, A., Chèneby, J., Kulkarni, S. R., Tan, G., et al. (2018). JASPAR 2018: update of the open-access database of transcription factor binding profiles and its web framework. Nucleic Acids Res. 46, D260-D266.
Kim, P., Ballester, L. Y., and Zhao, Z. (2017). Domain retention in transcription factor fusion genes and its biological and clinical implications: a pan-cancer study. Oncotarget 8, 110103-110117.
Latysheva, N. S., Oates, M. E., Maddox, L., Buljan, M., Weatheritt, R. J., Madan Babu, M., Flock, T., and Gough, J. (2016). Molecular Principles of Gene Fusion Mediated Rewiring of Protein Interaction Networks in Cancer. Mol. Cell 63, 579-592.
Lech, K., Anderson, K., and Brent, R. (1988). DNA-bound Fos proteins activate transcription in yeast. Cell 52, 179-184.
van der Lee, R., Buljan, M., Lang, B., Weatheritt, R. J., Daughdrill, G. W., Dunker, A. K., Fuxreiter, M., Gough, J., Gsponer, J., Jones, D. T., et al. (2014). Classification of intrinsically disordered regions and proteins. Chem. Rev. 114, 6589-6631.
Lin, Y., Protter, D. S. W., Rosen, M. K., and Parker, R. (2015). Formation and Maturation of Phase-Separated Liquid Droplets by RNA-Binding Proteins. Mol. Cell 60, 208-219.
Liu, J., Perumal, N. B., Oldfield, C. J., Su, E. W., Uversky, V. N., and Dunker, A. K. (2006). Intrinsic Disorder in Transcription Factors^{†}. Biochemistry 45, 6873-6888.
Liu, W.-L., Coleman, R. A., Ma, E., Grob, P., Yang, J. L., Zhang, Y., Dailey, G., Nogales, E., and Tjian, R. (2009). Structures of three distinct activator-TFIID complexes. Genes Dev. 23, 1510-1521.
Malik, S., and Roeder, R. G. (2010). The metazoan Mediator co-activator complex as an integrative hub for transcriptional regulation. Nat. Rev. Genet. 11, 761-772.
Manavathi, B., Samanthapudi, V. S. K., and Gajulapalli, V. N. R. (2014). Estrogen receptor coregulators and pioneer factors: the orchestrators of mammary gland cell fate and development. Front. Cell Dev. Biol. 2, 34.
Merika, M., Williams, A. J., Chen, G., Collins, T., and Thanos, D. (1998). Recruitment of CBP/p300 by the IFN beta enhanceosome is required for synergistic activation of transcription. Mol. Cell 1, 277-287.
Meyer, K. D., Donner, A. J., Knuesel, M. T., York, A. G., Espinosa, J. M., and Taatjes, and D. J. (2008). Cooperative activity of cdk8 and GCNSL within Mediator directs tandem phosphoacetylation of histone H3. EMBO J. 27, 1447-1457.
Mitchell, P. J., and Tjian, R. (1989). Transcriptional regulation in mammalian cells by sequence-specific DNA binding proteins. Science 245, 371-378.
Nabet, B., Roberts, J. M., Buckley, D. L., Paulk, J., Dastjerdi, S., Yang, A., Leggett, A. L., Erb, M. A., Lawlor, M. A., Souza, A., et al. (2018). The dTAG system for immediate and target-specific protein degradation. Nat. Chem. Biol. 14, 431-441.
Nott, T. J., Petsalaki, E., Farber, P., Jervis, D., Fussner, E., Plochowietz, A., Craggs, T. D., Bazett-Jones, D. P., Pawson, T., Forman-Kay, J. D., et al. (2015). Phase Transition of a Disordered Nuage Protein Generates Environmentally Responsive Membraneless Organelles. Mol. Cell 57, 936-947.
Oates, M. E., Romero, P., Ishida, T., Ghalwash, M., Mizianty, M. J., Xue, B., Dosztányi, Z., Uversky, V. N., Obradovic, Z., Kurgan, L., et al. (2013). D²P²: database of disordered protein predictions. Nucleic Acids Res. 41, D508-16.
Oldfield, C. J., and Dunker, A. K. (2014). Intrinsically Disordered Proteins and Intrinsically Disordered Protein Regions. Annu. Rev. Biochem. 83, 553-584.
Oliner, J. D., Andresen, J. M., Hansen, S. K., Zhou, S., and Tjian, R. (1996). SREBP transcriptional activity is mediated through an interaction with the CREB-binding protein. Genes Dev. 10, 2903-2911.
Oliviero, S., Robinson, G. S., Struhl, K., and Spiegelman, B. M. Yeast GCN4 as a probe for oncogenesis by AP-1. transcription factors: transcnpuonal activation through AP-1 sites is not sufficient for cellular transformation.
Panne, D., Maniatis, T., and Harrison, S. C. (2007). An Atomic Model of the Interferon-β Enhanceosome. Cell 129, 1111-1123.
Patel, A., Lee, H. O., Jawerth, L., Maharana, S., Jahnel, M., Hein, M. Y., Stoynov, S., Mahamid, J., Saha, S., Franzmann, T. M., et al. (2015). A Liquid-to-Solid Phase Transition of the ALS Protein FUS Accelerated by Disease Mutation. Cell 162, 1066-1077.
Plaschka, C., Nozawa, K., and Cramer, P. (2016). Mediator Architecture and RNA Polymerase II Interaction. J. Mol. Biol. 428, 2569-2574.
Ransone, L. J., Wamsley, P., Morley, K. L., and Verma, I. M. (1990). Domain swapping reveals the modular nature of Fos, Jun, and CREB proteins. Mol. Cell. Biol. 10, 4565-4573.
Reiter, F., Wienerroither, S., and Stark, A. (2017). Combinatorial function of transcription factors and cofactors. Curr. Opin. Genet. Dev. 43, 73-81.
Roberts, S. G. (2000). Mechanisms of action of transcription activation and repression domains. Cell. Mol. Life Sci. 57, 1149-1160.
Sabari, B., Dall'Agnese, A., Boija, A., Klein, I. A., Coffey, E. L., Shrinivas, K., Abraham, B. J., Hannett, N. M., Zamudio, A. V., Manteiga, J., et al. (2018). Coactivator condensation at super-enhancers links phase separation and gene control. Science (80-).
Sadowski, I., Ma, J., Triezenberg, S., and Ptashne, M. (1988). GAL4-VP16 is an unusually potent transcriptional activator. Nature 335, 563-564.
Saint-andré, V., Federation, A. J., Lin, C. Y., Abraham, B. J., Reddy, J., Lee, T. I., Bradner, J. E., and Young, R. A. Models of human core transcriptional regulatory circuitries. 385-396.
Shin, Y., and Brangwynne, C. P. (2017). Liquid phase condensation in cell physiology and disease. Science (80-). 357, eaaf4382.
Sigler, P. B. (1988). Acid blobs and negative noodles. Nature 333, 210-212.
Soutourina, J. (2017). Transcription regulation by the Mediator complex. Nat. Rev. Mol. Cell Biol. 19, 262-274.
Staby, L., O'Shea, C., Willemoës, M., Theisen, F., Kragelund, B. B., and Shiver, K. (2017a). Eukaryotic transcription factors: paradigms of protein intrinsic disorder. Biochem. J. 474, 2509-2532.
Staby, L., O'Shea, C., Willemoës, M., Theisen, F., Kragelund, B. B., and Shiver, K. (2017b). Eukaryotic transcription factors: paradigms of protein intrinsic disorder. Biochem. J. 474, 2509-2532.
Staller, M. V., Holehouse, A. S., Swain-Lenz, D., Das, R. K., Pappu, R. V., and Cohen, B. A. (2018). A High-Throughput Mutational Scan of an Intrinsically Disordered Acidic Transcriptional Activation Domain. Cell Syst. 6, 444-455.e6.
Struhl, K. (1988). The JUN oncoprotein, a vertebrate transcription factor, activates transcription in yeast. Nature 332, 649-650.
Taatjes, D. J. (2010). The human Mediator complex: a versatile, genome-wide regulator of transcription. Trends Biochem. Sci. 35, 315-322.
Taatjes, D. J. (2017). Transcription Factor-Mediator Interfaces: Multiple and Multi-Valent. J. Mol. Biol. 429, 2996-2998.
Tompa, P., and Fuxreiter, M. (2008). Fuzzy complexes: polymorphism and structural disorder in protein-protein interactions. Trends Biochem. Sci. 33, 2-8.
Tora, L., White, J., Brou, C., Tasset, D., Webster, N., Scheer, E., and Chambon, P. (1989). The human estrogen receptor has two independent nonacidic transcriptional activation functions. Cell 59, 477-487.
Triezenberg, S. J. (1995). Structure and function of transcriptional activation domains. Curr. Opin. Genet. Dev. 5, 190-196.
Tuttle, L. M., Pacheco, D., Warfield, L., Luo, J., Ranish, J., Hahn, S., and Klevit, R. E. (2018). Gcn4-Mediator Specificity Is Mediated by a Large and Dynamic Fuzzy Protein-Protein Complex. Cell Rep. 22, 3251-3264.
Uversky, V. N. (2017). Intrinsically disordered proteins in overcrowded milieu: Membrane-less organelles, phase separation, and intrinsic disorder. Curr. Opin. Struct. Biol. 44, 18-30.
Vaquerizas, J. M., Kummerfeld, S. K., Teichmann, S. A., and Luscombe, N. M. (2009). A census of human transcription factors: function, expression and evolution. Nat. Rev. Genet. 10, 252-263.
Warfield, L., Tuttle, L. M., Pacheco, D., Klevit, R. E., and Hahn, S. (2014). A sequence-specific transcription activator motif and powerful synthetic variants that bind Mediator using a fuzzy protein interface. Proc. Natl. Acad. Sci. 111, E3506-E3513.
Weintraub, A. S., Li, C. H., Zamudio, A. V., Sigova, A. A., Hannett, N. M., Day, D. S., Abraham, B. J., Cohen, M. A., Nabet, B., Buckley, D. L., et al. (2017). YY1 Is a Structural Regulator of Enhancer-Promoter Loops. Cell 171, 1573-1588.e28.
Wheeler, R. J., and Hyman, A. A. (2018). Controlling compartmentalization by non-membrane-bound organelles. Philos. Trans. R. Soc. Lond. B. Biol. Sci. 373.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Winters, A. C., and Bernt, K. M. (2017). MLL-Rearranged Leukemias—An Update on Science and Clinical Approaches. Front. Pediatr. 5, 4.
Wright, P. E., and Dyson, H. J. (2015). Intrinsically disordered proteins in cellular signalling and regulation. Nat. Rev. Mol. Cell Biol. 16, 18-29.
Yin, J., and Wang, G. (2014). The Mediator complex: a master coordinator of transcription and cell lineage development. Development 141, 977-987.
Yuan, W., Condorelli, G., Caruso, M., Felsani, A., and Giordano, A. (1996). Human p300 protein is a coactivator for the transcription factor MyoD. J. Biol. Chem. 271, 9009-9013.

TABLE S3
Table of reported transcription factor-mediator subunit
interactions.

| Mediator | | | | | | | | | |
|---|---|---|---|---|---|---|---|---|---|
| subunits | Transcription factors | | | | | | | | |
| MED1 | OCT3/4 [symbol] | SOX2 [symbol] | GATA2 [symbol] | MYC [symbol] | P53 [symbol] | PPARG [symbol] | RARA [symbol] | ER1 [symbol] | CEBPA [symbol] |
| | VDR [symbol] | RSRA [symbol] | GATA1 [symbol] | THRA/THRB [symbol] | HNF4 [symbol] | AHR [symbol] | AR [symbol] | SREBP1 [symbol] | NR113 [symbol] |
| | RORa [symbol] | GR [symbol] | FXR [symbol] | Ps-1 [symbol] | PPARA [symbol] | ER2 [symbol] | KLF4 [symbol] | | |
| MED12 | ER1 | SOX9 [symbol] | THRA [symbol] | GLI3 [symbol] | NANOG [symbol] | ER2 [symbol] | JUNB [symbol] | VDR [symbol] | SREBF [symbol] |
| | RTA [symbol] | B-CATENIN [symbol] | AICD [symbol] | | | | | | |
| MED14 | PPARG [symbol] | ER1 [symbol] | VDR [symbol] | HNF4 [symbol] | ER2 [symbol] | JUNB [symbol] | NR113 [symbol] | GATA1 [symbol] | SREBF1 [symbol] |
| | GR [symbol] | | | | | | | | |
| MED15 | P65 [symbol] | JUNB [symbol] | SREBF1 [symbol] | | | | | | |
| MED16 | THRA [symbol] | DIF [symbol] | VDR [symbol] | MYC [symbol] | JUNB [symbol] | | | | |
| MED17 | SREBF1 [symbol] | P65 [symbol] | FOS [symbol] | HNF4A [symbol] | DIF [symbol] | RXR [symbol] | JUNB [symbol] | NR113 [symbol] | HSF [symbol] |
| | VP16 [symbol] | | | | | | | | |
| MED19 | REST | | | | | | | | |
| MED21 | SREBF1 [symbol] | P53 [symbol] | VDR [symbol] | THRA/THRB [symbol] | | | | | |
| MED23 | SREBF1 [symbol] | ELF3 [symbol] | NR113 [symbol] | RB1 [symbol] | DIF [symbol] | HIF [symbol] | VDR [symbol] | CrEBP [symbol] | P65 [symbol] |
| | ELK1 [symbol] | H3F [symbol] | ESX [symbol] | | | | | | |
| MED25 | VP16 [symbol] | DIF [symbol] | HSF [symbol] | RARA [symbol] | HNF4 [symbol] | SOX9 [symbol] | | | |
| MED26 | SREBF1 [symbol] | | | | | | | | |
| MED29 | JUNB [symbol] | DSX [symbol] | | | | | | | |
| CDKB | THRA [symbol] | MYC [symbol] | | | | | | | |

Adopted from Borggrefe and Xue, 2011⁵⁷. [symbol]
indicates data missing or illegible when filed

### REFERENCES CITED IN TABLE

1. Apostolou, E. et al. Genome-wide chromatin interactions of the Nanog locus in pluripotency, differentiation, and reprogramming. Cell Stem Cell 12, 699-712 (2013).
2. Gordon, D. F. et al. MED220/thyroid receptor-associated protein 220 functions as a transcriptional coactivator with Pit-1 and GATA-2 on the thyrotropin-beta promoter in thyrotropes. Mol. Endocrinol. 20, 1073-89 (2006).
3. Liu, X., Vorontchikhina, M., Wang, Y.-L., Faiola, F. & Martinez, E. STAGA recruits Mediator to the MYC oncoprotein to stimulate transcription and cell proliferation. Mol. Cell. Biol. 28, 108-21 (2008).
4. Meyer, K. D., Lin, S., Bernecky, C., Gao, Y. & Taatjes, D. J. p53 activates transcription by directing structural shifts in Mediator. Nat. Struct. Mol. Biol. 17, 753-760 (2010).
5. Drané, P., Barel, M., Balbo, M. & Frade, R. Identification of RB18A, a 205 kDa new p53 regulatory protein which shares antigenic and functional properties with p53. Oncogene 15, 3013-3024 (1997).
6. Frade, R., Balbo, M. & Barel, M. RB18A, whose gene is localized on chromosome 17q12-q21.1, regulates in vivo p53 transactivating activity. Cancer Res. 60, 6585-9 (2000).
7. Ge, K. et al. Transcription coactivator TRAP220 is required for PPARγ2-stimulated adipogenesis. Nature 417, 563-567 (2002).
8. Yuan, C. X., Ito, M., Fondell, J. D., Fu, Z. Y. & Roeder, R. G. The TRAP220 component of a thyroid hormone receptor-associated protein (TRAP) coactivator complex interacts directly with nuclear receptors in a ligand-dependent fashion. Proc. Natl. Acad. Sci. U.S.A 95, 7939-44 (1998).
9. Zhu, X. G., McPhie, P., Lin, K. H. & Cheng, S. Y. The differential hormone-dependent transcriptional activation of thyroid hormone receptor isoforms is mediated by interplay of their domains. J. Biol. Chem. 272, 9048-54 (1997).
10. Kang, Y. K., Guermah, M., Yuan, C.-X. & Roeder, R. G. The TRAP/Mediator coactivator complex interacts directly with estrogen receptors and through the TRAP220 subunit and directly enhances estrogen receptor function in vitro. Proc. Natl. Acad. Sci. 99, 2642-2647 (2002).
11. Jiang, P. et al. Key roles for MED1 LxxLL motifs in pubertal mammary gland development and luminal-cell differentiation. Proc. Natl. Acad. Sci. U.S.A 107, 6765-70 (2010).
12. Burakov, D., Wong, C. W., Rachez, C., Cheskis, B. J. & Freedman, L. P. Functional interactions between the estrogen receptor and DRIP205, a subunit of the heteromeric DRIP coactivator complex. J. Biol. Chem. 275, 20928-34 (2000).
13. Li, H. et al. The Med1 Subunit of Transcriptional Mediator Plays a Central Role in Regulating CCAAT/Enhancer-binding Protein-β-driven Transcription in Response to Interferon-γ. J. Biol. Chem. 283, 13077-13086 (2008).
14. Rachez, C. et al. Ligand-dependent transcription activation by nuclear receptors requires the DRIP complex. Nature 398, 824-8 (1999).
15. Stumpf, M. et al. The mediator complex functions as a coactivator for GATA-1 in erythropoiesis via subunit Med1/TRAP220. Proc. Natl. Acad. Sci. 103, 18504-18509 (2006).
16. Crawford, S. E. et al. Defects of the Heart, Eye, and Megakaryocytes in Peroxisome Proliferator Activator Receptor-binding Protein (PBP) Null Embryos Implicate GATA Family of Transcription Factors. J. Biol. Chem. 277, 3585-3592 (2002).
17. Malik, S., Wallberg, A. E., Kang, Y. K. & Roeder, R. G. TRAP/SMCC/mediator-dependent transcriptional activation from DNA and chromatin templates by orphan nuclear receptor hepatocyte nuclear factor 4. Mol. Cell. Biol. 22, 5626-37 (2002).
18. Wang, S., Ge, K., Roeder, R. G. & Hankinson, O. Role of mediator in transcriptional activation by the aryl hydrocarbon receptor. J. Biol. Chem. 279, 13593-600 (2004).
19. Wang, Q., Sharma, D., Ren, Y. & Fondell, J. D. A Coregulatory Role for the TRAP-Mediator Complex in Androgen Receptor-mediated Gene Expression. J. Biol. Chem. 277, 42852-42858 (2002).
20. Naar, A. M. et al. Composite co-activator ARC mediates chromatin-directed transcriptional activation. Nature 398, 828-32 (1999).
21. Hittelman, A. B., Burakov, D., Iñiguez-Lluhí, J. A., Freedman, L. P. & Garabedian, M. J. Differential regulation of glucocorticoid receptor transcriptional activation via AF-1-associated proteins. EMBO J. 18, 5380-5388 (1999).
22. Atkins, G. B. et al. Coactivators for the Orphan Nuclear Receptor RORα. Mol. Endocrinol. 13, 1550-1557 (1999).
23. Chen, W. & Roeder, R. G. The Mediator subunit MED1/TRAP220 is required for optimal glucocorticoid receptor-mediated transcription activation. Nucleic Acids Res. 35, 6161-9 (2007).
24. Pineda Torra, I., Freedman, L. P. & Garabedian, M. J. Identification of DRIP205 as a Coactivator for the Farnesoid X Receptor. J. Biol. Chem. 279, 36184-36191 (2004).
25. Zhou, T. & Chiang, C.-M. Sp1 and AP2 regulate but do not constitute TATA-less human TAF(II)55 core promoter activity. Nucleic Acids Res. 30, 4145-57 (2002).
26. Ito, M. et al. Identity between TRAP and SMCC complexes indicates novel pathways for the function of nuclear receptors and diverse mammalian activators. Mol. Cell 3, 361-70 (1999).
27. Zhou, H., Kim, S., Ishii, S. & Boyer, T. G. Mediator Modulates Gli3-Dependent Sonic Hedgehog Signaling. Mol. Cell. Biol. 26, 8667-8682 (2006).
28. Tutter, A. V et al. Role for Med12 in regulation of Nanog and Nanog target genes. J. Biol. Chem. 284, 3709-18 (2009).
29. Hein, M. Y. et al. A human interactome in three quantitative dimensions organized by stoichiometries and abundances. Cell 163, 712-23 (2015).
30. Gwack, Y. et al. Principal role of TRAP/mediator and SWI/SNF complexes in Kaposi's sarcoma-associated herpesvirus RTA-mediated lytic reactivation. Mol. Cell. Biol. 23, 2055-67 (2003).
31. Kim, S., Xu, X., Hecht, A. & Boyer, T. G. Mediator is a transducer of Wnt/beta-catenin signaling. J. Biol. Chem. 281, 14066-75 (2006).
32. Xu, X., Zhou, H. & Boyer, T. G. Mediator is a transducer of amyloid-precursor-protein-dependent nuclear signalling. EMBO Rep. 12, 216-222 (2011).
33. Grøntved, L., Madsen, M. S., Boergesen, M., Roeder, R. G. & Mandrup, S. MED14 tethers mediator to the N-terminal domain of peroxisome proliferator-activated receptor gamma and is required for full transcriptional activity and adipogenesis. Mol. Cell. Biol. 30, 2155-69 (2010).
34. Huttlin, E. L. et al. The BioPlex Network: A Systematic Exploration of the Human Interactome. Cell 162, 425-440 (2015).
35. Yang, F. et al. An ARC/Mediator subunit required for SREBP control of cholesterol and lipid homeostasis. Nature 442, 700-704 (2006).
36. Kim, T. W. et al. MED16 and MED23 of Mediator are coactivators of lipopolysaccharide- and heat-shock-induced transcriptional activators. Proc. Natl. Acad. Sci. U.S.A 101, 12153-8 (2004).
37. Taatjes, D. J., Naar, A. M., Andel, F., Nogales, E. & Tjian, R. Structure, function, and activator-induced conformations of the CRSP coactivator. Science 295, 1058-62 (2002).
38. van Essen, D., Engist, B., Natoli, G. & Saccani, S. Two Modes of Transcriptional Activation at Native Promoters by NF-κB p65. PLoS Biol. 7, e1000073 (2009).
39. Park, J. M. et al. Signal-induced transcriptional activation by Dif requires the dTRAP80 mediator module. Mol. Cell. Biol. 23, 1358-67 (2003).
40. Park, J. M., Werner, J., Kim, J. M., Lis, J. T. & Kim, Y. J. Mediator, not holoenzyme, is directly recruited to the heat shock promoter by HSF upon heat shock. Mol. Cell 8, 9-19 (2001).
41. Ding, N. et al. MED19 and MED26 are synergistic functional targets of the RE1 silencing transcription factor in epigenetic silencing of neuronal gene expression. J. Biol. Chem. 284, 2648-56 (2009).
42. Gu, W. et al. A novel human SRB/MED-containing cofactor complex, SMCC, involved in transcription regulation. Mol. Cell 3, 97-108 (1999).
43. Nevado, J., Tenbaum, S. P. & Aranda, A. hSrb7, an essential human Mediator component, acts as a coactivator for the thyroid hormone receptor. Mol. Cell. Endocrinol. 222, 41-51 (2004).
44. Asada, S. et al. External control of Her2 expression and cancer cell growth by targeting a Ras-linked coactivator. Proc. Natl. Acad. Sci. U.S.A 99, 12747-52 (2002).
45. Lambert, J.-P., Tucholska, M., Go, C., Knight, J. D. R. & Gingras, A.-C. Proximity biotinylation and affinity purification are complementary approaches for the interactome mapping of chromatin-associated protein complexes. J. Proteomics 118, 81-94 (2015).
46. Galbraith, M. D. et al. HIF1A employs CDK8-mediator to stimulate RNAPII elongation in response to hypoxia. Cell 153, 1327-39 (2013).
47. Mo, X., Kowenz-Leutz, E., Xu, H. & Leutz, A. Ras induces mediator complex exchange on C/EBP beta. Mol. Cell 13, 241-50 (2004).
48. Cantin, G. T., Stevens, J. L. & Berk, A. J. Activation domain-mediator interactions promote transcription preinitiation complex assembly on promoter DNA. Proc. Natl. Acad. Sci. U.S.A 100, 12003-8 (2003).
49. Stevens, J. L. et al. Transcription Control by E1A and MAP Kinase Pathway via Sur2 Mediator Subunit. Science (80-). 296, 755-758 (2002).
50. Mittler, G. et al. A novel docking site on Mediator is critical for activation by VP16 in mammalian cells. EMBO J. 22, 6494-504 (2003).
51. Yang, F., DeBeaumont, R., Zhou, S. & Näär, A. M. The activator-recruited cofactor/Mediator coactivator subunit ARC92 is a functionally important target of the VP16 transcriptional activator. Proc. Natl. Acad. Sci. U.S.A 101, 2339-44 (2004).
52. Lee, H.-K., Park, U.-H., Kim, E.-J. & Um, S.-J. MED25 is distinct from TRAP220/MED1 in cooperating with CBP for retinoid receptor activation. EMBO J. 26, 3545-3557 (2007).
53. Rana, R., Surapureddi, S., Kam, W., Ferguson, S. & Goldstein, J. A. Med25 is required for RNApolymerase II recruitment to specific promoters, thus regulating xenobiotic and lipid metabolism in human liver. Mol. Cell. Biol. 31, 466-81 (2011).
54. Nakamura, Y. et al. Wwp2 is essential for palatogenesis mediated by the interaction between Sox9 and mediator subunit 25. Nat. Commun. 2, 251 (2011).
55. Garrett-Engele, C. M. et al. intersex, a gene required for female sexual development in Drosophila, is expressed in both sexes and functions together with doublesex to regulate terminal differentiation. Development 129, 4661-75 (2002).
56. Eberhardy, S. R. & Farnham, P. J. Myc Recruits P-TEFb to Mediate the Final Step in the Transcriptional Activation of the cad Promoter. J. Biol. Chem. 277, 40156-40162 (2002).
57. Borggrefe, T. & Yue, X. Interactions between subunits of the Mediator complex with gene-specific transcription factors. Semin. Cell Dev. Biol. 22, 759-768 (2011).

Star Methods

Experimental Model and Subject Details

Cells

V6.5 murine embryonic stem were a gift from R. Jaenisch of the Whitehead Institute. V6.5 are male cells derived from a C57BL/6(F)×129/sv(M) cross. HEK293T cells were purchased from ATCC (ATCC CRL-3216). Cells were negative for mycoplasma.

Cell Culture Conditions

V6.5 murine embryonic stem (mES) cells were grown in 2i+LIF conditions. mES cells were always grown on 0.2% gelatinized (Sigma, G1890) tissue culture plates. The media used for 2i+LIF media conditions is as follows: 967.5 mL DMEM/F12 (GIBCO 11320), 5 mL N2 supplement (GIBCO 17502048), 10 mL B27 supplement (GIBCO 17504044), 0.5 mML-glutamine (GIBCO 25030), 0.5× non-essential amino acids (GIBCO 11140), 100 U/mL Penicillin-Streptomycin (GIBCO 15140), 0.1 mM b-mercaptoethanol (Sigma), 1 uM PD0325901 (Stemgent 04-0006), 3 uM CHIR99021 (Stemgent 04-0004), and 1000 U/mL recombinant LIF (ESGRO ESG1107). For differentiation mESCs were cultured in serum media as follows: DMEM (Invitrogen, 11965-092) supplemented with 15% fetal bovine serum (Hyclone, characterized SH3007103), 100 mM nonessential amino acids (Invitrogen, 11140-050), 2 mM L-glutamine (Invitrogen, 25030-081), 100 U/mL penicillin, 100 mg/mL streptomycin (Invitrogen, 15140-122), and 0.1 mM b-mercaptoethanol (Sigma Aldrich). HEK293T cells were purchased from ATCC (ATCC CRL-3216) and cultured in DMEM, high glucose, pyruvate (GIBCO 11995-073) with 10% fetal bovine serum (Hyclone, characterized SH3007103), 100 U/mL Penicillin-Streptomycin (GIBCO 15140), 2 mM L-glutamine (Invitrogen, 25030-081). Cells were negative for mycoplasma.

Method Details

Immunofluorescence with RNA FISH

Coverslips were coated at 37° C. with 5 ug/mL poly-L-ornithine (Sigma-Aldrich, P4957) for 30 minutes and 5 μg/mL of Laminin (Corning, 354232) for 2 hours. Cells were plated on the pre-coated cover slips and grown for 24 hours followed by fixation using 4% paraformaldehyde, PFA, (VWR, BT140770) in PBS for 10 minutes. After washing cells three times in PBS, the coverslips were put into a humidifying chamber or stored at 4° C. in PBS. Permeabilization of cells were performed using 0.5% triton X100 (Sigma Aldrich, X100) in PBS for 10 minutes followed by three PBS washes. Cells were blocked with 4% IgG-free Bovine Serum Albumin, BSA, (VWR, 102643-516) for 30 minutes and indicated primary antibody (see table S4) was added at a concentration of 1:500 in PBS for 4-16 hours. Cells were washed with PBS three times followed by incubation with secondary antibody at a concentration of 1:5000 in PBS for 1 hour. After washing twice with PBS, cells were fixed using 4% paraformaldehyde, PFA, (VWR, BT140770) in PBS for 10 minutes. After two washes of PBS, Wash buffer A (20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) was added to cells and incubated for 5 minutes. 12.5 μM RNA probe (Table S6, Stellaris) in Hybridization buffer (90% Stellaris RNA FISH Hybridization Buffer (Biosearch Technologies, SMF-HB1-10) and 10% Deionized Formamide) was added to cells and incubated overnight at 37 C. After washing with Wash buffer A for 30 minutes at 37° C., the nuclei was stained in 20 μm/mL Hoechst 33258 (Life Technologies, H3569) for 5 minutes, followed by a 5 minute wash in Wash buffer B (Biosearch Technologies, SMF-WB1-20). Cells were washed once in water followed by mounting the coverslip onto glass slides with Vectashield (VWR, 101098-042) and finally sealing the cover slip with nail polish (Electron Microscopy Science Nm, 72180). Images were acquired at the RPI Spinning Disk confocal microscope with 100× objective using MetaMorph acquisition software and a Hammamatsu ORCA-ER CCD camera (W.M. Keck Microscopy Facility, MIT). Images were post-processed using Fiji Is Just ImageJ (FIJI).

Immunofluorescence with DNA FISH

Immunofluorescence was performed as previously above. After incubating the cells with the secondary antibodies, cells were washed three times in PBS for 5 min at RT, fixed with 4% PFA in PBS for 10 min and washed three times in PBS. Cells were incubated in 70% ethanol, 85% ethanol and then 100% ethanol for 1 minute at RT. Probe hybridization mixture was made mixing 7 μL of FISH Hybridization Buffer (Agilent G9400A), 1 μl of FISH probes (see below for region) and 2 μL of water. 5 μL of mixture was added on a slide and coverslip was placed on top (cell-side toward the hybridization mixture). Coverslip was sealed using rubber cement. Once rubber cement solidified, genomic DNA and probes were denatured at 78° C. for 5 minutes and slides were incubated at 16° C. in the dark O/N. The coverslip was removed from slide and incubated in pre-warmed Wash buffer 1 (Agilent, G9401A) at 73° C. for 2 minutes and in Wash Buffer 2 (Agilent, G9402A) for 1 minute at RT. Air dry slides and stain nuclei with Hoechst in PBS for 5 minutes at RT. Coverslips were washed three times in PBS, mounted on slide using Vectashield and sealed with nail polish. Images were acquired at the RPI Spinning Disk confocal microscope with 100× objective using MetaMorph acquisition software and a Hammamatsu ORCA-ER CCD camera (W.M. Keck Microscopy Facility, MIT).

DNA FISH probes were custom designed and generated by Agilent to target Nanog and MiR290 super enhancers.

Nanog

Design Input Region—mm9

chr6 122605249-122705248

Design Region—mm9

chr6: 122605985-122705394

Mir290

Design Region—mm10

chr7: 3141151-3241381

Tissue Culture

V6.5 murine embryonic stem cells (mESCs) were a gift from the Jaenisch lab. Cells were grown on 0.2% gelatinized (Sigma, G1890) tissue culture plates in 2i media, DMEM-F12 (Life Technologies, 11320082), 0.5× B27 supplement (Life Technologies, 17504044), 0.5× N2 supplement (Life Technologies, 17502048), an extra 0.5 mM L-glutamine (Gibco, 25030-081), 0.1 mM b-mercaptoethanol (Sigma, M7522), 1% Penicillin Streptomycin (Life Technologies, 15140163), 0.5× nonessential amino acids (Gibco, 11140-050), 1000 U/ml LIF (Chemico, ESG1107), 1 μM PD0325901 (Stemgent, 04-0006-10), 3 μM CHIR99021 (Stemgent, 04-0004-10). Cells were grown at 37° C. with 5% CO2 in a humidified incubator. For confocal imaging, cells were grown on glass coverslips (Carolina Biological Supply, 633029), coated with 5 μg/mL of poly-L-ornithine (Sigma Aldrich, P4957) for 30 minutes at 37° C. and with 5 μg/ml of Laminin (Corning, 354232) for 2 hrs-16 hrs at 37° C. For passaging, cells were washed in PBS (Life Technologies, AM9625), 1000 U/mL LIF. TrypLE Express Enzyme (Life Technologies, 12604021) was used to detach cells from plates. TrypLE was quenched with FBS/LIF-media (DMEM K/O (Gibco, 10829-018), 1× nonessential amino acids, 1% Penicillin Streptomycin, 2 mM L-Glutamine, 0.1 mM b-mercaptoethanol and 15% Fetal Bovine Serum, FBS, (Sigma Aldrich, F4135)). Cells were spun at 1000 rpm for 3 minutes at RT, resuspended in 2i media and 5×10⁶ cells were plated in a 15 cm dish. For differentiation of mESCs, 6000 cells were plated per well of a 6 well tissue culture dish, or 1000 cells were plated per well of a 24 well plate with a laminin coated glass coverslip. After 24 hours, 2i media was replaced with FBS media (above) without LIF. Media was changed daily for 5 days, cells were then harvested.

Western Blot

Cells were lysed in Cell Lytic M (Sigma-Aldrich C2978) with protease inhibitors (Roche, 11697498001). Lysate was run on a 3%-8% Tris-acetate gel or 10% Bis-Tris gel or 3-8% Bis-Tris gels at 80 V for ˜2 hrs, followed by 120 V until dye front reached the end of the gel. Protein was then wet transferred to a 0.45 μm PVDF membrane (Millipore, IPVH00010) in ice-cold transfer buffer (25 mM Tris, 192 mM glycine, 10% methanol) at 300 mA for 2 hours at 4° C. After transfer the membrane was blocked with 5% non-fat milk in TBS for 1 hour at room temperature, shaking. Membrane was then incubated with 1:1,000 of the indicated antibody (Table S4) diluted in 5% non-fat milk in TBST and incubated overnight at 4° C., with shaking. In the morning, the membrane was washed three times with TBST for 5 minutes at room temperature shaking for each wash. Membrane was incubated with 1:5,000 secondary antibodies for 1 hr at RT and washed three times in TBST for 5 minutes. Membranes were developed with ECL substrate (Thermo Scientific, 34080) and imaged using a CCD camera or exposed using film or with high sensitivity ECL.

Chromatin Immunoprecipitation (ChIP) qPCR and Sequencing

mES were grown to 80% confluence in 2i media. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1000 g for 3 minutes at 4° C., flash frozen in liquid nitrogen and stored at −80° C. All buffers contained freshly prepared cOmplete protease inhibitors (Roche, 11873580001). Frozen crosslinked cells were thawed on ice and then resuspended in lysis buffer I (50 mM HEPES-KOH, pH 7.5, 140 mM NaCl, 1 mM EDTA, 10% glycerol, 0.5% NP-40, 0.25% Triton X-100, 1 3 protease inhibitors) and rotated for 10 minutes at 4° C., then spun at 1350 rcf, for 5 minutes at 4° C. The pellet was resuspended in lysis buffer II (10 mM Tris-HCl, pH 8.0, 200 mM NaCl, 1 mM EDTA, 0.5 mM EGTA, 1 3 protease inhibitors) and rotated for 10 minutes at 4° C. and spun at 1350 rcf. for 5 minutes at 4° C. The pellet was resuspended in sonication buffer (20 mM Tris-HCl pH 8.0, 150 mM NaCl, 2 mM EDTA pH 8.0, 0.1% SDS, and 1% Triton X-100, 1 3 protease inhibitors) and then sonicated on a Misonix 3000 sonicator for 10 cycles at 30 s each on ice (18-21 W) with 60 s on ice between cycles. Sonicated lysates were cleared once by centrifugation at 16,000 rcf. for 10 minutes at 4° C. Input material was reserved and the remainder was incubated overnight at 4° C. with magnetic beads bound with antibody (Table S4) to enrich for DNA fragments bound by the indicated factor. Beads were washed twice with each of the following buffers: wash buffer A (50 mM HEPES-KOH pH 7.5, 140 mM NaCl, 1 mM EDTA pH 8.0, 0.1% Na-Deoxycholate, 1% Triton X-100, 0.1% SDS), wash buffer B (50 mM HEPES-KOH pH 7.9, 500 mM NaCl, 1 mM EDTA pH 8.0, 0.1% Na-Deoxycholate, 1% Triton X-100, 0.1% SDS), wash buffer C (20 mM Tris-HCl pH8.0, 250 mM LiCl, 1 mM EDTA pH 8.0, 0.5% Na-Deoxycholate, 0.5% IGEPAL C-630, 0.1% SDS), wash buffer D (TE with 0.2% Triton X-100), and TE buffer. DNA was eluted off the beads by incubation at 65° C. for 1 hour with intermittent vortexing in elution buffer (50 mM Tris-HCl pH 8.0, 10 mM EDTA, 1% SDS). Cross-links were reversed overnight at 65° C. To purify eluted DNA, 200 μL TE was added and then RNA was degraded by the addition of 2.5 μL of 33 mg/mL RNase A (Sigma, R4642) and incubation at 37° C. for 2 hours. Protein was degraded by the addition of 10 μL of 20 mg/mL proteinase K (Invitrogen, 25530049) and incubation at 55° C. for 2 hours. A phenol:chloroform:isoamyl alcohol extraction was performed followed by an ethanol precipitation. The DNA was then resuspended in 50 pt TE and used for either qPCR or sequencing. For ChIP-qPCR experiments, qPCR was performed using Power SYBR Green mix (Life Technologies #4367659) on either a QuantStudio 5 or a QuantStudio 6 System (Life Technologies).

RNA-Seq

RNA-Seq was performed in the indicated cell line with the indicated treatment, and used to determine expressed genes. RNA was isolated by AllPrep Kit (Qiagen 80204) and stranded polyA selected libraries was prepared using the TruSeq Stranded mRNA Library Prep Kit (Illumina, RS-122-2101) according to manufacturer's protocol and single-end sequenced on a Hi-seq 2500 instrument.

Protein Purification

cDNA encoding the genes of interest or their IDRs were cloned into a modified version of a T7 pET expression vector. The base vector was engineered to include a 5′ 6×HIS followed by either mEGFP or mCherry and a 14 amino acid linker sequence “GAPGSAGSAAGGSG.” (SEQ ID NO: 14). NEBuilder® HiFi DNA Assembly Master Mix (NEB E2621S) was used to insert these sequences (generated by PCR) in-frame with the linker amino acids. Vectors expressing mEGFP or mCherry alone contain the linker sequence followed by a STOP codon. Mutant sequences were synthesized as geneblocks (IDT) and inserted into the same base vector as described above. All expression constructs were sequenced to ensure sequence identity. For protein expression plasmids were transformed into LOBSTR cells (gift of Chessman Lab) and grown as follows. A fresh bacterial colony was inoculated into LB media containing kanamycin and chloramphenicol and grown overnight at 37° C. Cells containing the MED1-IDR constructs were diluted 1:30 in 500 ml room temperature LB with freshly added kanamycin and chloramphenicol and grown 1.5 hours at 16° C. IPTG was added to 1 mM and growth continued for 18 hours. Cells were collected and stored frozen at −80° C. Cells containing all other constructs were treated in a similar manner except they were grown for 5 hours at 37° C. after IPTG induction.

Pellets of 500 ml of cMyc and Nanog cells were resuspended in 15 ml of denaturing buffer (50 mM Tris 7.5, 300 mM NaCl, 10 mM imidazole, 8M Urea) containing cOmplete protease inhibitors (Roche, 11873580001) and sonicated (ten cycles of 15 seconds on, 60 sec off). The lysates were cleared by centrifugation at 12,000 g for 30 minutes and added to 1 ml of Ni-NTA agarose (Invitrogen, R901-15) that had been pre-equilibrated with 10 volumes of the same buffer. Tubes containing this agarose lysate slurry were rotated for 1.5 hours. The slurry was poured into a column, washed with 15 volumes of the lysis buffer and eluted 4× with denaturing buffer containing 250 mM imidazole. Each fraction was run on a 12% gel and proteins of the correct size were dialyzed first against buffer (50 mM Tris pH 7.5, 125 Mm NaCl, 1 Mm DTT and 4M Urea), followed by the same buffer containing 2M Urea and lastly 2 changes of buffer with 10% Glycerol, no Urea. Any precipitate after dialysis was removed by centrifugation at 3.000 rpm for 10 minutes. All other proteins were purified in a similar manner. 500 ml cell pellets were resuspended in 15 ml of Buffer A (50 mM Tris pH7.5, 500 mM NaCl) containing 10 mM imidazole and cOmplete protease inhibitors, sonicated, lysates cleared by centrifugation at 12,000 g for 30 minutes at 4° C., added to 1 ml of pre-equilibrated Ni-NTA agarose, and rotated at 4° C. for 1.5 hours. The slurry was poured into a column, washed with 15 volumes of Buffer A containing 10 mM imidazole and protein was eluted 2× with Buffer A containing 50 mM imidazole, 2× with Buffer A containing 100 mM imidazole, and 3× with Buffer A containing 250 mM imidazole. Alternatively, the resin slurry was centrifuged at 3,000 rpm for 10 minutes, washed with 15 volumes of Buffer and proteins were eluted by incubation for 10 or more minutes rotating with each of the buffers above (50 mM, 100 mM and 250 mM imidazole) followed by centrifugation and gel analysis. Fractions containing protein of the correct size were dialyzed against two changes of buffer containing 50 mM Tris 7.5, 125 mM NaCl, 10% glycerol and 1 mM DTT at 4° C.

In Vitro Droplet Assay

Recombinant GFP or mCherry fusion proteins were concentrated and desalted to an appropriate protein concentration and 125 mM NaCl using Amicon Ultra centrifugal filters (30K MWCO, Millipore). Recombinant proteins were added to solutions at varying concentrations with indicated final salt and 10% PEG-8000 as crowding agent in Droplet Formation Buffer (50 mM Tris-HCl pH 7.5, 10% glycerol, 1 mM DTT). The protein solution was immediately loaded onto a homemade chamber comprising a glass slide with a coverslip attached by two parallel strips of double-sided tape. Slides were then imaged with an Andor confocal microscope with a 150× objective. Unless indicated, images presented are of droplets settled on the glass coverslip. For experiments with fluorescently labeled polypeptides, the indicated decapeptides were synthesized by the Koch Institute/MIT Biopolymers & Proteomics Core Facility with a TMR fluorescent tag. The protein of interest was added Buffer D with 125 mM NaCl and 10% Peg-8000 with the indicated polypeptide and imaged as described above. For FRAP of in vitro droplets 5 pulses of laser at a 50 us dwell time was applied to the droplet, and recovery was imaged on an Andor microscope every is for the indicated time periods. For estrogen stimulation experiments, fresh B-Estradiol (E8875 Sigma) was reconstituted to 10 mM in 100% EtOH then diluted in 125 mM NaCl droplet formation buffer to 100 uM. One microliter of this concentrated stock was used in a 10 uL droplet formation reaction to achieve a final concentration of 10 uM.

Genome Editing and Protein Degradation

The CRISPR/Cas9 system was used to genetically engineer ESC lines. Target-specific oligonucleotides were cloned into a plasmid carrying a codon-optimized version of Cas9 with GFP (gift from R. Jaenisch). The sequences of the DNA targeted (the protospacer adjacent motif is underlined) are listed in the same table. For the generation of the endogenously tagged lines, 1 million Med1-mEGFP tagged mES cells were transfected with 2.5 mg Cas9 plasmid containing the guide sequence below (pX330-GFP-Oct4) and 1.25 mg non-linearized repair plasmid 1 (pUC19-Oct4-FKBP-BFP) and 1.25 mg non-linearized repair plasmid 2 (pUC19-Oct4-FKBP-mcherry) (Table S5). Cells were sorted after 48 hours for the presence of GFP. Cells were expanded for five days and then sorted again for double positive mCherry and BFP cells. Forty thousand mCherry+/BFP+ sorted cells were plated in a six-well plate in a serial dilution. The cells were grown for approximately one week in 2i medium and then individual colonies were picked using a stereoscope into a 96-well plate. Cells were expanded and genotyped by PCR, degradation was confirmed by western blot and IF. Clones with a homozygous knock-in tag were further expanded and used for experiments. A clonal homozygous knock-in line expressing FKBP tagged Oct4 was used for the degradation experiments. Cells were grown in 2i and then treated with dTAG-47 at a concentration of 100 nM for 24 hours, then harvested.

Oct4 Guide Sequence

(SEQ ID NO: 15)
tgcattcaaactgaggcacc*NGG(PAM)

GAL4 Transcription Assay

Transcription factor constructs were assembled in a mammalian expression vector containing an SV40 promoter driving expression of a GAL4 DNA-binding domain. Wild type and mutant activation domains of Oct4 and Gcn4 were fused to the C-terminus of the DNA-binding domain by Gibson cloning (NEB 2621S), joined by the linker GAPGSAGSAAGGSG (SEQ ID NO: 16). These transcription factor constructs were transfected using Lipofectamine 3000 (Thermofisher L3000015) into HEK293T cells (ATCC CRL-3216) or V6.5 mouse embryonic stem cells, that were grown in white flat-bottom 96-well assay plates (Costar 3917). The transcription factor constructs were co-transfected with a modified version of the PGL3-Basic (Promega) vector containing five GAL4 upstream activation sites upstream of the firefly luciferase gene. Also co-transfected was pRL-SV40 (Promega), a plasmid containing the Renilla luciferase gene driven by an SV40 promoter. 24 hours after transfection, luminescence generated by each luciferase protein was measured using the Dual-glo Luciferase Assay System (Promega E2920). The data as presented has been controlled for Renilla luciferase expression.

Lac Binding Assay

Constructs were assembled by NEB HIFI cloning in pSV2 mammalian expression vector containing an SV40 promoter driving expression of a CFP-LacI fusion protein. The activation domains and mutant activation domains of Gcn4 were fused by the c-terminus to this recombinant protein, joined by the linker sequence GAPGSAGSAAGGSG (SEQ ID NO: 17). U2OS-268 cells containing a stably integrated array of ˜51,000 Lac-repressor binding sites (a gift of the Spector laboratory) were transfected using lipofectamine 3000 (Thermofisher L3000015). 24 hours after transfection, cells were plated on fibronectin-coated glass coverslips. After 24 hours on glass coverslips, cells were fixed for immunofluorescence with a MED1 antibody (Table S4) as described above and imaged, by spinning disk confocal microscopy.

Purification of CDK8-Mediator

The CDK8-Mediator samples were purified as described (Meyer et al., 2008) with modifications. Prior to affinity purification, the P0.5M/QFT fraction was concentrated, to 12 mg/mL, by ammonium sulfate precipitation (35%). The pellet was resuspended in pH 7.9 buffer containing 20 mM KCl, 20 mM HEPES, 0.1 mM EDTA, 2 mM MgCl₂, 20% glycerol and then dialyzed against pH 7.9 buffer containing 0.15M KCl, 20 mM HEPES, 0.1 mM EDTA, 20% glycerol and 0.02% NP-40 prior to the affinity purification step. Affinity purification was carried out as described (Meyer et al., 2008), eluted material was loaded onto a 2.2 mL centrifuge tube containing 2 mL 0.15M KCl HEMG (20 mM HEPES, 0.1 mM EDTA, 2 mM MgCl₂, 10% glycerol) and centrifuged at 50K RPM for 4 h at 4° C. This served to remove excess free GST-SREBP and to concentrate the CDK8-Mediator in the final fraction. Prior to droplet assays, purified CDK8-Mediator was concentrated using Microcon-30 kDa Centrifugal Filter Unit with Ultracel-30 membrane (Millipore MRCFOR030) to reach ˜300 nM of Mediator complex. Concentrated CDK8-Mediator was added to the droplet assay to a final concentration of ˜200 nM with or without 10 μM indicated GFP-tagged protein. Droplet reactions contained 10% PEG-8000 and 140 mM salt.

Quantification and Statistical Analysis

Experimental Design

All experiments were replicated. For the specific number of replicates done see either the figure legends or the specific section below. No aspect of the study was done blinded. Sample size was not predetermined and no outliers were excluded.

Average Image and Radial Distribution Analysis

For analysis of RNA FISH with immunofluorescence custom in-house MATLAB™ scripts were written to process and analyze 3D image data gathered in FISH (RNA/DNA) and IF channels. FISH foci were manually identified in individual z-stacks through intensity thresholds, centered along a box of size l=2.9 and stitched together in 3-D across z-stacks. The called FISH foci are cross-referenced against a manually curated list of FISH foci to remove false positives, which arise due to extra-nuclear signal or blips. For every RNA FISH focus identified, signal from the corresponding location in the IF channel is gathered in the l×l square centered at the RNA FISH focus at every corresponding z-slice. The IF signal centered at FISH foci for each FISH and IF pair are then combined and an average intensity projection is calculated, providing averaged data for IF signal intensity within a l×l square centered at FISH foci. The same process was carried out for the FISH signal intensity centered on its own coordinates, providing averaged data for FISH signal intensity within a l×l square centered at FISH foci. As a control, this same process was carried out for IF signal centered at randomly selected nuclear positions. Randomly selected nuclear positions were identified for each image set by first identifying nuclear volume and then selecting positions within that volume. Nuclear volumes were determined from DAPI staining through the z-stack image, which was then processed through a custom CellProfiler pipeline (included as auxiliary file). Briefly, this pipeline rescales the image intensity, condenses the image to 20% of original size for speed of processing, enhances detected speckles, filters median signal, thresholds bodies, removes holes, filters the median signal, dilates the image back to original size, watersheds nuclei, and converts the resulting objects into a black and white image. This black and white image is used as input for a custom R script that uses readTIFF and im (from spatstat) to select 40 random nuclear voxels per image set. These average intensity projections were then used to generate 2D contour maps of the signal intensity or radial distribution plots. Contour plots are generated using in-built functions in MATLAB™. The intensity radial function ((r)) is computed from the average data. For the contour plots, the intensity-color ranges presented were customized across a linear range of colors (n!=15). For the FISH channel, black to magenta was used. For the IF channel, we used chroma.js (an online color generator) to generate colors across 15 bins, with the key transition colors chosen as black, blueviolet, mediumblue, lime. This was done to ensure that the reader's eye could more readily detect the contrast in signal. The generated colormap was employed to 15 evenly spaced intensity bins for all IF plots. The averaged IF centered at FISH or at randomly selected nuclear locations are plotted using the same color scale, set to include the minimum and maximum signal from each plot. For DNA FISH analysis FISH foci were manually identified in individual z-stacks through intensity thresholds in FIJI and marked as a reference area. The reference areas were then transferred to the MED1 IF channel of the image and the average IF signal within the FISH focus was determined. The average signal across 5 images comprising greater than 10 cells per image was averaged to calculate the mean MED1 IF intensity associated with the DNA FISH focus.

Chromatin Immunoprecipitation PCR and Sequencing (ChIP) Analysis

Values displayed in the figures were normalized to the input. The average WT norm values and standard deviation are displayed. The primers used are listed below. ChIP values at the region of interest (ROI) were normalized to input values (fold input) and for the mir290 enhancer an additional negative region (negative norm) Values are displayed as normalized to the ES state in differentiation experiments and to DMSO control in OCT4 degradation experiments (control normalization). qPCR reactions were performed in technical triplicate.

$$Fold input = {2}^{( Ct\_input - Ct\_ChIP )}$$

$$Negative norm = \frac{Fold {input}_{ROI}}{Fold {input}_{n e g}}$$

$$Control norm ( Differentiation ) = \frac{Neg {norm}_{Differentiated}}{Neg {norm}_{E S}}$$

CUP qPCR Primers

Mir290

mir290_Neg_F
SEQ ID NO: 16
GGACTCCATCCCTAGTATTTGC
mir290_Neg_R
SEQ ID NO: 17
GCTAATCACAAATTTGCTCTGC
mir290_OCT4_F
SEQ ID NO: 18
CCACCTAAACAAAGAACAGCAG
mir290_OCT4_R
SEQ ID NO: 19
TGTACCCTGCCACTCAGTTTAC
mir290_MED1_F
SEQ ID NO: 20
AAGCAGGGTGGTAGAGTAAGGA
mir290_MED1_R
SEQ ID NO: 21
ATTCCCGATGTGGAGTAGAAGT

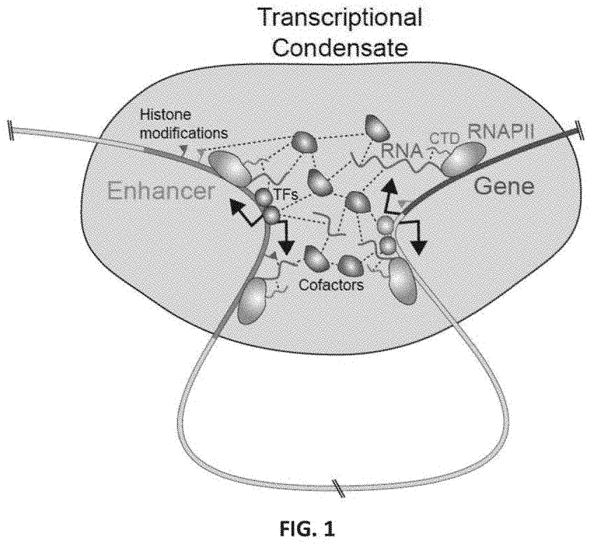

ChIP-Seq data were aligned to the mm9 version of the mouse reference genome using bowtie with parameters -k 1 -m 1 -best and -l set to read length. Wiggle files for display of read coverage in bins were created using MACS with parameters -w -S -space=50 -nomodel -shiftsize=200, and read counts per bin were normalized to the millions of mapped reads used to make the wiggle file. Reads-per-million-normalized wiggle files were displayed in the UCSC genome browser. ChIP-Seq tracks shown in FIG. 1 are derived from GSM1082340 (OCT4) and GSM560348 (MED1) from Whyte et al., 2013. Super-enhancers and typical enhancers and their associated genes in cells grown in 2i conditions were downloaded from Sabari et al., 2018. Distributions of occupancy fold-changes were calculated using bamToGFF (github.com/BradnerLab/pipeline) to quantify coverage in super-enhancers and typical enhancers from cells grown in 2i conditions. Reads overlapping each typical and super-enhancer were determined using bamToGFF with parameters -e 200 -f 1 -t TRUE and were subsequently normalized to the millions of mapped reads (RPM). RPM-normalized input read counts from each condition were then subtracted from RPM-normalized ChIP-Seq read counts from the corresponding condition. Values from regions wherein this subtraction resulted in a negative number were set to 0. Log 2 fold-changes were calculated between DMSO-treated (normal OCT4 amount) and dTAG-treated (depleted OCT4); one pseudocount was added to each condition.

Super-Enhancer Identification

Super-enhancers were identified as described in Whyte et al. Peaks of enrichment in MED1 were identified using MACS with -p 1e-9 -keep-dup=1 and input control. MED1 aligned reads from the untreated condition and corresponding peaks of MED1 were used as input for ROSE (bitbucket.org/young_computation/) with parameters -s 12500 -t 2000 -g mm9 and input control. A custom gene list was created by adding D7Ertd143e, and removing Mir290, Mir291a, Mir291b, Mir292, Mir293, Mir294, and Mir295 to prevent these nearby microRNAs that are part of the same transcript from being multiply counted. Stitched enhancers (super-enhancers and typical enhancers) were assigned to the single expressed RefSeq transcript whose promoter was nearest the center of the stitched enhancer. Expressed transcripts were defined as above.

RNA-Seq Analysis

For analysis, raw reads were aligned to the mm9 revision of the mouse reference genome using hisat2 with default parameters. Gene name-level read count quantification was performed with htseq-count with parameters -I gene_id -stranded=reverse -f bam -m intersection-strict and a GTF containing transcript positions from Refseq, downloaded 6/6/18. Normalized counts, normalized fold-changes, and differential expression p values were determined using DEseq2 using the standard workflow and both replicates of each condition.

Enrichment and Charge Analysis of OCT4

Amino acid composition plots were generated using R by plotting the amino acid identity of each residue along the amino acid sequence of the protein. Net charge per residue for OCT4 was determined by computing the average amino acid charge along the OCT4 amino acid sequence in a 5 amino acid sliding window using the localCIDER package (Holehouse et al., 2017).

Disorder Enrichment Analysis

A list of human transcription factors protein sequences is used for all analysis on TFs, as defined in (Saint-andré et al.). The reference human proteome (Uniprot UP000005640) is used to distill the list (down to ˜1200 proteins), mostly removing non-canonical isoforms. Transcriptional coactivators and Pol II associated proteins were identified in humans using the GO enrichments IDS GO:0003713 and GO:0045944. The reference human proteome defined above was used to generate list of all human proteins, and peroxisome and golgi proteins were identified from Uniprot reviewed lists. For each protein, D2P2 was used to assay disorder propensity for each amino acid. An amino acid in a protein is considered disordered if at least 75% of the algorithms employed by D2P2 (Oates et al., 2013) predict the residue to be disordered. Additionally, for transcription factors, all annotated PFAM domains were identified (5741 in total, 180 unique domains). Cross-referencing PFAM annotation for known DNA-binding activity, a subset of 45 unique high-confidence DNA-binding domains were identified, accounting for ˜85% of all identified domains. The vast majority of TFs (>95%) had at least one identified DNA-binding domain. Disorder scores were computed for all DNA-binding regions in every TF, as well as the remaining part of the sequence, which includes most identified trans-activation domains.

Imaging Analysis of In Vitro Droplets

To analyze in-vitro phase separation imaging experiments, custom MATLAB™ scripts were written to identify droplets and characterize their size and shape. For any particular experimental condition, intensity thresholds based on the peak of the histogram and size thresholds (2 pixel radius) were employed to segment the image. Droplet identification was performed on the “scaffold” channel (MED1 in case of MED1+TFs, GCN4 for GCN4+MED15), and areas and aspect ratios were determined. To calculate enrichment for the in vitro droplet assay, droplets were defined as a region of interest in FIJI by the scaffold channel, and the maximum signal of the client within that droplet was determined. Scaffolds chosen were MED1, Mediator complex, or GCN4. This was divided by the background client signal in the image to generate a Cin/out. Enrichment scores were calculated by dividing the Cin/out of the experimental condition by the Cin/out of a control fluorescent protein (either GFP or mCherry).

Data and Software Availability

Datasets

| Figure | Dataset type | IP target | Sample | GEO |
|---|---|---|---|---|
| 21B | ChIP-Seq | OCT4 | Oct4-degron + DMSO | GSM3401065 |
| 21B | ChIP-Seq | OCT4 | Oct4-degron + dTag | GSM3401066 |
| 21B | ChIP-Seq | MED1 | Oct4-degron + DMSO | GSM3401067 |
| 21B | ChIP-Seq | MED1 | Oct4-degron + dTag | GSM3401068 |
| 21B | ChIP-Seq Input | N/A | Oct4-degron + DMSO | GSM3401069 |
| 21B | ChIP-Seq Input | N/A | Oct4-degron + dTag | GSM3401070 |
| 21B | RNA-Seq | N/A | Oct4-degron + DMSO | GSM3401252 |
| | | | | GSM3401253 |
| 21B | RNA-Seq | N/A | Oct4-degron + dTag | GSM3401254 |
| | | | | GSM3401255 |
| 21H | RNA-Seq | N/A | ES Cell | GSM3401256 |
| | | | | GSM3401257 |
| 21H | RNA-Seq | N/A | Differentiating ES Cell | GSM3401258 |
| | | | | GSM3401259 |

### Overall Accession:

GSE120476

Key Resources Table

| | | |
|---|---|---|
| REAGENT or RESOURCE | SOURCE | IDENTIFIER |

Antibodies

| | | |
|---|---|---|
| MED1 | Abcam | ab64965 |
| OCT4 | Santa Cruz | sc-5279X |
| Goat anti-Rabbit IgG Alexa Fluor 488 | Life Technologies | A11008 |
| Goat anti-Rabbit IgG Alexa Fluor 568 | Life Technologies | A11011 |
| Goat anti-Mouse IgG Alexa Fluor 674 | Thermo Fisher | A21235 |
| Med1 | Bethyl | A300-793A-4 |
| Oct4 | Santa Cruz | sc-8628x |
| Beta-Actin | Santa Cruz | sc-7210 |
| HA | abcam | ab9110 |

Bacterial and Virus Strains

| | | |
|---|---|---|
| LOBSTR cells | Cheeseman Lab | N/A |
| | (WI/MIT) | |
| Biological Samples | | |

Chemicals, Peptides, and Recombinant Proteins

| | | |
|---|---|---|
| Beta-Estradiol | Sigma | E8875 |
| TMR-Poly-P Peptide | MIT core facility | N/A |
| TMR-Poly-E Peptide | MIT core facility | N/A |

Critical Commercial Assays

| | | |
|---|---|---|
| Dual-glo Luciferase Assay System | Promega | E2920 |
| AllPrep DNA/RNA Mini Kit | Qiagen | 80204 |
| NEBuilder® HiFi DNA Assembly Master Mix | NEB | E2621S |
| Power SYBR Green mix | Life Technologies | 4367659 |

Deposited Data

| | | |
|---|---|---|
| Oct4-degron + DMSO ChIP-seq | This application | GSM3401065 |
| Oct4-degron + dTag ChIP-seq | This application | GSM3401066 |
| Oct4-degron + DMSO ChIP-seq | This application | GSM3401067 |
| Oct4-degron + dTag ChIP-seq | This application | GSM3401068 |
| Oct4-degron + DMSO ChIP-Seq Input | This application | GSM3401069 |
| Oct4-degron + dTag ChIP-Seq Input | This application | GSM3401070 |
| Oct4-degron + DMSO RNA-seq | This application | GSM3401252 |
| | | GSM3401253 |
| Oct4-degron + dTag RNA-seq | This application | GSM3401254 |
| | | GSM3401255 |
| ES Cell RNA-seq | This application | GSM3401256 |
| | | GSM3401257 |
| Differentiating ES Cell RNA-seq | This application | GSM3401258 |
| | | GSM3401259 |
| Oct4 ChIP-Seq | Whyte et al., 2013 | GSM1082340 |
| Med1 ChIP-seq | Whyte et al., 2013 | G5M560348 |

Experimental Models: Cell Lines

| | | |
|---|---|---|
| V6.5 murine embryonic stem cells | Jaenisch laboratory | N/A |
| HEK293T cells | ATCC | CRL-3216 |
| U2OS-268 cells | Spector laboratory | N/A |
| Experimental Models: Organisms/Strains | | |

Oligonucleotides

| | | |
|---|---|---|
| mir290_Neg_F GGACTCCATCCCTAGTATTTGC | Operon | N/A |
| mir290_Neg_R GCTAATCACAAATTTGCTCTGC | Operon | N/A |
| mir290_OCT4_F CCACCTAAACAAAGAACAGCAG | Operon | N/A |
| mir290_OCT4_R TGTACCCTGCCACTCAGTTTAC | Operon | N/A |
| mir290_MED1_F AAGCAGGGTGGTAGAGTAAGGA | Operon | N/A |
| mir290_MED1_R ATTCCCGATGTGGAGTAGAAGT | Operon | N/A |

Recombinant DNA

| | | |
|---|---|---|
| pETEC-OCT4-GFP | This application | N/A |
| pETEC-MED1-IDR-GFP | Sabari et al., 2018. | N/A |
| pETEC-MED1-IDR-mCherry | Sabari et al., 2018. | N/A |
| pETEC-MED1-IDRXL-mCherry | This application | N/A |
| pETEC-OCT4-aromaticmutant-GFP | This application | N/A |
| pETEC-OCT4-acidicmutant-GFP | This application | N/A |
| pETEC-p53-GFP | This application | N/A |
| pETEC-yeast-MED15-mCherry | This application | N/A |
| pETEC-GCN4-GFP | This application | N/A |
| pETEC-GCN4-aromaticmutant-GFP | This application | N/A |
| pETEC-cMYC-GFP | This application | N/A |
| pETEC-NANOG-GFP | This application | N/A |
| pETEC-SOX2-GFP | This application | N/A |
| pETEC-RARa-GFP | This application | N/A |
| pETEC-GATA2-GFP | This application | N/A |
| pETEC-ER-GFP | This application | N/A |
| Lac-CFP-Empty | This application | N/A |
| Lac-GFP-Gcn4-AD | This application | N/A |
| Lac-GFP-Gcn4-AD-aromaticmutant | This application | N/A |
| pGL3BEC | Modified from Promega | N/A |
| pRLSV40 | Promega | N/A |
| pGal-DBD | This application | N/A |
| pGal-DBD-Oct4-C-AD | This application | N/A |
| pGal-DBD-Oct4-C-AD-acidicmutant | This application | N/A |
| pGal-DBD-GCN4-AD | This application | N/A |
| pGal-DBD-GCN4-AD-aromaticmutant | This application | N/A |
| pUC19-OCT4-FKBP-BFP | This application | N/A |
| pUC19-OCT4-FKBP-mcherry | This application | N/A |
| pX330-GFP-OCT4 | This application | N/A |

Software and Algorithms

| | | |
|---|---|---|
| Fiji image processing package | Schindelin et al., 2012 | https://fiji.sc/ |
| MetaMorph acquisition software | Molecular Devices | https://www.moleculardevices.com/ |
| | | products/cellular-imaging- |
| | | systems/acquisition- |
| | | and-analysis- |
| | | software/metamorph- |
| | | microscopy |
| localCIDER package | Holehouse et al., 2017 | N/A |
| PONDR | www.pondr.com | N/A |

Other

| | | |
|---|---|---|
| Esrrb RNA FISH probe | Stellaris | N/A |
| Nanog RNA FISH probe | Stellaris | N/A |
| miR290 RNA FISH probe | Stellaris | N/A |
| Trim28 RNA FISH probe | Stellaris | N/A |
| Nanog DNA FISH probe | Agilent | N/A |
| Mir290 DNA FISH probe | Agilent | N/A |

TABLE S4
Table of antibodies

| | | |
|---|---|---|
| IF Primary Antibodies | | |
| MED1 | Abcam ab64965 | 1:500 dilution |
| Oct4 | Santa Cruz sc-5279X | 1:500 dilution |
| p53 | Santa Cruz sc-47698 | 1:500 dilution |
| myc | Abcam ab32072 | 1:500 dilution |
| IF Secondary Antibodies | | |
| Goat anti-Rabbit IgG | Life Technologies A11008 | 1:500 dilution |
| Alexa Fluor 488 | | |
| Goat anti-Rabbit 6 IgG | Life Technologies A11011 | 1:500 dilution |
| Alexa Fluor 568 | | |
| Chip Antibodies | | |
| Med1 | Bethyl A300-793A-4 | |
| Oct4 | Santa Cruz sc-8628x | |
| PolII | Abcam ab817 | |
| Western Blot Antibodies | | |
| Oct4 | Santa Cruz sc-5279X | 1:1000 dilution |
| Med1 | Abeam ab64965 | 1:1000 dilution |
| p53 | Santa Cruz sc47698 | 1:500 dilution |
| myc | Santa Cruz sc40x | 1:1000 dilution |

TABLE S5
Constructs. All sequences of proteins are human unless otherwise indicated

| | | Contains |
|---|---|---|
| | | Amino Acids |
| | Source | ##-## |
| Vectors for OCT4-Degron Cell Line Generation | | |
| pUC19-OCT4-FKBP-BFP | This application | n/a |
| pUC19-OCT4-FKBP-mcherry | This application | n/a |
| pX330-GFP-OCT4 | This application | n/a |
| Protein Production in E Coli | | |
| pETEC-OCT4-GFP | This application | Full length |
| pETEC-MED1-IDR-GFP | Sabari et al., 2018. | 948-1574 |
| pETEC-MED1-IDR-mCherry | Sabari et al., 2018. | 948-1574 |
| pETEC-MED1-IDRXL-mCherry | This application | 600-1574 |
| pETEC-OCT4-aromaticmutant-GFP | This application | Full length |
| pETEC-OCT4-acidicmutant-GFP | This application | Full length |
| pETEC-p53-GFP | This application | Full length |
| pETEC-yeast-MED15-mCherry | This application | 6-651 |
| pETEC-GCN4-aromaticmutant-GFP | This application | Full length |
| pETEC-cMYC-GFP | This application | Full length |
| pETEC-NANOG-GFP | This application | Full length |
| pETEC-SOX2-GFP | This application | Full length |
| pETEC-RARa-GFP | This application | Full length |
| pETEC-GATA2-GFP | This application | Full length |
| pETEC-ER-GFP | This application | Full length |
| Lac Binding Assay In U2OS Cells | | |
| Lac-CFP-Empty | Modified from Promega | n/a |
| Lac-GFP-Gcn4-AD | This application | 1-133 |
| Lac-GFP-Gcn4-AD-aromaticmutant | This application | 1-133 |
| Gal4 Transcription Activation Assay | | |
| pGL3BEC | modified from promega | n/a |
| pRLSV40 | promega | n/a |
| pUC19 | addgene | n/a |
| pGal-DBD | This application | n/a |
| pGal-DBD-Oct4-C-AD | This application | 295-360 |
| pGal-DBD-Oct4-C-AD-acidicmutant | This application | 295-360 |
| pGal-DBD-GCN4-AD | This application | 1-133 |
| pGal-DBD-GCN4-AD-aromaticmutant | This application | 1-133 |

TABLE S6
Sequence of RNA FISH probes

| Esrrb | Nanog |
|---|---|
| tcaggagacttctagagcac(SEQ ID NO: 30) | gttcttcggggactgaattc(SEQ ID NO: 78) |
| gaaatccttgtctaggatcc(SEQ ID NO: 31) | ttttttctactcttacccta(SEQ ID NO: 79) |
| aatagtagcacctattcctc(SEQ ID NO: 32) | agaagcaataacccttcagc(SEQ ID NO: 80) |
| cctttctacaggtgtgatta(SEQ ID NO: 33) | cccgcttatgttaatgacta(SEQ ID NO: 81) |
| actcccaaacacattcatgg(SEQ ID NO: 34) | gggtttccagaagagtgata(SEQ ID NO: 82) |
| gactggatccaccattatta(SEQ ID NO: 35) | cagactagaaggccaacgta(SEQ ID NO: 83) |
| ccagaaagaatatcgcccag(SEQ ID NO: 36) | ttatattgctccgtcctgtg(SEQ ID NO: 84) |
| gaagcattaggagtctcgtt(SEQ ID NO: 37) | taggatgttaggtctccctg(SEQ ID NO: 85) |
| tcagttaagtgttcaccact(SEQ ID NO: 38) | aaatggggtgctcattccaa(SEQ ID NO: 86) |
| acagaatcaccctagggaag(SEQ ID NO: 39) | ctaactgtataacctcacca(SEQ ID NO: 87) |
| gcctccaaatggttaagtag(SEQ ID NO: 40) | aaacggccatttgggcaaat(SEQ ID NO: 88) |
| aagagctggttcaagtgtca(SEQ ID NO: 41) | aatgctaactgcttctgctg(SEQ ID NO: 89) |
| gtaaagacggcgatcggaga(SEQ ID NO: 42) | taagtgacatccatattccc(SEQ ID NO: 90) |
| taggtgtggtggtgatagac(SEQ ID NO: 43) | tgagctcacaaacccagaac(SEQ ID NO: 91) |
| ggtatagagcagcaaaagcc(SEQ ID NO: 44) | ctccagatgctagctataag(SEQ ID NO: 92) |
| attcatttcaccttgaggtc(SEQ ID NO: 45) | agacaatgagcttcagacct(SEQ ID NO: 93) |
| aagagacacaactgtctgcc(SEQ ID NO: 46) | tgagtactgggctgactctg(SEQ ID NO: 94) |
| ctcaatgtaagctctaggca(SEQ ID NO: 47) | ctcttggttctaccatttac(SEQ ID NO: 95) |
| caaggtcacttcccaattta(SEQ ID NO: 48) | catcacaacacgcacctgag(SEQ ID NO: 96) |
| tgtttacagatcttccctag(SEQ ID NO: 49) | tcacttacaaaggctatccc(SEQ ID NO: 97) |
| cttttcacggtagcacgtaa(SEQ ID NO: 50) | aaattatgccatctgctggc(SEQ ID NO: 98) |
| tcagccaacttctaggaaga(SEQ ID NO: 51) | ccctgaaagcagcttctaaa(SEQ ID NO: 99) |
| cgagtcctgtaatgagttca(SEQ ID NO: 52) | ctgcagtctagcaaataagt(SEQ ID NO: 100) |
| tacagggcgatagcaatctt(SEQ ID NO: 53) | tgatggcaatgctgaggtta(SEQ ID NO: 101) |
| aaaccatcccagagaattgc(SEQ ID NO: 54) | tgaagacatctgtgctccac(SEQ ID NO: 102) |
| ggaatgtctaggtgattgct(SEQ ID NO: 55) | aggtagaagacacctcctac(SEQ ID NO: 103) |
| gaagtttaggttccagtctg(SEQ ID NO: 56) | caacatttcctagatccagc(SEQ ID NO: 104) |
| gttccatagaactctagctt(SEQ ID NO: 57) | tcagcaagagacaagtgctc(SEQ ID NO: 105) |
| actggaagggatagcagagt(SEQ ID NO: 58) | tcttatccttgaccctctag(SEQ ID NO: 106) |
| ttctgtaaacttccttcctt(SEQ ID NO: 59) | tttcggttaaccaaattcgt(SEQ ID NO: 107) |
| caaagtctgtcatcacgtgc(SEQ ID NO: 60) | cagagggtccagttaattat(SEQ ID NO: 108) |
| cagacagctgtttcaactca(SEQ ID NO: 61) | taggaatgcacagtcctgag(SEQ ID NO: 109) |
| aactgatctgtctacctagc(SEQ ID NO: 62) | tccagggttaaatcacttgt(SEQ ID NO: 110) |
| tagtgtggtcaaggttgact(SEQ ID NO: 63) | tactctactaccactgagtc(SEQ ID NO: 111) |
| ggtaaagacttagaggctcc(SEQ ID NO: 64) | aatagaatcctgttgggacc(SEQ ID NO: 112) |
| gttatcctaagggctggaaa(SEQ ID NO: 65) | ctagatttttgcatggtgct(SEQ ID NO: 113) |
| tcaggaaatcagaccagtgc(SEQ ID NO: 66) | tttggggggacttttatctc(SEQ ID NO: 114) |
| aaagtggaaggaagccagcg(SEQ ID NO: 67) | gaggtttatccaaagactca(SEQ ID NO: 115) |
| cgataaagtctaccccacaa(SEQ ID NO: 68) | cagcagaggatctagtctat(SEQ ID NO: 116) |
| tagctcgaaaggctggcaaa(SEQ ID NO: 69) | agaatttgagatcagcccgt(SEQ ID NO: 117) |
| agttgaagtgttgggagtca(SEQ ID NO: 70) | ctgctccagtagctgagatg(SEQ ID NO: 118) |
| attttagtaccctcaggatt(SEQ ID NO: 71) | acagtgggtagcacaaatct(SEQ ID NO: 119) |
| gtgcaatgattggcactcaa(SEQ ID NO: 72) | acactgtaaacctctgatcc(SEQ ID NO: 120) |
| aacttaccctgagagctatt(SEQ ID NO: 73) | tcttcattagaaccgtgacc(SEQ ID NO: 121) |
| cagaacaacccatcagtcat(SEQ ID NO: 74) | tgtagtctgctctttccaat(SEQ ID NO: 122) |
| gctccattttaacagactct(SEQ ID NO: 75) | tatacaattagaccctggga(SEQ ID NO: 123) |
| gactctcaccaagtcaaagc(SEQ ID NO: 76) | ccggctatatttactttcaa(SEQ ID NO: 124) |
| atggctcagtttcagcaata(SEQ ID NO: 77) | |
| Mir290-295 | Trim28 |
| gctagcctgccttttaaaaa(SEQ ID NO: 125) | aaaccagcaggcctacttaa(SEQ ID NO: 173) |
| gagcgaggaaggctgagttc(SEQ ID NO: 126) | agacctggtaacgggcattg(SEQ ID NO: 174) |
| aatgtcttctttggagacca(SEQ ID NO: 127) | tctgatttcttgacatctcc(SEQ ID NO: 175) |
| actctttttccacacacatt(SEQ ID NO: 128) | agatttcccacaggacatac(SEQ ID NO: 176) |
| ttcctcccttgaaattatgt(SEQ ID NO: 129) | cagacactgagaccgcataa(SEQ ID NO: 177) |
| tactcactttccccacatag(SEQ ID NO: 130) | aatgcactcaaatctgtgcc(SEQ ID NO: 178) |
| taactcctagctttggtttc(SEQ ID NO: 131) | cttgccagtaaacacaagct(SEQ ID NO: 179) |
| aatgtactgcatagactccc(SEQ ID NO: 132) | tagaacaggcagacctaacc(SEQ ID NO: 180) |
| cttaaaattcactccaacct(SEQ ID NO: 133) | gagtgatagaaaggtggggg(SEQ ID NO: 181) |
| ccaggaggaaagaacgtgga(SEQ ID NO: 134) | ccaacagcctacaaatccaa(SEQ ID NO: 182) |
| gcggtccagacgttaaaaca(SEQ ID NO: 135) | tgtcaggttcctgaaaatcc(SEQ ID NO: 183) |
| gctggtaaatgtgccagata(SEQ ID NO: 136) | caaagtctgctcctgaaacc(SEQ ID NO: 184) |
| cagttaacccggaacacgtg(SEQ ID NO: 137) | agacttcctagtaccaatgg(SEQ ID NO: 185) |
| tttcttcgaatccgtactca(SEQ ID NO: 138) | ttatgctaagtgacccacta(SEQ ID NO: 186) |
| tcgctatactcagtctcatt(SEQ ID NO: 139) | ttcgttctagcctttactag(SEQ ID NO: 187) |
| tacaacgaccacctcagtta(SEQ ID NO: 140) | accaccaactgcaaagatgg(SEQ ID NO: 188) |
| taacagctccaagcagcgac(SEQ ID NO: 141) | caactaccttccactatctt(SEQ ID NO: 189) |
| gcgtcagatgcaaagctatg(SEQ ID NO: 142) | catctatcctgtaagtgcag(SEQ ID NO: 190) |
| taaactccaagcctaaaccc(SEQ ID NO: 143) | actaaaagagcagtcctgca(SEQ ID NO: 191) |
| aactgaaccgccctctttag(SEQ ID NO: 144) | aaccaagcccaaactatgga(SEQ ID NO: 192) |
| acgactgccttacatccatc(SEQ ID NO: 145) | ctacccaatgctaatccaat(SEQ ID NO: 193) |
| caatctacaatgcacctgga(SEQ ID NO: 146) | agactaacaaatcagtcccc(SEQ ID NO: 194) |
| ttagttcttagccgttttga(SEQ ID NO: 147) | gcgccaccaaaatagaaagt(SEQ ID NO: 195) |
| agaaatgcaaccccagtgaa(SEQ ID NO: 148) | accagcactcactgtcaaaa(SEQ ID NO: 196) |
| gactcaaacccacatgtgac(SEQ ID NO: 149) | ttcccaaataaacaaggccc(SEQ ID NO: 197) |
| aacgcggaaagcctttagta(SEQ ID NO: 150) | cccactcaccaatgaacaac(SEQ ID NO: 198) |
| tccaacttccaagacctgag(SEQ ID NO: 151) | aagtccttactatttcctgg(SEQ ID NO: 199) |
| aggtaagcgattccaggttg(SEQ ID NO: 152) | tctaggtctggaagcttttt(SEQ ID NO: 200) |
| agcacacatacctgtttcaa(SEQ ID NO: 153) | cttggcccatttattgataa(SEQ ID NO: 201) |
| tagccagtggcaacgaattc(SEQ ID NO: 154) | ggaaacaggaattatgccct(SEQ ID NO: 202) |
| taatatggcggccacgtgag(SEQ ID NO: 155) | ataatggtttccaactaccc(SEQ ID NO: 203) |
| gcaactacagtagtcaagca(SEQ ID NO: 156) | cacaaaagagtgagcctgca(SEQ ID NO: 204) |
| ccaactacagtagtcaagca(SEQ ID NO: 157) | caagcaaggataaccttgcc(SEQ ID NO: 205) |
| ttaaagtcagctacagccag(SEQ ID NO: 158) | acagtctcgttagggaaagc(SEQ ID NO: 206) |
| aagcttgtttgtgctaggag(SEQ ID NO: 159) | tgaatgaagcccaccactac(SEQ ID NO: 207) |
| ttatgggtattatctacccg(SEQ ID NO: 160) | aaggtcttaaggtgctgagg(SEQ ID NO: 208) |
| ctgggctattgtaaagccaa(SEQ ID NO: 161) | aatgggggagagggtgcaaa(SEQ ID NO: 209) |
| agattatgcttagggcacac(SEQ ID NO: 162) | ataaatactgcctcacctca(SEQ ID NO: 210) |
| gctaggcaggattacattca(SEQ ID NO: 163) | taagagaattcccattgggc(SEQ ID NO: 211) |
| ttgaaggcaagtaagtaccc(SEQ ID NO: 164) | tttccaaggcacaactactt(SEQ ID NO: 212) |
| ccacagatgacacccaaatg(SEQ ID NO: 165) | aagacagagacggggtactc(SEQ ID NO: 213) |
| cacctcagcttttacttttg(SEQ ID NO: 166) | tattcctaccacaccaatac(SEQ ID NO: 214) |
| ctgtcaaatctgggtcactt(SEQ ID NO: 167) | tgtatcttgtcatgagctca(SEQ ID NO: 215) |
| gccaaaaggataaatgcagc(SEQ ID NO: 168) | taaggaccatcctgtacatc(SEQ ID NO: 216) |
| ttcgctagatccaaacatgc(SEQ ID NO: 169) | atcttagggtgacaggtttc(SEQ ID NO: 217) |
| gttgattgaagttccgatgc(SEQ ID NO: 170) | tggaaagcttcagctactgg(SEQ ID NO: 218) |
| gatgagcaagcaaggagtct(SEQ ID NO: 171) | aacatagacattgagggggg(SEQ ID NO: 219) |
| aaagcagccgacctgtgaat(SEQ ID NO: 172) | gaatacacacgtgagtgggt(SEQ ID NO: 220) |

### Example 4

Mammalian heterochromatin is controlled by two major epigenetic pathways that are characterized by distinct chromatin modifications, histone H3 lysine 9 trimethylation (H3K9me3) and DNA methylation. These modifications are specifically recognized and bound by reader proteins with repressive activities. Most notably, HP1α is a reader of the H3K9me3 modification, while MeCP2 is a reader of DNA methylation. HP1α and MeCP2 are general chromatin regulators that are implicated in global gene control. Both proteins are essential for normal development, broadly expressed in many tissues, and mediate their effects via a multitude of interacting partners.

Heterochromatin has been traditionally viewed as a static and inaccessible structure in the nucleus. A prevalent view of transcriptional silencing is that chromatin compaction in heterochromatin excludes proteins such as RNA polymerases from the underlying DNA and thereby represses transcription. Some observations, however, have suggested that heterochromatin is a more dynamic assembly that permits rapid exchange of certain proteins. For example, heterochromatin protein HP1α, which recruits chromatin modifiers such as H3K9 methyltransferases and histone deacetylases to chromatin, rapidly exchanges between different heterochromatin domains as well as between chromatin-bound and nucleoplasm forms.

Liquid-liquid phase-separated (LLPS) is a physical phenomenon characterized by molecules de-mixing into distinct liquid phases with disparate concentrations. Formation of the dense liquid phase is driven by weak, multivalent intermolecular interactions such as those engendered by the low complexity and intrinsically disordered domains of proteins. LLPS has emerged as a mechanism in cellular organization, driving the formation of membrane-less organelles called condensates, which compartmentalize and concentrate biomolecules into membraneless bodies.

We wondered if MeCP2 contributes to a phase-separated heterochromatin compartment. Furthermore, severe neurological syndromes are caused by both loss of function and overexpression of MeCP2, and a condensate model has the potential to explain why both reduced and elevated levels might cause related syndromes. Here we show that MeCP2 forms dynamic liquid condensates by phase separation and that this property contributes to heterochromatin function. MeCP2 forms nuclear condensates with dynamic liquid-like properties at heterochromatin. The protein can form phase-separated liquid droplets in vitro that can incorporate repressive factors. The C-terminal intrinsically disordered domain of MeCP2 is essential for condensate formation in vitro, for heterochromatin association in vivo and for heterochromatin gene repression. These results suggest that MeCP2 functions to compartmentalize and concentrate repressive factors in heterochromatin.

Results

MeCP2 and HP1α Reside in Liquid-Like Heterochromatin Condensates

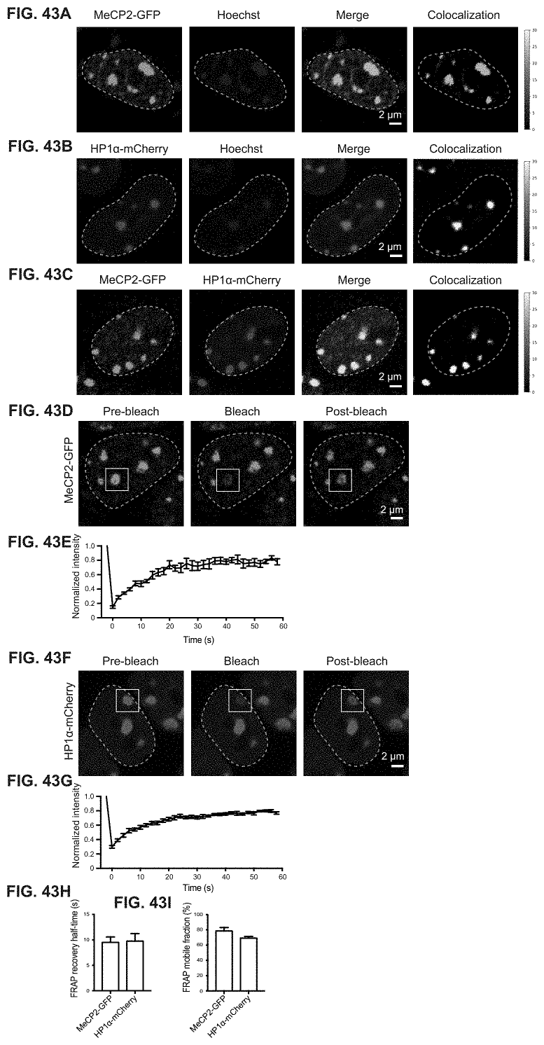

We sought to determine whether MeCP2 might contribute to the dynamic liquid condensate properties of mammalian heterochromatin by investigating its dynamic behavior in heterochromatin. To study MeCP2 in live cells at endogenous levels, we engineered murine embryonic stem cells (mESCs) to tag MeCP2 with monomeric enhanced green fluorescent protein (GFP) using the CRISPR/Cas9 system. To compare the dynamics of MeCP2 and HP1α in the same cell type, we additionally engineered mESCs to tag HP1α with mCherry. Live-cell fluorescence microscopy of both MeCP2-GFP and HP1α-mCherry cells revealed discrete nuclear bodies that overlapped with DNA dense heterochromatin foci (FIG. 43A and FIG. 43B). Comparison of MeCP2-GFP and HP1α-mCherry signal in the same nuclei showed that they both occur in the same heterochromatin condensates in mESCs (FIG. 43C). Analysis of live-cell images showed that there are 14.9±2.7 MeCP2 condensates per nucleus with a volume of 1.04±1.47 μm³ per condensate (mean±standard deviation). These results indicate that, when expressed at normal levels in mESCs, MeCP2 and HP1α are shared components of heterochromatin condensates.

We next sought to determine whether MeCP2 condensates display characteristic features of liquid condensates formed by phase separation. A key characteristic of condensates formed by liquid-liquid phase separation is the dynamic internal rearrangement and internal-external exchange of molecules (Hyman et al. 2014; Banani et al. 2017; Shin & Brangwynne 2017), which can be measured using fluorescence recovery after photobleaching (FRAP) experiments. To investigate the dynamics of MeCP2 condensates in live cells, we performed FRAP experiments on endogenously tagged MeCP2-GFP mESCs. MeCP2-GFP condensates recovered fluorescence after photobleaching on the time scale of seconds (FIG. 43D and FIG. 43E). FRAP of HP1α-mCherry mESCs showed similar recovery kinetics (FIG. 43F and FIG. 43G). Quantitative analysis showed that the recovery half-time for MeCP2-GFP was ˜10 s with a mobile fraction of ˜80% (FIG. 43H and FIG. 43I). Thus, both MeCP2 and HP1α show dynamic liquid-like properties in heterochromatin condensates.

MeCP2 Forms Phase-Separated Liquid Droplets In Vitro

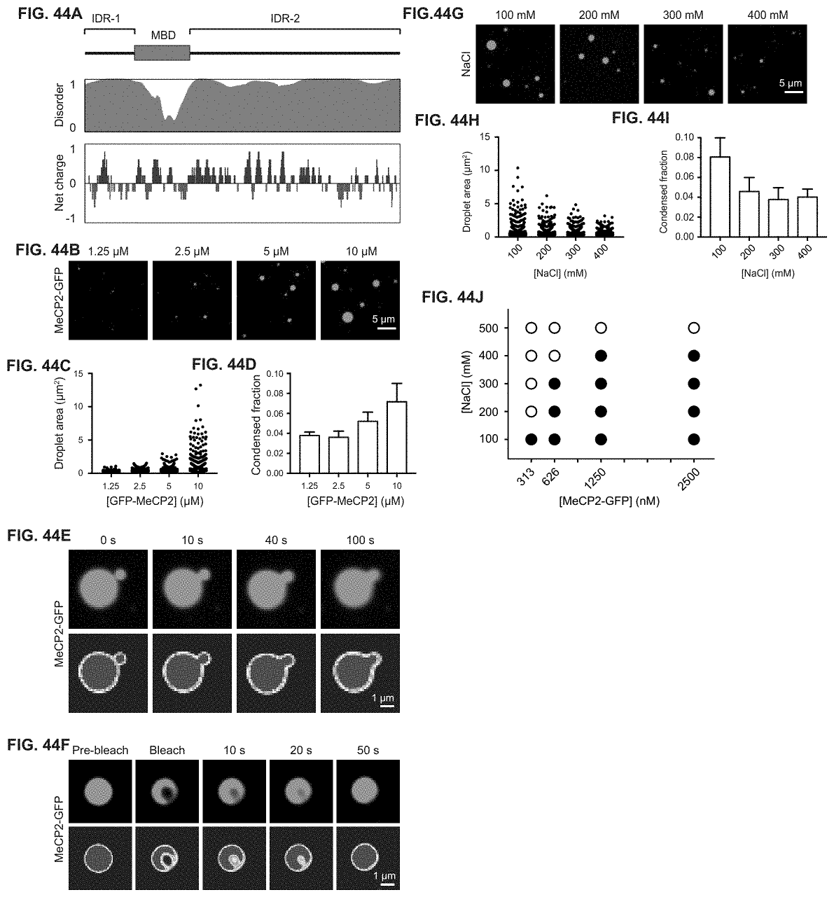

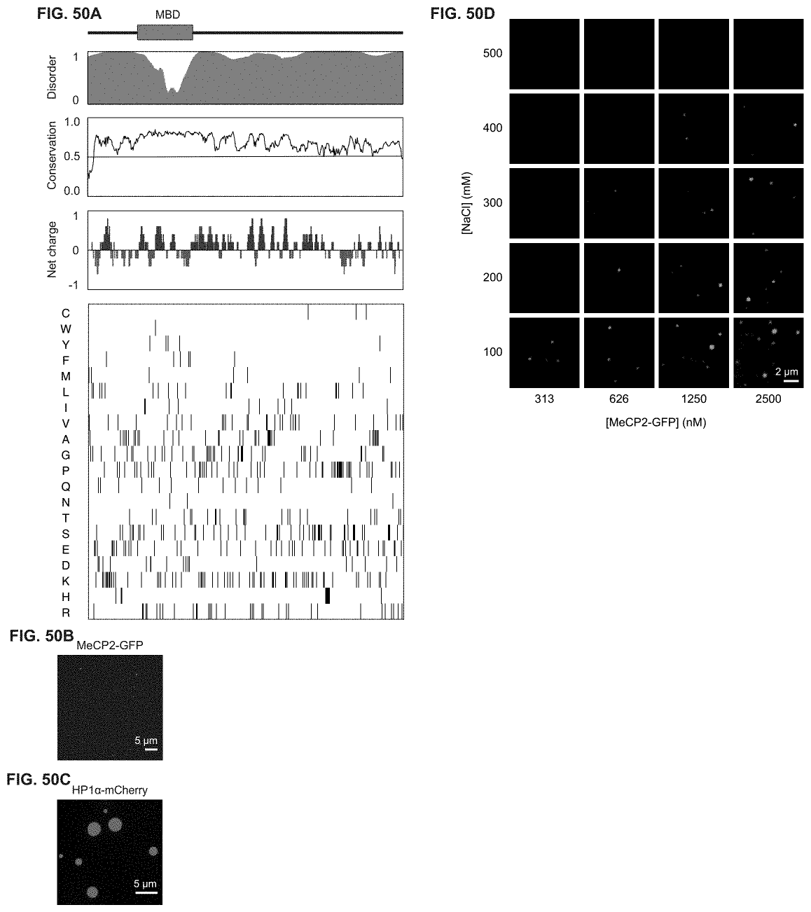

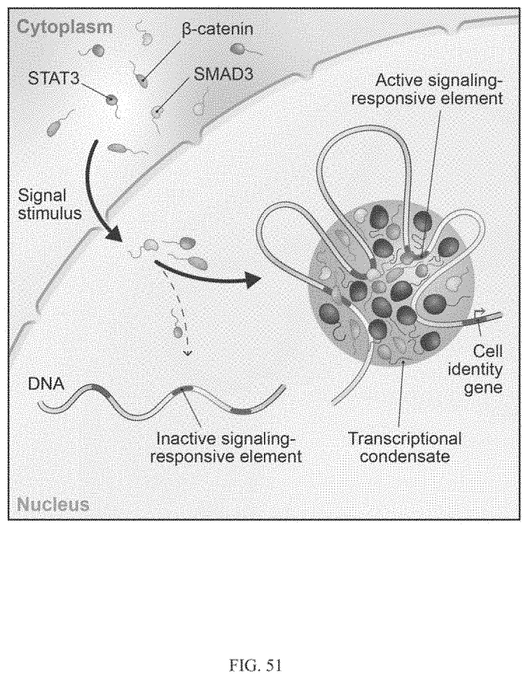

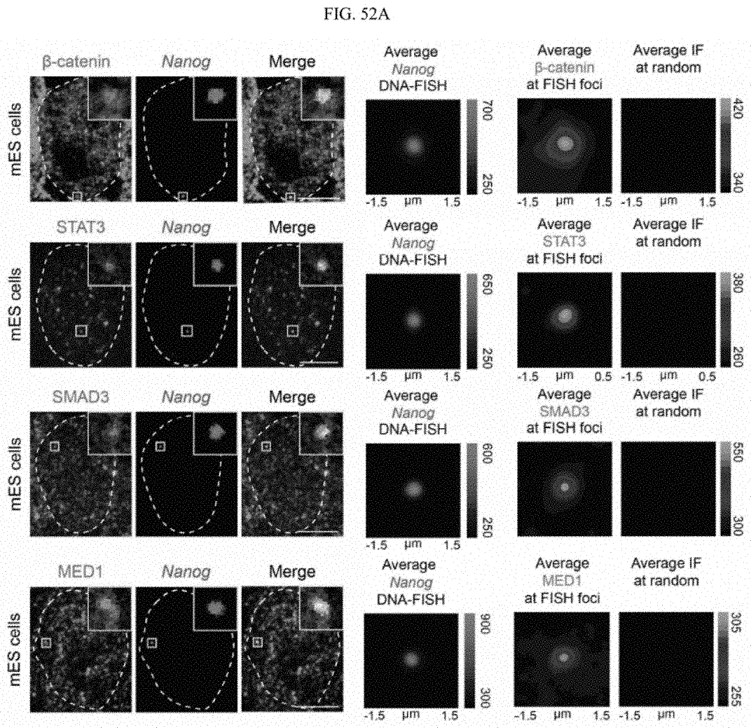

MeCP2 contains two conserved intrinsically disordered regions (IDRs) that flank its structured methyl-binding domain (MBD) (FIG. 44A and FIG. 50A)(Ghosh et al. 2010; Wakefield et al. 1999; Nan et al. 1993; Adams et al. 2007). Proteins involved in condensate formation often contain IDRs and when purified can form phase-separated liquid droplets in vitro (Burke et al. 2015; Nott et al. 2015; Lin et al. 2015; Kato et al. 2012; Sabari et al. 2018). In order to determine whether MeCP2 is capable of forming phase-separated droplets, recombinant MeCP2-GFP fusion protein was purified and studied in droplet formation assays. Addition of protein to a buffer containing a crowding agent to mimic the high concentration of factors in the nucleus induced formation of spherical droplets enriched for MeCP2-GFP, which were detected using fluorescence microscopy (FIG. 44B). Phase separated droplets typically scale in size with the concentration of the components in the system (Brangwynne 2013). MeCP2-GFP was found to form droplets at concentrations ranging from 160 nM to 10 μM and the droplets increased in size with increased protein concentrations (FIG. 44B-D and FIG. 50B). Liquid droplets are capable of fusion, and droplet fusion was observed with MeCP2-GFP (FIG. 44E). FRAP of MeCP2-GFP droplets showed recovery indicating dynamic rearrangement of molecules within MeCP2-GFP droplets (FIG. 44F). HP1α-mCherry was also found to form phase-separated droplets (FIG. 50C), confirming prior reports (Strom et al. 2017; Larson et al. 2017). These results demonstrate that MeCP2 can undergo phase separation to form liquid droplets, which leads us to conclude that both MeCP2 and HP1α are components of heterochromatin that have the capacity to undergo phase separation in vitro.

Phase separation can be driven by multivalent weak intermolecular interactions between amino acid residues within protein IDRs; both charged residues and aromatic residues have been shown to contribute to phase separation. Examination of the amino acid content of the two large IDRs of MeCP2 revealed a striking abundance of charged residues, but only a few aromatic residues (FIG. 44A and FIG. 50A). If electrostatic interactions contribute to MeCP2 phase separation, the ability of MeCP2 to form droplets should be diminished by increasing the salt concentration in the droplet formation assay, which will disrupt ionic interactions. Indeed, MeCP2 droplets were diminished by increasing salt concentrations (FIG. 44G-FIG. 44I), suggesting that electrostatic interactions contribute to the ability of MeCP2 to form phase-separated droplets. By examining MeCP2-GFP droplet formation capability at a variety of salt and protein concentrations, a phase diagram for MeCP2-GFP droplet formation was generated (FIG. 44J and FIG. 50D).

Condensate Formation, Heterochromatin Association and Gene Repression are Dependent on MeCP2 C-Terminal IDR

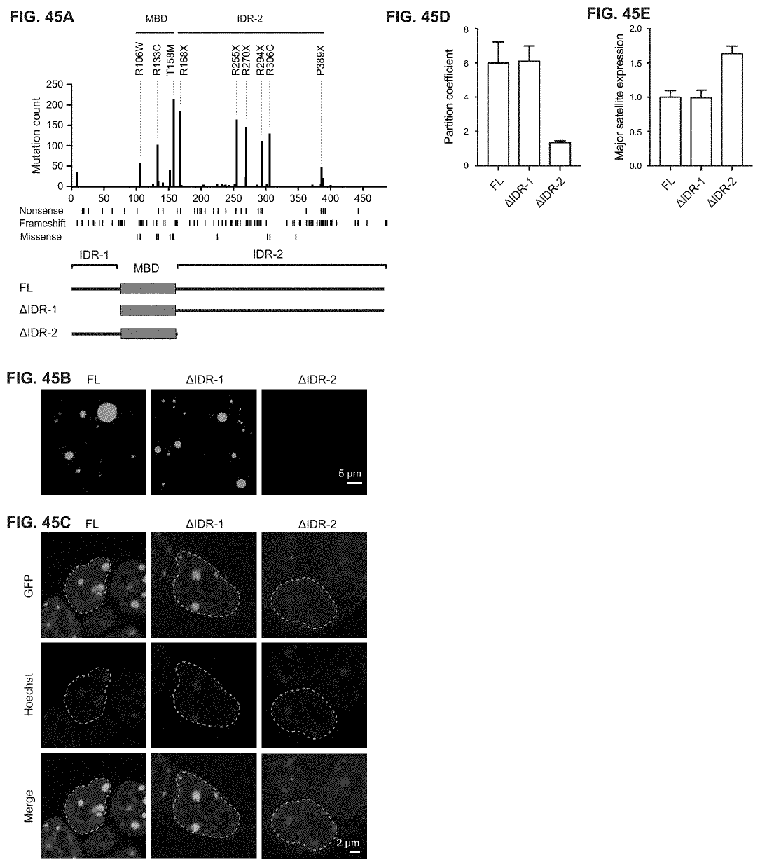

To determine whether the ability of MeCP2 to form phase-separated droplets depends on one or both of its IDRs, we purified recombinant MeCP2-GFP deletion mutants lacking either the N-terminal IDR (AIDR-1) or the C-terminal IDR (AIDR-2) (FIG. 45A) and examined their abilities to form droplets in vitro. Droplet assays revealed that the mutant lacking the N-terminal IDR (AIDR-1) remained capable of forming droplets but the mutant lacking the C-terminal IDR (AIDR-2) had lost this ability (FIG. 45B). These results indicate that the ability of MeCP2 to form phase-separated droplets in vitro is dependent on its C-terminal IDR.

We next investigated the ability of MeCP2-GFP mutants lacking either the N-terminal IDR (AIDR-1) or the C-terminal IDR (AIDR-2) to associate with heterochromatin in cells by using mESCs that were engineered to express these proteins from the endogenous Mecp2 locus. Live-cell fluorescence microscopy revealed that AIDR-1 MeCP2 localized to and displayed similar enrichment at heterochromatin as full-length MeCP2 (FIG. 45C and FIG. 45D). In contrast, AIDR-2 MeCP2 displayed reduced localization and enrichment at heterochromatin (FIG. 45C and FIG. 45D). These results indicate that both condensate formation in vitro and heterochromatin association in vivo depend on the C-terminal IDR of MeCP2.

If MeCP2 functions to facilitate gene repression through localization and concentration in heterochromatin condensates, we would expect that loss of IDR-2 would affect repetitive element silencing. Indeed, there was a significant increase in major satellite repeat expression in AIDR-2 MeCP2 cells when compared to full length MeCP2 cells (FIG. 45E). Taken together, these results suggest that condensate formation, heterochromatin localization and gene silencing are mutually dependent on MeCP2's C-terminal IDR.

MeCP2 Condensates can Compartmentalize Heterochromatin Factors

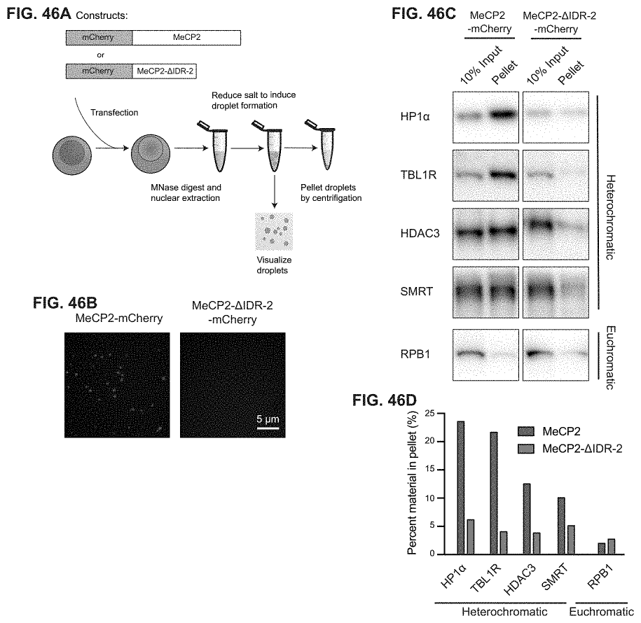

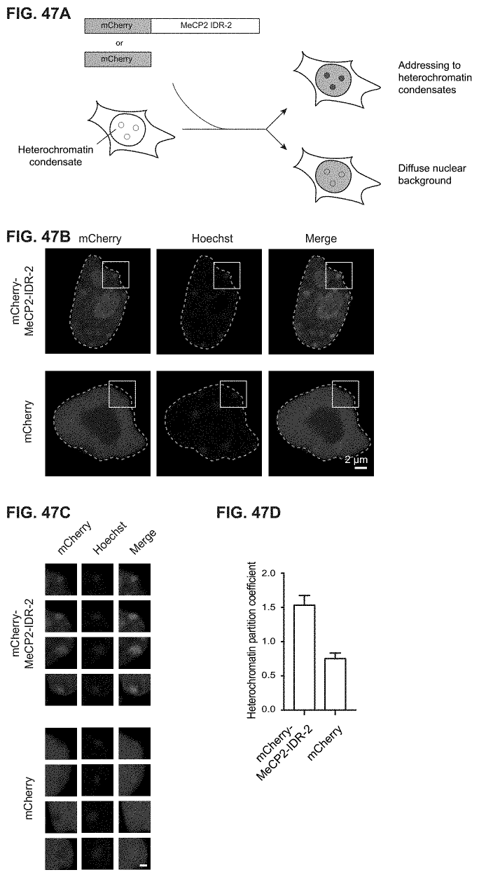

Condensates are thought to function to compartmentalize and concentrate factors within the condensed liquid phase. We used a droplet formation assay with nuclear extracts to investigate whether MeCP2 can compartmentalize into droplets various factors known to be associated with heterochromatin (FIG. 46A). Nuclear extracts were used because these contain all the components of the nucleus and condensate formation can occur without the addition of artificial crowding agents. Nuclear extracts were prepared from HEK293 cells expressing either MeCP2-mCherry or MeCP2-ΔIDR-2-mCherry under high salt conditions, and droplet formation was induced by reducing the salt concentration of the nuclear extracts. We found that droplets were formed in the nuclear extracts from cells expressing MeCP2-mCherry but not MeCP2-ΔIDR-2-mCherry (FIG. 46B). Condensates concentrate protein components and are thus more dense than the surrounding phase, so the nuclear extracts were subjected to centrifugation to spin down dense material and this material was analyzed by western blot. The results revealed that repressive factors known to be associated with heterochromatin, including HP1α, TBL1R (transducin beta-like protein), HDAC3 (histone deacetylase 3) and SMRT (silencing mediator of retinoic and thyroid receptor), were enriched in the MeCP2-mCherry extracts but not MeCP2-ΔIDR-2-mCherry extracts (FIG. 46C and FIG. 46D). In contrast, components of euchromatin, such as RNA polymerase II (RPB1) were not enriched (FIG. 46C and FIG. 46D). These results indicate that MeCP2 can form droplets in nuclear extracts that can compartmentalize and concentrate repressive factors associated with heterochromatin.

MeCP2 IDR-2 can Partition into Heterochromatin Condensates

The IDRs of condensate forming proteins have been proposed to address proteins to specific condensates, but there is little direct evidence for such an addressing function (Banani et al. 2017). We therefor studied whether the MeCP2 IDR-2 is sufficient to address mCherry protein to heterochromatin in cells (FIG. 47A). The MeCP2 IDR-2 fused to mCherry (mCherry-MeCP2-IDR-2) and control mCherry were ectopically expressed in mESCs and their localization was examined by microscopy. The mCherry-MeCP2-IDR-2 preferentially localized to DNA-dense heterochromatin and nucleoli, another nuclear body formed by phase separation (FIG. 47B-FIG. 47D). In contrast, mCherry alone was not enriched in heterochromatin or in nucleoli (FIG. 47B-FIG. 47C). These results suggest that the MeCP2-IDR-2 displays a degree of specific partitioning behavior in cells, consistent with the idea that preferential partitioning could contribute to proper addressing of factors to specific condensates.

MeCP2 is Concentrated in Heterochromatin of Neurons of Mouse Brain

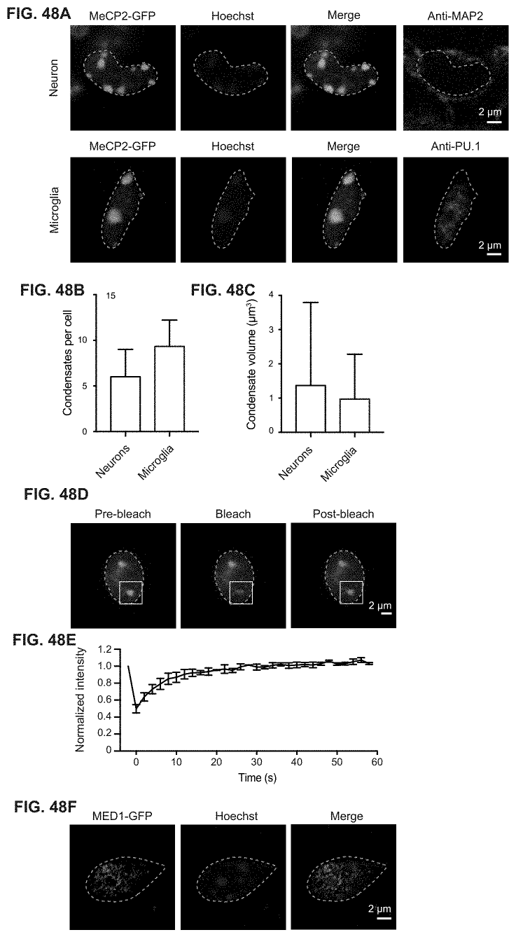

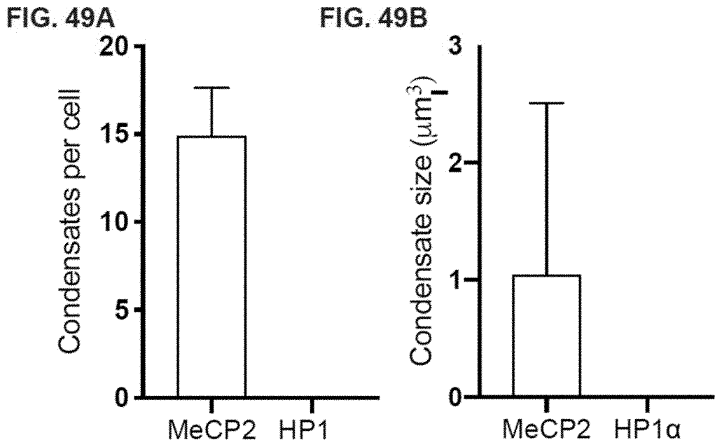

MeCP2 has been studied intensively because MECP2 loss of function mutations cause Rett syndrome and gene duplications cause MECP2 duplication syndrome; both of these syndromes involve neurological disorders characterized by severe intellectual disability. MeCP2 is expressed in all animal tissues but it is expressed at especially high levels in neurons (Skene et al. 2010). For these reasons, we sought to determine whether MeCP2 is also concentrated in liquid-like condensates in the neurons of the murine brain. Mouse models of Rett syndrome faithfully reproduce the phenotypes observed in the human syndrome. High-grade chimeric mice were generated from MECP2-GFP and MED1-GFP constructs integrated into the endogenous locus of reporter ES cells. At 2 months of age, following fixation by formalin perfusion, murine brains were sectioned into 10 μm slices. Fluorescence microscopy revealed that MeCP2 formed discrete nuclear bodies at DNA-dense heterochromatin foci in Map2-expressing neurons and PU.1-expressing microglia (FIG. 48A-FIG. 48C). FRAP experiments with freshly prepared live brain tissue sections showed that MeCP2-GFP is highly dynamic in these heterochromatin condensates (FIG. 48D and FIG. 48E). As expected, MED1-GFP puncta were smaller and more numerous, and were not associated with heterochromatin (FIG. 48F). These results indicate that MeCP2 is concentrated in the heterochromatin of live murine neurons and suggests that heterochromatin in these tissues behaves as a dynamic condensate.

Discussion

We show here that MeCP2 is a component of dynamic heterochromatin condensates in both ES cells and in neurons in brain tissue. The C-terminal IDR of MeCP2 is essential for its condensate forming properties and its ability to compartmentalize repressive factors in vitro, and for heterochromatin association and gene silencing in vivo. This MeCP2 IDR, expressed independently of the rest of the protein, is sufficient to address and incorporate the domain into heterochromatin condensates in cells. Our results thus show that MeCP2 is a component of dynamic heterochromatin condensates in multiple cell types and suggest that MeCP2's interaction with heterochromatin may be mediated by both its methyl DNA-binding and its condensate association properties.

The observation that MeCP2 and HP1α are both components of heterochromatin condensates is consistent with prior evidence that the two proteins are essential for normal development, are broadly expressed in many tissues, and are involved in gene repression (Allshire & Madhani 2018; Ip et al. 2018; Ausió et al. 2014; Lyst & Bird 2015; Guy et al. 2011). Prior studies have reported that crosstalk occurs between DNA methylation, H3K9 methylation and binding proteins MeCP2 and Hp1α. For example, in heterochromatinization of pericentromeric satellite repeats and in POU5F1 gene silencing after embryo implantation, the histone methyltransferase G9a trimethylates histone H3K9, which enables HP1α binding, and binds DNMT3, which methylates DNA, leading to MeCP2 binding. Both MeCP2 and HP1α can recruit additional partners involved in gene silencing, such as histone deacetylases. Our results, taken together with those described previously for HP1α, suggest that both MeCP2 and HP1α compartmentalize and concentrate these repressive factors to maintain the silent state of the heterochromatin compartment.

The observation that phase separation of heterochromatin proteins can function to concentrate and compartmentalize repressive factors provides a simplifying model to explain the diverse interactions ascribed to these proteins. Heterochromatin is associated with hundreds of protein factors. Both MeCP2 and HP1α have been observed to interact with numerous diverse interacting partners. How these interacting partners physically interact and stably associate with heterochromatin bodies is difficult to reconcile under a classic lock-and-key model of protein-protein interactions. The ability of MeCP2 and HP1α to form phase-separated heterochromatin condensates that concentrate and compartmentalize repressive factors within a dynamic meshwork of interactions better explains these observations. Notably, the ability of heterochromatin condensates to specifically concentrate repressive components and not the active transcriptional apparatus suggests a mechanism by which active and repressive factors are specifically compartmentalized into distinct condensates via the phase-separation properties of these condensates.

This model would explain why MeCP2 mutations that cause Rett syndrome can occur either in the DNA-binding domain or in the C-terminal IDR, where most mutations cause loss or truncation of the IDR (FIG. 48A).

Mutations that disrupt genes encoding heterochromatin proteins occur in a number of diseases. It is interesting to speculate whether these mutations may result in disease phenotypes via disruption of heterochromatin phase separation. Notably, missense and nonsense mutations in MECP2 cause Rett syndrome, a neurodevelopmental disorder that affects 1 in 10,000 young girls (Amir et al. 1999). These mutations often affect the IDRs of MeCP2 and may perturb the ability of MeCP2 to undergo phase separation at heterochromatin or to compartmentalize key factors within heterochromatin condensates. Additionally, pathogenic increases in MECP2 gene dosage cause MECP2 duplication syndrome, a related neurodevelopmental disorder in young males (Van Esch et al. 2005). Phase separated systems can be sensitive to small changes in the concentration of component factors, suggesting an aberrant increase or decrease in gene dosage could have substantial impacts on condensate behavior. Understanding the implications of disease mutations on heterochromatin phase separation may be important to understanding the molecular pathology and identifying new therapeutic opportunities to treat these diseases.

Methods

Cell Culture Conditions

Cell Culture

V6.5 murine embryonic stem cells (ESCs) were cultured in 2i/LIF media on tissue culture treated plates coated with 0.2% gelatin (Sigma G1890). ESCs were grown in a humidified incubator with 5% CO₂ at 37° C. Cells were passaged every 2-3 days by dissociation using TrypLE Express (Gibco 12604). The dissociation reaction was quenched using serum/LIF media. Cells were tested regularly for mycoplasma using the MycoAlert Mycoplasma Detection Kit (Lonza LT07-218) and found to be negative.

HEK293T cells were acquired from ATCC, and were cultured in DMEM (GIBCO) with high glucose, 10% fetal bovine serum (Hyclone, characterized SH3007103) 2 mM L-glutamine and 100U/mL penicillin-Streptomycin (GIBCO 15140).

Media Composition

The composition of 2i/LIF media is as follows: DMEM/F12 (Gibco 11320) supplemented with 0.5× N2 supplement (Gibco 17502), 0.5× B27 supplement (Gibco 17504), 2 mM L-glutamine (Gibco 25030), 1×MEM non-essential amino acids (Gibco 11140), 100 U/mL penicillin-streptomycin (Gibco 15140), 0.1 mM 2-mercaptoethanol (Sigma M7522), 3 μM CHIR99021 (Stemgent 04-0004), 1 μM PD0325901 (Stemgent 04-0006), and 1000 U/mL leukemia inhibitor factor (LIF) (ESGRO ESG1107).

The composition of serum/LIF media is as follows: KnockOut DMEM (Gibco 10829) supplemented with 15% fetal bovine serum (Sigma F4135), 2 mM L-glutamine (Gibco 25030), 1×MEM non-essential amino acids, 100 U/mL penicillin-streptomycin (Gibco 15140), 0.1 mM 2-mercaptoethanol (Sigma M7522), and 1000 U/mL leukemia inhibitor factor (LIF) (ESGRO ESG1107).

Genome Editing

The CRISPR/Cas9 system was used to generate genetically modified ESC lines. Target-specific sequences were cloned in to a plasmid containing sgRNA backbone, a codon-optimized version of Cas9, and mCherry or BFP (gift from R. Jaenisch). For generation of the MeCP2-mEGFP and HP1α-mCherry endogenously tagged lines, homology directed repair templates were cloned into pUC19 using NEBuilder HiFi DNA Master Mix (NEB E2621S). The homology repair template consisted of mEGFP or mCherry cDNA sequence flanked on either side by 800 bp homology arms amplified from genomic DNA using PCR.

To generate cell lines, 750,000 cells were transfected with 833 ng Cas9 plasmid and 1666 ng non-linearized homology repair template using Lipofectamine 3000 (Invitrogen L3000). Cells were sorted 48 hours after transfection for the presence of either mCherry or BFP fluorescence proteins encoded on the Cas9 plasmid to enrich for transfected cells. This population was allowed to expand for 1 week before sorting a second time for the presence of GFP or mCherry. 40,000 GFP positive cells were plated in serial dilution in a 6-well plate and allowed to expand for a week before individual colonies were manually picked into a 96-well plate. 24 colonies were screened for successful targeting using PCR genotyping to confirm insertion.

Live-Cell Imaging

Live-Cell Imaging Conditions

Cells were grown on 35 mm glass plates (Mattek Corporation P35G-1.5-20-C) and imaged in 2i/LIF media using an LSM880 confocal microscope with Airyscan detector (Zeiss, Thornwood, N.Y.). Cells were imaged on a 37° C. heated stage supplemented with 37° C. humidified air. Additionally, the microscope was enclosed in an incubation chamber heated to 37° C. ZEN black edition version 2.3 (Zeiss, Thornwood N.Y.) was used for acquisition. Images were acquired with the Airyscan detector in super-resolution (SR) mode with a Plan-Apochromat 63×/1.4 oil objective. Raw Airyscan images were processed using ZEN 2.3 (Zeiss, Thornwood N.Y.).

Fluorescence Recovery after Photobleaching (FRAP)

FRAP was performed on LSM880 Airyscan microscope with 488 nm and 561 nm lasers. Bleaching was performed at 100% laser power and images were collected every two seconds. Each image utilizes the LSM880 Airyscan averaging capacity and is the averaged result of two images. The combined image was then processed using ZEN2.3.

Recovery after photobleaching was calculated by first subtracting background values, and then quantifying fluorescence intensity lost within the bleached condensate normalized to signal within a condensate in a separate, neighboring cell to account for photobleaching. The MATLAB script FRAPPA Profiler was used to calculate intensity values in images, though normalizations were performed using custom analysis.

Calculation of MeCP2 Condensate Volumes

Z-stack images were taken using the ZEN 2.3 software. Cells were treated with SiR-DNA dye (Spirochrome SC007) to stain DNA for simplified focusing procedure. Far-red (SiR-DNA) signal was used to determine the upper- and lower-z boundaries of the nucleus. Then, images were taken in both the either the 488 or 561 channel and the 643 channel at 0.19 micron steps up through the nucleoplasm. Images are the result of a single Airyscan image, processed using the ZEN 2.3 software.

To quantify volume of MeCP2 condensates, The SiR-DNA signal was used to define nuclear-boundaries for a given cell. This boundary was used to mask non-nuclear signal in the 488 or 561 image. Once non-nuclear signal was masked, 488 and 561 images were subjected to a median filter of 7.0 pixels, and objects were counted and quantified using FIJI 3D Object counter, with a threshold of 154.

Calculation of Partition Coefficients

Partition coefficients in live-cell imaging were calculated using Fiji. Using a single focal plane per cell, average signal intensity within a condensate was quantified and compared to the average signal intensity from 8-12 non-heterochromatic regions within the nuclear boundary. Limitations of heterochromatic regions and nuclear boundaries were defined in the Hoechst channel. Cells that had >3 heterochromatin foci in the selected plane had a partition coefficient calculated. This individual coefficient represents a single n in the experiment.

Protein Purification

Protein Expression Vector Cloning

Human cDNA was cloned into a modified version of a T7 pET expression vector. The base vector was engineered to include sequences encoding a N-terminal 6×His followed by either mEGFP or mCherry and a 14 amino acid linker sequence “GAPGSAGSAAGGSG.” (SEQ ID NO: 14) cDNA sequences, generated by PCR, were inserted in-frame after the linker sequence using NEBuilder HiFi DNA Assembly Master Mix (NEB E2621S). Vector expressing mEGFP alone contains the linker sequence followed by a STOP codon. Mutant cDNA sequences were generated by PCR and inserted into the same base vector as described above. All expression constructs were sequenced to confirm sequence identity.

Protein Purification

For protein expression, plasmids were transformed into LOBSTR cells and grown as follows. A fresh bacterial colony was inoculated into LB media containing kanamycin and chloramphenicol and grown overnight at 37° C. Cells were diluted 1:30 in 500 mL prewarmed LB with freshly added kanamycin and chloramphenicol and grown 1.5 hours at 37° C. To induce expression, IPTG was added to the bacterial culture at 1 mM final concentration and growth continued for 4 hours. Induced bacteria were then pelleted by centrifugation and bacterial pellets were stored at −80° C. until ready to use.

The 500 mL cell pellets were resuspended in 15 ml of Lysis Buffer (50 mM Tris-HCl pH 7.5, 500 mM NaCl, and 1× cOmplete protease inhibitors) followed by sonication of ten cycles of 15 seconds on, 60 seconds off. Lysates were cleared by centrifugation at 12,000×g for 30 minutes at 4° C., added to 1 mL of pre-equilibrated Ni-NTA agarose, and rotated at 4° C. for 1.5 hours. The slurry was centrifuged at 3,000 rpm for 10 minutes, washed with 10 volumes of lysis buffer and proteins were eluted by incubation for 10 or more minutes rotating with lysis buffer containing 50 mM imidazole, 100 mM imidazole, or 3×250 mM imidazole followed by centrifugation and gel analysis. Fractions containing protein of the correct size were dialyzed against two changes of buffer containing 50 mM Tris-HCl pH 7.5, 125 mM NaCl, 10% glycerol and 1 mM DTT at 4° C. Protein concentration of purified proteins was determined using the Pierce BCA Protein Assay Kit (Thermo Scientific 23225).

In Vitro Droplet Assay

In Vitro Droplet Assays

Proteins were stored in 10% glycerol, 50 mM Tris-HCl pH 7.5, 500 mM NaCl, 1 mM DTT. Amicon Ultra Centrifugal filters (30K or 50K MWCO, Millipore) were used to concentrate proteins to desired concentrations. Reaction conditions for specific droplet assays are displayed for individual reaction throughout the manuscript. Droplet assays were performed in 8-tube PCR strip. Recombinant protein phase separation was induced in Droplet Formation Buffer composed of 10% PEG-8000, 10% glycerol, 50 mM Tris-HCl pH 7.5, 1 mM DTT and varying salt ranging from 0 mM to 500 mM NaCl. Next, the desired amount of protein was added to induce a phase transition, and the solution was mixed by pipetting. The reaction was then loaded onto either a custom slide chamber created from a glass coverslip mounted on two parallel strips of double-sided tape mounted on a glass microscopy slide or a glass-bottom 384 well-plate. The reaction was then imaged on an Andor confocal microscope with a 100× objective. Unless otherwise indicated, images presented are of droplets that have settled on the glass coverslip or the glass bottom of the 384 well-plate.

Data Analysis

To analyze in-vitro phase separation imaging experiments, custom MATLAB scripts were written to identify droplets and characterize their size, aspect ratio, condensed fraction and partition factor. For any particular experimental condition, intensity thresholds based on the peak of the histogram and size thresholds (2-pixel radius) were employed to segment the image, at which point regions of interest were defined and signal intensity could be quantified in and out of droplets.

Droplet Assays in Nuclear Extract

Preparation of Nuclear Extract

Nuclear extracts were prepared from HEK293Tcells. Cells were removed from culture plates vigorous pipetting, at which point they were pelleted at 1,000×g. The pellet was resuspended in TMSD50 buffer (20 mM HEPES, 5 mM MgCl₂ 250 mM sucrose, 1 mM DTT, 50 mM NaCl) with fresh protease inhibitors added. Cells were agitated for 30 minutes at 4 degrees Celsius in TMSD50 buffer to extract nuclei. The solution was then spun at 3,500×g for 10 minutes. Nuclei were washed in Mnase buffer (20 mM HEPES, 100 mM NaCl, 5 mM MgCl₂, 5 mM CaCl₂, protease inhibitors) and spun again at 3,500×g. Nuclei were then resuspended in one pellet volume of Mnase buffer and treated with 1U Mnase for 15 minutes at 37 degrees Celsius. Reaction was stopped with one pellet volume of stop buffer (20 mM HEPES, 500 mM NaCl, 5 mM MgCl₂, 20% glycerol, 15 mM EGTA, protease inhibitors). Digested nuclei were then sonicated 20 times at amplitude 20 on a tip sonicator and spun down twice at 2,700×g to remove debris.

Nuclear Extract Droplet Formation

Droplet formation assays with nuclear extract were performed by diluting stock nuclear extract 1:2 into Buffer B (10% glycerol, 20 mM HEPES) to reduce total salt to 150 mM NaCl. Assays were performed in 8-well PCR strips, where reactions were incubated for 15 minutes before being loaded onto a glass-bottom 384 well-plate. Droplets were allowed to settle onto the glass-bottom of the plate for 15 minutes before imaging on an Andor confocal microscope at 150×.

Nuclear Extract Pelleting

Droplets were formed as above in 1.5 mL Eppendorf tubes and incubated for 10 minutes. At this point, reactions were centrifuged at 2,700×g for 10 minutes. All supernatant was removed. The tubes were then gently washed with 1 mL droplet formation buffer (20 mM HEPES, 15% glycerol, 150 mM NaCl, 6.6 mM MgCl₂, 5 mM EGTA, 1.7 mM CaCl₂). After wash solution was removed, 25% βME, 25% XT buffer (Bio-rad), 50% water was added to the tube to prepare pellet fraction for western blotting. 10% of the material used for droplet formation was also combined with βME, XT buffer and water for western blotting.

Western Blot Analysis

Protein solutions described above were run on a 10% Bis-Tris gel (Bio-Rad) at 80V for 15 minutes, followed by 150V for ˜1.5 hrs. Protein was then transferred to a 0.45 μm PVDF membrane (Millipore, IPVH00010) in 4 degree Celsius transfer buffer (25 mM Tris, 192 mM glycine, 10% methanol) for 2 hours at 260 mA. Membrane was then blocked for 1 hr at room temperature in 5% non-fat milk in TBST. Membrane was then incubated with antibodies against the indicated protein in 5% milk in TBST overnight at 4 degrees Celsius while shaking. Membrane was then washed 3 times with TBST for 10 minutes each, incubated with secondary antibodies for 1 hr at room temperature, washed another 3 times with TBST and imaged on a Bio-Rad chemidoc using ECL or fempto-ECL substrate (Thermo Scientific).

qPCR Analysis

RNA was harvested using RNeasy kits (Qiagen). A reverse transcriptase reaction was then performed using Superscript3 (Invitrogen). qPCRs were performed using the following TaqMan probes:

(SEQ ID NO: 221)
mL1-Orf2a_1f-cctccattgaggtgggatt;
(SEQ ID NO: 222)
mL1-Orf2a_2r-ggaaccgccagactgatttc;
(SEQ ID NO: 223)
mGapdh_1f-ccatgtagttgaggtcaatgaagg;
(SEQ ID NO: 224)
mGapdh_2r-tggtgaaggtcggtgtgaa.

Immunofluorescence

Murine ESCs were plated on glass coverslips coated with poly-L-ornithine and laminin. After 24 hours, cells were fixed with 4% paraformaldehyde in PBS. Cells were then washed 3 times with PBS, Permeabilized with 0.5% Triton-X100 in PBS. Cells were then washed 3 times with PBS. Cells were blocked for 1 hr in 4% IgG-free BSA in PBS, and then stained over night with the indicated antibody in 4% IgG-free BSA at room temperature in a humidified chamber. Cells were then washed 3 times with PBS. Secondary antibodies were added to cells in 4% IgG-free BSA and incubated for 1 hr at room temperature. Cells were then washed 2 times in PBS. Cells were stained with Hoecsht dye in milliQ water for 5 minutes, and then mounted in Vectashield mounting media. Imaging was performed on an RPI spinning disk confocal at 100× magnification.

Transfection of IDR Expression Vectors

Cells were transfected using Lipofectamine 3000 (Life Technologies). 750,000 murine ESCs were counted and plated onto gelatinized 6-well dishes. Immediately after plating, DNA mixes prepared according to the Lipofectamine 3000 kit instructions were added to cells. 24 hours later, cells were trypsonized and split onto poly-L-ornithine and laminin-coated 35 mm glass-bottom dishes (Matek) for imaging.

### REFERENCES

Adams, V. H. et al., 2007. Intrinsic disorder and autonomous domain function in the multifunctional nuclear protein, MeCP2. Journal of Biological Chemistry, 282(20), pp. 15057-15064.
Allshire, R. C. & Madhani, H. D., 2018. Ten principles of heterochromatin formation and function. Nature Reviews Molecular Cell Biology, 19(4), pp. 229-244.
Amir, R. E. et al., 1999. Rett syndrome is caused by mutations in X-linked MECP2, encoding methyl-CpG-binding protein 2. Nature Genetics, 23(october), pp. 185-188.
Ausió, J., de Paz, A. M. artíne. & Esteller, M., 2014. MeCP2: the long trip from a chromatin protein to neurological disorders. Trends in molecular medicine, 20(9), pp. 487-498.
Banani, S. F. et al., 2017. Biomolecular condensates: organizers of cellular biochemistry. Nature Reviews Molecular Cell Biology, 18(5), pp. 285-298.
Bannister, A. J. et al., 2001. Selective recognition of methylated lysine 9 on histone H3 by the HP1 chromo domain. Nature, 410, pp. 120-124.
Brangwynne, C. P. et al., 2009. Germline P granules are liquid droplets that localize by controlled dissolution/condensation. Science, 5(June), pp. 1729-1732.
Brangwynne, C. P., 2013. Phase transitions and size scaling of membrane-less organelles. Journal of Cell Biology, 203(6), pp. 875-881.
Burke, K. A. et al., 2015. Residue-by-Residue View of In Vitro FUS Granules that Bind the C-Terminal Domain of RNA Polymerase II. Molecular Cell, 60(2), pp. 231-241.
Cheutin, T. et al., 2003. Maintenance of stable heterochromatin domains by dynamic HP1 binding. Science, 299(5607), pp. 721-725.
Chiolo, I. et al., 2011. Double-strand breaks in heterochromatin move outside of a dynamic HP1α domain to complete recombinational repair. Cell, 144(5), pp. 732-744.
Van Esch, H. et al., 2005. Duplication of the MECP2 Region Is a Frequent Cause of Severe Mental Retardation and Progressive Neurological Symptoms in Males. The American Journal of Human Genetics, 77(3), pp. 442-453.
Festenstein, R. et al., 2003. Modulation of Heterochromatin Protein 1 Dynamics in Primary Mammalian Cells. Science, 299(5607), pp. 719-721.
Ghosh, R. P. et al., 2010. Unique physical properties and interactions of the domains of methylated DNA binding protein 2. Biochemistry, 49(20), pp. 4395-4410.
Grewal, S. I. S. & Jia, S., 2007. Heterochromatin revisited. Nature Reviews Genetics, 8(1), pp. 35-46.
Guy, J. et al., 2011. The Role of MeCP2 in the Brain. Annual Review of Cell and Developmental Biology, 27(1), pp. 631-652.
Hendrich, B. & Bird, A., 1998. Identification and Characterization of a Family of Mammalian Methyl-CpG Binding Proteins. Molecular and Cellular Biology, 18(11), pp. 6538-6547.
Hyman, A. A., Weber, C. A. & \ddot{J}licher, F., 2014. Liquid-Liquid Phase Separation in Biology. Annual Review of Cell and Developmental Biology, 30(1), pp. 39-58.
Imbeault, M., Helleboid, P. Y. & Trono, D., 2017. KRAB zinc-finger proteins contribute to the evolution of gene regulatory networks. Nature, 543(7646), pp. 550-554.
Ip, J. P. K., Mellios, N. & Sur, M., 2018. Rett syndrome: insights into genetic, molecular and circuit mechanisms. Nature Reviews Neuroscience.
Kato, M. et al., 2012. Cell-free formation of RNA granules: Low complexity sequence domains form dynamic fibers within hydrogels. Cell, 149(4), pp. 753-767.
Lachner, M. et al., 2001. Methylation of histone H3 lysine 9 creates a binding site for HP1 proteins. Nature, 410(6824), pp. 116-120.
Larson, A. G. et al., 2017. Liquid droplet formation by HP1α suggests a role for phase separation in heterochromatin. Nature, 547(7662), pp. 236-240.
Lewis, J. D. et al., 1992. Purification, sequence, and cellular localization of a novel chromosomal protein that binds to Methylated DNA. Cell, 69(6), pp. 905-914.
Lin, Y. et al., 2015. Formation and Maturation of Phase-Separated Liquid Droplets by RNA-Binding Proteins. Molecular Cell, 60(2), pp. 208-219.
Lyst, M. J. & Bird, A., 2015. Rett syndrome: A complex disorder with simple roots. Nature Reviews Genetics, 16(5), pp. 261-274.
Meehan, R. R., Lewis, J. D. & Bird, A. P., 1992. Characterization of Mecp2, a Vertebrate Dna-Binding Protein With Affinity for Methylated Dna. Nucleic Acids Research, 20(19), p. 5085-5092 ST—CHARACTERIZATION OF MECP2, A VERTE.
Nakano, M. et al., 2008. Inactivation of a Human Kinetochore by Specific Targeting of Chromatin Modifiers. Developmental Cell, 14(4), pp. 507-522.
Nan, X., Meehan, R. R. & Bird, A., 1993. Dissection of the methyl-CpG binding domain from the chromosomal protein MeCP2. Nucleic Acids Research, 21(21), pp. 4886-4892.
Nott, T. J. et al., 2015. Phase Transition of a Disordered Nuage Protein Generates Environmentally Responsive Membraneless Organelles. Molecular Cell, 57(5), pp. 936-947.
Sabari, B. R. et al., 2018. Coactivator condensation at super-enhancers links phase separation and gene control. Science, 361(6400).
Shin, Y. & Brangwynne, C. P., 2017. Liquid phase condensation in cell physiology and disease. Science, 357(6357).
Skene, P. J. et al., 2010. Neuronal MeCP2 Is Expressed at Near Histone-Octamer Levels and Globally Alters the Chromatin State. Molecular Cell, 37(4), pp. 457-468.
Soufi, A., Donahue, G. & Zaret, K. S., 2012. Facilitators and impediments of the pluripotency reprogramming factors' initial engagement with the genome. Cell, 151(5), pp. 994-1004.
Strom, A. R. et al., 2017. Phase separation drives heterochromatin domain formation. Nature, 547(7662), pp. 241-245.
Tate, P., Skarnes, W. & Bird, A., 1996. The methyl-CpG binding protein MeCP2 is essential for embryonic development in the mouse. Nat Genet, 12, pp. 205-208.
Wakefield, R. I. D. et al., 1999. The solution structure of the domain from MeCP2 that binds to methylated DNA. Journal of Molecular Biology, 291(5), pp. 1055-1065.
Wang, J., Jia, S. T. & Jia, S., 2016. New Insights into the Regulation of Heterochromatin. Trends in Genetics, 32(5), pp. 284-294.

### Example 5

The gene expression programs that define each cell's identity are controlled by master transcription factors (TFs), which establish cell-type specific enhancers, and signaling factors, which bring extracellular stimuli to such enhancers. Signaling factors are expressed in diverse cell types and have little DNA binding sequence specificity, but are recruited to cell-type specific enhancers by mechanisms that are poorly understood. Recent studies have revealed that master TFs form phase-separated condensates with coactivators at enhancers. Here we present evidence that signaling factors for the WNT, TGF-β and JAK/STAT pathways employ their intrinsically disordered regions (IDRs) to enter and concentrate in Mediator condensates at super-enhancer driven genes. We propose that the cell-type specificity of the response to signaling is mediated, in part, by the IDRs of the signaling factors, which cause these factors to partition into condensates established by the master TFs and Mediator at genes with prominent roles in cell identity.

Several mechanisms have been described to account for the ability of signaling factors to preferentially bind the active enhancers and super-enhancers of a given cell type. Signaling factors bind with weak affinity to a relatively small sequence motif that is present at high frequency in the mammalian genome (Farley et al., 2015), and the preferred binding to sequences in active enhancers may reflect, in part, access to the “open chromatin” associated with active enhancers (Mullen et al., 2011). The signaling factors may also prefer to bind such sites due to structural changes in the DNA mediated by binding of other TFs at these enhancers (Hallikas et al., 2006; Zhu et al., 2018) or bind cooperatively through direct protein-protein interactions with master TFs (Kelly et al., 2011).

Recent studies have revealed that master TFs and the Mediator coactivator form phase-separated condensates at super-enhancers, which compartmentalize and concentrate the transcription apparatus at key cell identity genes (Boija et al., 2018; Cho et al., 2018; Sabari et al., 2018). Signaling factors have been shown to have a special preference for cell type-specific super-enhancers (Hnisz et al., 2015), leading us to postulate that signaling factors might have properties that lead them to partition into transcriptional condensates at super-enhancers, a previously uncharacterized mechanism for cell type-specific enhancer association. Here we report that signaling factors phase separate with coactivators in response to signaling stimuli at super-enhancer driven genes in a cell type-specific fashion. We propose that phase separation helps achieve the context-dependent specificity of signaling by addressing signaling factors to master TF-driven transcriptional condensates.

Results

Signal-Dependent Incorporation of Signaling Factors into Condensates at Super-Enhancers

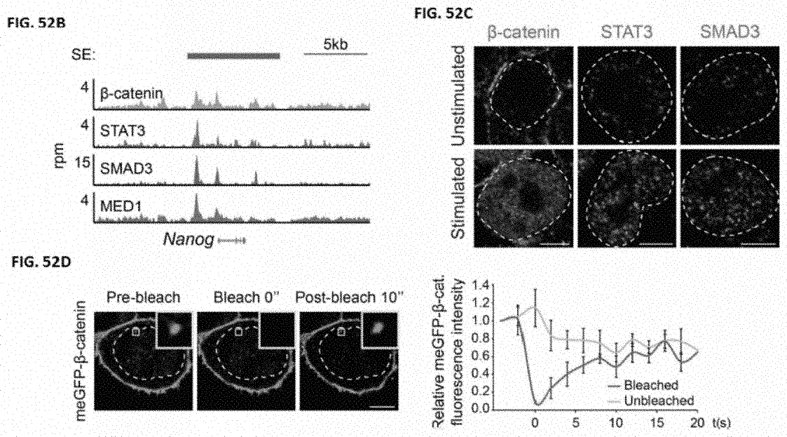

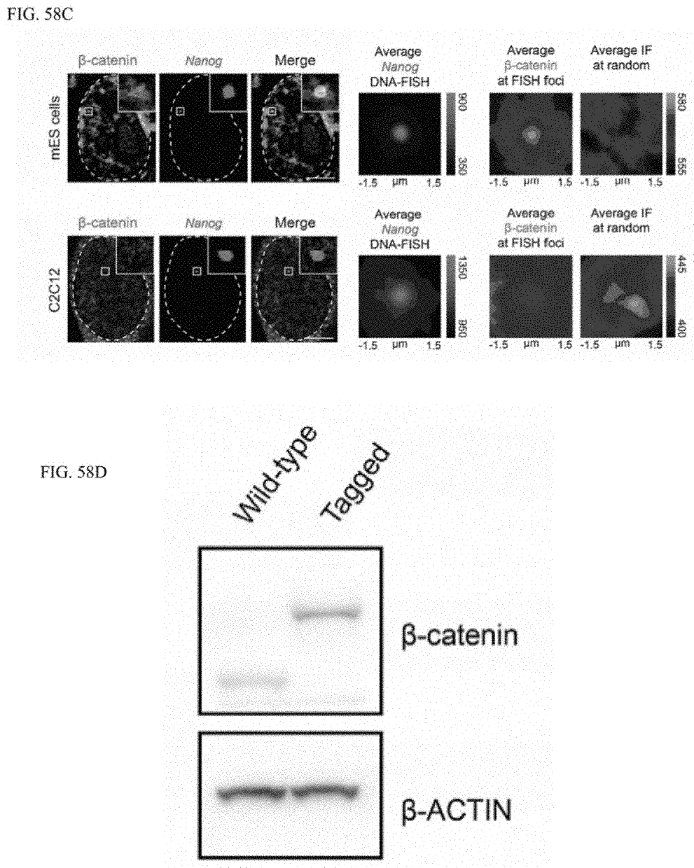

Recent studies have shown that TFs and Mediator form phase-separated condensates at super-enhancers (Boija et al., 2018; Cho et al., 2018; Sabari et al., 2018) and the terminal signaling factors of the WNT, JAK/STAT and TGF-β pathways (β-catenin, STAT3 and SMAD3, respectively) have been shown to preferentially occupy super-enhancers (Hnisz et al., 2015). To test whether these signaling factors are incorporated into condensates at super-enhancer associated genes, we performed RNA FISH for Nanog in combination with immunofluorescence for each of the three signaling factors (FIG. 52A). Nanog, a gene important for pluripotency, is associated with a super-enhancer occupied by these three signaling factors and Mediator in mouse embryonic stem cells (mESCs) as shown by ChIP-sequencing (FIG. 52B). We found that condensed foci could be observed for all three factors at the Nanog locus in individual cells (FIG. 52A), suggesting that all three factors are incorporated into super-enhancer associated condensates. Similar results were obtained at an additional super-enhancer locus where transcriptional condensates have been demonstrated to occur in mESCs (Boija et al, 2018; Sabari et al., 2018) (FIG. 58A, B). To confirm that the association of signaling factors with this locus is cell type-specific, we investigated whether β-catenin condensed foci overlapped with Nanog in C2C12 myoblast cells using a combination of immunofluorescence and DNA FISH; no β-catenin signal was detected at this locus in C2C12 cells (FIG. 58C). These results are consistent with the idea that signaling factors are incorporated into cell type-specific super-enhancer condensates. To confirm that the β-catenin, STAT3 and SMAD3 signaling factors are incorporated into nuclear condensates upon pathway stimulation, we performed immunofluorescence for those factors in mESCs in the presence or absence of the stimulus for each signaling pathway. We found that all three signaling factors were detected as condensed nuclear foci by immunofluorescence when their respective signaling pathways were activated (FIG. 52C). These results indicate that β-catenin, SMAD3 and STAT3 are incorporated into nuclear condensates upon pathway activation.

The condensates formed by transcription factors and Mediator at super-enhancers exhibit liquid-like behavior (Boija et al., 2018; Cho et al., 2018; Sabari et al., 2018). A hallmark of liquid-liquid phase-separated condensates is dynamic internal re-organization and rapid exchange kinetics (Banani et al., 2017; Hyman et al., 2014; Shin and Brangwynne, 2017), which can be interrogated by measuring the rate of fluorescence recovery after photobleaching (FRAP). To test whether signaling factors exhibit this type of behavior, we introduced a mEGFP-tag at the endogenous locus of the β-catenin gene in constitutive WNT-activated HCT116 cells, confirmed that the levels of mEGFP-tagged β-catenin expressed in these cells were similar to those normally expressed in these cells (FIG. 58D), and examined the behavior of these condensates by FRAP. The β-catenin nuclear puncta recovered on a time-scale of seconds (FIG. 52D), with an approximate apparent diffusion coefficient of 0.004±0.003 μm²/s. These values are similar to those of previously described components of liquid-like condensates (Nott et al., 2015; Pak et al., 2016, Sabari et al., 2018), indicating that condensates containing β-catenin exhibit liquid-like properties.

Purified Signaling Factors can Form Condensates In Vitro

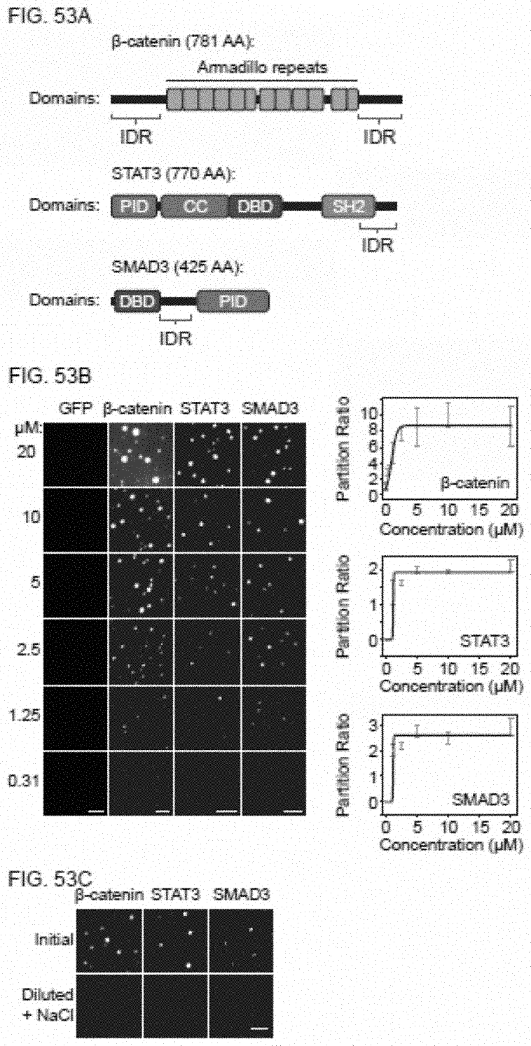

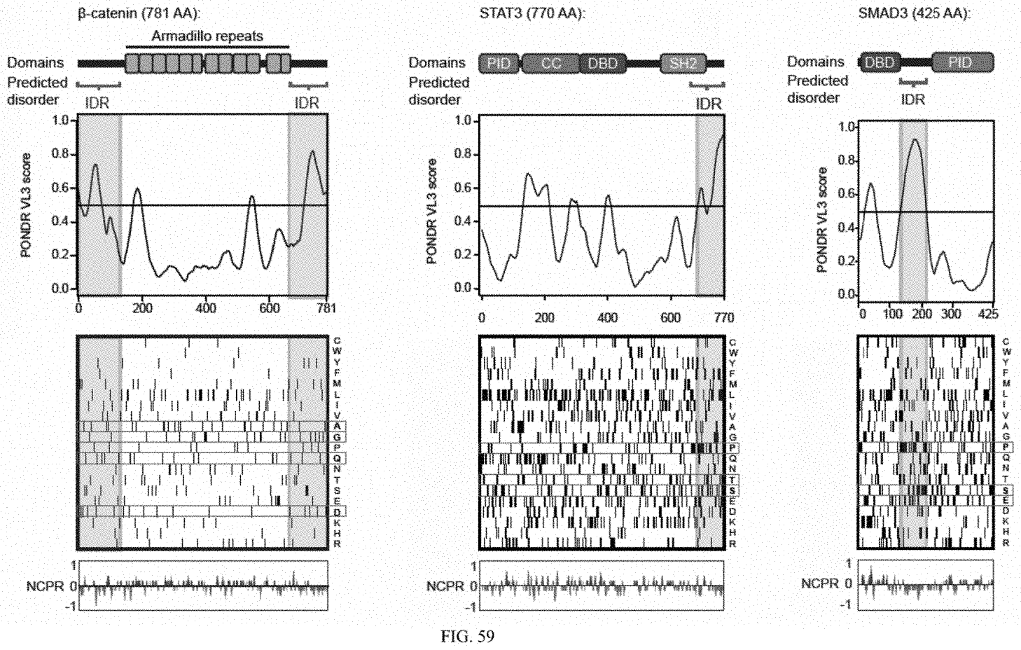

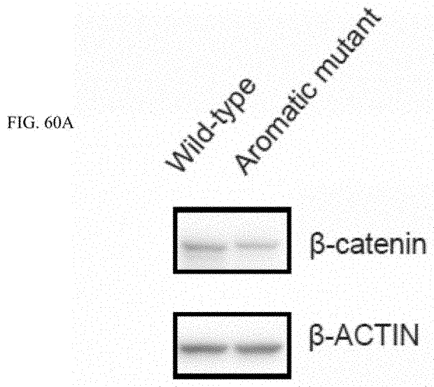

An analysis of the amino acid sequences of β-catenin, STAT3 and SMAD3 revealed that they contain intrinsically disordered regions (IDRs) (FIG. 53A, FIG. 59). Because IDRs are capable of forming dynamic networks of weak interactions and have been implicated in condensate formation (Burke et al., 2015; Lin et al., 2015; Nott et al., 2015), we investigated whether these signaling proteins could form phase-separated droplets in vitro. Indeed, purified recombinant mEGFP-β-catenin, mEGFP-STAT3 and mEGFP-SMAD3, formed concentration-dependent droplets (FIG. 53B). The droplets were spherical, micron-sized and freely moved in solution. The droplet forming behavior of these proteins exhibited a switch in partition ratio between the dense and dilute phases at micromolar concentrations, consistent with behavior of proteins that undergo phase separation (FIG. 53B). Further characterization of these droplets revealed that they were reversible by dilution and sensitive to increased salt concentration (FIG. 53C), behaviors characteristic of liquid-liquid phase-separated droplets.

Purified Signaling Factors are Incorporated into Mediator Condensates In Vitro

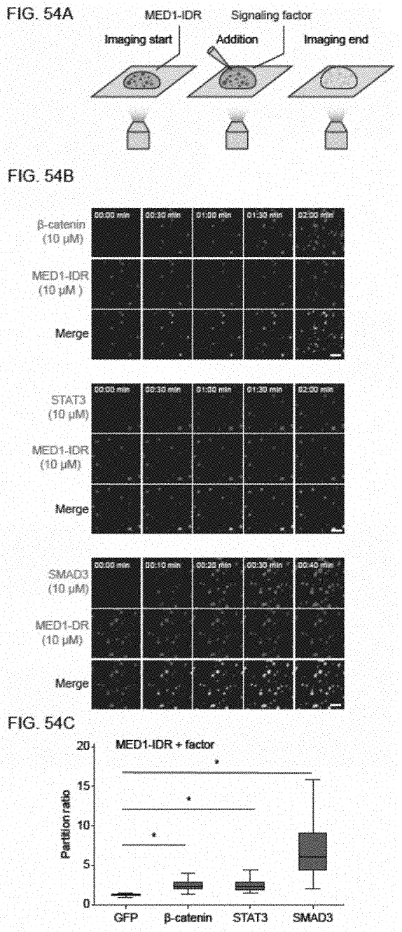

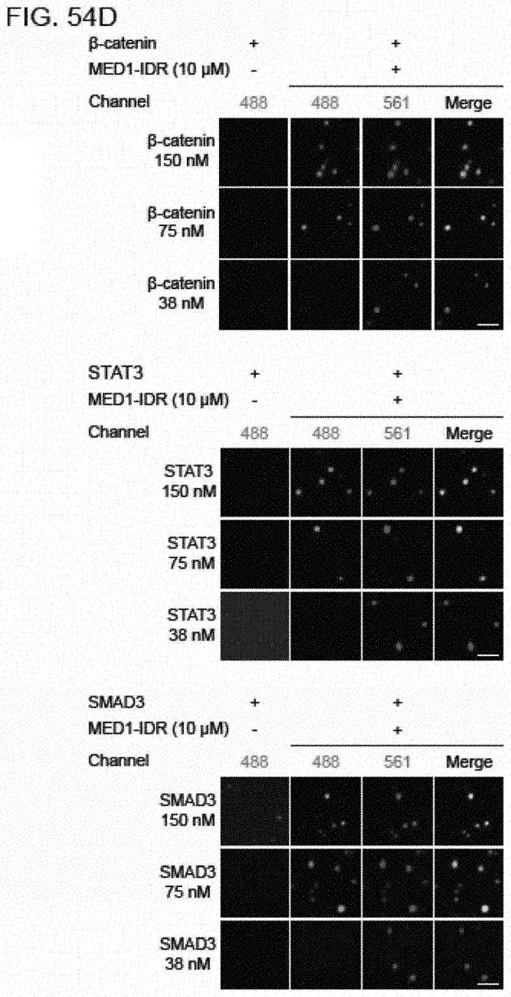

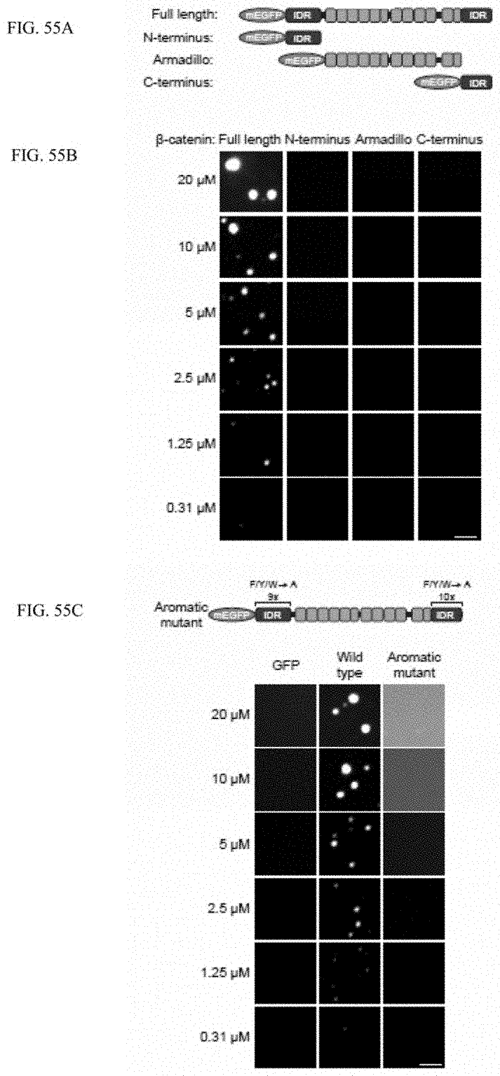

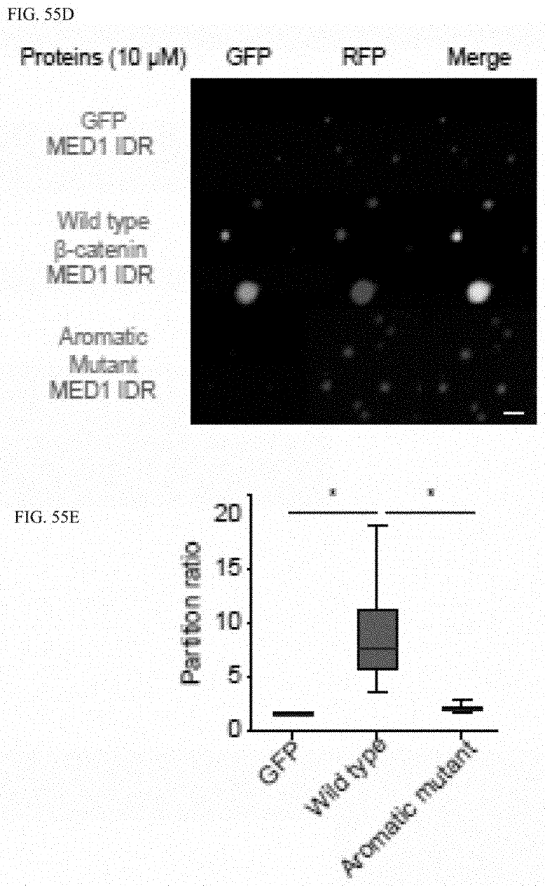

The transcriptional condensates formed at super-enhancers contain high concentrations of the Mediator coactivator, and transcription factors interact with Mediator through the same residues that are important for phase separation of their activation domains (Sabari et al., 2018; Boija et al., 2018). Given the droplet forming properties of β-catenin, SMAD3 and STAT3 and their localization in vivo, we reasoned that these signaling proteins might also interact with, and be concentrated into, Mediator condensates. To test this idea we used MED1-IDR, a surrogate for Mediator complex (Boija et al., 2018), to form droplets in PEG-8000, added dilute signaling factors to the solution, and monitored the incorporation of signaling factors into MED1-IDR droplets (FIG. 54A). We found that β-catenin, SMAD3 and STAT3 were incorporated and concentrated in MED1-IDR droplets (FIG. 54B, C).

β-catenin, SMAD3 and STAT3 are found at nanomolar concentrations in mammalian cells (Beck et al., 2017), but the concentrations at which the recombinant signaling proteins form droplets in vitro are in the micromolar range (FIG. 53B). This led us to investigate if signaling factors can form droplets at nanomolar concentrations in the presence of Mediator, where they do not form detectable droplets of their own. In these assays, the signaling factors were also efficiently partitioned into MED1-IDR droplets (FIG. 54D). These results are consistent with the possibility that partitioning of signaling factors into Mediator condensates contributes to the localization of signaling factors to transcriptional condensates at super-enhancers.

Phase Separation of β-Catenin and Activation of Target Genes are Dependent on Aromatic Amino Acids

If the enrichment of signaling factors at super-enhancers occurs, through the phase separation properties of their IDRs and incorporation into Mediator condensates, then mutations in the IDRs that affect their ability to form phase-separated droplets in vitro would be expected to affect their ability to target and activate genes in vivo. To test this hypothesis, we focused further studies on β-catenin and sought to identify portions of the protein responsible for its phase separation properties. β-catenin consists of a central, structured domain with Armadillo repeats surrounded by an N-terminal IDR and a C-terminal IDR (FIG. 55A). Droplet assays showed that recombinant proteins containing only the Armadillo repeats or the N-terminal or C-terminal IDRs were not capable of phase separating at any of the concentrations tested (FIG. 55B), suggesting that these components alone do not contribute to the phase separation properties of the intact protein and that both IDRs are required for this behavior.

We next focused attention on the amino acid residues within the two IDRs that might contribute to condensation, and noted an abundance of aromatic residues (FIG. 59). We generated a mutant form of β-catenin where the aromatic residues in both IDRs were substituted with alanines (FIG. 55C). These types of mutations perturb pi-cation interactions, which play an important role in the phase separation capacity of multiple proteins (Frey et al., 2018; Wang et al., 2018). When tested in a droplet formation assay, the mutant form of β-catenin was unable to form droplets except at very high concentrations, where very small droplets were observed (FIG. 55C). When tested in a heterotypic droplet forming assay with MED1-IDR, the mutant β-catenin protein failed to incorporate and concentrate into MED1-IDR droplets (FIG. 55D, E). These results suggest that the aromatic residues in the IDRs of β-catenin contribute to its phase separation behavior.

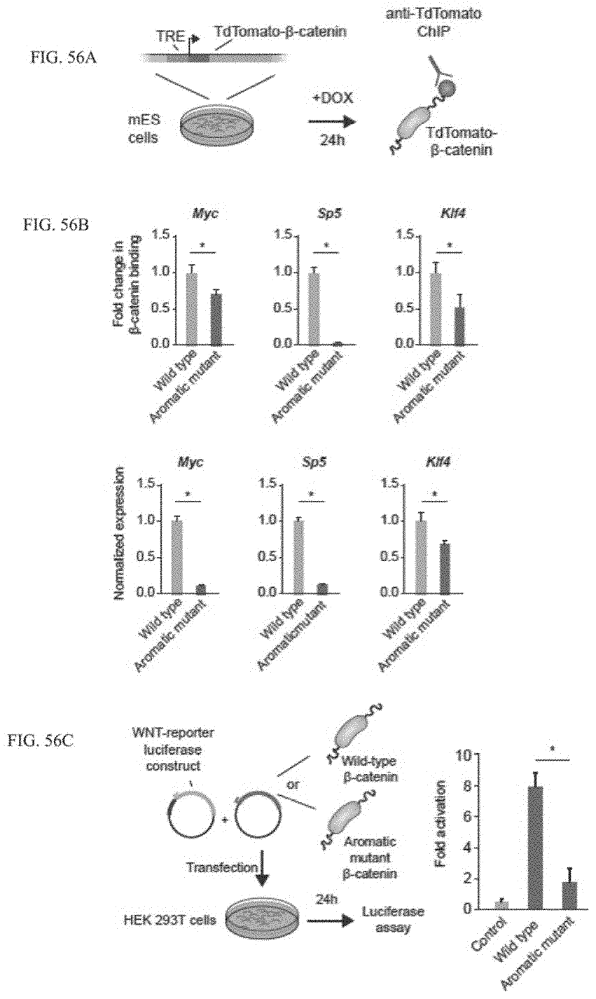

To test whether the aromatic residues in the IDRs contribute to β-catenin's function in vivo, constructs encoding TdTomato-tagged wild type and mutant forms of β-catenin, under control of a doxycycline-inducible promoter, were integrated into the genome of mESCs (FIG. 56A) and ChIP-qPCR for β-catenin was performed after activation by doxycycline. Wild type β-catenin was found to occupy the WNT-responsive genes Myc, Spy and Klf4, as expected, while lower levels of the aromatic mutant were found at these enhancers (FIG. 56B). This differential occupancy was reflected in lower levels of expression from these genes (FIG. 56B). These results suggest that the aromatic amino acids in the β-catenin IDRs are necessary for both condensate formation and for β-catenin's proper association and function at enhancers in vivo.

We independently tested the ability of the β-catenin aromatic mutant to transactivate a WNT-responsive reporter gene in a luciferase assay with wild type and mutant forms of β-catenin (FIG. 56C). Expression of wild type β-catenin stimulated an 8-fold increase in luciferase activity, whereas expression of the aromatic mutant had little effect on the luciferase reporter (FIG. 56C). These results further support the notion that β-catenin amino acids necessary for condensate formation with Mediator in vitro are also important for gene activation in vivo.

Sequences of Beta-Catenin Used Herein:

Beta-Catenin N-terminal IDR sequence:
(SEQ ID NO: 249)
Gctactcaagctgatttgatggagttggacatggccatggaaccagacag
aaaagcggctgttagtcactggcagcaacagtcttacctggactctggaa
tccattctggtgccactaccacagctccttctctgagtggtaaaggcaat
cctgaggaagaggatgtggatacctcccaagtcctgtatgagtgggaaca
gggattttctcagtccttcactcaagaacaagtagctgatattgatggac
agtatgcaatgactcgagctcagagggtacgagctgctatgttccctgag
acattagatgagggcatgcagatcccatctacacagtttgatgctgctca
tcccactaatgtccagcgtttggctgaaccatcacagatgctg
>Beta-catenin C-terminal IDR Sequence:
(SEQ ID NO: 250)
Ccacaagattacaagaaacggctttcagttgagctgaccagctctctctt
cagaacagagccaatggcttggaatgagactgctgatcttggacttgata
ttggtgcccagggagaaccccttggatatcgccaggatgatcctagctat
cgttcttttcactctggtggatatggccaggatgccttgggtatggaccc
catgatggaacatgagatgggtggccaccaccctggtgctgactatccag
ttgatgggctgccagatctggggcatgcccaggacctcatggatgggctg
cctccaggtgacagcaatcagctggcctggtttgatactgacctg
>Beta-catenin N-terminal IDR with Aromatic
residues converted to Alanine:
(SEQ ID NO: 251)
Gctactcaagctgatttgatggagttggacatggccatggaaccagacag
aaaagcggctgttagtcacgcgcagcaacagtctgccctggactctggaa
tccattctggtgccactaccacagctccttctctgagtggtaaaggcaat
cctgaggaagaggatgtggatacctcccaagtcctggctgaggcggaaca
gggagcttctcagtccgccactcaagaacaagtagctgatattgatggac
aggctgcaatgactcgagctcagagggtacgagctgctatggcccctgag
acattagatgagggcatgcagatcccatctacacaggctgatgctgctca
tcccactaatgtccagcgtttggctgaaccatcacagatgctg
>Beta-catenin C--terminal IDR with Aromatic
residues converted to Alanine:
(SEQ ID NO: 252)
Ccacaagatgccaagaaacggctttcagttgagctgaccagctctctcgc
cagaacagagccaatggctgcgaatgagactgctgatcttggacttgata
ttggtgcccagggagaaccccttggagctcgccaggatgatcctagcgct
cgttctgctcactctggtggagctggccaggatgccttgggtatggaccc
catgatggaacatgagatgggtggccaccaccctggtgctgacgctccag
ttgatgggctgccagatctggggcatgcccaggacctcatggatgggctg
cctccaggtgacagcaatcagctggccgcggctgatactgacctg

β-Catenin-Condensate Interaction can Occur Independently of TCF Factors

β-catenin does not have DNA-binding activity and the conventional model for β-catenin recruitment to genes involves a structured interaction between its Armadillo repeats and a TCF/LEF family DNA-binding transcription factor. If β-catenin is recruited to Mediator condensates through dynamic interactions that allow β-catenin to condense in vivo, then this should occur in the absence of TCF/LEF factors. We developed a series of assays to test this idea.

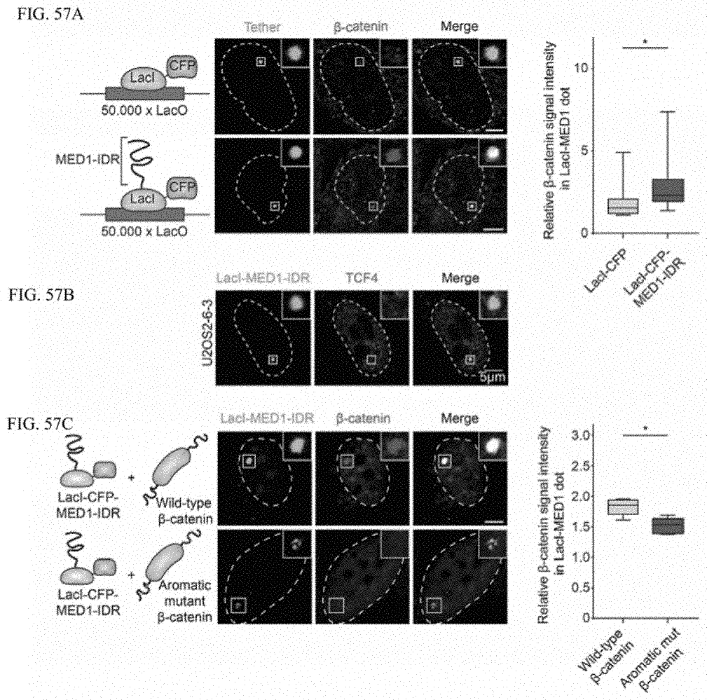

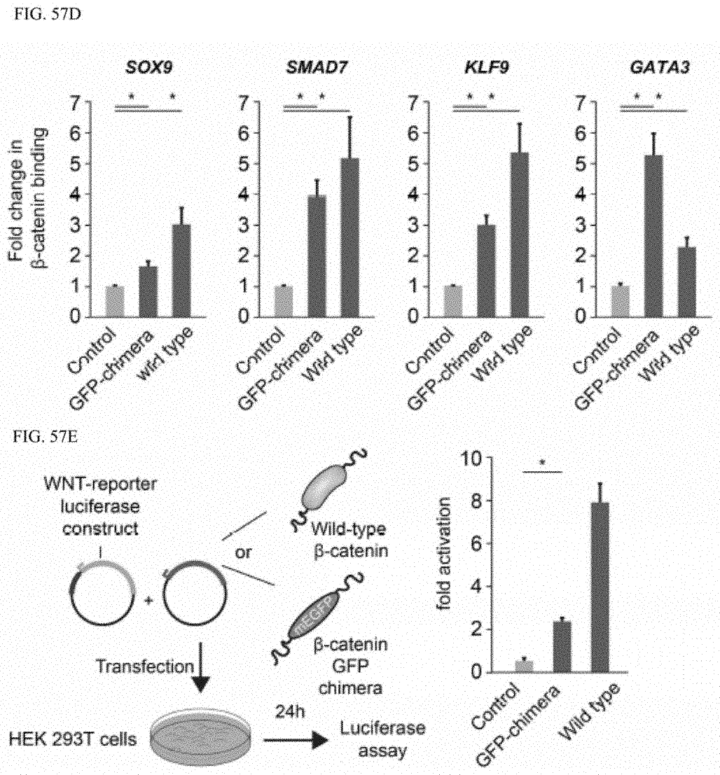

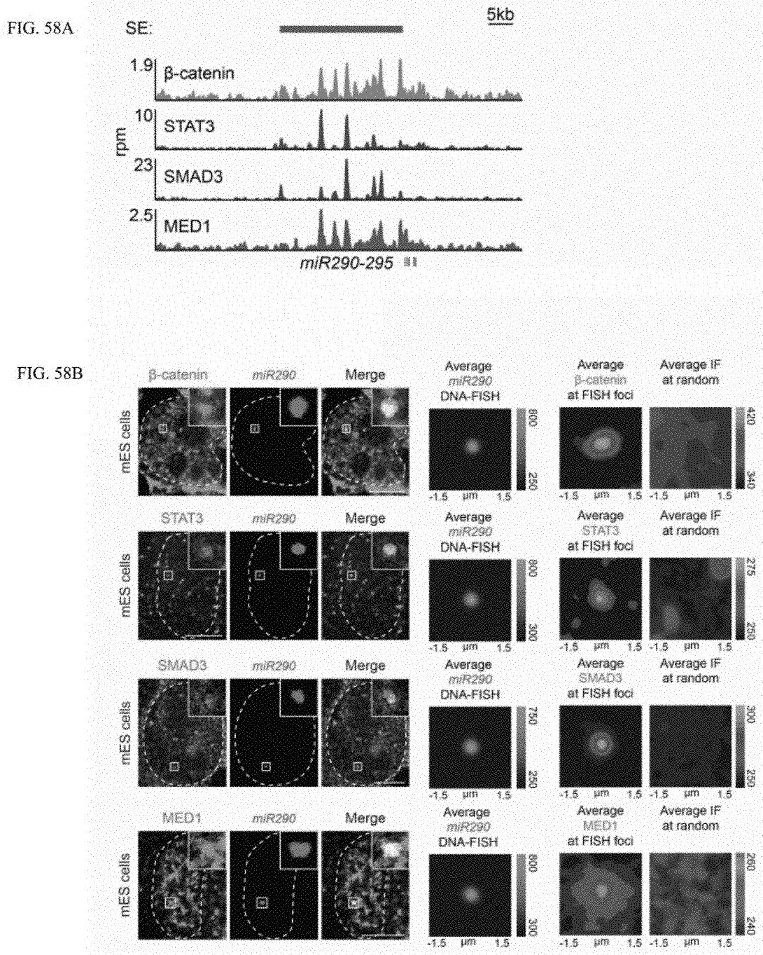

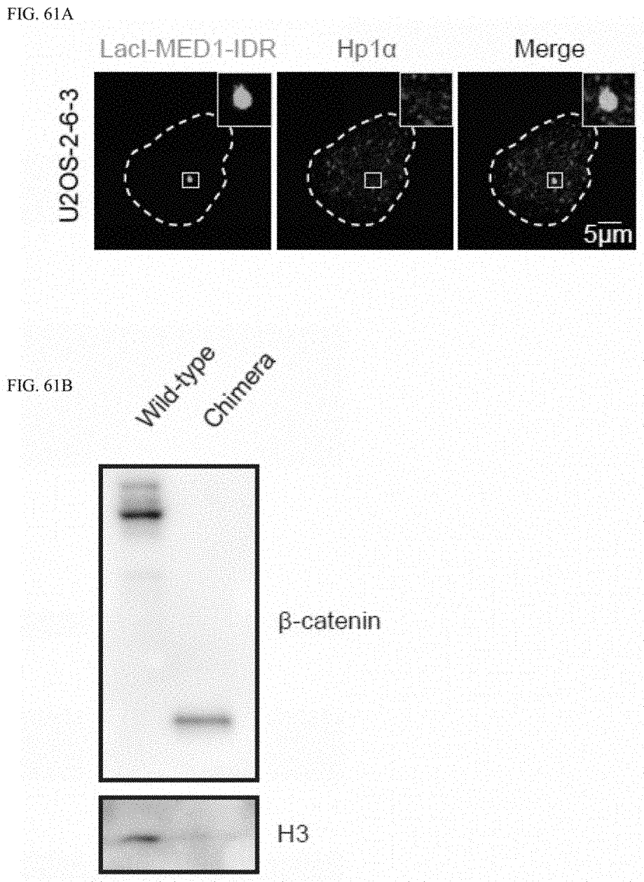

We first investigated whether β-catenin could be incorporated into MED1 condensates in vivo by using a condensate assay that was originally developed to study nuclear speckles (Janicki et al., 2004) (FIG. 57A). The MED1-IDR was tethered to an array of Lad binding sites in U2OS cells, which have a constitutively activated WNT signaling pathway (Chen et al., 2015) and thus have detectable levels of β-catenin in the nucleus. Cells were transiently transfected with either LacI-MED1-IDR or control LacI. The LacI-MED1-IDR, but not Lad alone, was found to recruit endogenous β-catenin to the lac array (FIG. 57A). This effect was likely not mediated through interactions with TCF/LEF and direct interaction with DNA because the lac array does not contain TCF motifs and no TCF4 was detected at the LacI-MED1-IDR foci by IF (FIG. 57B). The heterochromatin binding protein HP1a served as a control and was not recruited to the array either (FIG. 61A). When TdTomato-labeled wild type and aromatic mutant β-catenin were ectopically expressed, the TdTomato-labeled wild type β-catenin accumulated at the MED1-IDR occupied lac array, while accumulation of the TdTomato-labeled aromatic mutant was significantly reduced (FIG. 57C). These results suggest that β-catenin is incorporated into MED1-IDR condensates in vivo in the absence of TCF4 and in a manner that is dependent on the same amino acids that are required for β-catenin to be incorporated and concentrated into MED1 condensates in vitro.

To further test if the regions of β-catenin that allow it to phase separate with Mediator are sufficient to address β-catenin to specific genomic loci in the absence of an interaction with TCF/LEF factors, we engineered a β-catenin-chimera protein where the armadillo repeats, including the TCF interaction domain, were replaced with mEGFP. The β-catenin-chimera was integrated into HEK293T cells under the control of a doxycycline inducible promoter. ChIP-qPCR for GFP showed enrichment for β-catenin-chimera at the WNT-driven genes SOX9, SMAD7, KLF9 and GATA3 indicating that the IDRs of β-catenin are sufficient to address mEGFP to specific genomic loci (FIG. 57D). This effect was not due to differences in expression of these factors as the chimera expressed at comparable levels as the wild type form of β-catenin (FIG. 61B). The C-terminal IDR of β-catenin contains its transactivation domain, so we sought to investigate if the β-catenin-chimera might also be able to activate transcription as well as localize to the correct genomic locations. When the β-catenin-chimera was over-expressed in a luciferase reporter assay it was able to activate a WNT-reporter, although this activation was lower than the wild type form of β-catenin (FIG. 57E). These data are consistent with the idea that β-catenin can be recruited to a Mediator condensate through its ability to interact with this condensate and independent of its classical interaction with TCF/LEF factors.

Discussion

Diverse cell types employ a small set of shared, developmentally-important signaling pathways to transmit extracellular information to adjust gene expression programs accordingly (Perrimon et al., 2012). In any one cell type, effector components of the WNT, TGF-β and JAK/STAT pathways connect to only a small subset of a large number of potential signal response elements, preferring to bind those in active enhancers formed by the master transcription factors of that cell type, thus producing cell type-specific responses (David and Massagué, 2018; Hnisz et al., 2015; Mullen et al., 2011; Trompouki et al., 2011). The mechanisms that have been described to account for this bias include preferential access to “open chromatin” (Mullen et al, 2011), to altered DNA structures caused by binding of other TFs, and cooperative protein-protein interactions with master TFs (Hallikas et al., 2006; Kelly et al., 2011). The observation that signaling factors have a special preference for cell type-specific super-enhancers (Hnisz et al., 2015), coupled with the finding that TFs and Mediator form phase-separated condensates at super-enhancers (Boija et al., 2018; Cho et al., 2018; Sabari et al., 2018), led us to investigate whether signaling factors have properties that facilitate partitioning into transcriptional condensates at super-enhancers. The evidence described here argues that the cell type-dependent specificity of signaling may be achieved, at least in part, by addressing signaling factors to transcriptional condensates through phase separation at super-enhancers. In this manner, multiple signaling factor molecules could be concentrated in such condensates and occupy appropriate sites on the genome.

We find that the signaling factors β-catenin, STAT3 and SMAD3 occur in condensed puncta at signal-responsive super-enhancers in ESCs, where transcriptional condensates have been reported to contain hundreds of molecules of Mediator and RNA polymerase II (Boija et al., 2018; Cho et al., 2018; Sabari et al., 2018). These signaling factors can be incorporated and concentrated into Mediator subunit condensates in vitro, suggesting that their ability to enter Mediator condensates might contribute to their preferential association with Mediator condensates found at super-enhancers in vivo. Indeed, tethering a Mediator subunit to an array of genomic sites forms a condensate that can recruit at least one of these signaling factors, β-catenin, to the condensate and does so in the absence of a structured interaction with its classic partner, the DNA-binding factor TCF4. Importantly, mutations in residues that reduce β-catenin-Mediator condensate incorporation in vitro likewise reduce the ability of β-catenin to enter Mediator subunit condensates in vivo and to activate transcription.

The model we describe for β-catenin entry into super-enhancer condensates may help explain additional conundrums in the signaling literature. For example, β-catenin has been reported to interact with a large number of different proteins (Schuijers et al., 2014) and this interaction promiscuity has resulted in the proposal that a large number of DNA-binding transcription factors have the capacity to recruit β-catenin in addition to the canonical recruiters of the TCF/LEF family (Nateri et al., 2005; Kouzmenko et al, 2004; Essers et al., 2005; Kaidi et al., 2007; Botrugno et al., 2004; Kelly et al., 2011; Sinner et al., 2004). However, the majority of these reported interactions were not supported by functional data and only binding to TCF has been supported by co-crystallization (Poy et al., 2001; Sampietro et al., 2006). Our model might explain how β-catenin could functionally interact with a large number of TFs in a transcriptional condensate, yet fail to activate transcription in an artificial system where such a condensate might not be assembled.

The condensate model described here may facilitate further understanding of pathological signaling in diseases such as cancer. Dysregulated transcription and signaling are in fact two hallmarks of cancer (Bradner et al., 2017). Cancer cells develop genomic alterations that create super-enhancers at driver oncogenes (Chapuy et al., 2013; Hnisz et al., 2013; Lin et al., 2016; Mansour et al., 2014; Zhang et al., 2016), and these oncogenes are especially responsive to oncogenic signaling (Hnisz et al., 2015). The signaling factors that contribute to oncogenic signaling may generally interact with super-enhancer condensates through properties that also promote phase separation. In this way, tumor cells dependent on a particular signaling pathway could acquire resistance to therapies by employing alternative signaling pathways whose signaling factors could incorporate into transcriptional condensates. Perhaps therapies that target both oncogenic signaling pathways and super-enhancer components will prove especially effective in tumor cells that have signaling and transcriptional dependencies.

Star Methods

KEY RESOURCES TABLE

| | | |
|---|---|---|
| REAGENT or RESOURCE | SOURCE | IDENTIFIER |

Antibodies

| | | |
|---|---|---|
| GFP | Abcam | ab290 |
| Med1 | Abcam | ab64965 |
| β-catenin | Abcam | ab22656 |
| STAT3 | Santa Cruz | SC-7993 |
| SMAD3 | Santa Cruz | SC-6202 |
| DsRed | Takara | 632496 |

Chemicals, Peptides, and Recombinant Proteins

| | | |
|---|---|---|
| mEGFP | This manuscript | |
| mEGFP-β-catenin | This manuscript | |
| mEGFP-STAT3 | This manuscript | |
| mEGFP-SMAD3 | This manuscript | |
| mCherry-MED1-IDR | This manuscript | |
| mEGFP-β-catenin-N-terminus | This manuscript | |
| mEGFP-β-catenin-Armadillo | This manuscript | |
| mEGFP-β-catenin-C-terminus | This manuscript | |
| mEGFP-β-catenin-Aromatic-Mutant | This manuscript | |
| CHIR99021 | Stemgent | 04-0004 |
| Leukemia Inhibitory Factor (LIF) | ESGRO | ESG1107 |
| Activin A | R&D systems | 338-AC-010 |
| IWP2 | Sigma Aldrich | I0536 |
| SB431542 | Tocris Bioscience | 16-141 |

Critical Commercial Assays

| | | |
|---|---|---|
| Dual-glo Luciferase Assay System | Promega | E2920 |
| NEBuilder HiFi DNA Assembly Master Mix | NEB | E2621S |
| Power SYBR Green mix | Life Technologies | 4367659 |
| TaqMan Universal PCR Master Mix | Applied Biosystems | 4304437 |
| RNeasy Plus Mini Kit | QIAGEN | 74136 |
| Sp5 probe | Taqman® | Mm00491634_m1 |
| Myc probe | Taqman® | Mm00487804_m1 |
| Gapdh probe | Taqman® | Mm99999915_g1 |

Deposited Data

| | | |
|---|---|---|
| Med1 ChIP-seq | This manuscript | GSMxxxx |
| GFP-β-catenin ChIP-seq | This manuscript | GSMxxxx |

Experimental Models: Cell Lines

| | |
|---|---|
| V6.5 cells | Rudolf Jaenisch |
| β-catenin-GFP-tagged V6.5 cells | This manuscript |
| β-catenin-GFP-tagged HCT116 cells | This manuscript |
| C2C12 cells | ATCC |
| HEK293T cells | ATCC |
| TdTomato-wild-type-β-catenin V6.5 cells | This manuscript |
| TdTomato-aromatic-mutant-β-catenin V6.5 | This manuscript |
| cells | |
| U2OS-2-6-3 cells | Spektor Lab |
| GFP-chimera HEK293T cells | This manuscript |

Oligonucleotides

| | |
|---|---|
| ChIP-qPCR | |
| ChIP-negative-FWD | ACACAACATCTG |
| | CCCAAACA (SEQ |
| | ID NO: 226) |
| ChIP-negative-REV | TGAGATCCTGGT |
| | GTGACCAA (SEQ |
| | ID NO: 227) |
| Klf4-1-FWD | AGGGTGATGAA |
| | TGGATCAGG |
| | (SEQ ID NO: 228) |
| Klf4-1-REV | CTCTCCCCACGA |
| | ATTAACGA (SEQ |
| | ID NO: 229) |
| Myc-1-FWD | CCAGTGAACAA |
| | AAGTGCAA (SEQ |
| | ID NO: 230) |
| Myc-1-REV | TCCAGGCACATC |
| | TCAGTTTG (SEQ |
| | ID NO: 231) |
| Sp5-1-FWD | GGAGCTCGCTTT |
| | AGTCCTCA (SEQ |
| | ID NO: 232) |
| Sp5-1-REV | CCCCCACTTGCA |
| | ATTAAAGA (SEQ |
| | ID NO: 233) |
| ChIP-negative-hu-FWD | CTCCCTTCCATC |
| | TTCCCTTC (SEQ |
| | ID NO: 234) |
| ChIP-negative-hu-REV | TGCTTTCTTGGG |
| | GCATTAAC (SEQ |
| | ID NO: 235) |
| SOX9-FWD | CTGTTGGGAATT |
| | CAGCCAAT (SEQ |
| | ID NO: 236) |
| SOX9-REV | AATGAAGGGAG |
| | TGCAGGATG |
| | (SEQ ID NO: 237) |
| SMAD7-FWD | AAATCCATCGG |
| | GTATCTGGA |
| | (SEQ ID NO: 238) |
| SMAD7-REV | AGGCGGCCTCTT |
| | TTGTTTAT (SEQ |
| | ID NO: 239) |
| KLF9-FWD | GCTCTGAAACCT |
| | GGCTCATC (SEQ |
| | ID NO: 240) |
| KLF9-REV | ATTCTCTTGTCG |
| | GGTTGCAG (SEQ |
| | ID NO: 241) |
| GATA3-FWD | GGCTGACATCAC |
| | CCAGAGAT (SEQ |
| | ID NO: 242) |
| GATA3-REV | ACAGAAAAGAA |
| | GCCGGGAAT |
| | (SEQ ID NO: 243) |
| RT-qPCR | |
| Gapdh-FWD | CCATGTAGTTGA |
| | GGTCAATGAAG |
| | G (SEQ ID NO: |
| | 244) |
| Gapdh-REV | TGGTGAAGGTC |
| | GGTGTGAAC |
| | (SEQ ID NO: 245) |
| Klf4-FWD | CTCCCGTCCTTC |
| | TCCACGTT (SEQ |
| | ID NO: 246) |
| Klf4-REV | TTCCTCACGCCA |
| | ACGGTTA (SEQ |
| | ID NO: 247) |

Recombinant DNA

| | |
|---|---|
| pJM101-PiggyBac-BetaCat-FL | This manuscript |
| pJM102-PiggyBac-BetaCat-AromaticMut | This manuscript |
| pJS-21-mEGFP-Bcat-repair-mo | This manuscript |
| pJS-22-mEGFP-Bcat-repair-hu | This manuscript |
| pX330-GFP-B-catenin | This manuscript |

Software and Algorithms

| | | |
|---|---|---|
| Fiji image processing package | Schindelin et al., | https://fiji.sc/ |
| | 2012 | |
| MetaMorph acquisition software | Molecular Devices | https://www.moleculardevices.com/ |
| | | products/cellular- |
| | | imaging- |
| | | systems/acquisition- |
| | | and-analysis- |
| | | software/metamorph- |
| | | microscopy |
| PONDR | http://www.pondr.com/ | N/A |
| MACS | Zhang et al., 2008 | |
| Bowtie | Langmead et al., | |
| | 2009 | |

Other

| | | |
|---|---|---|
| Nanog RNA FISH probe | Stellaris | N/A |
| miR290 RNA FISH probe | Stellaris | N/A |
| Nanog DNA FISH probe | Agilent | N/A |

Experimental Model and Subject Details

Cell Lines

V6.5 murine embryonic stem cells were a gift from Jaenisch lab. HEK293T and HCT116 cells were obtained from ATCC. U2OS cells were obtained from the Spector lab. Cells were routinely tested for mycoplasm.

Cell Culture Conditions

V6.5 murine embryonic stem cells were grown on 2i+LIF conditions on 0.2% gelatinized (Sigma, G1890) tissue culture plates. The media used for 2i+LIF media conditions is as follows: 967.5 mL DMEM/F12 (GIBCO 11320), 5 mL N2 supplement (GIBCO 17502048), 10 mL B27 supplement (GIBCO 17504044), 0.5 mM L-glutamine (GIBCO 25030), 0.5× non-essential amino acids (GIBCO 11140), 100 U/mL Penicillin-Streptomycin (GIBCO 15140), 0.1 mM β-mercaptoethanol (Sigma), 1 uM PD0325901 (Stemgent 04-0006), 3 uM CHIR99021 (Stemgent 04-0004), and 1000 U/mL recombinant LIF (ESGRO ESG1107). HEK293T, U2OS and HCT116 cells were cultured in DMEM, high glucose, pyruvate (GIBCO 11995-073) with 10% fetal bovine serum (Hyclone, characterized SH3007103), 100 U/mL Penicillin-Streptomycin (GIBCO 15140), 2 mM L-glutamine (Invitrogen, 25030-081).

Cell Line Stimulation

For WNT: Cells were treated with either CHIR99021 or IWP2 (Sigma Aldrich 10536) for 24 hrs in 2i+LIF medium without CHIR (mES) or with CHIR in 10% FBS DMEM medium (HEK293).

For SMAD3: Cells were treated with ActivinA (R&D systems 338-AC-010) or SB431542 (Tocis Bioscience 16-141) for 24 hours in 2i+LIF medium. For STAT3: Cells were treated with 2i+LIF or 2i−LIF medium for 24 hours

Cell Line Generation

V6.5 murine embryonic stem cells, HCT116 colorectal cancer cells or HEK293T embryonic kidney cells were genetically modified using the CRISPR-Cas9 system. A guide targeting the N-terminus of beta catenin was cloned into a px330 vector with an mCherry selectable marker and the following sequence: CTGCGTGGACAATGGCTACT (SEQ ID NO: 248). A repair template with 800 bp homology to the endogenous locus flanking an mEGFP-tag was cloned into a pUC19 vector. Cells were transfected with 2.5 μg of both constructs and sorted for mCherry two days post-transfection and sorted again for mEGFP one week post-transfection. Cells were serially diluted and colonies were picked to obtain clonal cell lines.

FRAP

FRAP was performed on LSM880 Airyscan microscope with 488 nm laser. Bleaching was performed over a r_{bleach}≈1 um using 100% laser power and images were collected every two seconds. Fluorescence intensity was measured using FIJI. Background intensity was subtracted and values are reported relative to pre-bleaching time points.

Custom MATLAB™ scripts were written to process the intensity data, accounting for background photobleaching and normalization to pre-bleach intensity. Post bleach FRAP recovery data was averaged over 9 replicates for each cell-line and condition. The FRAP recovery curve was fit to:

$$FRAP ( t ) = M ( 1 - exp ( - \frac{t}{τ} ) )$$

Immunofluorescence

Cells were fixed in 4% paraformaldehyde for 10 mins at RT as described in Sabari et al. 2018. Cells were then washed three times and permeabilized with 0.5 TritonX 100 in PBS for 5 min at RT. Following three washes in PBS cells were blocked in 4% Bovine Serum Albumin for 15 mins at RT and incubated with primary antibodies in 4% BSA overnight at room temperature. After three washes in PBS, cells were incubated in secondary antibodies in 4% BSA in the dark for 1 hour. Cells were washed three times with PBS followed by an incubation with Hoechst for 5 mins at RT in the dark. Slides were mounted with Vectashield H-1000 and coverslips were sealed with transparent nail polish and stored at 4 C. Images were acquired using an RPI Spinning Disk confocal microscope with a 100× objective using a Metamorph software and a CCD camera.

Co-Immunofluorescence with DNA FISH

Immunofluorescence was performed as described earlier with modifications to the protocol following incubation with secondary antibodies. After secondary antibodies cells were washed 3 times in PBS at RT and then fixed with 4% PFA in PBS for 20 mins and washed three times with PBS. Cells were incubated in 70% ethanol, 85% ethanol and then 100% ethanol for 1 min at RT. Probe hybridization mixture was made with 7 μl of FISH Hybridization Buffer (Agilent G9400A), 1 μl of FISH probes and 2 μl of water. 5 μl of mixture was added on a slide and coverslip was placed on top. Coverslip was sealed using rubber cement. Once rubber cement solidified genomic DNA and probes were denatured at 78 C for 5 mins and slides were incubated at 16 C in the dark overnight. Coverslips were removed from the slide and incubated in a pre-warmed Wash Buffer 1 at 73 C for 3 mins and in Wash Buffer 2 for 1 min at RT. Slides were air dried and nuclei stained with Hoechst in PBS for 5 mins at RT. Coverslips were washed three times in PBS, mounted on a slide using Vectashield H-1000 and sealed with nail polish. Images were acquired using an RPI Spinning DIsk confocal microscope with a 100× objective using the MetaMorph acquisition software and a Hammamatsu ORCA-ER CCD Camera. DNA FISH probes were custom designed and generated by Agilent to target the Nanog locus.

Co-Immunofluorescence with RNA FISH

Immunofluorescence was performed as previously described (Sabari et al., 2018) with the small modifications. Immunofluorescence was performed in an RNase-free environment, pipettes and bench were treated with RNaseZap (Life Technologies, AM9780). RNase free PBS was used and antibodies were diluted in RNase-free PBS at all times. After immunofluorescence completion, cells were post-fixed with 4% PFA in PBS for 10 min at RT. Cells were washed twice with RNase-free PBS. Cells were washed once with 20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) for 5 min at RT. Cells were hybridized with 90% Stellaris RNA FISH Hybridization Buffer (Biosearch Technologies, SMF-HB1-10), 10% Deionized Formamide, 12.5 μM Stellaris RNA FISH probes designed to hybridize introns of the transcripts of SE-associated genes. Hybridization was performed overnight at 37° C. Cells were then washed with Wash Buffer A for 30 min at 37° C. and nuclei were stained with 20 μm/ml HOESCHT in Wash Buffer A for 5 min at RT. After one 5-min was with Stellaris RNA FISH Wash Buffer B (Biosearch Technologies, SMF-WB1-20) at room temperature. Coverslips were mounted as described for immunofluorescence. Images were acquired at the RPI Spinning Disk confocal microscope with 100× objective using MetaMorph acquisition software and a Hammamatsu ORCA-ER CCD camera. Primary antibodies used were anti-MED1 Abcam ab64965 1:500 dilution, anti-b catenin Abcam ab22656 1:500 dilution, anti-pSTAT3 Santa Cruz 1:20 dilution, anti-SMAD2/3 Santa Cruz 1:20 dilution). Secondary antibodies used were anti-Rabbit IgG, anti-goat IgG and anti-mouse IgG.

Average Image Analysis

For analysis of RNA FISH with immunofluorescence, custom MATLAB™ scripts were written to process and analyze 3D image data gathered in RNA FISH and IF channels. FISH foci were identified in individual z-stacks through intensity and size thresholds, centered along a box of size l=2.9 μm and stitched together in 3-D across z-stacks. For every FISH focus identified, signal from the corresponding location in the IF channel is gathered in the l×l square centered at the RNA FISH focus at every corresponding z-slice. The IF signal centered at FISH foci for each FISH and IF pair are then combined and an average intensity projection is calculated, providing averaged data for IF signal intensity within a l×l square centered at FISH foci. The same process was carried out for the FISH signal intensity centered on its own coordinates, providing averaged data for FISH signal intensity within a l×l square centered at FISH foci. As a control, this same process was carried out for IF signal centered at randomly selected nuclear positions. For each replicate, 40 random nuclear points were generated from the interior of the nuclear envelope, identified from the DAPI channel by a combination of large size (200 voxels) and intensity (DNA dense) thresholds. These average intensity projections were then used to generate 2D contour maps of the signal intensity. Contour plots are generated using built-in functions in MATLAB™. For the contour plots, the intensity-color ranges presented were customized across a linear range of colors (n!=15). For the FISH channel, black to magenta was used. For the IF channel, we used chroma.js (an online color generator) to generate colors across 15 bins, with the key transition colors chosen as black, blueviolet, mediumblue, lime. This was done to ensure that the reader's eye could more readily detect the contrast in signal. The generated colormap was employed to 15 evenly spaced intensity bins for all IF plots. The averaged IF centered at FISH or at randomly selected nuclear locations are plotted using the same color scale, set to include the minimum and maximum signal from each plot.

Protein Purification

cDNA encoding the genes of interest or their IDRs were cloned into a modified version of a T7 pET expression vector. The base vector was engineered to include a 5′ 6×HIS followed by either mEGFP or mCherry and a 14 amino acid linker sequence “GAPGSAGSAAGGSG.” (SEQ ID NO: 14) NEBuilder® HiFi DNA Assembly Master Mix (NEB E2621S) was used to insert these sequences (generated by PCR) in-frame with the linker amino acids. Vectors expressing mEGFP or mCherry alone contain the linker sequence followed by a STOP codon. Mutant sequences were synthesized as geneblocks (IDT) and inserted into the same base vector as described above. All expression constructs were sequenced to ensure sequence identity.

For protein expression plasmids were transformed into LOBSTR cells (gift of Chessman Lab) and grown as follows. A fresh bacterial colony was inoculated into LB media containing kanamycin and chloramphenicol and grown overnight at 37° C. Cells containing the MED1-IDR constructs were diluted 1:30 in 500 ml room temperature LB with freshly added kanamycin and chloramphenicol and grown 1.5 hours at 16° C. IPTG was added to 1 mM and growth continued for 18 hours. Cells were collected and stored frozen at −80° C. Cells containing all other constructs were treated in a similar manner except they were grown for 5 hours at 37° C. after IPTG induction.

Pellets of 500 ml of Beta Catenin mutant cells were resuspended in 15 ml of denaturing buffer (50 mM Tris 7.5, 300 mM NaCl, 10 mM imidazole, 8M Urea) containing cOmplete protease inhibitors (Roche, 11873580001) and sonicated (ten cycles of 15 seconds on, 60 sec off). The lysates were cleared by centrifugation at 12,000 g for 30 minutes and added to 1 ml of pre-equilibrated Ni-NTA agarose (Invitrogen, R901-15). Tubes containing this agarose lysate slurry were rotated for 1.5 hours at room temperature. The slurry was centrifuged at 3,000 rpm for 10 minutes in a Thermo Legend XTR swinging bucket rotor. The pellets were washed 2× with 5 ml of lysis buffer followed by centrifugation 10 minutes at 3,000 rpm as above. Protein was eluted 3× with 2 ml of the lysis buffer with 250 mM imidazole. For each cycle the elution buffer was added and rotated at least 10 minutes and centrifuged as above. Eluates were analyzed on a 12% acrylamide gel stained with Coomassie. Fractions containing protein of the expected size were pooled, diluted 1:1 with the 250 mM imidazole buffer and dialyzed first against buffer containing 50 mM Tris pH 7.5, 125 Mm NaCl, 1 mM DTT and 4M Urea, followed by the same buffer containing 2M Urea and lastly 2 changes of buffer with 10% Glycerol, no Urea. Any precipitate after dialysis was removed by centrifugation at 3.000 rpm for 10 minutes. MED1-IDR and WT Beta Catenin were purified in a similar manner except the lysis buffer contained no urea, the incubations were done at 4 C and dialysis was into 2 changes of 50 mM Tris pH7.5, 125 mM NaCl, 10% glycerol and 1 mM DTT.

In Vitro Droplet Formation Assay

Recombinant GFP or mCherry fusion proteins were concentrated and desalted to an appropriate protein concentration and 125 mM NaCl using Amicon Ultra centrifugal filters (30K MWCO, Millipore). Recombinant proteins were added to solutions at varying concentrations with indicated final salt and 10% PEG-8000 as crowding agent in Droplet Formation Buffer (50 mM Tris-HCl pH 7.5, 10% glycerol, 1 mM DTT). The protein solution was immediately loaded onto a homemade chamber comprising a glass slide with a coverslip attached by two parallel strips of double-sided tape. Slides were then imaged with an Andor confocal microscope with a 150× objective. Unless indicated, images presented are of droplets settled on the glass coverslip.

Coverslips were coated with PEG-silane in order to neutralize charge. In brief, coverslips were washed with 2% Helmanex III for 2 hours, washed with H₂O three times and washed with ethanol once before being incubated in 0.5% PEG-silane in ethanol with 1% Acetic Acid over night. They were then washed with ethanol once and sonicated in a water bath sonicator for 15 minutes in ethanol, washed with H₂O for three times before being rinsed with ethanol and dried to the air.

Heterotypic Droplet Analysis

To analyze in vitro droplet experiments, custom Python scripts using the scikit-image package were written to identify droplets and characterize their size, shape, and intensity. Droplets were segmented from average images of captured channels on various criteria: (1) an intensity threshold three standard deviations above the mean of the image, (2) size thresholds (9 pixel minimum droplet size), (3) and a minimum circularity

$$( circularity = 4 π * \frac{area}{{perimiter}^{2}} )$$

of 0.8 (1 being a perfect circle). After segmentation, mean intensity for each droplet was calculated while excluding pixels near the phase interface (Banani et al., 2016). Hundreds of droplets identified in typically 5-10 independent fields of view were quantified. The mean intensity within the droplets (C-in) and in the bulk (C-out) were calculated for each channel. The partition ratio was computed as (C-in)/(C-out). The box plots show the distributions of all droplets. The measured datasets for partition ratio versus the protein concentration in FIG. 2b were fitted by the logistic equation (Wang et al., 2018):

$$f = \frac{a}{1 + {e}^{\frac{( x - x 0 )}{b}}}$$

Where f is the partition ratio and x is the corresponding protein concentration.

RT-qPCR

RNA was isolated using the Rneasy Plus Mini Kit (QIAGEN, 74136) according to manufacturer's instructions. cDNA was generated using SuperScript II Reverse Transcriptase (Invitrogen, 18080093) with oligo-dT primers (Promega, C1101) according to manufacturer's instructions. Quantitative real-time PCR was performed on Applied Biosystems 7000, QuantStudio5 and QuantStudio6 instruments using TaqMan probes for SE genes.

ChIP

Cells were plated at a density of 4-5 million cells per plate and harvested 24-48 hours after. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1500 g for 5 minutes at 4° C., resuspended in LB1 (50 mM Hepes-KOH, pH7.9, 140 mM NaCl, 1 mM EDTA 0.5 mL 0.5M, 10% glycerol, 0.5% NP40, 1% TritonX-100, 1× protease inhibitor) and incubate for 20 minutes rotating at 4° C. Cells were pelleted for 5 minutes at 1350 g, resuspended in LB2 (10 mM Tris pH 8.0, 200 mM NaCl, 1 mM EDTA, 0.5 mM EGTA, lx protease inhibitor) and incubated for 5 minutes rotating at 4° C. Pellet was resuspended in LB3 (10 mM Tris pH 8.0, 100 mM NaCl, 1 mM EDTA, 0.5 mM EGTA, 0.1% sodium-deoxycholate, 0.5% sodium lauroyl sarcosinate, 1% TritonX-100, lx protease inhibitor) at a concentration of 30-50 million cells/ml. Cells were sonicated using Covaris S220 for 12 minutes using the manufacturer's instructions followed by spinning at 20 000 g for 30 minutes at 4° C. Dynabeads pre-blocked with 0.5% BSA were incubated with GFP antibody (Abcam, ab290), Med1 antibody (Abcam, ab64965) or dsRed (Takara, 632496) antibody for 6 hours. Chromatin was added to antibody-bead complex and incubated rotating overnight at 4° C. Beads were washed three times with each Wash buffer 1 (50 mM Hepes pH7.5, 500 mM NaCl, 1 mM EDTA, 1 mM EGTA, 1% Triton, 0.1% NaDoc, 0.1% SDS) and Wash Buffer 2 (20 mM Tris pH 8, 1 mM EDTA, 250 mM LiCl, 0.5% NP40, 0.5% NaDoc) at 4° C., followed by washing one time with TE at room temperature. Chromatin was eluted by adding Elution buffer (50 mM, Tris pH 8.0, 10 mM EDTA, 1% sodium dodecyl sulfate, 20 ug/ml RNaseA) to the beads and incubated shaking at 60° C. for 30 minutes. Reversal of crosslinking was performed for 4 hours at 58° C. Proteinase K was added and incubated for 1-2 hours at 37° C. for protein removal. DNA was purified using Qiagen PCR purification kit and resuspended in 10 mM Tris-HCL. ChIP Libraries were prepared with the Swift Biosciences Accel-NGS® 2S Plus DNA Library Kit according to kit instructions with an additional size selection step on the PippinHT system from Sage Science. Following library prep, ChIP libraries were run on a 2% gel on the PippinHT with a size collection window of 200-600 bases. Final libraries were quantified by qPCR with the KAPA Library Quantification kit from Roche and sequenced in single-read mode for 40 bases on an Illumina HiSeq 2500.

ChIP-Seq Analysis

ChIP-Seq data were aligned to the mm9 version of the mouse reference genome using bowtie with parameters -k 1 -m 1 -best and -l set to read length. Wiggle files for display of read coverage in bins were created using MACS with parameters -w -S space=50 -nomodel -shiftsize=200, and read counts per bin were normalized to the millions of mapped reads used to make the wiggle file (Zhang et al., 2008). Reads-per-million normalized wiggle files were displayed in the UCSC genome browser (Kent et al., 2002).

### REFERENCES

Banani, S. F., Lee, H. O., Hyman, A. A., and Rosen, M. K. (2017). Biomolecular condensates: Organizers of cellular biochemistry. Nat. Rev. Mol. Cell Biol. 18, 285-298.
Beck, M., Schmidt, A., Malmstroem, J., Claassen, M., Ori, A., Szymborska, A., Herzog, F., Rinner, O., Ellenberg, J., and Aebersold, R. (2011). The quantitative proteome of a human cell line. Mol. Syst. Biol. 7, 1-8.
Boija, A., Klein, I. A., Sabari, B. R., Dall'Agnese, A., Coffey, E. L., Zamudio, A. V., Li, C. H., Shrinivas, K., Manteiga, J. C., Hannett, N. M., et al. (2018). Transcription Factors Activate Genes through the Phase-Separation Capacity of Their Activation Domains. Cell 1-14.
Botrugno, O. A., Fayard, E., Annicotte, J.-S., Haby, C., Brennan, T., Wendling, O., Tanaka, T., Kodama, T., Thomas, W., Auwerx, J., et al. (2004). Synergy between LRH-1 and beta-catenin induces G1 cyclin-mediated cell proliferation. Mol. Cell 15, 499-509.
Bradner, J. E., Hnisz, D., and Young, R. A. (2017). Transcriptional Addiction in Cancer. Cell 168, 629-643.
Burke, K. A., Janke, A. M., Rhine, C. L., and Fawzi, N. L. (2015). Residue-by-Residue View of In Vitro FUS Granules that Bind the C-Terminal Domain of RNA Polymerase II. Mol. Cell 60, 231-241.
Chapuy, B., McKeown, M. R., Lin, C. Y., Monti, S., Roemer, M. G. M., Qi, J., Rahl, P. B., Sun, H. H., Yeda, K. T., Doench, J. G., et al. (2013). Discovery and characterization of super-enhancer-associated dependencies in diffuse large B cell lymphoma. Cancer Cell 24, 777-790.
Chen, C., Zhao, M., Tian, A., Zhang, X., Yao, Z., and Ma, X. (2015). Aberrant activation of Wnt/B-catenin signaling drives proliferation of bone sarcoma cells. Oncotarget 6, 17570-17583.
Cho, W. K., Spille, J. H., Hecht, M., Lee, C., Li, C., Grube, V., and Cisse, I. I. (2018). Mediator and RNA polymerase II clusters associate in transcription-dependent condensates. Science (80-). 361, 412-415.
Darnell, J., Kerr, I., and Stark, G. (1994). Jak-STAT pathways and transcriptional activation in response. Science (80-). 264, 1415-1421.
David, C. J., and Massagué, J. (2018). Contextual determinants of TGFβ action in development, immunity and cancer. Nat. Rev. Mol. Cell Biol. 19, 419-435.
Essers, M. A. G., de Vries-Smits, L. M. M., Barker, N., Polderman, P. E., Burgering, B. M. T., and Korswagen, H. C. (2005). Functional interaction between beta-catenin and FOXO in oxidative stress signaling. Science 308, 1181-1184.
Farley, E. K., Olson, K. M., Zhang, W., Brandt, A. J., Rokhsar, D. S., and Levine, M. S. (2015). Suboptimization of developmental enhancers. Science 350, 325-328.
Frey, S., Rees, R., Schünemann, J., Ng, S. C., Fünfgeld, K., Huyton, T., and Görlich, D. (2018). Surface Properties Determining Passage Rates of Proteins through Nuclear Pores. Cell 174, 202-217.e9.
Hallikas, O., Palin, K., Sinjushina, N., Rautiainen, R., Partanen, J., Ukkonen, E., and Taipale, J. (2006). Genome-wide prediction of mammalian enhancers based on analysis of transcription-factor binding affinity. Cell 124, 47-59.
Hnisz, D., Abraham, B. J., Lee, T. I., Lau, A., Saint-André, V., Sigova, A. A., Hoke, H. A., and Young, R. A. (2013). Super-enhancers in the control of cell identity and disease. Cell 155, 934-947.
Hnisz, D., Schuijers, J., Lin, C. Y., Weintraub, A. S., Abraham, B. J., Lee, T. I., Bradner, J. E., and Young, R. A. (2015). Convergence of Developmental and Oncogenic Signaling Pathways at Transcriptional Super-Enhancers. Mol. Cell 58, 362-370.
Hyman, A. A., Weber, C. A., and Jülicher, F. (2014). Liquid-Liquid Phase Separation in Biology. Annu. Rev. Cell Dev. Biol. 30, 39-58.
Janicki, S. M., Tsukamoto, T., Salghetti, S. E., Tansey, W. P., Sachidanandam, R., Prasanth, K. V., Ried, T., Shav-Tal, Y., Bertrand, E., Singer, R. H., et al. (2004). From silencing to gene expression: Real-time analysis in single cells. Cell 116, 683-698.
Kaidi, A., Williams, A. C., and Paraskeva, C. (2007). Interaction between β-catenin and HIF-1 promotes cellular adaptation to hypoxia. Nat. Cell Biol. 9, 210-217.
Kelly, K. F., Ng, D. Y., Jayakumaran, G., Wood, G. A., Koide, H., and Doble, B. W. (2011). β-Catenin Enhances Oct-4 Activity and Reinforces Pluripotency through a TCF-Independent Mechanism. Cell Stem Cell 8, 214-227.
Kent W J, Sugnet C W, Furey T S, Roskin K M, Pringle T H, Zahler A M, Haussler D. (2002). The human genome browser at UCSC. Genome Res. 12(6), 996-1006.
Kouzmenko, A. P., Takeyama, K. I., Ito, S., Furutani, T., Sawatsubashi, S., Maki, A., Suzuki, E., Kawasaki, Y., Akiyama, T., Tabata, T., et al. (2004). Wnt/β-catenin and estrogen signaling converge in vivo. J. Biol. Chem. 279, 40255-40258.
Langmead, B., Trapnell, C., Pop, M., and Salzberg, S. L. (2009). Ultrafast and memory-efficient alignment of short DNA sequences to the human genome. Genome Biol. 10.
Lee, T. I., and Young, R. A. (2013). Transcriptional regulation and its misregulation in disease. Cell 152, 1237-1251.
Lin, C. Y., Erkek, S., Tong, Y., Yin, L., Federation, A. J., Zapatka, M., Haldipur, P., Kawauchi, D., Risch, T., Warnatz, H.-J., et al. (2016). Active medulloblas-toma enhancers reveal subgroup-specific cellular origins. Nature 530, 57-62.
Lin, Y., Protter, D. S. W., Rosen, M. K., and Parker, R. (2015). Formation and Maturation of Phase-Separated Liquid Droplets by RNA-Binding Proteins. Mol. Cell 60, 208-219.
Mansour, M. R., Abraham, B. J., Anders, L., Berezovskaya, A., Gutierrez, A., Durbin, A. D., Etchin, J., Lee, L., Sallan, S. E., Silverman, L. B., et al. (2014). An oncogenic super-enhancer formed through somatic mutation of a noncoding intergenic element. Science (80-). 346, 1373-1377.
Molenaar, M., Van De Wetering, M., Oosterwegel, M., Peterson-Maduro, J., Godsave, S., Korinek, V., Roose, J., Destrée, O., and Clevers, H. (1996). XTcf-3 transcription factor mediates β-catenin-induced axis formation in xenopus embryos. Cell 86, 391-399.
Mullen, A. C., Orlando, D. A., Newman, J. J., Lovén, J., Kumar, R. M., Bilodeau, S., Reddy, J., Guenther, M. G., Dekoter, R. P., and Young, R. A. (2011). Master transcription factors determine cell-type-specific responses to TGF-β signaling. Cell 147, 565-576.
Mullen, A. C., and Wrana, J. L. (2017). TGF-β family signaling in embryonic and somatic stem-cell renewal and differentiation. Cold Spring Harb. Perspect. Biol. 9
Nateri, A. S., Spencer-Dene, B., and Behrens, A. (2005). Interaction of phosphorylated c-Jun with TCF4 regulates intestinal cancer development. Nature 437, 281-285.
Nott, T. J., Petsalaki, E., Farber, P., Jervis, D., Fussner, E., Plochowietz, A., Craggs, T. D., Bazett-Jones, D. P., Pawson, T., Forman-Kay, J. D., et al. (2015). Phase Transition of a Disordered Nuage Protein Generates Environmentally Responsive Membraneless Organelles. Mol. Cell 57, 936-947.
Nusse, R., and Clevers, H. (2017). Wnt/β-Catenin Signaling, Disease, and Emerging Therapeutic Modalities. Cell 169, 985-999.
Nüsslein-volhard, C., and Wieschaus, E. (1980). Mutations affecting segment number and polarity in drosophila. Nature 287, 795-801.
Pak, C. W., Kosno, M., Holehouse, A. S., Padrick, S. B., Mittal, A., Ali, R., Yunus, A. A., Liu, D. R., Pappu, R. V., and Rosen, M. K. (2016). Sequence Determinants of Intracellular Phase Separation by Complex Coacervation of a Disordered Protein. Mol. Cell 63, 72-85.
Perrimon, N., Pitsouli, C., and Shilo, B. (2012). Signaling Mechanisms Controlling Cell Fate and Embryonic Patterning. Cold Spring Harb. Perspect. Biol. 4, 1-18.
Poy, F., Lepourcelet, M., Shivdasani, R. A., and Eck, M. J. (2001). Structure of a human Tcf4-β-catenin complex. Nat. Struct. Biol. 8, 1053-1057.
Rawlings, J. S. (2004). The JAK/STAT signaling pathway. J. Cell Sci. 117, 1281-1283.
Sabari, B. R., Dall'Agnese, A., Boija, A., Klein, I. A., Coffey, E. L., Shrinivas, K., Abraham, B. J., Hannett, N. M., Zamudio, A. V, Manteiga, J. C., et al. (2018). Coactivator condensation at super-enhancers links phase separation and gene control. Science 361, eaar3958.
Sampietro, J., Dahlberg, C. L., Cho, U. S., Hinds, T. R., Kimelman, D., and Xu, W. (2006). Crystal Structure of a β-Catenin/BCL9/Tcf4 Complex. Mol. Cell 24, 293-300.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: An open-source platform for biological-image analysis. Nat. Methods 9, 676-682.
Schuijers, J., Mokry, M., Hatzis, P., Cuppen, E., and Clevers, H. (2014). Wnt-induced transcriptional activation is exclusively mediated by TCF/LEF. EMBO J. 33, 146-156.
Shin, Y., and Brangwynne, C. P. (2017). Liquid phase condensation in cell physiology and disease. Science 357, 2415-2423.
Small, S., Blair, A., and Levine, M. (1992). Regulation of even-skipped stripe 2 in the Drosophila embryo. EMBO J. 11, 4047-4057.
Sinner, D., Rankin, S., Lee, M., and Zorn, A. M. (2004). Sox17 and beta-catenin cooperate to regulate the transcription of endodermal genes. Development 131, 3069-3080.
Takahashi, K., and Yamanaka, S. (2016). A decade of transcription factor-mediated reprogramming to pluripotency. Nat. Rev. Mol. Cell Biol. 17, 183-193.
Takahashi, K., Yamanaka, S., Zhang, Y., Li, Y., Feng, C., Li, X., Lin, L., Guo, L., Wang, H., Liu, C., et al. (2006). Induction of Pluripotent Stem Cells from Mouse Embryonic and Adult Fibroblast Cultures by Defined Factors. Cell 126, 663-676.
Theunissen, T. W., and Jaenisch, R. (2014). Molecular control of induced pluripotency. Cell Stem Cell 14, 720-734.
Trompouki, E., Bowman, T. V., Lawton, L. N., Fan, Z. P., Wu, D. C., Dibiase, A., Martin, C. S., Cech, J. N., Sessa, A. K., Leblanc, J. L., et al. (2011). Lineage regulators direct BMP and Wnt pathways to cell-specific programs during differentiation and regeneration. Cell 147, 577-589.
Wang, J., Choi, J. M., Holehouse, A. S., Lee, H. O., Zhang, X., Jahnel, M., Maharana, S., Lemaitre, R., Pozniakovsky, A., Drechsel, D., et al. (2018). A Molecular Grammar Governing the Driving Forces for Phase Separation of Prion-like RNA Binding Proteins. Cell 1-12.
Weintraub, H., Tapscott, S. J., Davis, R. L., Thayer, M. J., Adam, M. A., Lassar, A. B., and Miller, A. D. (1989). Activation of muscle-specific genes in pigment, nerve, fat, liver, and fibroblast cell lines by forced expression of MyoD. Proc. Natl. Acad. Sci. 86, 5434-5438.
van de Wetering, M., Cavallo, R., Dooijes, D., van Beest, M., van Es, J., Loureiro, J., Ypma, A., Hursh, D., Jones, T., Bejsovec, A., et al. (1997). Armadillo Coactivates Transcription Driven by the Product of the Drosophila Segment Polarity Gene dTCF. Cell 88, 789-799.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Yan, R., Small, S., Desplan, C., Dearolf, C. R., and Darnell, J. E. (1996). Identification of a Stat gene that functions in Drosophila development. Cell 84, 421-430.
Yingling, J. M., Datto, M. B., Wong, C., Frederick, J. P., Liberati, N. T., and Wand, X.-F. (1997). Tumor suppressor, Smad-4, is a TGF-beta inducible, DNA binding protein. Mol Cell Biol 17, 7019-7028.
Zhang, X., Choi, P. S., Francis, J. M., Imielinski, M., Watanabe, H., Cherniack, A. D., and Meyerson, M. (2016). Identification of focally amplified lineage-specific super-enhancers in human epithelial cancers. Nat. Genet. 48, 176-182.
Zhang, Y., Liu, T., Meyer, C. A., Eeckhoute, J., Johnson, D. S., Sv. D., Bernstein, E. C., Nusbaum, B., Myers, R. M., Brown, M., Li, W., Liu, X. S. (2008). Model-based analysis of ChIP-Seq (MACS). Genome Biol. 9, R137.
Zhu, F., Farnung, L., Kaasinen, E., Sahu, B., Yin, Y., Wei, B., Dodonova, S. O., Nitta, K. R., Morgunova, E., Taipale, M., et al. (2018). The interaction landscape between transcription factors and the nucleosome. Nature 562, 76-81.

### Example 6

Both transcription initiation machinery and splicing machinery can form phase-separated condensates containing large numbers of component molecules; hundreds of Pol II and Mediator complexes are concentrated in condensates at super-enhancers^{8,9} and large numbers of splicing factors are concentrated in nuclear speckles, some of which occur at highly active transcription sites¹⁰⁻¹⁷. Here we investigate whether phosphorylation of the CTD regulates its incorporation into phase-separated condensates associated with transcription initiation and splicing. We find that the hypophosphorylated Pol II CTD is incorporated into Mediator condensates and that phosphorylation by regulatory CDKs causes its eviction. We also find that the phosphorylated CTD is preferentially incorporated into condensates formed by splicing factors. These results suggest that Pol II CTD phosphorylation drives an exchange from condensates involved in transcription initiation to those involved in RNA processing and implicates phosphorylation as a mechanism to regulate condensate preference.

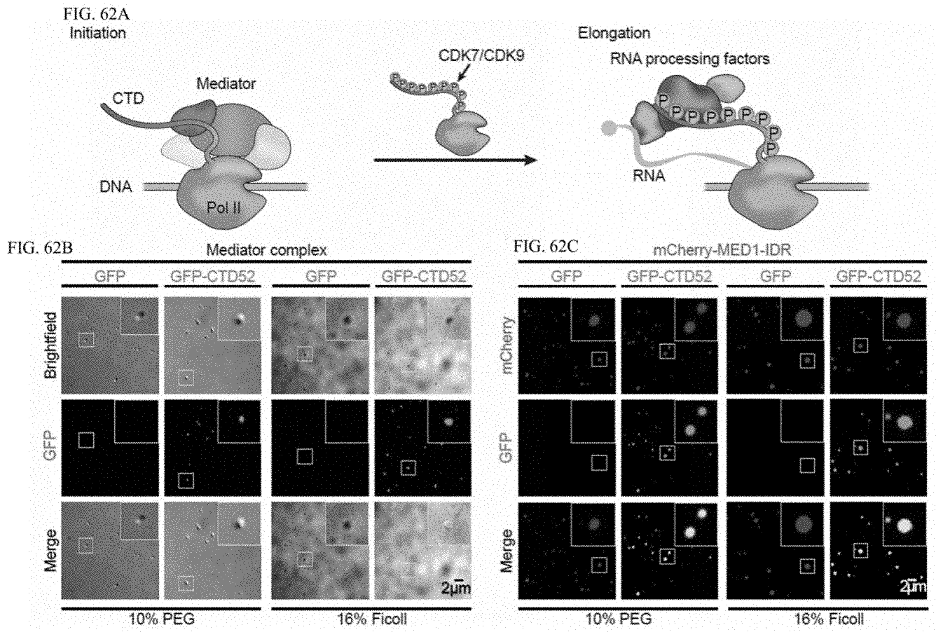

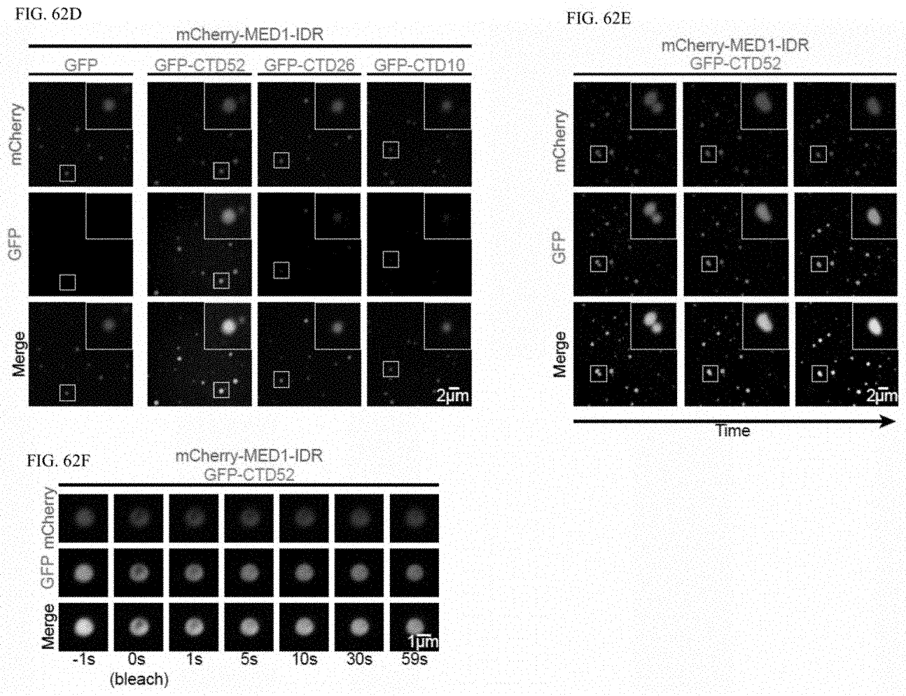

Studies have shown that the hypophosphorylated Pol II CTD can interact with Mediators⁵⁻⁷ and that Pol II and Mediator occur in condensates at super-enhancers^{8,9}. To investigate whether the Pol II CTD is incorporated into Mediator condensates, we purified the human Mediator complex and measured condensate formation in an in vitro droplet assay. Mediator droplets incorporated and concentrated human full-length CTD fused to GFP (GFP-CTD) but not control GFP (FIG. 62B). Crowding agents were used in these assays to mimic the crowded protein environment in cells, and to ensure that the observations were not specific to the agents used, we performed the same experiments and achieved identical results in the presence of two chemically distinct crowding agents (FIG. 62B). These results are consistent with the idea that the Pol II CTD contributes to its incorporation into Mediator condensates.

We further investigated the interaction of the CTD with Mediator by focusing our experiments on MED1, the largest subunit of the Mediator complexes¹⁸. We selected MED1 for further study because MED1 has proven to be a useful surrogate for the Mediator condensate in previous studies⁹. In addition, MED1 has an exceptionally large intrinsically disordered region (IDR) that contributes to condensate formation⁹ and MED1 has been shown to preferentially associate with Pol II in human cells¹⁹. Droplet assays revealed that MED1-IDR condensates incorporated and concentrated GFP-CTD (FIG. 62C), as observed for the Mediator complex (FIG. 62B). The ability of the CTD to enter the MED1-IDR condensates was impaired when the number of CTD heptapeptide repeats was reduced (FIG. 62D), as expected for an interaction that involves a high valency component, which is a feature of condensate-forming biomolecules^{20,21}. The Pol II CTD/MED1-IDR condensates exhibited liquid-like fusion behavior (FIG. 62E) and showed evidence of dynamic internal rearrangement and internal-external exchange of molecules by fluorescence recovery after photobleaching (FRAP; FIG. 62F), consistent with liquid-liquid phase-separated condensates.

The transition of Pol II from initiation to elongation is accompanied by phosphorylation of the CTD heptapeptide repeat by CDK7 and CDK9²²⁻²⁵. Phosphorylation of the CTD has been shown to affect its interaction with hydrogels formed by the low-complexity domains of PET (FUS/EWS/TAF15) proteins²⁶, suggesting that phosphorylation may affect the condensate interacting properties of the CTD.

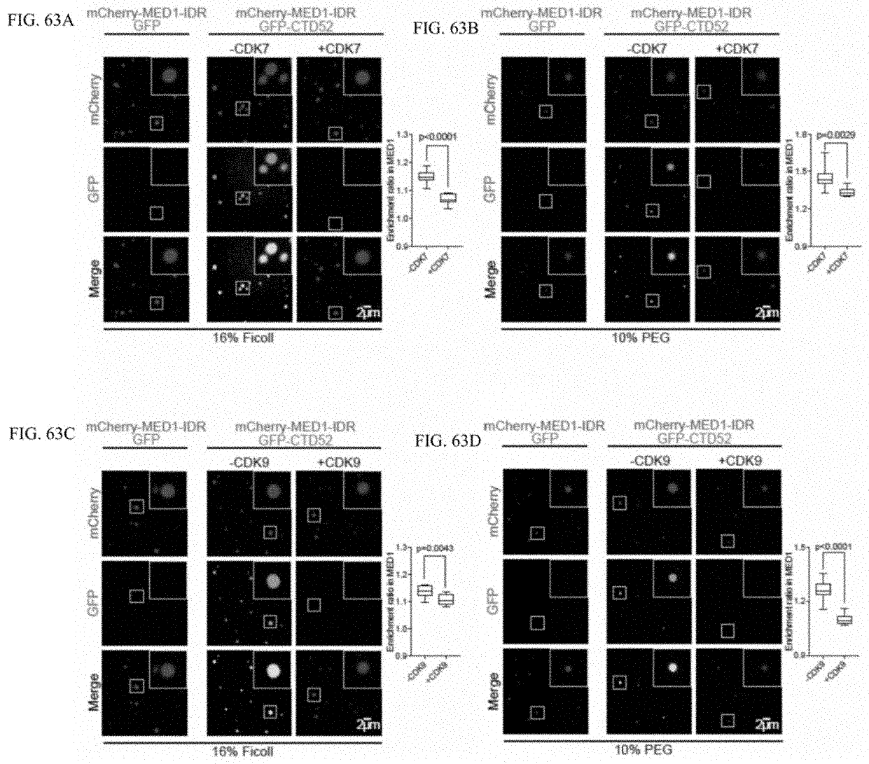

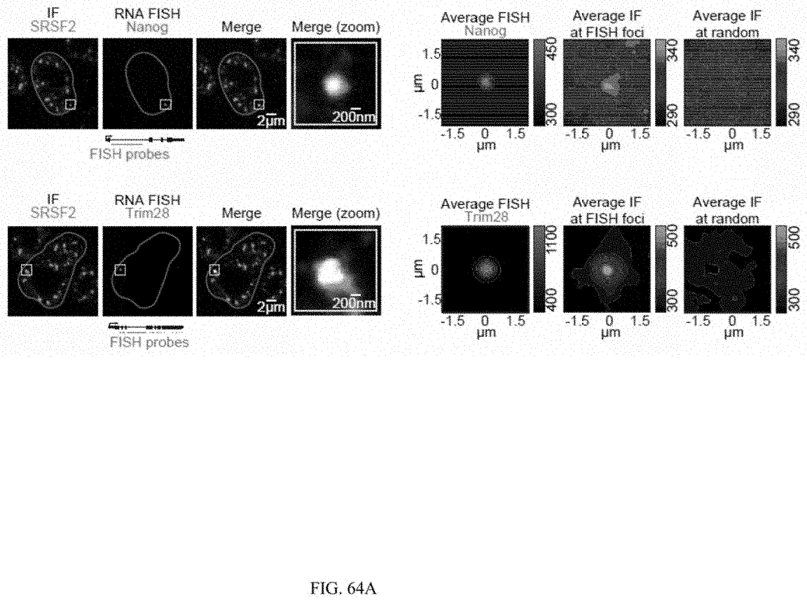

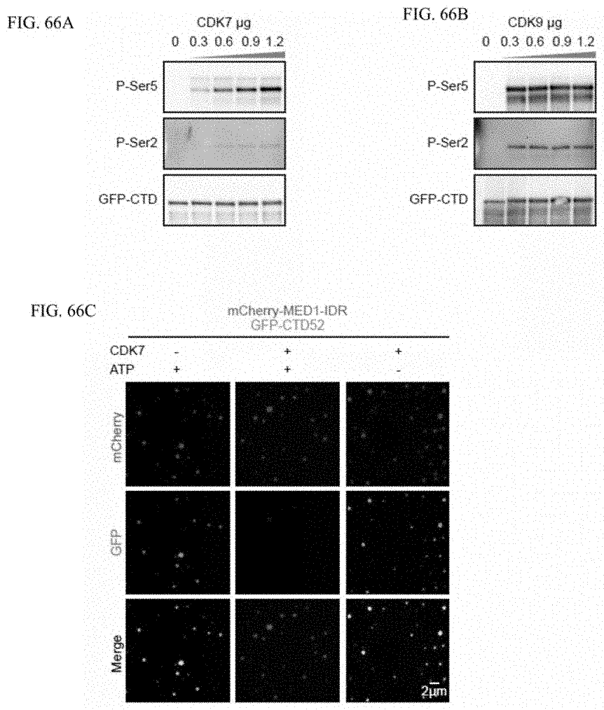

We investigated whether phosphorylation of the CTD by CDK7 or CDK9 would affect its incorporation into MED1-IDR condensates. CTD phosphorylation assays showed that CDK7 and CDK9 preparations could phosphorylate both serine 2 and 5 of recombinant CTD in vitro, with CDK7 showing a preference for serine 5 phosphorylation (FIG. 66A,B), in agreement with published results²²⁻²⁵. We found that CTD phosphorylation by CDK7 caused a significant reduction in CTD incorporation into MED1-IDR droplets (FIG. 63A,B; FIG. 66C), and this effect was independent of the crowding agent used (FIG. 63A,B). Similarly, phosphorylation by CDK9 caused a significant reduction in CTD incorporation into MED1-IDR droplets, and this was independent of the crowding agent used in the reaction (FIG. 63A,B). These results are consistent with the model that Pol II CTD phosphorylation causes eviction from a Mediator condensate.

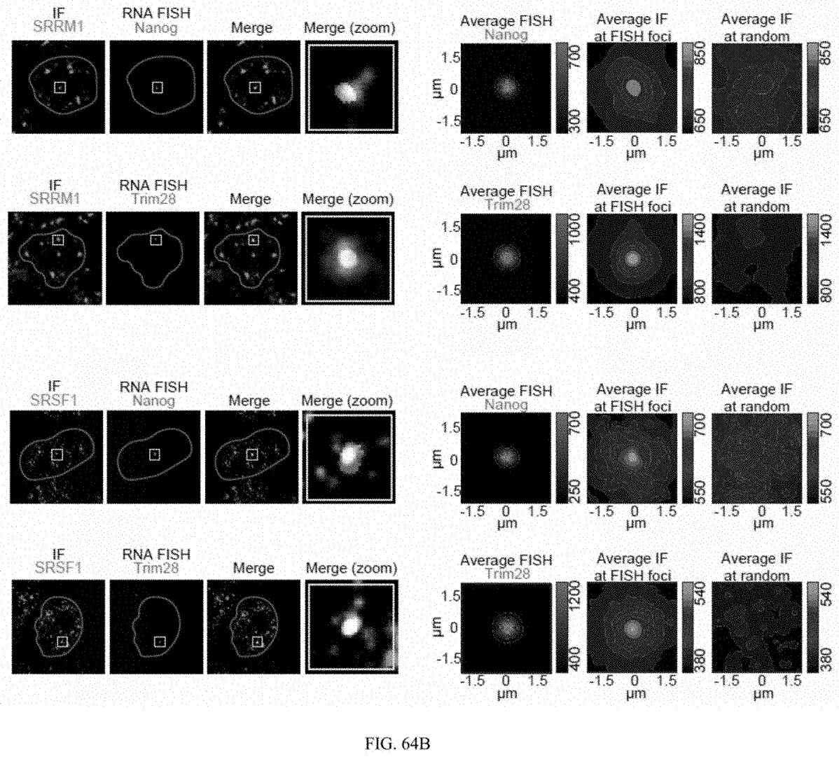

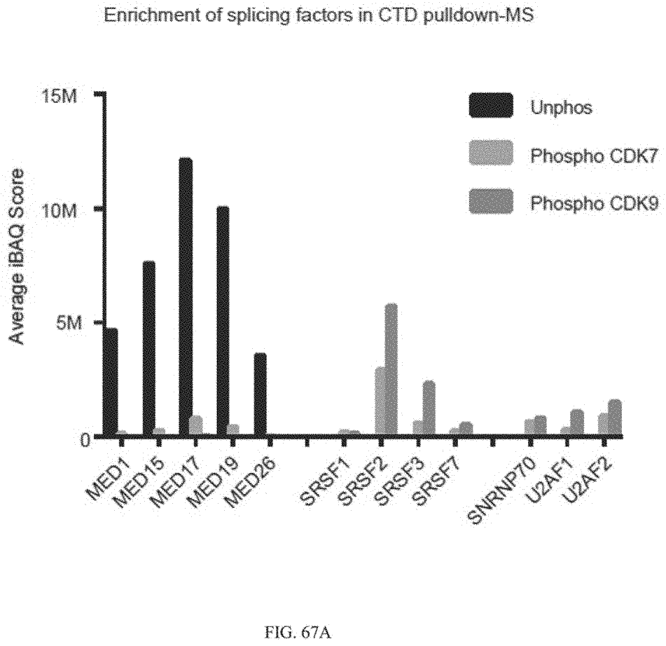

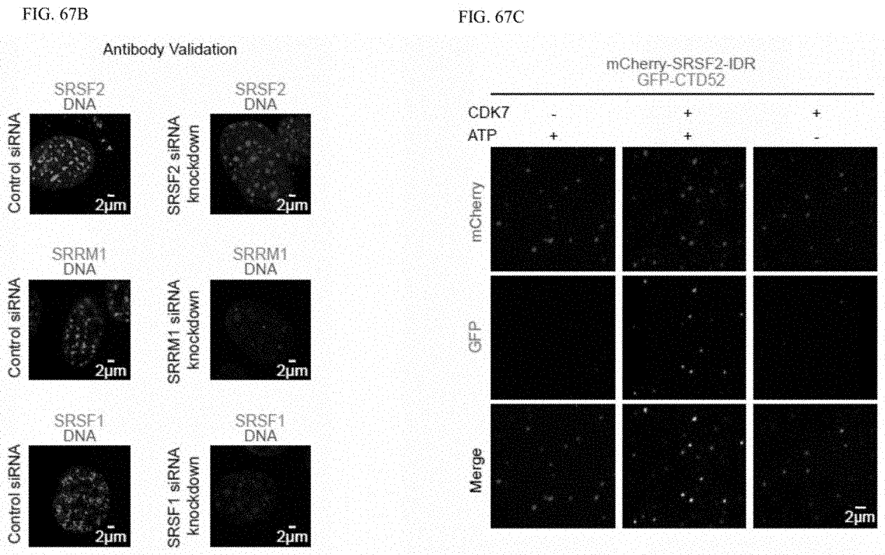

The phosphorylated Pol II CTD has been reported to interact with many components of the splicing machinery²⁷⁻³⁰, and the serine/arginine-rich (SR) protein SRSF2 is among the most enriched of these splicing factors (FIG. 66A)⁷. SRSF2 facilitates recruitment of the spliceosome to splice sites³¹ and can be found associated with the pre-mRNA splicing machinery in nuclear speckles¹⁰. Using SRSF2 as a surrogate for the splicing machinery, we investigated whether splicing-associated condensates could be found at active super-enhancer associated genes in mouse embryonic stem cells (mESCs) (FIG. 64). Immunofluorescence microscopy using antibodies specific for SRSF2 (FIG. 67B) with concurrent nascent RNA FISH revealed discrete puncta of SRSF2 at the Nanog and Trim28 genes, which are super-enhancer associated genes that encode key ESC pluripotency transcription factors (FIG. 64A). Analysis of multiple images of Nanog and Trim28 FISH foci (see methods) showed that SRSF2 was enriched at nascent RNA FISH foci at both genes (FIG. 64A). We verified that two additional SR proteins required for splicing, SRRM1 and SRSF1^{32,33}, were also enriched at nascent RNA FISH foci at both Nanog and Trim28 genes (FIG. 64B). These results indicate that SRSF2 and other proteins associated with the splicing machinery are components of condensates located at these actively transcribed genes.

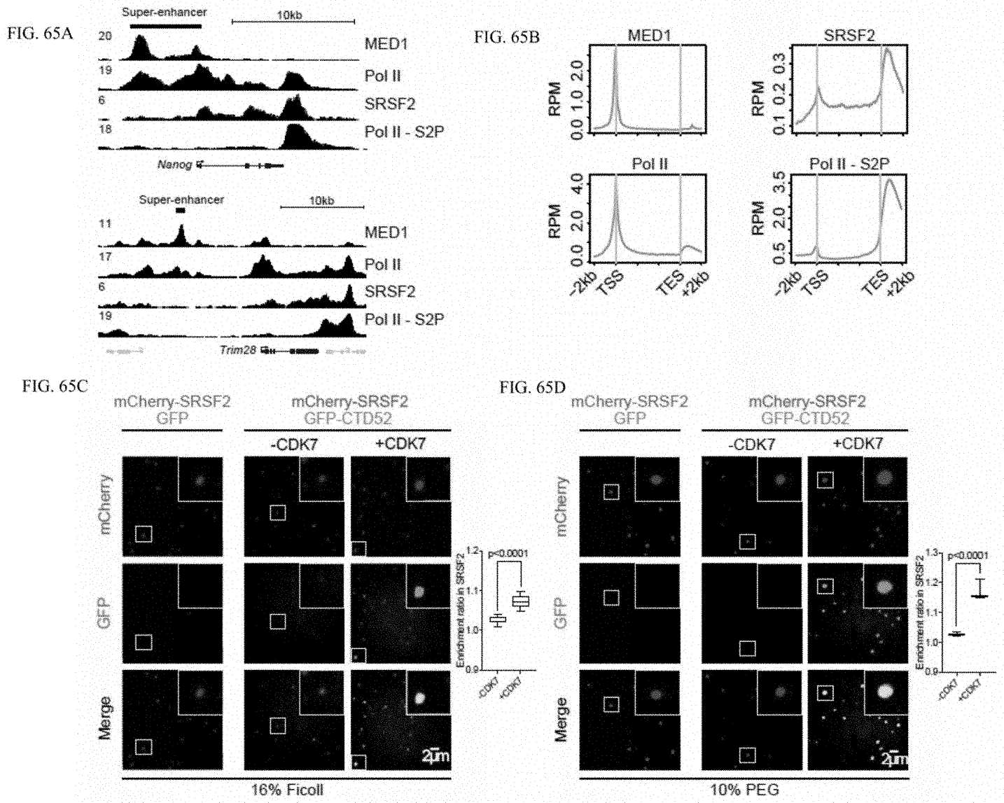

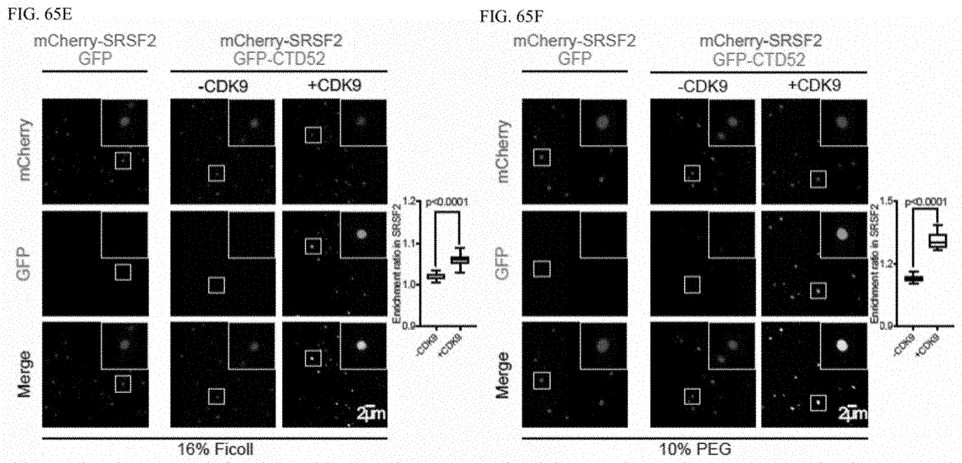

We next investigated whether phosphorylated Pol II is associated with SRSF2 on chromatin. ChIP-seq was performed with antibodies against MED1, SRSF2, the unphosphorylated Pol II CTD and the Pol II CTD phosphorylated at serine 2 (S2P) to obtain clues to the relative occupancy of these components at various loci genome-wide (FIG. 4a, b). As expected, MED1 occupied super-enhancers and promoters together with Pol II containing unphosphorylated CTD FIG. 65A,B). Pol II containing serine 2 phosphorylated CTD was observed most predominantly at the 3′ ends of transcribed genes and exhibited strong overlap with SRSF2 (FIG. 65A,B). These results suggest that portions of the genome occupied by SRSF2 tend to be co-occupied by Pol II with a phosphorylated CTD.

To directly test whether phosphorylation of the CTD influences its incorporation into splicing factor condensates, we sought to model these condensates in vitro using recombinant SRSF2. Full-length human SRSF2 fused to mCherry was purified and found to form phase-separated droplets (FIG. 65C,D). While unphosphorylated CTD was not efficiently incorporated into SRSF2 droplets, CDK7- or CDK9-phosphorylated CTD was incorporated and concentrated in SRSF2 droplets (FIG. 65C,D,E,F and FIG. 67C). This selectivity for incorporation of phosphorylated Pol II CTD by SRSF2 droplets was independent of the crowding agent used in the experiment (FIG. 65C,D,E,F). These results show that phosphorylation of the Pol II CTD leads to a switch in its capacity to interact with SRSF2 condensates.

Our results indicate that Pol II CTD phosphorylation alters its condensate partitioning behavior and may thus drive an exchange of Pol II from condensates involved in transcription initiation to those involved in RNA splicing. This model is consistent with evidence from previous studies that large clusters of Pol II can fuse with Mediator condensates in cells⁸, that phosphorylation dissolves CTD-mediated Pol II clusters³⁴, that CDK9/Cyclin T can interact with the CTD through a phase separation mechanism³⁵, that Pol II is no longer associated with Mediator during transcription elongation¹⁸, and that nuclear speckles containing splicing factors can be observed at loci with high transcriptional activity^{10-17.} Previous studies have shown that the CTD can interact with components of the transcription initiation apparatus and RNA processing machinery in a phosphoform-specific manner⁵⁻⁷, but did not explore the possibility that these components occur in condensates or that phosphorylation of the Pol II CTD alters its partitioning behavior between these condensates. Our results reveal that Mediator and splicing factor condensates occur at the same super-enhancer driven genes and suggest that the transition of Pol II from interactions with components involved in initiation to those involved in splicing can be mediated through a CTD phosphorylation regulated condensate partitioning switch. These results also suggest that phosphorylation may be among the mechanisms that regulate condensate partitioning of proteins in processes where protein function involves eviction from one condensate and migration to another.

Methods

Cell Culture

V6.5 murine embryonic stem cells (mESCs) were a gift from the Jaenisch lab. Cells were grown on 0.2% gelatinized (Sigma, G1890) tissue culture plates in 2i media, DMEM-F12 (Life Technologies, 11320082), 0.5× B27 supplement (Life Technologies, 17504044), 0.5× N2 supplement (Life Technologies, 17502048), an extra 0.5 mM L-glutamine (Gibco, 25030-081), 0.1 mM beta-mercaptoethanol (Sigma, M7522), 1% Penicillin Streptomycin (Life Technologies, 15140163), 1× nonessential amino acids (Gibco, 11140-050), 1000 U/ml LIF (Chemico, ESG1107), 1 μM PD0325901 (Stemgent, 04-0006-10), 3 μM CHIR99021 (Stemgent, 04-0004-10). Cells were grown at 37° C. with 5% CO2 in a humidified incubator. For confocal imaging, cells were grown on glass coverslips (Carolina Biological Supply, 633029), coated with 5 μg/mL of poly-L-ornithine (Sigma Aldrich, P4957) for at least 30 min at 37° C. and with 5 μg/ml of Laminin (Corning, 354232) for 2 hrs-16 hrs at 37° C. For passaging, cells were washed in PBS (Life Technologies, AM9625), 1000 U/mL LIF. TrypLE Express Enzyme (Life Technologies, 12604021) was used to detach cells from plates. TrypLE was quenched with FBS/LIF-media (DMEM K/O (Gibco, 10829-018), 1× nonessential amino acids, 1% Penicillin Streptomycin, 2 mM L-Glutamine, 0.1 mM beta-mercaptoethanol and 15% Fetal Bovine Serum, FBS, (Sigma Aldrich, F4135).

Western Blot

Purified phosphorylated CTD was mixed in 1×XT buffer (Bio-Rad) and run on 10% Criterion™ XT Bis-Tris Precast Gels (Bio-Rad) at 100 V until the dye front reached the end of the gel. Protein was then wet transferred to a 0.45 μm PVDF membrane (Millipore, IPVH00010) in ice-cold transfer buffer (25 mM Tris, 192 mM glycine, 10% methanol) at 250 mA for 2 hours at 4° C. After transfer, the membrane was blocked with 5% non-fat milk in TBS for 1 hour at room temperature, with shaking. The membrane was then incubated with a 1:2,000 dilution of anti-GFP (Abcam #ab290), anti-Pol II phospho-Ser5 (Millipore #04-1572) or anti-Pol II phospho-Ser2 (Millipore #04-1571) antibodies in 5% non-fat milk in TBST overnight at 4° C., with shaking. The membrane was washed three times with TBST for 10 min at room temperature with shaking. The membrane was incubated with 1:10,000 secondary antibodies (GE health) for 1 hr at RT and washed three times in TBST for 5 mins. Membranes were developed with Femto ECL substrate (Thermo Scientific, 34095) and imaged using a CCD camera.

Immunofluorescence with RNA FISH

Coverslips were coated at 37° C. with 5 ug/mL poly-L-ornithine (Sigma-Aldrich, P4957) for 30 minutes and 5 μg/mL of Laminin (Corning, 354232) for 2 hours. Cells were plated on the pre-coated cover slips and grown for 24 hours followed by fixation using 4% paraformaldehyde, PFA, (VWR, BT140770) in PBS for 10 minutes. After washing cells three times in PBS, the coverslips were put into a humidifying chamber or stored at 4° C. in PBS. Permeabilization of cells was performed using 0.5% triton X100 (Sigma Aldrich, X100) in PBS for 10 minutes followed by three PBS washes. Cells were blocked with 4% IgG-free Bovine Serum Albumin, BSA, (VWR, 102643-516) for 30 minutes. Cells were then incubated with the indicated primary antibody at a concentration of 1:500 in PBS for 4-16 hours. Cells were washed with PBS three times followed by incubation with secondary antibody at a concentration of 1:5000 in PBS for 1 hour. After washing twice with PBS, cells were fixed using 4% paraformaldehyde, PFA, (VWR, BT140770) in PBS for 10 minutes. After two washes of PBS, Wash buffer A (20% Stellaris RNA FISH Wash Buffer A (Biosearch Technologies, Inc., SMF-WA1-60), 10% Deionized Formamide (EMD Millipore, S4117)) in RNase-free water (Life Technologies, AM9932) was added to cells and incubated for 5 minutes. 12.5 μM RNA probe in Hybridization buffer (90% Stellaris RNA FISH Hybridization Buffer (Biosearch Technologies, SMF HB1-10) and 10% Deionized Formamide) was added to cells and incubated overnight at 37° C. After washing with Wash buffer A for 30 minutes at 37° C., the nuclei were stained in 20 μm/mL Hoechst 33258 (Life Technologies, H3569) for 5 minutes, followed by a 5 minute wash in Wash buffer B (Biosearch Technologies, SMFWB1-20). Cells were washed once in water followed by mounting the coverslip onto glass slides with Vectashield (VWR, 101098-042) and finally sealing the cover slip with nail polish (Electron Microscopy Science Nm, 72180). Images were acquired on the RPI Spinning Disk confocal microscope with 100× objective using MetaMorph acquisition software and a Hammamatsu ORCA-ER CCD camera (W.M. Keck Microscopy Facility, MIT). Images were post-processed using Fiji Is Just ImageJ (FIJI). RNA FISH probes were custom designed and generated by Agilent to target Nanog and Trim28 intronic regions to visualize nascent RNA.

Protein Purification

Human cDNA was cloned into a modified version of a T7 pET expression vector. The base vector was engineered to include a 5′ 6×HIS followed by either mEGFP or mCherry and a 14 amino acid linker sequence “GAPGSAGSAAGGSG.” (SEQ ID NO: 14). NEBuilder® HiFi DNA Assembly Master Mix (NEB E2621S) was used to insert these sequences (generated by PCR) in-frame with the linker amino acids. Vector expressing mEGFP alone contains the linker sequence followed by a STOP codon. Mutant sequences were generated by PCR and inserted into the same base vector as described above. All expression constructs were sequenced to ensure sequence identity.

For protein expression, plasmids were transformed into LOBSTR cells (gift of Chessman Lab) and grown as follows. A fresh bacterial colony was inoculated into LB media containing kanamycin and chloramphenicol and grown overnight at 37 degrees. Cells were diluted 1:30 in 500 ml room temperature LB with freshly added kanamycin and chloramphenicol and grown 1.5 hours at 16 degrees. IPTG was added to 1 mM and growth continued for 20 hours. Cells were collected and stored frozen at −80 degrees. Cells containing GFP alone and GFP-SRSF2 were treated in a similar manner except they were grown for 5 hours at 37 degrees after IPTG induction.

Pellets of 500 ml of mCherry-SRSF2 expressing cells were resuspended in 15 ml of denaturing buffer (50 mM Tris 7.5, 300 mM NaCl, 10 mM imidazole, 8 M Urea) with cOmplete protease inhibitors (Roche, 11873580001) and sonicated (ten cycles of 15 seconds on, 60 sec off). The lysates were cleared by centrifugation at 12,000 g for 30 minutes and added to 1 ml of Ni-NTA agarose (Invitrogen, R901-15) that had been pre-equilibrated with 10 volumes of the same buffer. Tubes containing this agarose lysate slurry were rotated for 1.5 hours at room temperature. The slurry was poured into a column, washed with 15 volumes of the lysis buffer and eluted 4×2 ml with denaturing buffer containing 250 mM imidazole. Each fraction was run on a 12% gel and proteins of the correct size were dialyzed first against buffer (50 mM Tris pH 7.5, 125 mM NaCl, 1 mM DTT and 4 M Urea), followed by the same buffer containing 2M Urea and lastly 2 changes of buffer with 10% Glycerol, no Urea. Any precipitate after dialysis was removed by centrifugation at 3,000 rpm for 10 minutes.

All other proteins were purified in a similar manner. About 500 ml cell pellets were resuspended in 15 ml of Buffer A (50 mM Tris pH7.5, 500 mM NaCl) containing 10 mM imidazole and cOmplete protease inhibitors, lysed by sonication, cleared by centrifugation at 12,000×g for 30 minutes at 4 degrees, added to 1 ml of pre-equilibrated Ni-NTA agarose, and rotated at 4 degrees for 1.5 hours. The slurry was poured into a column, washed with 15 volumes of lysis buffer containing 10 mM imidazole and protein was eluted 2× with buffer containing 50 mM imidazole, 2× with buffer containing 100 mM imidazole, and 3× with buffer containing 250 mM imidazole. Alternatively, the resin slurry was centrifuged at 3,000 rpm for 10 minutes, washed with 10 volumes of 10 mM imidazole buffer and proteins were eluted by incubation for 10 or more minutes rotating with each of the buffers above followed by centrifugation and gel analysis. Fractions containing protein of the correct size were dialyzed against two changes of buffer containing 50 mM Tris 7.5, 125 mM NaCl, 10% glycerol and 1 mM DTT at 4 degrees.

Purification of Mediator

The Mediator samples were purified as previously described³⁶ with modifications. Prior to affinity purification, the P0.5M/QFT fraction was concentrated, to 12 mg/mL, by ammonium sulfate precipitation (35%). The pellet was resuspended in pH 7.9 buffer containing 20 mM KCl, 20 mM HEPES, 0.1 mM EDTA, 2 mM MgCl₂, 20% glycerol and then dialyzed against pH 7.9 buffer containing 0.15 M KCl, 20 mM HEPES, 0.1 mM EDTA, 20% glycerol and 0.02% NP-40 prior to the affinity purification step. Affinity purification was carried out as described³⁶, eluted material was loaded onto a 2.2 mL centrifuge tube containing 2 mL 0.15M KCl HEMG (20 mM HEPES, 0.1 mM EDTA, 2 mM MgCl2, 10% glycerol) and centrifuged at 50K RPM for 4 h at 4° C. This served to remove excess free GST-SREBP and to concentrate the Mediator in the final fraction. Prior to droplet assays, purified Mediator was further concentrated using Microcon-30 kDa Centrifugal Filter Unit with Ultracel-30 membrane (Millipore MRCFOR030) to reach ˜300 nM of Mediator complex. Concentrated Mediator was added to the droplet assay to a final concentration of ˜200 nM with or without 10 μM indicated GFP-tagged protein. Droplet reactions contained 10% PEG-8000 or 16% Ficoll-400 and 140 mM salt.

Chromatin Immunoprecipitation Sequencing (ChIP-Seq)

mES were grown to 80% confluence in 2i media. 1% formaldehyde in PBS was used for crosslinking of cells for 15 minutes, followed by quenching with Glycine at a final concentration of 125 mM on ice. Cells were washed with cold PBS and harvested by scraping cells in cold PBS. Collected cells were pelleted at 1000 g for 3 minutes at 4° C., flash frozen in liquid nitrogen and stored at −80° C. All buffers contained freshly prepared cOmplete protease inhibitors (Roche, 11873580001). For ChIPs using phospho-specific antibodies, all buffers contained freshly prepared PhosSTOP phosphatase inhibitor cocktail (Roche, 4906837001). Frozen crosslinked cells were thawed on ice and then resuspended in LB1 (50 mM Hepes-KOH, pH7.9, 140 mM NaCl, 1 mM EDTA 0.5 mL 0.5M, 10% glycerol, 0.5% NP-40, 1% TritonX-100, lx protease inhibitor) and incubated for 20 minutes rotating at 4° C. Cells were pelleted for 5 minutes at 1350 g, resuspended in LB2 (10 mM Tris pH 8.0, 200 mM NaCl, 1 mM EDTA, 0.5 mM EGTA, 1× protease inhibitor) and incubated for 5 minutes rotating at 4° C. Pellets were resuspended in LB3 (10 mM Tris pH 8.0, 100 mM NaCl, 1 mM EDTA, 0.5 mM EGTA, 0.1% sodium-deoxycholate, 0.5% sodium lauroyl sarcosinate, 1% TritonX-100, 1× protease inhibitor) at a concentration of 30-50 million cells/ml. Cells were sonicated using Covaris S220 for 12 minutes (Duty cycle: 5%, intensity: 4, cycles per burst: 200). Sonicated material was clarified by spinning at 20000×g for 30 minutes at 4° C. The supernatant is the soluble chromatin used for the ChIP. Dynabeads pre-blocked with 0.5% BSA were incubated with indicated antibodies for 2 hours. Chromatin was added to antibody-bead complex and incubated rotating overnight at 4° C. Beads were washed three times each with Wash buffer 1 (50 mM Hepes pH7.5, 500 mM NaCl, 1 mM EDTA, 1 mM EGTA, 1% Triton, 0.1% NaDoc, 0.1% SDS) and Wash Buffer 2 (20 mM Tris pH 8, 1 mM EDTA, 250 mM LiCl, 0.5% NP-40, 0.5% NaDoc) at 4° C., followed by washing one time with TE at room temperature. Chromatin was eluted by adding Elution buffer (50 mM, Tris pH 8.0, 10 mM EDTA, 1% sodium dodecyl sulfate) to the beads and incubated shaking at 60° C. for 30 minutes. Reversal of crosslinking was performed overnight at 58° C. RNaseA was added and incubated for 1 hour at 50° C. for RNA removal. Proteinase K was added and incubated for 1 hour at 60° C. for protein removal. DNA was purified using Qiagen PCR purification kit, as per manufacturer's instructions, and eluted in 50 μL 10 mM Tris-HCl, pH 8.5, which was used for quantitation and ChIP library preparation. ChIP Libraries were prepared with the Swift Biosciences Accel-NGS® 2S Plus DNA Library Kit according to kit instructions with an additional size selection step on the PippinHT system from Sage Science. Following library prep, ChIP libraries were run on a 2% gel on the PippinHT with a size collection window of 200-600 bases. Final libraries were quantified by qPCR with the KAPA Library Quantification kit from Roche and sequenced in single-read mode for 40 bases on an Illumina HiSeq 2500.

ChIP-Seq data were aligned to the mm9 version of the mouse reference genome using bowtie with parameters -k 1 -m 1 -best and -l set to read length. Wiggle files for display of read coverage in bins were created using MACS with parameters -w -S -space=50 -nomodel -shiftsize=200, and read counts per bin were normalized to the millions of mapped reads used to make the wiggle file. Reads-per-million-normalized wiggle files were displayed in the UCSC genome browser. Metagene plots were made using ngs.plot37 (v2.61) using default parameters. Top 20% of expressed genes were calculated from a published RNA-seq dataset (GSE112807)9. SRSF2 and Ser2-P Pol II ChIP-seqs were generated in this study using antibodies against SRSF2 (Abcam ab11826) and Pol II Ser2 phospho CTD (Millipore 04-1571), whereas MED1 and total Pol II ChIP-seqs were previously published (GSE112808)9.

Average Image Analysis

For analysis of RNA FISH with immunofluorescence custom in-house MATLAB™ scripts were written to process and analyze 3D image data gathered in RNA FISH and IF channels. FISH foci were identified in individual z-stacks through intensity and size thresholds, centered along a box of size l=2.9 μm and stitched together in 3-D across z-stacks. For every FISH focus identified, signal from the corresponding location in the IF channel is gathered in the l×l square centered at the RNA FISH focus at every corresponding z-slice. The IF signal centered at FISH foci for each FISH and IF pair are then combined and an average intensity projection is calculated, providing averaged data for IF signal intensity within a l×l square centered at FISH foci. The same process was carried out for the FISH signal intensity centered on its own coordinates, providing averaged data for FISH signal intensity within a l×l square centered at FISH foci. The number of replicates per average intensity projection is provided for each image set within the figure legends. As a control, this same process was carried out for IF signal centered at randomly selected nuclear positions. For each replicate, 40 random nuclear points were generated from the interior of the nuclear envelope, identified from the DAPI channel by a combination of large size (200 voxels) and intensity (DNA dense) thresholds.

These average intensity projections were then used to generate 2D contour maps of the signal intensity. Contour plots are generated using built-in functions in MATLAB™. For the contour plots, the intensity-color ranges presented were customized across a linear range of colors (n!=15). For the FISH channel, black to magenta was used. For the IF channel, we used chroma.js (an online color generator) to generate colors across 15 bins, with the key transition colors chosen as black, blueviolet, mediumblue, lime. This was done to ensure that the reader's eye could more readily detect the contrast in signal. The generated colormap was employed to 15 evenly spaced intensity bins for all IF plots. The averaged IF centered at FISH or at randomly selected nuclear locations are plotted using the same color scale, set to include the minimum and maximum signal from each plot.

In Vitro Droplet Assay

Recombinant GFP or mCherry fusion proteins were concentrated and desalted to an appropriate protein concentration and 125 mM NaCl using Amicon Ultra centrifugal filters (30K MWCO, Millipore). Recombinant proteins were added to solutions at varying concentrations with 100-125 mM final salt and 16% Ficoll-400 or 10% PEG-8000 as crowding agent in Droplet Formation Buffer (50 mM Tris-HCl pH 7.5, 10% glycerol, 1 mM DTT) as described in figure legends. The protein solution was immediately loaded onto a homemade chamber comprising a glass slide with a coverslip attached by two parallel strips of double-sided tape. Slides were then imaged with the Andor confocal microscope with a 150× objective. Unless indicated, images presented are of droplets settled on the glass coverslip. For FRAP of in vitro droplets, 2 pulses of laser (20% power) at a 20 us dwell time were applied to the droplet, and recovery was imaged on the Andor microscope every is for the indicated time periods. For CDK7 or CDK9 mediated CTD phosphorylation, commercially available active CDK7/MAT1/CCNH (CAK complex; Millipore 14-476) or CDK9/Cyclin T1 (Millipore 14-685) was used to phosphorylate GFP-CTD52 in kinase reaction buffer (20 mM MOPs-NaOH pH 7.0, 1 mM EDTA, 0.001% NP-40, 2.5% glycerol, 0.05% beta-mercaptoethanol, 10 mM MgAc, 10 uM ATP) at room temperature for 2-3 hours. The CTD to enzyme ratio is ˜1 uM CTD to ˜4.8 ng/ul CDK7 or CDK9.

Imaging Analysis of In Vitro Droplets

To analyze in-vitro phase separation imaging experiments, custom MATLAB™ scripts were written to identify droplets and characterize their size and shape. For any particular experimental condition, intensity thresholds based on the peak of the histogram and size thresholds (9 pixels per z-slice) were employed to segment the image. Droplet identification was performed on the “scaffold” channel (MED1-IDR in case of MED1-IDR+CTD, SRSF2 for SRSF2+CTD), and areas and aspect ratios were determined. Hundreds of droplets identified in typically 5-10 independent fields of view were quantified. Average intensity within the droplets (C-in) and in the bulk (C-out) were calculated for the GFP channel (i.e. GFP-CTD). The partition coefficient/enrichment ratio for GFP-CTD was computed as (C-in)/(C-out). Enrichment scores were calculated by dividing the Cin/out of the experimental condition by the Cin/out of a control GFP fluorescent protein.

Data Availability

Datasets generated in this study have been deposited in the Gene Expression Omnibus under accession number GSE120656.

### REFERENCES

1 Adelman, K. & Lis, J. T. Promoter-proximal pausing of RNA polymerase II: emerging roles in metazoans. Nat Rev Genet 13, 720-731, doi:10.1038/nrg3293 (2012).
2 Harlen, K. M. & Churchman, L. S. The code and beyond: transcription regulation by the RNA polymerase II carboxy-terminal domain. Nat Rev Mol Cell Biol 18, 263-273, doi:10.1038/nrm.2017.10 (2017).
3 Levine, M., Cattoglio, C. & Tjian, R. Looping back to leap forward: transcription enters a new era. Cell 157, 13-25, doi:10.1016/j.cell.2014.02.009 (2014).
4 Sainsbury, S., Bernecky, C. & Cramer, P. Structural basis of transcription initiation by RNA polymerase II. Nat Rev Mol Cell Biol 16, 129-143, doi:10.1038/nrm3952 (2015).
5 Eick, D. & Geyer, M. The RNA polymerase II carboxy-terminal domain (CTD) code. Chem Rev 113, 8456-8490, doi:10.1021/cr400071f (2013).
6 Jeronimo, C., Bataille, A. R. & Robert, F. The writers, readers, and functions of the RNA polymerase II C-terminal domain code. Chem Rev 113, 8491-8522, doi:10.1021/cr4001397 (2013).
7 Ebmeier, C. C. et al. Human TFIIH Kinase CDK7 Regulates Transcription-Associated Chromatin Modifications. Cell Rep 20, 1173-1186, doi:10.1016/j.celrep.2017.07.021 (2017).
8 Cho, W. K. et al. Mediator and RNA polymerase II clusters associate in transcription-dependent condensates. Science 361, 412-415, doi:10.1126/science.aar4199 (2018).
9 Sabari, B. R. et al. Coactivator condensation at super-enhancers links phase separation and gene control. Science 361, doi:10.1126/science.aar3958 (2018).
10 Spector, D. L. & Lamond, A. I. Nuclear speckles. Cold Spring Harb Perspect Biol 3, doi:10.1101/cshperspect.a000646 (2011).
11 Chen, Y. et al. Mapping 3D genome organization relative to nuclear compartments using TSA-Seq as a cytological ruler. J Cell Biol, doi:10.1083/jcb.201807108 (2018).
12 Quinodoz, S. A. et al. Higher-Order Inter-chromosomal Hubs Shape 3D Genome Organization in the Nucleus. Cell 174, 744-757 e724, doi:10.1016/j.cell.2018.05.024 (2018).
13 Shopland, L. S., Johnson, C. V., Byron, M., McNeil, J. & Lawrence, J. B. Clustering of multiple specific genes and gene-rich R-bands around SC-35 domains: evidence for local euchromatic neighborhoods. J Cell Biol 162, 981-990, doi:10.1083/jcb.200303131 (2003).
14 Xing, Y., Johnson, C. V., Moen, P. T., Jr., McNeil, J. A. & Lawrence, J. Nonrandom gene organization: structural arrangements of specific pre-mRNA transcription and splicing with SC-35 domains. J Cell Biol 131, 1635-1647 (1995).
15 Moen, P. T., Jr. et al. Repositioning of muscle-specific genes relative to the periphery of SC-35 domains during skeletal myogenesis. Mol Biol Cell 15, 197-206, doi:10.1091/mbc.e03-06-0388 (2004).
16 Hu, Y., Kireev, I., Plutz, M., Ashourian, N. & Belmont, A. S. Large-scale chromatin structure of inducible genes: transcription on a condensed, linear template. J Cell Biol 185, 87-100, doi:10.1083/jcb.200809196 (2009).
17 Khanna, N., Hu, Y. & Belmont, A. S. HSP70 transgene directed motion to nuclear speckles facilitates heat shock activation. Curr Biol 24, 1138-1144, doi:10.1016/j.cub.2014.03.053 (2014).
18 Allen, B. L. & Taatjes, D. J. The Mediator complex: a central integrator of transcription. Nat Rev Mol Cell Biol 16, 155-166, doi:10.1038/nrm3951 (2015).
19 Zhang, X. et al. MED1/TRAP220 exists predominantly in a TRAP/Mediator subpopulation enriched in RNA polymerase II and is required for ER-mediated transcription. Mol Cell 19, 89-100, doi:10.1016/j.molcel.2005.05.015 (2005).
20 Banani, S. F., Lee, H. O., Hyman, A. A. & Rosen, M. K. Biomolecular condensates: organizers of cellular biochemistry. Nat Rev Mol Cell Biol 18, 285-298, doi:10.1038/nrm.2017.7 (2017).
21 Hnisz, D., Shrinivas, K., Young, R. A., Chakraborty, A. K. & Sharp, P. A. A Phase Separation Model for Transcriptional Control. Cell 169, 13-23, doi:10.1016/j.cell.2017.02.007 (2017).
22 Akhtar, M. S. et al. TFIIH kinase places bivalent marks on the carboxy-terminal domain of RNA polymerase II. Mol Cell 34, 387-393, doi:10.1016/j.molcel.2009.04.016 (2009).
23 Glover-Cutter, K. et al. TFIIH-associated Cdk7 kinase functions in phosphorylation of C-terminal domain Ser7 residues, promoter-proximal pausing, and termination by RNA polymerase II. Mol Cell Biol 29, 5455-5464, doi:10.1128/MCB.00637-09 (2009).
24 Czudnochowski, N., Bosken, C. A. & Geyer, M. Serine-7 but not serine-5 phosphorylation primes RNA polymerase II CTD for P-TEFb recognition. Nat Commun 3, 842, doi:10.1038/ncomms1846 (2012).
25 Jones, J. C. et al. C-terminal repeat domain kinase I phosphorylates Ser2 and Ser5 of RNA polymerase II C-terminal domain repeats. J Biol Chem 279, 24957-24964, doi:10.1074/jbc.M402218200 (2004).
26 Kwon, I. et al. Phosphorylation-regulated binding of RNA polymerase II to fibrous polymers of low-complexity domains. Cell 155, 1049-1060, doi:10.1016/j.cell.2013.10.033 (2013).
27 Bentley, D. L. Coupling mRNA processing with transcription in time and space. Nat Rev Genet 15, 163-175, doi:10.1038/nrg3662 (2014).
28 Braunschweig, U., Gueroussov, S., Plocik, A. M., Graveley, B. R. & Blencowe, B. J. Dynamic integration of splicing within gene regulatory pathways. Cell 152, 1252-1269, doi:10.1016/j.cell.2013.02.034 (2013).
29 Herzel, L., Ottoz, D. S. M., Alpert, T. & Neugebauer, K. M. Splicing and transcription touch base: co-transcriptional spliceosome assembly and function. Nat Rev Mol Cell Biol 18, 637-650, doi:10.1038/nrm.2017.63 (2017).
30 Hsin, J. P. & Manley, J. L. The RNA polymerase II CTD coordinates transcription and RNA processing. Genes Dev 26, 2119-2137, doi:10.1101/gad.200303.112 (2012).
31 Long, J. C. & Caceres, J. F. The SR protein family of splicing factors: master regulators of gene expression. Biochem J 417, 15-27, doi:10.1042/BJ20081501 (2009).
32 Blencowe, B. J., Issner, R., Nickerson, J. A. & Sharp, P. A. A coactivator of pre-mRNA splicing. Genes Dev 12, 996-1009 (1998).
33 Kramer, A. & Keller, W. Purification of a protein required for the splicing of pre-mRNA and its separation from the lariat debranching enzyme. EMBO J 4, 3571-3581 (1985).
34 Boehning, M. et al. RNA polymerase II clustering through carboxy-terminal domain phase separation. Nat Struct Mol Biol, doi:10.1038/s41594-018-0112-y (2018).
35 Lu, H. et al. Phase-separation mechanism for C-terminal hyperphosphorylation of RNA polymerase II. Nature 558, 318-323, doi:10.1038/s41586-018-0174-3 (2018).
36 Meyer, K. D. et al. Cooperative activity of cdk8 and GCNSL within Mediator directs tandem phosphoacetylation of histone H3. EMBO J 27, 1447-1457, doi:10.1038/emboj.2008.78 (2008).
37 Shen, L., Shao, N., Liu, X. & Nestler, E. ngs.plot: Quick mining and visualization of next-generation sequencing data by integrating genomic databases. BMC Genomics 15, 284, doi:10.1186/1471-2164-15-284 (2014).

### Example 7

Phase separation is a physicochemical process by which biomolecules separate into dilute and concentrated phases, thereby forming “membraneless organelles” (1-5). Recent studies have shown that TFs and the Mediator coactivator can form phase-separated condensates to compartmentalize and concentrate the transcription apparatus at genes with prominent roles in normal cell identity (6-10). Transcriptional dysregulation is a well-described feature of malignancy, but we have a limited understanding of the roles that condensates play in cancer (11-16). Thus, we sought to discover whether transcriptional condensates drive oncogenic transcriptional programs, if they are perturbed by cancer therapy, and if they are altered in the drug-resistant state.

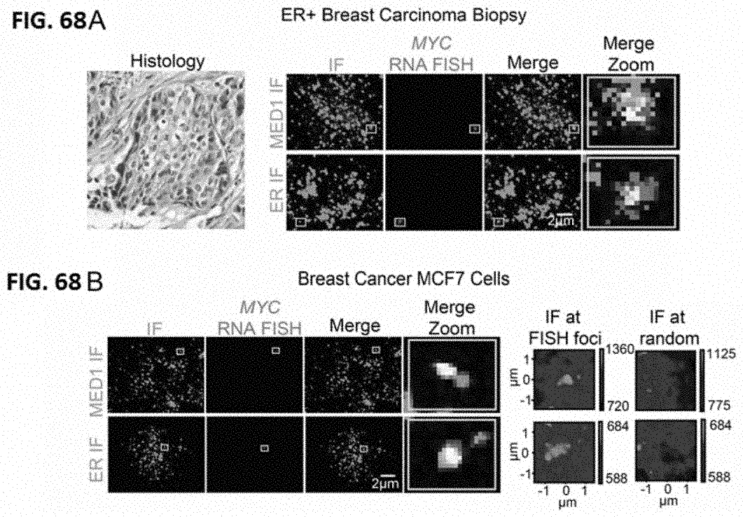

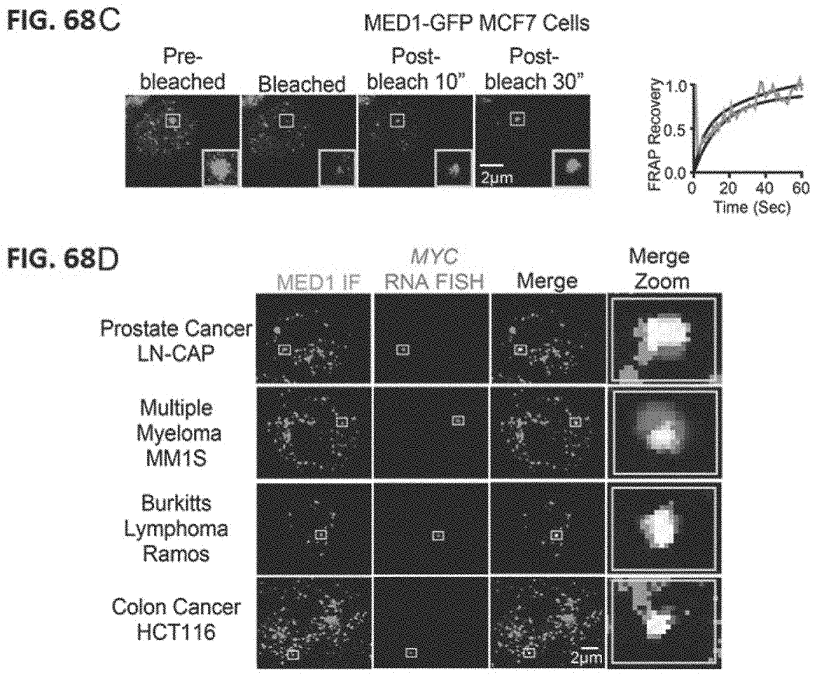

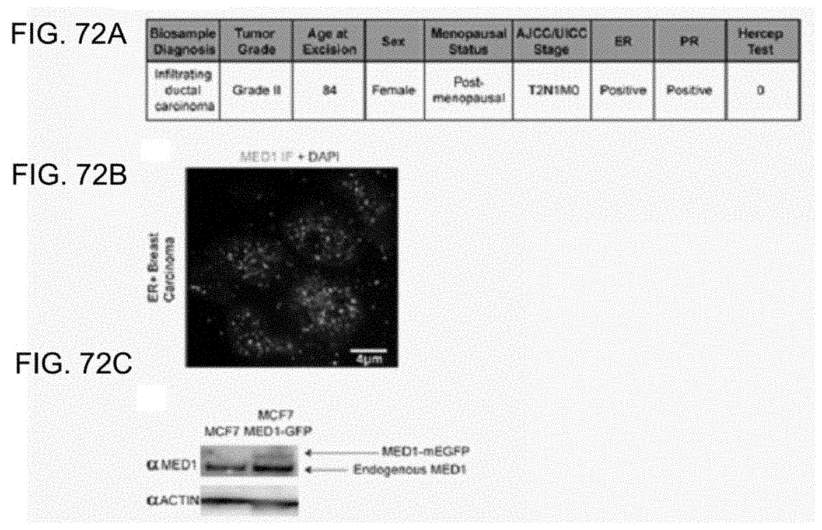

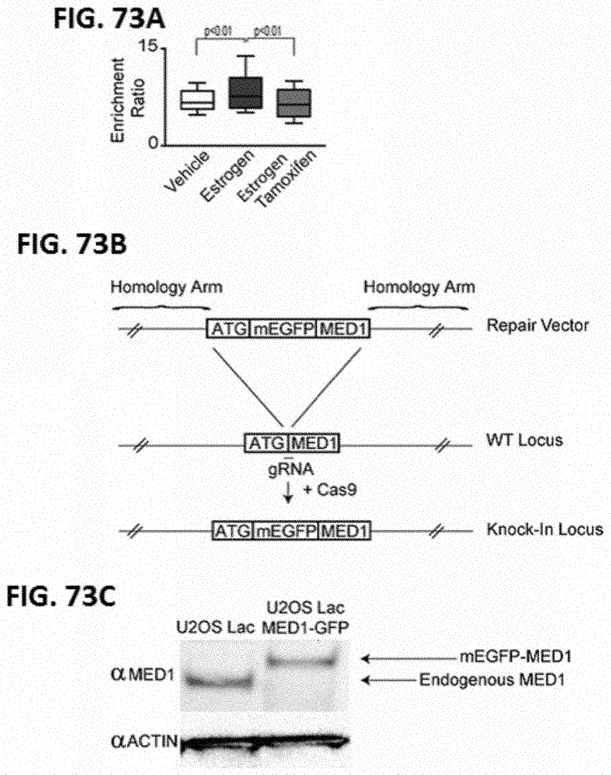

Breast cancer is the most common malignancy and the majority of cases are driven by ER, an oncogenic TF (17). ER interacts with the transcription apparatus to drive expression of estrogen responsive genes, including the MYC oncogene (18-20). To determine whether transcriptional condensates occur at MYC in human tumor tissue, we performed immunofluorescence (IF) against the MED1 subunit of Mediator and ER, together with RNA FISH, on an ER+ invasive ductal carcinoma biopsy (FIG. 68A, FIG. 72A). We found that ER and MED1 are components of nuclear puncta occurring at active MYC loci in human tumor tissue, consistent with our expectation for transcriptional condensates (FIG. 68A, FIG. 72B). We expanded our study to the more experimentally tractable ER+ breast cancer cell line MCF7 and confirmed that MED1 and ER puncta form at sites of active MYC transcription in the presence of estrogen (FIG. 68B). MED1 puncta in MCF7 cells engineered to produce mEGFP-tagged MED1 demonstrated rapid fluorescence recovery after photobleaching (FRAP) (FIG. 68C, FIG. 72C), consistent with properties expected for liquid-like condensates. These results suggest that ER and Mediator form transcriptional condensates at the MYC oncogene in breast cancer cells.

Expression of the MYC oncogene is dysregulated and drives tumorigenesis in a wide variety of cancers (21). Mediator is a coactivator of several TFs, thus one might expect Mediator condensates to be present at MYC in many cancer cell types (22). Indeed, MED1 puncta were found at transcriptionally active MYC loci in prostate cancer, multiple myeloma, Burkitt's lymphoma, and colon cancer cell lines (FIG. 68D). Taken together, these results suggest that MYC is occupied by Mediator condensates in tumor tissue and cancer cells where this gene is an oncogenic driver.

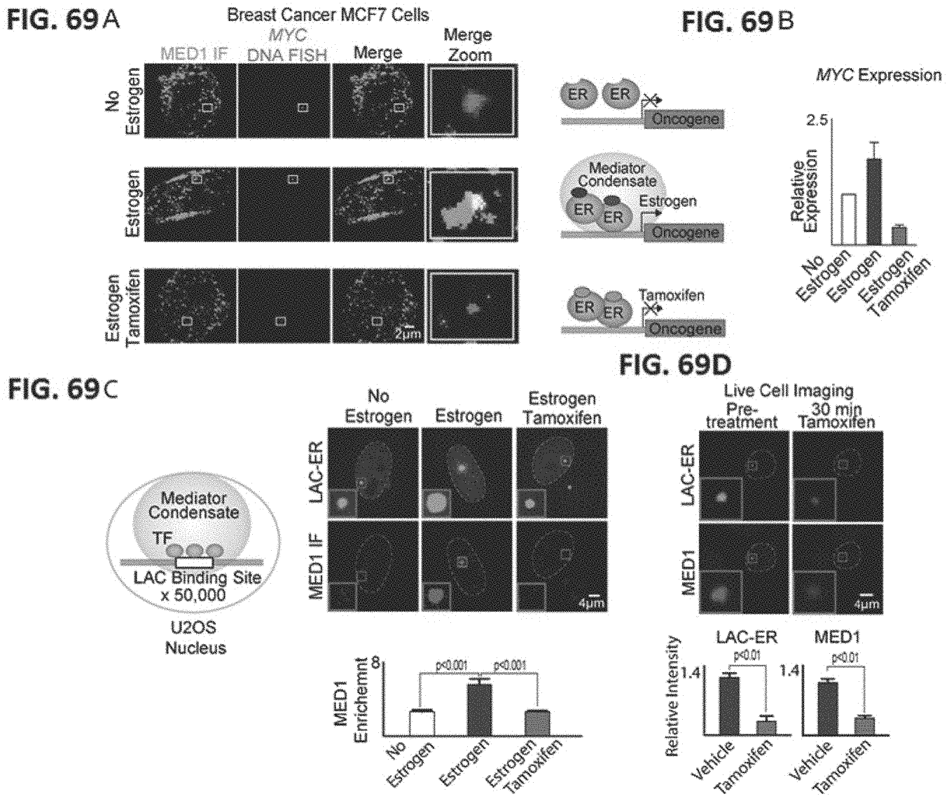

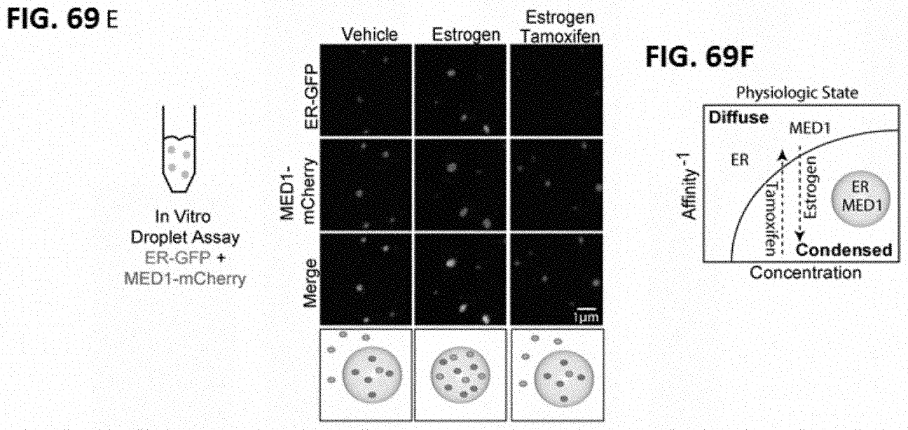

In ER+ breast cancer cells, estrogen binding to ER leads to enhanced activation of ER target genes (23). To assess whether estrogen enhances Mediator condensate formation at an ER target gene, we performed IF for MED1 together with DNA FISH at the MYC locus in MCF7 cells. MED1 signal was enhanced at MYC upon estrogen stimulation (FIG. 69A) and this was accompanied by an increase in MYC RNA expression (FIG. 69B). Tamoxifen is an anti-estrogen therapeutic that binds to the ER ligand-binding domain (LBD), resulting in a conformational shift that decreases ER's activation potential and affinity for MED1 (24). Tamoxifen treatment reduced MED1 signal at MYC (FIG. 69A), coincident with reduced MYC RNA expression (FIG. 69B). These results are consistent with a model in which estrogen stimulates coactivator condensate formation and transcription at an oncogene, and tamoxifen suppresses the estrogen-dependent stimulus of both condensate formation and transcription (FIG. 69A).

To further investigate whether the effects of estrogen and tamoxifen are due to ER LBD-dependent formation and dissolution of coactivator condensates, we used an engineered system in which formation of phase-separated condensates can be monitored when the ER LBD is tethered to a Lac array in cells (FIG. 69C) (25, 26). We found that the tethered ER LBD generated a MED1-containing condensate when cells were exposed to estrogen, and this condensate formation was prevented by tamoxifen (FIG. 69C). Live cell imaging of these cells containing endogenously tagged MED1-mEGFP (FIG. 73A, FIG. 73B) revealed that tamoxifen dissolves the ER LBD-MED1 condensate, confirming the dynamic nature expected for this assembly (FIG. 69D). These results indicate that the estrogen-dependent, tamoxifen-sensitive transactivation functions of the ER LBD correlate with the formation of estrogen-dependent, tamoxifen-sensitive MED1-containing condensates in cells.

To further study the effects of estrogen and tamoxifen on ER-MED1 condensates, we used an in vitro droplet formation assay with purified recombinant ER-GFP and truncated MED1-mCherry fusion proteins. As previously reported, MED1-mCherry formed phase-separated droplets, in which ER incorporation was enhanced by estrogen (FIG. 69E, FIG. 73C) (6). Estrogen stimulated incorporation of ER into MED1 condensates was counteracted by tamoxifen (FIG. 69E, FIG. 73C). These results are consistent with a model in which activation of estrogen-responsive oncogenes occurs through enhanced Mediator condensation, and drugs with therapeutic benefit in breast cancer can counteract formation of these condensates (FIG. 69F).

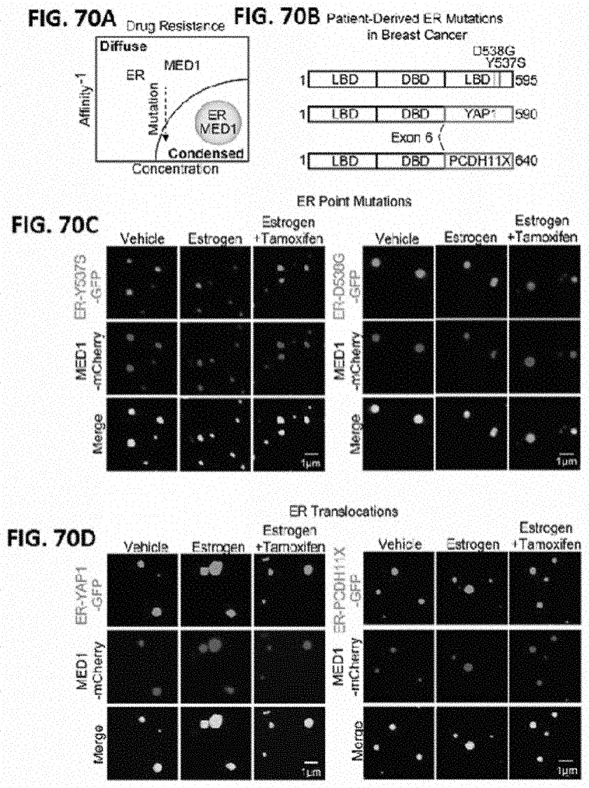

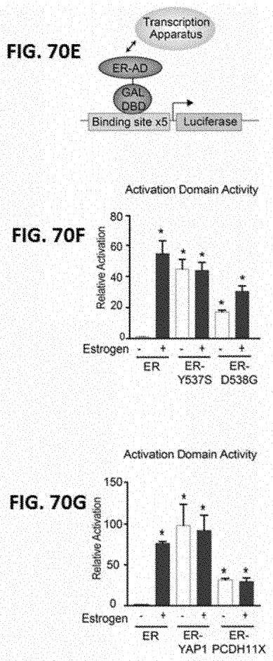

While antiestrogens such as tamoxifen are highly effective treatments for breast cancer, resistance remains a major challenge (17). Resistance may occur by multiple mechanisms, many of which result in hormone-independent interactions between ER and coactivators, with consequent gene activation and tumor growth (27). We reasoned that if the capacity of ER to condense with coactivators is essential for tumor growth and survival, antiestrogen resistance might be achieved by altering the ability of the transcription factor and the cofactor to transition across the boundary between a dilute and condensed phase. As illustrated in FIG. 70A, a shift across the phase separation boundary for a TF-Mediator condensate could occur by altering the affinity between components that compose the condensate (28).

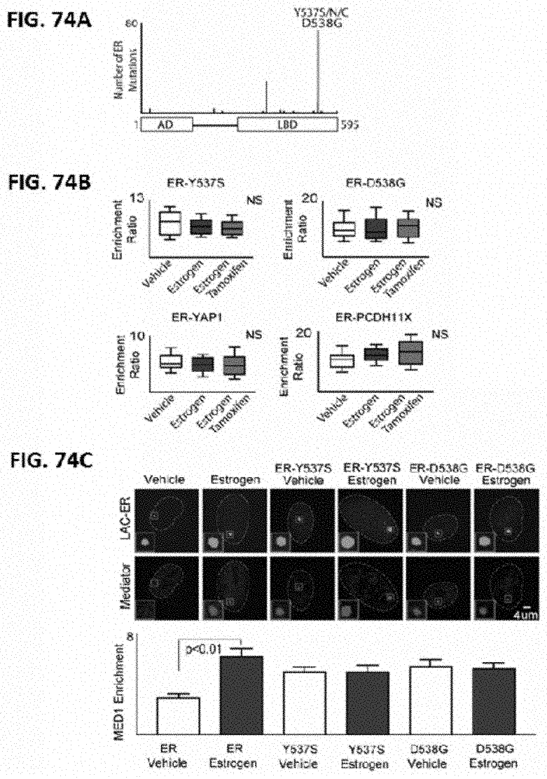

Diverse genetic alterations of ER are found in antiestrogen-resistant breast cancer patients, including mutations in the LBD that stabilize a structural conformation suitable for coactivator interaction (Y537S and D538G) (29) and translocations to diverse genes including the coactivator YAP1 and the cell surface protein PCDH11X (FIG. 70B, FIG. 74A) (30). To examine the condensate forming properties of these ER mutants, we generated recombinant ER Y537S, ER D538G, ER-YAP1, and ER-PCDH11X GFP fusion proteins. In contrast to the results with wild type ER, for which incorporation into MED1 droplets was enhanced by estrogen and counteracted by tamoxifen, all four mutant ER proteins formed estrogen-independent, tamoxifen-insensitive condensates with MED1 (FIG. 70C-D, FIG. 74B). The altered phase separation capacity of these mutant ER proteins correlated with their estrogen-independent transactivation potential (FIG. 70E-G) (29, 30). To examine their condensate forming properties in cells, the ER LBD point mutants were tethered to the Lac array in cells (FIG. 69C); normal ER generated a MED1 condensate to the genomic locus only in the presence of estrogen, while the ER mutants formed a MED1 condensate both in the presence and absence of estrogen (FIG. 74C). Taken together, these data demonstrate that acquired genetic alterations found in antiestrogen-resistant patients allow for estrogen-independent condensation of ER and MED1, with consequent gene activation and tumor growth.

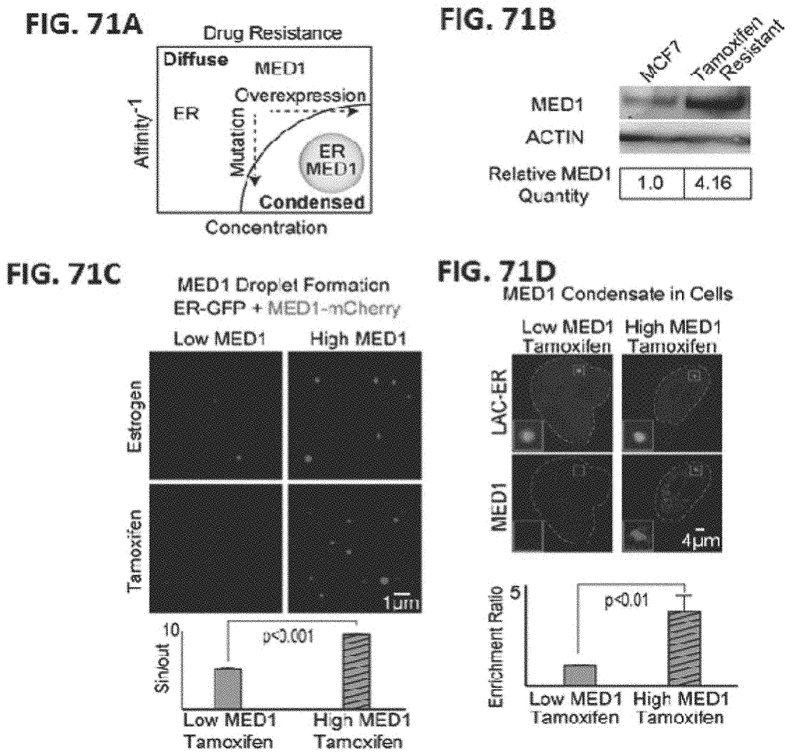

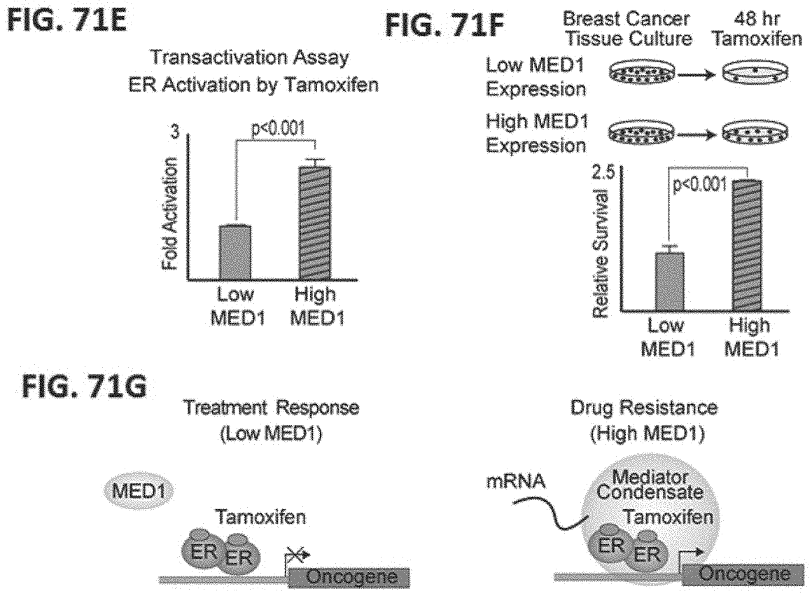

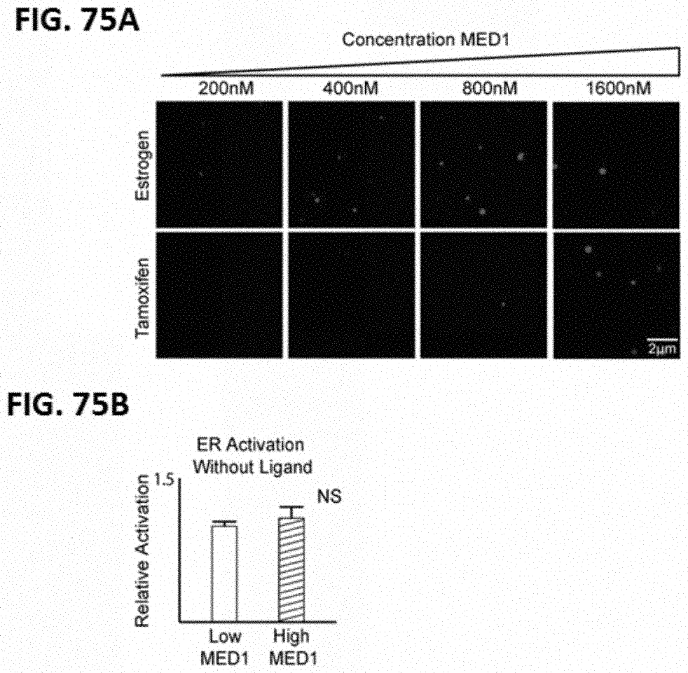

A shift across the phase separation boundary for a TF-Mediator condensate could also occur by altering the concentration of a condensate component, such as MED1 (FIG. 71A) (8, 28). Tamoxifen-bound ER has a reduced affinity for coactivators as compared to estrogen-bound ER (31). However, MED1 overexpression appears to compensate for this reduced affinity; patients with MED1-overexpressing tumors are likely to experience recurrence despite tamoxifen treatment (32). Consistent with this, MCF7 cells selected for tamoxifen resistance overexpress MED1 by greater than 4-fold (FIG. 71B). This led us to hypothesize that in the presence of high MED1 concentrations, tamoxifen-bound ER, despite a lower affinity for coactivators, can form ER-MED1 condensates, activate genes, and effect cancer cell survival. To test the idea that an elevated concentration of MED1 can facilitate condensate formation with tamoxifen-bound ER, we performed in vitro droplet experiments at different concentrations of MED1. At low MED1 concentration, estrogen-bound ER facilitated the formation of MED1 condensates, while tamoxifen-bound ER did not (FIG. 71C, FIG. 75A). However, at higher MED1 concentration, both estrogen-bound and tamoxifen-bound ER allowed for MED1 condensation (FIG. 71C, FIG. 75A). To test whether this also occurs in cells, we altered MED1 levels in cells with the ER LBD tethered to the Lac array. The ER LBD did not generate a MED1 condensate in the presence of tamoxifen with normal MED1 levels (FIG. 71D); in contrast, the tamoxifen-bound ER LBD generated a MED1 condensate when MED1 was overexpressed (FIG. 71D). To examine the functional consequences of MED1 overexpression, a GAL4 transactivation assay was used with tamoxifen-bound ER, which showed activation in the presence of elevated MED1 levels (FIG. 71E and FIG. 75B). To confirm that MED1 overexpression can contribute to drug resistance in breast cancer cells, we generated MCF7 cells overexpressing MED1, which displayed a reduced sensitivity to tamoxifen (FIG. 71F). These data suggest that overexpression of MED1 may mediate antiestrogen resistance by enhancing condensate formation, thereby implicating modulation of protein expression and concentration-dependent phase separation as a mechanism of drug resistance in cancer (FIG. 71G).

Our results suggest that transcriptional condensates compartmentalize and concentrate the transcriptional apparatus to drive oncogene expression in cancer, that these oncogenic condensates can be perturbed by clinically effective drugs, and that the evolution of diverse drug resistance mechanisms can converge on modulation of transcriptional condensate behaviors. These ideas are consistent with prior evidence that tumor cells acquire super-enhancers (SEs) at driver oncogenes (33), that oncogenic SEs can be acquired with only a small change in TF-DNA interaction (34), and that some oncogene SEs are unusually prone to disruption by certain drugs (11). Characteristic features of condensates, including sharp transitions of formation and dissolution, high component concentrations, and the potential for differential partitioning of specific chemistries, may account for these observations. Further advances in our understanding of condensate behaviors and their modulation by small molecule chemistries may thus prove to be beneficial in the setting of cancer.

Materials and Methods

Cell Culture

MCF7 cells (a gift of the Weinberg laboratory), HCT116 cells (ATCC CCL-247), U2OS-268 cells containing a stably integrated array of ˜50,000 Lac-repressor binding sites, hereafter referred to as “U2OS-Lac cells” (a gift of the Spector laboratory), and HEK293T cells (ATCC CRL-3216) were grown in complete DMEM media (DMEM (Life Technologies 11995073), 10% Fetal Bovine Serum, FBS, (Sigma Aldrich, F4135), 1% L-glutamine (GIBCO, 25030-081), 1% Penicillin Streptomycin (Life Technologies, 15140163)). For estrogen deprivation, cells were grown in Estrogen-free DMEM ((Phenol-red free DMEM (Life Technologies, 31053028), Charcoal-stripped Fetal Bovine Serum, FBS, (Sigma-Aldrich F6765), 1% L-glutamine (GIBCO, 25030-081), 1% Penicillin Streptomycin (Life Technologies, 15140163)) for the indicated amount of time.

LN-CAP (ATCC CRL-1740), MM1S (ATCC CRL-2974), and Ramos (ATCC CRL-1596) cells were grown in complete RPMI media (RPMI-1640 (Life Technologies. 61870127), 1% Penicillin Streptomycin (Life Technologies, 15140163), 10% Fetal Bovine Serum, FBS, (Sigma Aldrich, F4135)).

TamR7 (ECACC 16022509) cells were grown in TAMR7 media (Phenol red-free DMEM/F12 (Life Technologies 21041025, 1% L-glutamine (GIBCO, 25030-081)1% Penicillin Streptomycin (Life Technologies, 15140163), 1% Fetal Bovine Serum, FBS, (Sigma Aldrich, F4135), 6 ng/mL insulin (Santa Cruz Biotechnology, sc-360248)).

For passaging, cells were washed in PBS (Life Technologies, AM9625). TrypLE Express Enzyme (Life Technologies, 12604021) was used to detach cells from plates. TrypLE was quenched with complete DMEM.

Tissue Samples

10 uM sections of fresh frozen untreated estrogen receptor positive, progesterone receptor positive, HER2/neu negative, infiltrating ductal carcinoma were provided by BioIVT. H&E staining was performed by the company from which samples were obtained.

Cell Line Generation

CRISPR/Cas9 was used to generate endogenously-mEGFP-tagged MED1 in U2OS-Lac cells. Oligonucleotides coding for 2 guide RNAs targeting the genomic sequence near the N-terminus of the protein were cloned into a px330 vector expressing Cas9 and mCherry (gift from R. Jaenisch). The sequences targeted for MED1 were 5′CCTTCAGGATGAAAGCTCAG 3′ (SEQ ID NO: 253) and 5′CCCCTGAGCTTTCATCCTGA 3′ (SEQ ID NO: 254). A repair template was cloned into a pUC19 vector (NEB) containing mEGFP, a 10 amino acid GS linker and 800 bp homology arms flanking the insert. 500 k cells were transfected with 1.25 μg px330 vector and 1.25 μg repair templates using Lipofectamine 3000. Cells were sorted 2 days after transfection for mCherry. 1 week after first sort, cells were sorted for mEGFP with a single cell per well of a 96-well plate. Cells were expanded and genotyped by PCR and clones with a homozygous knock-in tag were used for experiments.

To generate MCF7 mEGFP-MED1 cells, a lentiviral construct containing the full length MED1 with a N-terminal mEGFP fusion connected by a 10 amino acid GS linker was cloned, containing a puromycin selection marker. Lentiviral particles were generated in HEK293T cells. 250,000 MCF7 cells were plated in one well of a 6 well plate and viral supernatant was added. 48 hours later puromycin was added at 1 ug/mL for 5 days for selection.

Protein Production

cDNA encoding the genes of interest or their IDRs were cloned into a modified version of a T7 pET expression vector. For ER and its variants, the full-length protein was used in all cases. For MED1, an extended IDR containing the LXXLL domains known to interact with ER, comprising amino acids 600-1582 was produced. The base vector was engineered to include a 5′ 6×HIS followed by either mEGFP or mCherry and a 14 amino acid linker sequence “GAPGSAGSAAGGSG.” (SEQ ID NO: 14) NEBuilder® HiFi DNA Assembly Master Mix (NEB E2621S) was used to insert these sequences (generated by PCR) in-frame with the linker amino acids. Vectors expressing mEGFP or mCherry alone contain the linker sequence followed by a STOP codon. Mutant sequences were synthesized as geneblocks (IDT) and inserted into the same base vector as described above. All expression constructs were sequenced to ensure sequence identity.

Protein expression plasmids were transformed into LOBSTR cells (a gift of Chessman laboratory). A fresh bacterial colony was inoculated into LB media containing kanamycin and chloramphenicol and grown overnight at 37° C. Cells containing the MED1-IDR constructs were diluted 1:30 in 500 ml room temperature LB with freshly added kanamycin and chloramphenicol and grown 1.5 hours at 16° C. IPTG was added to 1 mM and growth continued for 20 hours. Cells were collected and stored frozen at −80° C. Cells containing all other constructs were treated in a similar manner except they were grown for 5 hours at 37 C after IPTG induction.

500 ml cell pellets were resuspended in 15 ml of Buffer A (50 mM Tris pH7.5, 500 mM NaCl, 10 mM imidazole, cOmplete protease inhibitors, Roche 11872580001) and sonicated for 10 cycles of 15 seconds on, 60 second off. Lysates were cleared by centrifugation at 12,000 g for 30 minutes at 4° C., added to 1 ml of pre-equilibrated Ni-NTA agarose (Invitrogen R901-15) and rotated at 4° C. for 1.5 hours. The slurry was centrifuged at 3,000 rpm for 10 minutes in a Thermo Legend XTR swinging bucket rotor. The resin pellets were washed 2× with 5 ml of Buffer A followed by centrifugation as above. Protein was eluted 3× with 2 ml of buffer A plus 250 mM imidazole. For each cycle the elution buffer was added and rotated at least 10 minutes at 4 C and centrifuged as above. Elutes were analyzed on a 12% acrylamide gel stained with Coomassie. Fractions containing protein of the expected size were pooled, diluted 1:1 with the 250 mM imidazole buffer and dialyzed against two changes of buffer containing 50 mM Tris 7.5, 125 mM NaCl, 10% glycerol and 1 mM DTT at 4 C. Protein concentration was measured by Thermo BCA Protein Assay Kit—Reducing Agent Compatible.

Immunofluorescence

Human tumor tissues sliced at 10 μm thickness or cells grown on Poly-L-Ornithine coated glass were washed once with PBS and fixed in 4% Paraformaldehyde, PFA, (VWR, BT140770) for 10 minutes. After three washes in PBS for 5 min, cells were stored at 4° C. or transferred to a humidifying chamber and processed for immunofluorescence. Permeabilization of cells was performed using 0.5% triton X100 (Sigma Aldrich, X100) in PBS for 10 minutes followed by three PBS washes. Cells were blocked with 4% IgG-free Bovine Serum Albumin, BSA, (VWR, 102643-516) for 30 minutes and the indicated primary antibody (ER ab32063, MED1 ab64965) was added at a concentration of 1:500 in 4% IgG-free Bovine Serum Albumin for 4-16 hours. If followed by RNA FISH or DNA FISH, primary antibody was diluted in PBS. Cells were washed with PBS three times followed by incubation with secondary antibody (Goat anti-Rabbit IgG Alexa Fluor 488, Life Technologies A11008) at a concentration of 1:500 in PBS for 1 hour.

Following two washes with PBS, the nuclei were stained in 20 μm/mL Hoechst 33258 (Life Technologies, H3569) for 5 minutes. Cells were then washed once in water followed by mounting the coverslip onto glass slides with Vectashield (VWR, 101098-042) and finally sealing the cover slip with nail polish (Electron Microscopy Science Nm, 72180). Images were acquired at the RPI Spinning Disk confocal microscope with 100× objective using MetaMorph acquisition software and a Hammamatsu ORCA-ER CCD camera (W.M. Keck Microscopy Facility, MIT). Images were post-processed using Fiji Is Just ImageJ (The worldwide web at //fiji.sc/).

Immunofluorescence with RNA FISH

Immunofluorescence was performed as described above. After incubating cells with the secondary antibodies, cells were washed three times in PBS for 5 min at RT and fixed with 4% PFA in PBS for 10 min. After two washes of PBS, Wash Buffer A (20% Stellaris RNA FISH Wash Buffer A (Biosearch Technologies, Inc., SMF-WA1-60), 10% Deionized Formamide (EMD Millipore, S4117) in RNase-free water (Life Technologies, AM9932) was added to cells and incubated for 5 minutes. 12.5 μM RNA probe (Custom Stellaris MYC probe Ref #SS4687950104) in Hybridization buffer (90% Stellaris RNA FISH Hybridization Buffer (Biosearch Technologies, SMF-HB1-10) and 10% Deionized Formamide) was added to cells and incubated overnight at 37° C. After washing with Wash Buffer A for 30 minutes at 37° C., the nuclei were stained in 20 μm/mL Hoechst 33258 (Life Technologies, H3569) in PBS for 5 minutes, followed by a 5 minute wash in Wash Buffer B (Biosearch Technologies, SMF-WB1-20). Cells were then washed once in water followed by mounting the coverslip onto glass slides, sealing, imaging, and post-processing as described above.

Immunofluorescence with DNA FISH

MCF7 cells were grown in estrogen-free DMEM for 3 days on Poly-L-ornithine coated coverslips in 24 well plates at an initial seeding density of 50,000 cells per well. Cells were then treated with vehicle, 10 uM estradiol, or 10 uM estradiol and 5 uM 4-hydroxytamoxifen for 45 minutes. Cells on cover slips were then fixed in 4% paraformaldehyde. Immunofluorescence was performed as described above. After incubating the cells with the secondary antibodies, cells were washed three times in PBS for 5 min at RT, fixed with 4% PFA in PBS for 10 min and washed three times in PBS. Cells were incubated in 70% ethanol, 85% ethanol and then 100% ethanol for 1 minute at RT. Probe hybridization mixture was made mixing 7 μL of FISH Hybridization Buffer (Agilent G9400A), 1 μl of FISH probes (SureFISH 8q24.21 MYC 294 kb G101211R-8) and 2 μL of water. 5 μL of mixture was added on a slide and coverslip was placed on top (cell-side toward the hybridization mixture). Coverslip was sealed using rubber cement. Once rubber cement solidified, genomic DNA and probes were denatured at 78° C. for 5 minutes and slides were incubated at 16° C. in the dark O/N. The coverslip was removed from slide and incubated in pre-warmed Wash Buffer 1 (Agilent, G9401A) at 73° C. for 2 minutes and in Wash Buffer 2 (Agilent, G9402A) for 1 minute at RT. Slides were air dried and nuclei were stained in 20 μm/mL Hoechst 33258 (Life Technologies, H3569) in PBS for 5 minutes at RT. Coverslips were washed three times in PBS, followed by mounting the coverslip onto glass slides, sealing, imaging, and post-processing as described above.

RT-qPCR

MCF7 cells were estrogen deprived for 3 days then stimulated with either 10 nM estrogen or 10 nM estrogen and 5 uM 4-hydroxytamoxifen for 24 hours. RNA was isolated by AllPrep Kit (Qiagen 80204) followed by cDNA synthesis using High-Capacity cDNA Reverse Transcription Kit (Applies Biosystems 4368814). qPCR was performed in biological and technical triplicate using Power SYBR Green mix (Life Technologies #4367659) on a QuantStudio 6 System (Life Technologies). The following oligos was used in the qPCR; Myc fwd AACCTCACAACCTTGGCTGA (SEQ ID NO: 255), MYC rev TTCTTTTATGCCCAAAGTCCAA (SEQ ID NO: 256), GAPDH fwd TGCACCACCAACTGCTTAGC (SEQ ID NO: 257), GAPDH rev GGCATGGACTGTGGTCATGAG (SEQ ID NO: 258). Fold change was calculated and MYC expression values were normalized to GAPDH expression.

LAC Binding Assay

Constructs were assembled by NEB HIFI cloning in pSV2 mammalian expression vector containing an SV40 promoter driving expression of a CFP-LacI fusion protein. The activation domains and mutant activation domains of ESR1 were fused by the c-terminus to this recombinant protein, joined by the linker sequence GAPGSAGSAAGGSG (SEQ ID NO: 14). For some experiments a variant plasmid with mCherry in place of CFP was used. U20S-Lac cells were estrogen deprived for 24 hours. Cells were then plated on fibronectin-coated glass coverslips and transfected using lipofectamine 3000 (Thermofisher L3000015). For high MED1 conditions, a construct with a mammalian expression vector containing a PGK promoter driving the expression of MED1 fused to GFP was co-transfected. 24 hours after transfection, cells were treated for 45 minutes with either DMSO, 10 nM of B-Estradiol (Sigma-Aldrich E8875) reconstituted in DMSO or 1 uM of 4-Hydroxytamoxifen (Sigma-Aldrich H7904) reconstituted in DMSO. Following treatment, cells were fixed and immunofluorescence was performed with a MED1 antibody as described above.

Lac Array Image Analysis

For analysis of Lac array data, custom Python scripts were written to process and analyze image data gathered in Lac and tagged-protein channels. Nuclear stains were blurred with a Gaussian filter (sigma=2.0) and clustered into 2 clusters (nuclei and background) by K-means. The nuclei were then labeled with the python scikit-image package using the measure.label function. To segment Lac spots, the Lac image channel was blurred with a Gaussian filter (sigma=2.0), and an intensity threshold (mean+1.5*std) was applied to the image. Segmented regions (also determined by measure.label) were then filtered based on minimum area (150 pixels), maximum area (2000 pixels), circularity (c=4pi*area/perimeter\hat{ }2; 0.8), and presence in a nucleus as defined by the mask described above. A norm enrichment ratio was calculated by determining the mean intensity of the tagged-protein in the segmented Lac spot and dividing it by the mean intensity of the tagged-protein present in the same whole nucleus.

Live Cell Imaging

For live-cell treatments of U20S-Lac cells, those with endogenously tagged GFP-MED1 were estrogen starved for 24 hours then plated onto poly-L-ornithine-coated (Sigma-Aldrich A-004) dishes and transfected with a plasmid with an mCherry-LacI-ESR1 fusion. 24 hours later, cells were treated with 10 nM B-Estradiol for 45 minutes. Cells were imaged pre-treatment and 30 minutes after treatment with a 1:1000 dilution of DMSO or 10 uM 4-Hydroxytamoxifen in Estrogen-free DMEM. Quantification was performed in FIJI; the instrument background was subtracted from the average signal intensity in the array, then divided by the instrument background subtracted from an average nuclear signal to yield the normalized signal intensity. The normalized signal intensity at 30 minutes was divided by that at time 0 to yield the relative intensity in either tamoxifen or vehicle treated specimens.

For live-cell FRAP experiments, the endogenously tagged U20S-Lac cells or MED1-mEGFP MCF7 cells were plated on Poly-L-Ornithine coated glass-bottom tissue culture plate. U20S-Lac cells were subjected to B-Estradiol treatment as described above. 20 pulses of laser at a 50 us dwell time were applied to the array, and recovery was imaged on an Andor microscope every 1 s for the indicated time periods. Quantification was performed in FIJI.

For the MCF7 MED1-mEGFP FRAP, the instrument background was subtracted from the average signal intensity in the bleached puncta then divided by the instrument background subtracted from a control puncta. For the U20S-Lac MED1-mEGFP FRAP, the instrument background was subtracted from the average signal intensity in the bleached portion of the MED1 signal at the lac array then divided by the instrument background subtracted from a control area in the nucleus. These values were plotted every second, and a best fit line with 95% confidence intervals was calculated.

In Vitro Droplet Assays and Quantification

Recombinant GFP or mCherry fusion proteins were concentrated and desalted to an appropriate protein concentration and 125 mM NaCl using Amicon Ultra centrifugal filters (30K MWCO, Millipore). Recombinant proteins were added to solutions at varying concentrations with indicated final salt and 10% PEG-8000 as crowding agent in Droplet Formation Buffer (50 mM Tris-HCl pH 7.5, 10% glycerol, 1 mM DTT). The protein solution was immediately loaded onto a homemade chamber comprising a glass slide with a coverslip attached by two parallel strips of double-sided tape. Slides were then imaged with an Andor confocal microscope with a 150× objective. Unless indicated, images presented are of droplets settled on the glass coverslip. B-Estradiol (E8875 Sigma) or 4-Hydroxytamoxifen (Sigma-Aldrich H7904) was reconstituted to 10 mM in 100% EtOH then diluted in 125 mM NaCl droplet formation buffer to 1 mM. One microliter of this concentrated stock was used in a 10 uL droplet formation reaction to achieve a final concentration of 100 uM. To calculate enrichment for the in vitro droplet assay, droplets were defined as a region of interest in FIJI by the MED1 scaffold channel, and the maximum signal of the ER client within that droplet was determined. Alternatively, maximum signal of MED1 was measured. In all cases, the maximum signal was divided by the background client signal in the image to generate a Cin/out.

Gal4 Transcription Assay

Transcription factor constructs were assembled in a mammalian expression vector containing an SV40 promoter driving expression of a GAL4 DNA-binding domain. Wild type and mutant activation domains of ESR1 were fused to the C-terminus of the DNA-binding domain by Gibson cloning (NEB 2621S), joined by the linker GAPGSAGSAAGGSG (SEQ ID NO: 14). HEK293T cells (ATCC CRL-3216) were estrogen deprived for 24 hours then plated on white flat-bottom 96-well assay plates (Costar 3917). The transcription factor constructs were transfected 24 hours later using Lipofectamine 3000 (Thermofisher L3000015). These constructs were co-transfected with a modified version of the PGL3-Basic (Promega) vector containing five GAL4 upstream activation sites upstream of the firefly luciferase gene. Also co-transfected was pRL-SV40 (Promega), a plasmid containing the Renilla luciferase gene driven by an SV40 promoter. For high MED1 conditions, a construct with a mammalian expression vector containing a PGK promoter driving the expression of MED1 fused to GFP was co-transfected. Upon transfection, cells were treated with 1:1000 dilution of DMSO, 10 nM B-estradiol, or 1 uM tamoxifen as indicated. For MED1 overexpression experiments, cells were treated with 10 nM Tamoxifen. 24 hours after transfection, luminescence generated by each luciferase protein was measured using the Dual-glo Luciferase Assay System (Promega E2920). The data as presented has been controlled for Renilla luciferase expression and normalized to the ER-LBD estrogen deprived condition.

High-Throughput Sequencing Data Sets and Visualization

MED1 and ESR1 ChIP-Seq from estrogen stimulated MCF cells (GEO accession number GSE60270) and MCF7 CTCF ChIA-PET (GEO accession number GSE92881) were obtained from public sources and visualized on the UCSC browser (https://genome.ucsc.edu/cgi-bin/hgGateway).

Cbioportal Data Acquisition

For frequency of patient mutations, cbioportal (http://www.cbioportal.org/) was queried for mutations in ESR1 that are present in any breast cancer sequencing data set.

Western Blot

Cells were lysed in Cell Lytic M (Sigma-Aldrich C2978) with protease inhibitors (Roche, 11697498001). Lysate was run on a 3%-8% Tris-acetate gel or 10% Bis-Tris gel or 3-8% Bis-Tris gels at 80 V for ˜2 hrs, followed by 120 V until dye front reached the end of the gel. Protein was then wet transferred to a 0.45 μm PVDF membrane (Millipore, IPVH00010) in ice-cold transfer buffer (25 mM Tris, 192 mM glycine, 10% methanol) at 300 mA for 2 hours at 4° C. After transfer the membrane was blocked with 5% non-fat milk in TBS for 1 hour at room temperature, shaking. Membrane was then incubated with 1:1,000 of the indicated antibody (ER ab32063, MED1 ab64965) diluted in 5% non-fat milk in TBST and incubated overnight at 4° C., with shaking. In the morning, the membrane was washed three times with TBST for 5 minutes at room temperature shaking for each wash. Membrane was incubated with 1:5,000 secondary antibodies for 1 hr at RT and washed three times in TBST for 5 minutes. Membranes were developed with ECL substrate (Thermo Scientific, 34080) and imaged using a CCD camera or exposed using film or with high sensitivity ECL. Quantification of western blot was performed using BioRad image lab.

MCF7 Survival Assay

MCF7 cells were transfected with PiggyBac transposase and PiggyBac integration vector containing MED1-mApple and grown in the presence of 2 ug/ml of doxycycline. After 5 days, cells were sorted for those expressing high levels of mApple. Parental MCF7 or MCF7 cells expressing MED1-mApple were then seeded at 50,000 cells per well in a 24 well plate in complete DMEM. 1 day later the medium was changed to that containing either vehicle (DMSO) or 25 uM 4-hydroxytamoxifen. After 48 hours wells were assayed by Cell Titer-Glo to quantify the amount of ATP in a white-bottom 96 well plate in a Tecan plate reader. Percent survival was calculated as the luciferase signal in the treated well divided by the signal in the vehicle treated well, data are presented as percent survival in treated divided by percent survival in vehicle to yield relative survival.

FISH-IF Average Image Analysis

For analysis of RNA/DNA FISH with immunofluorescence, custom Python scripts were written to process and analyze 3D image data gathered in FISH and IF channels. Nuclear stains were blurred with a Gaussian filter (sigma=2.0), maximally projected in the z plane, and clustered into 2 clusters (nuclei and background) by K-means. FISH foci were either manually called with ImageJ or automatically called using the scipy ndimage package. For automatic detection, an intensity threshold (mean+3*standard deviation) was applied to the FISH channel. The ndimage find_objects function was then used to call contiguous FISH foci in 3D. These FISH foci were filtered by various criteria, including size (minimum 100 voxels), circularity of a max z-projection (circularity=4pi*area/perimeter\hat{ }2; 0.7), and being present in a nucleus (determined by nuclear mask described above). For manual calling, FISH foci were identified in maximum z-projections of the FISH channel, and the x and y coordinates were used as reference points to guide the automatic detection described above. The FISH foci were then centered in a 3D-box (length size l=3.0 μm). The IF signal centered at FISH foci for each FISH and IF pair are then combined and an average intensity projection is calculated, providing averaged data for IF signal intensity within a l×l square centered at FISH foci. As a control, this same process was carried out for IF signal centered at an equal number of randomly selected nuclear positions. These average intensity projections were then used to generate 2D contour maps of the signal intensity. Contour plots are generated using the matplotlib python package. For the contour plots, the intensity-color ranges presented were customized across a linear range of colors (n!=15). For the FISH channel, black to magenta was used. For the IF channel, we used chroma.js (an online color generator) to generate colors across 15 bins, with the key transition colors chosen as black, blueviolet, medium-blue, lime. This was done to ensure that the reader's eye could more readily detect the contrast in signal. The generated colormap was employed to 15 evenly spaced intensity bins for all IF plots. The averaged IF centered at FISH or at randomly selected nuclear locations are plotted using the same color scale, set to include the minimum and maximum signal from each plot.

### REFERENCES

1. S. Alberti, The wisdom of crowds: regulating cell function through condensed states of living matter. J Cell Sci 130, 2789-2796 (2017).
2. S. F. Banani, H. O. Lee, A. A. Hyman, M. K. Rosen, Biomolecular condensates: organizers of cellular biochemistry. Nat Rev Mol Cell Biol 18, 285-298 (2017).
3. A. A. Hyman, C. A. Weber, F. Julicher, Liquid-liquid phase separation in biology. Annu Rev Cell Dev Biol 30, 39-58 (2014).
4. Y. Shin, C. P. Brangwynne, Liquid phase condensation in cell physiology and disease. Science 357, (2017).
5. R. J. Wheeler, A. A. Hyman, Controlling compartmentalization by non-membrane-bound organelles. Philos Trans R Soc Lond B Biol Sci 373, (2018).
6. A. Boija et al., Transcription Factors Activate Genes through the Phase-Separation Capacity of Their Activation Domains. Cell 175, 1842-1855 e1816 (2018).
7. D. Hnisz, K. Shrinivas, R. A. Young, A. K. Chakraborty, P. A. Sharp, A Phase Separation Model for Transcriptional Control. Cell 169, 13-23 (2017).
8. B. R. Sabari et al., Coactivator condensation at super-enhancers links phase separation and gene control. Science 361, (2018).
9. W. K. Cho et al., Mediator and RNA polymerase II clusters associate in transcription-dependent condensates. Science 361, 412-415 (2018).
10. L. M. Tuttle et al., Gcn4-Mediator Specificity Is Mediated by a Large and Dynamic Fuzzy Protein-Protein Complex. Cell Rep 22, 3251-3264 (2018).
11. J. E. Bradner, D. Hnisz, R. A. Young, Transcriptional Addiction in Cancer. Cell 168, 629-643 (2017).
12. J. J. Bouchard et al., Cancer Mutations of the Tumor Suppressor SPOP Disrupt the Formation of Active, Phase-Separated Compartments. Mol Cell 72, 19-36 e18 (2018).
13. G. Boulay et al., Cancer-Specific Retargeting of BAF Complexes by a Prion-like Domain. Cell 171, 163-178 e119 (2017).
14. J. S. Roe et al., Enhancer Reprogramming Promotes Pancreatic Cancer Metastasis. Cell 170, 875-888 e820 (2017).
15. S. Rahman et al., Activation of the LMO2 oncogene through a somatically acquired neomorphic promoter in T-cell acute lymphoblastic leukemia. Blood 129, 3221-3226 (2017).
16. Y. Wang et al., CDK7-dependent transcriptional addiction in triple-negative breast cancer. Cell 163, 174-186 (2015).
17. A. G. Waks, E. P. Winer, Breast Cancer Treatment: A Review. JAMA 321, 288-300 (2019).
18. Y. K. Kang, M. Guermah, C. X. Yuan, R. G. Roeder, The TRAP/Mediator coactivator complex interacts directly with estrogen receptors alpha and beta through the TRAP220 subunit and directly enhances estrogen receptor function in vitro. Proc Natl Acad Sci USA 99, 2642-2647 (2002).
19. D. Dubik, T. C. Dembinski, R. P. Shiu, Stimulation of c-myc oncogene expression associated with estrogen-induced proliferation of human breast cancer cells. Cancer Res 47, 6517-6521 (1987).
20. Y. Shang, X. Hu, J. DiRenzo, M. A. Lazar, M. Brown, Cofactor dynamics and sufficiency in estrogen receptor-regulated transcription. Cell 103, 843-852 (2000).
21. C. E. Nesbit, J. M. Tersak, E. V. Prochownik, MYC oncogenes and human neoplastic disease. Oncogene 18, 3004-3016 (1999).
22. T. Borggrefe, X. Yue, Interactions between subunits of the Mediator complex with gene-specific transcription factors. Semin Cell Dev Biol 22, 759-768 (2011).
23. J. S. Carroll et al., Genome-wide analysis of estrogen receptor binding sites. Nat Genet 38, 1289-1297 (2006).
24. A. K. Shiau et al., The structural basis of estrogen receptor/coactivator recognition and the antagonism of this interaction by tamoxifen. Cell 95, 927-937 (1998).
25. S. M. Janicki et al., From silencing to gene expression: real-time analysis in single cells. Cell 116, 683-698 (2004).
26. S. Chong et al., Imaging dynamic and selective low-complexity domain interactions that control gene transcription. Science 361, (2018).
27. C. K. Osborne, R. Schiff, Mechanisms of endocrine resistance in breast cancer. Annu Rev Med 62, 233-247 (2011).
28. S. F. Banani et al., Compositional Control of Phase-Separated Cellular Bodies. Cell 166, 651-663 (2016).
29. S. W. Fanning et al., Estrogen receptor alpha somatic mutations Y537S and D538G confer breast cancer endocrine resistance by stabilizing the activating function-2 binding conformation. Elife 5, (2016).
30. J. T. Lei et al., Functional Annotation of ESR1 Gene Fusions in Estrogen Receptor-Positive Breast Cancer. Cell Rep 24, 1434-1444 e1437 (2018).
31. M. S. Ozers et al., Analysis of ligand-dependent recruitment of coactivator peptides to estrogen receptor using fluorescence polarization. Mol Endocrinol 19, 25-34 (2005).
32. A. Nagalingam et al., Med1 plays a critical role in the development of tamoxifen resistance. Carcinogenesis 33, 918-930 (2012).
33. D. Hnisz et al., Super-enhancers in the control of cell identity and disease. Cell 155, 934-947 (2013).
34. M. R. Mansour et al., Oncogene regulation. An oncogenic super-enhancer formed through somatic mutation of a noncoding intergenic element. Science 346, 1373-1377 (2014).

1-352. (canceled)

353. A method of identifying a test agent that modulates one or more of a condensate physical property, composition, regulation, dissolution, formation, stability, activity, and morphology, the method comprising:
(a) providing a composition comprising a condensate or a component thereof,
(b) contacting the composition with a test agent, and
(c) assessing whether the test agent causes a change in the one or more of condensate physical property, composition, regulation, dissolution, formation, stability, activity, and morphology as compared to in the absence of the test agent.

354. The method of claim 353, wherein the physical property is one or more of size, concentration, permeability, morphology, and viscosity.

355. The method of claim 353, wherein the condensate component is selected from the group consisting of a transcription factor, a co-activator, a signaling factor, a methyl-DNA binding protein, a splicing factor, a gene silencing factor, an RNA polymerase, a nuclear receptor, and a fragment thereof comprising an intrinsically disordered region (IDR).

356. The method of claim 353, wherein the condensate component has a mutation.

357. The method of claim 356, wherein the mutation is associated with or characterizes a disease or condition.

358. The method of claim 353, wherein the condensate comprises, or the condensate component is, a signaling factor or a fragment thereof comprising an IDR necessary for transcription activation of a gene.

359. The method of claim 358, wherein the signaling factor is β-catenin or STAT3.

360. The method of claim 353, wherein the condensate comprises, or the condensate component is, a methyl-DNA binding protein or a fragment thereof comprising a C-terminal IDR, or a suppressor or a fragment thereof comprising an IDR.

361. The method of claim 360, wherein the methyl-DNA binding protein is MeCP2.

362. The method of claim 353, wherein the condensate comprises, or the condensate component is, a transcription factor or a fragment thereof comprising an IDR.

363. The method of claim 362, wherein the transcription factor is MYC.

364. The method of claim 353, wherein the test agent alters or disrupts an interaction between the condensate component and a second component of the condensate.

365. The method of claim 353, wherein assessing whether the test agent modulates the one or more of condensate physical property, composition, regulation, dissolution, formation, stability, activity, and morphology comprises assessing whether the condensate component becomes sequestered in a second condensate after contacting with the test agent.

366. The method of claim 353, wherein assessing whether the test agent modulates the one or more of condensate physical property, composition, regulation, dissolution, formation, stability, activity, and morphology comprises assessing whether the condensate sequesters a condensate component associated with a disease.

367. The method of claim 366, wherein the condensate component associated with the disease is a mutant version of a wild-type protein, or a protein over-expressed in the disease.

368. The method of claim 367, wherein the wild-type protein is not sequestered in the condensate.

369. The method of claim 353, wherein the condensate or component thereof has a detectable tag, and wherein the detectable tag is used to assess whether contacting with the test agent modulates the one or more of condensate physical property, composition, regulation, dissolution, formation, stability, activity, and morphology.

370. The method of claim 369, wherein the detectable tag is a fluorescent tag.

371. The method of claim 353, wherein an antibody specifically recognizing the condensate or component thereof is used to assess whether contacting with the test agent modulates the one or more of condensate physical property, composition, regulation, dissolution, formation, stability, activity, and morphology.

372. The method of claim 353, wherein assessing whether the test agent modulates the one or more of condensate physical property, composition, regulation, dissolution, formation, stability, activity, and morphology comprises using microscopy.

373. The method of claim 366, wherein sequestration of the condensate component associated with the disease changes the normal location of the condensate component associated with the disease.

374. The method of claim 366, wherein sequestration of the condensate component associated with the disease in the condensate modulates a second condensate by restricting access of the condensate component associated with the disease to the second condensate.

375. The method of claim 367, further comprising assessing whether the wild-type version of the condensate component associated with the disease is sequestered in the condensate.

376. The method of claim 357, wherein the disease or condition is cancer.

377. The method of claim 353, wherein the composition comprises a cell comprising the condensate or component thereof.

378. The method of claim 353, further comprising assessing the one or more of condensate physical property, composition, regulation, dissolution, formation, stability, activity, and morphology in the absence of the test agent.

379. A method of modulating transcription of one or more genes, comprising modulating formation, composition, maintenance, dissolution, activity, stability, physical property, and/or regulation of a condensate associated with the one or more genes,
wherein the condensate is modulated by sequestration of one or more components of the condensate in a second condensate; and
wherein the condensate is a transcriptional condensate, a heterochromatin condensate, or a condensate physically associated with an mRNA initiation or elongation complex.

380. The method of claim 379, wherein the sequestered component is a mutant version of a wild-type protein.

381. The method of claim 380, wherein the wild-type protein is not sequestered in the second condensate.

382. The method of claim 379, wherein the sequestered component is a phosphorylated component or a de-phosphorylated component.

383. The method of claim 379, wherein the sequestered component is a component over-expressed in a disease state.
